(19) United States
(12) Patent Application Publication
(10) Pub. No.: US 2024/0023429 A1
(43) Pub. Date: Jan. 18, 2024

(54) MONOAMINE COMPOUND AND LIGHT EMITTING ELEMENT INCLUDING THE SAME

(71) Applicant: Samsung Display Co., LTD., Yongin-si (KR)

(72) Inventors: Ichinori TAKADA, Yokohama (JP); Akinori YAMATANI, Yokohama (JP); Ichiro IMADA, Yokohama (JP); Hiroaki ITOI, Yokohama (JP)

(21) Appl. No.: 18/162,471

(22) Filed: Jan. 31, 2023

Related U.S. Application Data
(63) Continuation-in-part of application No. 17/143,554, filed on Jan. 7, 2021, now Pat. No. 11,575,086.
Application No. 17/143,554 is a continuation of application No. 15/789,274, filed on Oct. 20, 2017, now Pat. No. 10,923,663.

(30) Foreign Application Priority Data
Oct. 21, 2016 (KR) 10-2016-0137914
May 26, 2017 (KR) 10-2017-0065359
Sep. 8, 2017 (KR) 10-2017-0114951

(51) Int. Cl. H10K 85/60; C07F 7/08; H10K 50/30; H10K 85/40; C07C 211/54; C07C 211/56; C07C 211/58; C07C 211/61; C07C 255/58; C07D 213/38; C07D 307/91; C07D 333/50; C07D 333/76

(52) U.S. Cl. CPC H10K 85/633; H10K 50/11; H10K 50/30; H10K 85/40; H10K 85/636; C07C 211/54; C07C 211/56; C07C 211/58; C07C 211/61; C07C 255/58; C07D 213/38; C07D 307/91; C07D 333/50; C07D 333/76; C07F 7/081

(57) ABSTRACT

Provided are a monoamine compound and a light emitting element including the same. The monoamine compound according to an example embodiment is represented by the following Formula 1

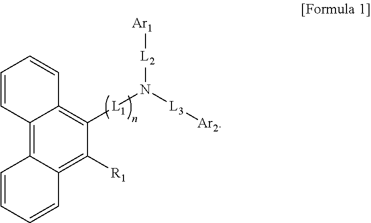

### CROSS-REFERENCE TO RELATED APPLICATIONS

This application is a continuation-in-part of U.S. patent application Ser. No. 17/143,554, filed Jan. 7, 2021, which is a continuation of U.S. patent application Ser. No. 1/789,274, filed on Oct. 20, 2017, now U.S. Pat. No. 10,923,663, which claims priority to Korean Patent Application Nos. 10-2016-0137914, filed on Oct. 21, 2016, 10-2017-0065359, filed on May 26, 2017, and 10-2017-0114951, filed on Sep. 8, 2017, which are each incorporated by reference herein in their entirety.

### BACKGROUND

### 1. Field

Embodiments relate to a compound and a light emitting element including the same.

### 2. Description of the Related Art

An organic electroluminescence display is a so called self-luminescent display. In the organic electroluminescence display, recombination of holes and electrons injected from a first electrode and a second electrode in an emission layer may create emitted light. A luminescent material, for example, an organic compound, may be in the emission layer.

### SUMMARY

Embodiments are directed to a monoamine compound represented by the following Formula 1:

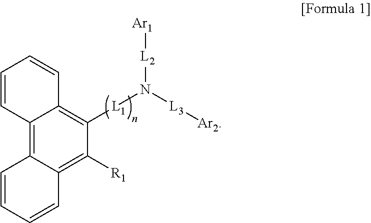

In Formula 1, L₁ may be a substituted or unsubstituted arylene group having 6 to 30 carbon atoms for forming a ring, or a substituted or unsubstituted heteroarylene group having 2 to 30 carbon atoms for forming a ring, n may be 1 or 2, L₂ and L₃ may each independently be a direct linkage, a substituted or unsubstituted arylene group having 6 to 30 carbon atoms for forming a ring, or a substituted or unsubstituted heteroarylene group having 2 to 30 carbon atoms for forming a ring, R₁ may be a substituted or unsubstituted alkyl group having 1 to 10 carbon atoms, a substituted or unsubstituted aryl group having 6 to 30 carbon atoms for forming a ring, a substituted or unsubstituted heteroaryl group having 2 to 30 carbon atoms for forming a ring, or a substituted or unsubstituted aryl silyl group, and Ar₁ and Ar₂ may each independently be a substituted or unsubstituted aryl group having 6 to 30 carbon atoms for forming a ring, or a substituted or unsubstituted heteroaryl group having 2 to 30 carbon atoms for forming a ring.

In an embodiment, the monoamine compound represented by Formula 1 may be represented by the following Formula 2-1 or 2-2:

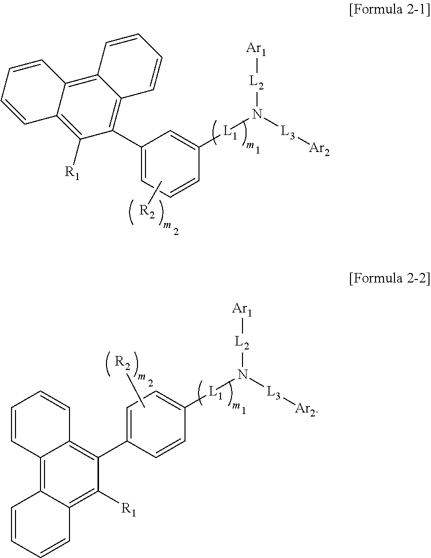

In Formulae 2-1 and 2-2, m₁ may be 0 or 1, m₂ may be an integer of 0 to 2, R₂ may be a hydrogen atom, a deuterium atom, a substituted or unsubstituted alkyl group having 1 to 10 carbon atoms, a substituted or unsubstituted aryl group having 6 to 30 carbon atoms for forming a ring, a substituted or unsubstituted heteroaryl group having 2 to 30 carbon atoms for forming a ring, or a substituted or unsubstituted aryl silyl group, or may be combined with an adjacent group to form a ring, and Ar₁, Ar₂, L₁, L₂, L₃, and R₁ are the same as described above.

In an embodiment, the monoamine compound represented by Formula 2-1 may be represented by one of the following Formulae 2-1-1 to 2-1-3:

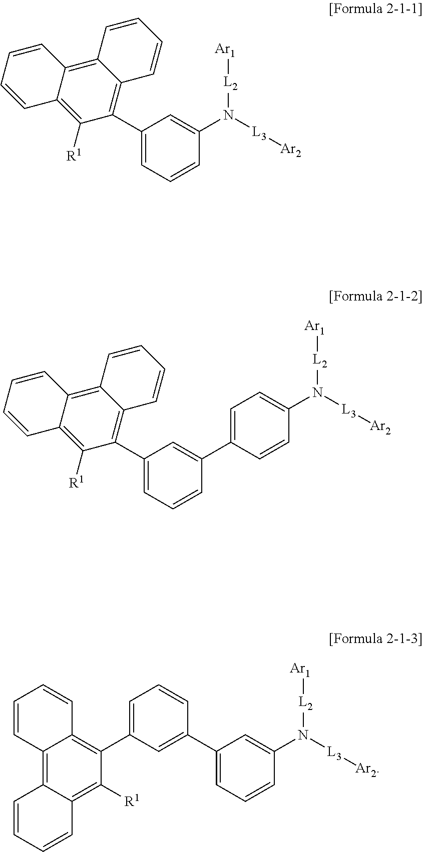

In Formulae 2-1-1 to 2-1-3, Ar₁ and Ar₂, L₂ and L₃, and R₁ are the same as described above.

In an embodiment, the monoamine compound represented by Formula 2-2 may be represented by one of the following Formulae 2-2-1 to 2-2-3.

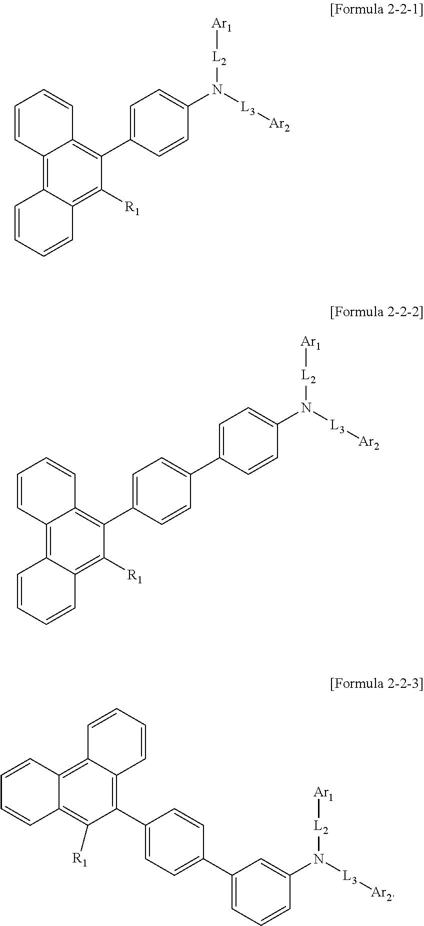

In Formulae 2-2-1 to 2-2-3, Ar₁ and Ar₂, L₂ and L₃ and R₁ are the same as described above.

R₁ may be a substituted or unsubstituted phenyl group, L₃ may be a substituted or unsubstituted phenylene group, and Ar₂ may be a substituted or unsubstituted naphthyl group.

L₂ may be a substituted or unsubstituted phenylene group, and Ar₁ may be a substituted or unsubstituted phenyl group.

L₂ may be a direct linkage, and Ar₁ may be a substituted or unsubstituted dibenzofuranyl group.

In an embodiment, L₁ may be a substituted or unsubstituted phenylene group, a substituted or unsubstituted divalent biphenyl group, or a substituted or unsubstituted naphthylene group.

In an embodiment, R₁ may be a substituted or unsubstituted phenyl group, a substituted or unsubstituted naphthyl group, a substituted or unsubstituted biphenyl group, or a substituted or unsubstituted nitrogen-containing heteroaryl group.

In an embodiment, Ar₁ and Ar₂ may each independently be a substituted or unsubstituted phenyl group, a substituted or unsubstituted biphenyl group, a substituted or unsubstituted terphenyl group, a substituted or unsubstituted naphthyl group, a substituted or unsubstituted phenanthryl group, or a substituted or unsubstituted fluorenyl group.

In an embodiment, Ar₁ and Ar₂ may each independently be a substituted or unsubstituted dibenzofuranyl group, a substituted or unsubstituted dibenzothiophenyl group, a substituted or unsubstituted benzonaphthofuranyl group, or a substituted or unsubstituted benzonaphthothiophenyl group.

In an embodiment, Ar₁ and Ar₂ may each independently be represented by the following Formula 3:

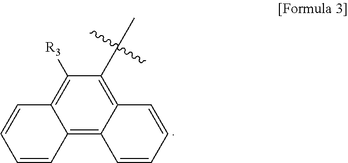

in case Ar₁ and Ar₂ are each independently represented by Formula 3, in formula 1, L₂ and L₃ may each independently be a substituted or unsubstituted arylene group having 6 to 30 carbon atoms for forming a ring, or a substituted or unsubstituted heteroarylene group having 2 to 30 carbon atoms for forming a ring In formula 3, R₃ is a substituted or unsubstituted alkyl group having 1 to 10 carbon atoms, a substituted or unsubstituted aryl group having 6 to 30 carbon atoms for forming a ring, a substituted or unsubstituted heteroaryl group having 2 to 30 carbon atoms for forming a ring, or a substituted or unsubstituted aryl silyl group.

In an embodiment, Ar₁ and Ar₂ may each independently be represented by the following Formula 4:

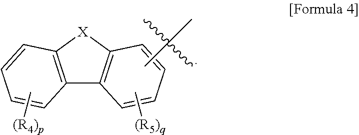

In Formula 4, X is O or S, R₄ and R₅ may each independently be a hydrogen atom, a deuterium atom, a substituted or unsubstituted alkyl group having 1 to 10 carbon atoms, a substituted or unsubstituted aryl group having 6 to 30 carbon atoms for forming a ring, a substituted or unsubstituted heteroaryl group having 2 to 30 carbon atoms for forming a ring, or a substituted or unsubstituted aryl silyl group, p may be an integer of 0 to 4, and q may be an integer of 0 to 3.

In an embodiment, L₂ and L₃ may each independently be a direct linkage, a substituted or unsubstituted phenylene group, a substituted or unsubstituted divalent biphenyl group, or a substituted or unsubstituted naphthylene group.

In an example embodiment, an organic electroluminescence device includes a first electrode, a hole transport region provided on the first electrode, an emission layer provided on the hole transport region, an electron transport region provided on the emission layer, and a second electrode provided on the electron transport region.

At least one of the hole transport region, the emission region, and the electron transport region includes a monoamine compound represented by the following Formula 1:

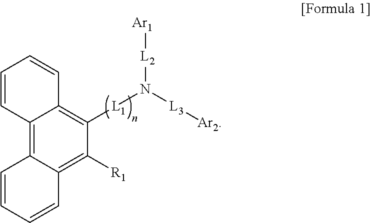

In Formula 1, L₁ may be a substituted or unsubstituted arylene group having 6 to 30 carbon atoms for forming a ring, or a substituted or unsubstituted heteroarylene group having 2 to 30 carbon atoms for forming a ring, n may be 1 or 2, L₂ and L₃ may each independently be a direct linkage, a substituted or unsubstituted arylene group having 6 to 30 carbon atoms for forming a ring, or a substituted or unsubstituted heteroarylene group having 2 to 30 carbon atoms for forming a ring, R₁ may be a substituted or unsubstituted alkyl group having 1 to 10 carbon atoms, a substituted or unsubstituted aryl group having 6 to 30 carbon atoms for forming a ring, a substituted or unsubstituted heteroaryl group having 2 to 30 carbon atoms for forming a ring, or a substituted or unsubstituted aryl silyl group, and Ar₁ and Ar₂ may each independently be a substituted or unsubstituted aryl group having 6 to 30 carbon atoms for forming a ring, or a substituted or unsubstituted heteroaryl group having 2 to 30 carbon atoms for forming a ring.

In an embodiment, the hole transport region may include the monoamine compound represented by Formula 1.

In an embodiment, the hole transport region may include a hole injection layer disposed on the first electrode and a hole transport layer disposed on the hole injection layer, and the hole transport layer may include the monoamine compound represented by Formula 1.

In an embodiment, the hole transport layer may make contact with the emission layer.

In an embodiment, the hole transport region may include a hole injection layer disposed on the first electrode, a first hole transport layer disposed on the hole injection layer, and a second hole transport layer disposed on the first hole transport layer and adjacent to the emission layer, wherein the second hole transport layer includes the monoamine compound represented by Formula 1.

### BRIEF DESCRIPTION OF THE FIGURES

Features will become apparent to those of skill in the art by describing in detail example embodiments with reference to the attached drawings in which:

FIG. 1 is a plan view of a display apparatus according to an embodiment of the inventive concept;

FIG. 2 is a cross-sectional view of a display apparatus according to an embodiment of the inventive concept;

FIG. 3 is a cross-sectional view schematically showing a light emitting device according to an embodiment of the inventive concept;

FIG. 4 is a cross-sectional view schematically showing a light emitting device according to an embodiment of the inventive concept;

FIG. 5 is a cross-sectional view schematically showing a light emitting device according to an embodiment of the inventive concept;

FIG. 6 is a cross-sectional view schematically showing a light emitting device according to an embodiment of the inventive concept;

FIG. 7 and FIG. 8 are cross-sectional views of display apparatuses according to embodiments of the inventive concept;

FIG. 9 is a cross-sectional view showing a display apparatus according to an embodiment of the inventive concept;

FIG. 10 is a cross-sectional view showing a display apparatus according to an embodiment of the inventive concept; and

FIG. 11 is a perspective view schematically showing an electronic apparatus including a display apparatus according to an embodiment of the inventive concept.

### DETAILED DESCRIPTION

The inventive concept may have various modifications and may be embodied in different forms, and example embodiments will be explained in detail with reference to the accompany drawings. The inventive concept may, however, be embodied in different forms and should not be construed as limited to the embodiments set forth herein. Rather, all modifications, equivalents, and substituents which are included in the spirit and technical scope of the inventive concept should be included in the inventive concept.

Like reference numerals refer to like elements throughout. In the drawings, the dimensions of structures are exaggerated for clarity of illustration. It will be understood that, although the terms first, second, etc. may be used herein to describe various elements, these elements should not be limited by these terms. These terms are only used to distinguish one element from another element. Thus, a first element could be termed a second element without departing from the teachings of the present invention. Similarly, a second element could be termed a first element. As used herein, the singular forms are intended to include the plural forms as well, unless the context clearly indicates otherwise.

In the description, it will be further understood that the terms “comprises” and/or “comprising,” when used in this specification, specify the presence of stated features, numerals, steps, operations, elements, parts, or the combination thereof, but do not preclude the presence or addition of one or more other features, numerals, steps, operations, elements, parts, or the combination thereof.

In the description, when a layer, a film, a region, a plate, etc. is referred to as being “on” or “above” another part, it can be “directly on” the other part, or intervening layers may also be present. On the contrary, when a layer, a film, a region, a plate, etc. is referred to as being “under” or “below” another part, it can be “directly under” the other part, or intervening layers may also be present. Also, when an element is referred to as being disposed “on” another element, it can be disposed under the other element.

In the description, the term “substituted or unsubstituted” corresponds to substituted or unsubstituted with at least one substituent selected from the group consisting of a deuterium atom, a halogen atom, a cyano group, a nitro group, an amine group, a silyl group, an oxy group, a thiol group, a sulfinyl group, a sulfonyl group, a carbonyl group, a boron group, a phosphine oxide group, a phosphine sulfide group, an alkyl group, an alkenyl group, an alkynyl group, a hydrocarbon ring group, an aryl group, and a heterocyclic group. In addition, each of the exemplified substituents may be substituted or unsubstituted. For example, a biphenyl group may be interpreted as an aryl group or a phenyl group substituted with a phenyl group.

In the description, the term “forming a ring via the combination with an adjacent group” may mean forming a substituted or unsubstituted hydrocarbon ring, or a substituted or unsubstituted heterocycle via the combination with an adjacent group.

The hydrocarbon ring includes an aliphatic hydrocarbon ring and an aromatic hydrocarbon ring. The heterocycle includes an aliphatic heterocycle and an aromatic heterocycle. The hydrocarbon ring and the heterocycle may be monocycles or polycycles. In addition, the ring formed via the combination with an adjacent group may be combined with another ring to form a spiro structure.

In the description, the term “adjacent group” may mean a substituent substituted for an atom which is directly combined with an atom substituted with a corresponding substituent, another substituent substituted for an atom which is substituted with a corresponding substituent, or a substituent sterically positioned at the nearest position to a corresponding substituent. For example, in 1,2-dimethylbenzene, two methyl groups may be interpreted as “adjacent groups” to each other, and in 1,1-diethylcyclopentene, two ethyl groups may be interpreted as “adjacent groups” to each other. In addition, in 4,5-dimethylphenanthrene, two methyl groups may be interpreted as “adjacent groups” to each other.

In the description, the halogen atom may be a fluorine atom, a chlorine atom, a bromine atom or an iodine atom.

In the description, the alkyl group may be a linear, branched or ring type.

The carbon number of the alkyl group may be 1 to 50, 1 to 30, 1 to 20, 1 to 10, or 1 to 6. Examples of the alkyl group may include methyl, ethyl, n-propyl, isopropyl, n-butyl, s-butyl, t-butyl, i-butyl, 2-ethylbutyl, 3,3-dimethylbutyl, n-pentyl, i-pentyl, neopentyl, t-pentyl, cyclopentyl, 1-methylpentyl, 3-methylpentyl, 2-ethylpentyl, 4-methyl-2-pentyl, n-hexyl, 1-methylhexyl, 2-ethylhexyl, 2-butylhexyl, cyclohexyl, 4-methylcyclohexyl, 4-t-butylcyclohexyl, n-heptyl, 1-methylheptyl, 2,2-dimethylheptyl, 2-ethylheptyl, 2-butylheptyl, n-octyl, t-octyl, 2-ethyloctyl, 2-butyloctyl, 2-hexyloctyl, 3,7-dimethyloctyl, cyclooctyl, n-nonyl, n-decyl, adamantyl, 2-ethyldecyl, 2-butyldecyl, 2-hexyldecyl, 2-octyldecyl, n-undecyl, n-dodecyl, 2-ethyldodecyl, 2-butyldodecyl, 2-hexyldocecyl, 2-octyldodecyl, n-tridecyl, n-tetradecyl, n-pentadecyl, n-hexadecyl, 2-ethylhexadecyl, 2-butylhexadecyl, 2-hexylhexadecyl, 2-octylhexadecyl, n-heptadecyl, n-octadecyl, n-nonadecyl, n-eicosyl, 2-ethyleicosyl, 2-butyleicosyl, 2-hexyleicosyl, 2-octyleicosyl, n-henicosyl, n-docosyl, n-tricosyl, n-tetracosyl, n-pentacosyl, n-hexacosyl, n-heptacosyl, n-octacosyl, n-nonacosyl, n-triacontyl, etc., without limitation.

In the description, an alkenyl group means a hydrocarbon group including one or more carbon double bonds in the middle or at the terminal of an alkyl group having a carbon number of 2 or more. The alkenyl group may be a linear chain or a branched chain. The carbon number is not specifically limited, but may be 2 to 30, 2 to 20, or 2 to 10. Examples of the alkenyl group include a vinyl group, a 1-butenyl group, a 1-pentenyl group, a 1,3-butadienyl aryl group, a styrenyl group, a styrylvinyl group, etc., without limitation.

In the description, an alkynyl group means a hydrocarbon group including one or more carbon triple bonds in the middle or at the terminal of an alkyl group having a carbon number of 2 or more. The alkynyl group may be a linear chain or a branched chain. The carbon number is not specifically limited, but may be 2 to 30, 2 to 20, or 2 to 10. Particular examples of the alkynyl group include an ethynyl group, a propynyl group, etc., without limitation.

In the description, a hydrocarbon ring group means an optional functional group or substituent derived from an aliphatic hydrocarbon ring. The hydrocarbon ring group may be a saturated hydrocarbon ring group of 5 to 20 ring-forming carbon atoms.

In the description, an aryl group means an arbitrary functional group or substituent derived from an aromatic hydrocarbon ring. The aryl group may be a monocyclic aryl group or a polycyclic aryl group. The carbon number for forming rings in the aryl group may be 6 to 30, 6 to 20, or 6 to 15. Examples of the aryl group may include phenyl, naphthyl, fluorenyl, anthracenyl, phenanthryl, biphenyl, terphenyl, quaterphenyl, quinquephenyl, sexiphenyl, triphenylenyl, pyrenyl, benzofluoranthenyl, chrysenyl, etc., without limitation.

In the description, a fluorenyl group may be substituted, and two substituents may be combined with each other to form a spiro structure. Examples of a substituted fluorenyl group are as follows, but an embodiment of the inventive concept is not limited thereto.

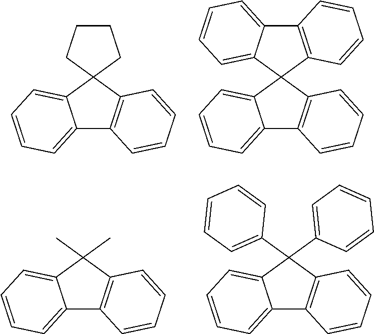

In the description, a heterocyclic group means an optional functional group or substituent derived from a ring including one or more among B, O, N, P, Si, and S as heteroatoms. The heterocyclic group includes an aliphatic heterocyclic group and an aromatic heterocyclic group. The aromatic heterocyclic group may be a heteroaryl group. The aliphatic heterocyclic group and the aromatic heterocyclic group may be a monocycle or a polycycle.

In the description, if the heterocyclic group includes two or more heteroatoms, two or more heteroatoms may be the same or different. The heterocyclic group may be a monocyclic heterocyclic group or a polycyclic heterocyclic group, and has the concept including a heteroaryl group. The carbon number for forming rings of the heterocyclic group may be 2 to 30, 2 to 20, and 2 to 10.

In the description, an aliphatic heterocyclic group may include one or more among B, O, N, P, Si, and S as heteroatoms. The number of ring-forming carbon atoms of the aliphatic heterocyclic group may be 2 to 30, 2 to 20, or 2 to 10. Examples of the aliphatic heterocyclic group may include an oxirane group, a thiirane group, a pyrrolidine group, a piperidine group, a tetrahydrofuran group, a tetrahydrothiophene group, a thiane group, a tetrahydropyran group, a 1,4-dioxane group, etc., without limitation.

In the description, a heteroaryl group may include one or more among B, O, N, P, Si, and S as heteroatoms. If the heteroaryl group includes two or more heteroatoms, two or more heteroatoms may be the same or different. The heteroaryl group may be a monocyclic heterocyclic group or polycyclic heterocyclic group. The carbon number for forming rings of the heteroaryl group may be 2 to 60, 2 to 30, 2 to 20, or 2 to 10. Examples of the heteroaryl group may include thiophene, furan, pyrrole, imidazole, pyridine, bipyridine, pyrimidine, triazine, triazole, acridyl, pyridazine, pyrazinyl, quinoline, quinazoline, quinoxaline, phenoxazine, phthalazine, pyrido pyridine, pyrido pyrazine, pyrazino pyrazine, isoquinoline, indole, carbazole, N-arylcarbazole, N-heteroarylcarbazole, N-alkylcarbazole, benzoxazole, benzoimidazole, benzothiazole, benzocarbazole, benzothiophene, dibenzothiophene, thienothiophene, benzofuran, phenanthroline, thiazole, isooxazole, oxazole, oxadiazole, thiadiazole, phenothiazine, dibenzosilole, dibenzofuran, etc., without limitation.

In the description, the same explanation on the above-described aryl group may be applied to an arylene group except that the arylene group is a divalent group.

The same explanation on the above-described heteroaryl group may be applied to a heteroarylene group except that the heteroarylene group is a divalent group.

In the description, a silyl group includes an alkyl silyl group and an aryl silyl group. Examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, etc., without limitation.

In the description, the carbon number of a carbonyl group is not specifically limited, but the carbon number may be 1 to 40, 1 to 30, or 1 to 20. For example, the carbonyl group may have the structures below, but is not limited thereto.

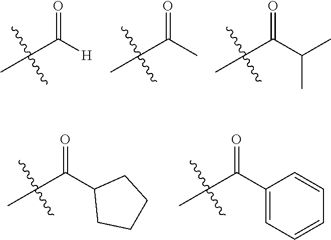

In the description, the carbon number of a sulfinyl group and sulfonyl group is not specifically limited, but may be 1 to 30. The sulfinyl group may include an alkyl sulfinyl group and an aryl sulfinyl group. The sulfonyl group may include an alkyl sulfonyl group and an aryl sulfonyl group.

In the description, a thio group may include an alkyl thio group and an aryl thio group. The thio group may mean the above-defined alkyl group or aryl group combined with a sulfur atom. Examples of the thio group include a methylthio group, an ethylthio group, a propylthio group, a pentylthio group, a hexylthio group, an octylthio group, a dodecylthio group, a cyclopentylthio group, a cyclohexylthio group, a phenylthio group, a naphthylthio group, etc., without limitation.

In the description, an oxy group may mean the above-defined alkyl group or aryl group which is combined with an oxygen atom. The oxy group may include an alkoxy group and an aryl oxy group. The alkoxy group may be a linear, branched or cyclic chain. The carbon number of the alkoxy group is not specifically limited but may be, for example, 1 to 20 or 1 to 10. Examples of the oxy group may include methoxy, ethoxy, n-propoxy, isopropoxy, butoxy, pentyloxy, hexyloxy, octyloxy, nonyloxy, decyloxy, phenoxy, benzyloxy, etc. However, an embodiment of the inventive concept is not limited thereto.

In the description, a boron group may mean the above-defined alkyl group or aryl group, combined with a boron atom. The boron group includes an alkyl boron group and an aryl boron group. Examples of the boron group include a dimethylboron group, a diethylboron group, a t-butylmethylboron group, a diphenylboron group, a phenylboron group, etc., without limitation.

In the description, an alkenyl group may be a linear chain or a branched chain. The carbon number is not specifically limited, but may be 2 to 30, 2 to 20 or 2 to 10. Examples of the alkenyl group includes a vinyl group, a 1-butenyl group, a 1-pentenyl group, a 1,3-butadienyl aryl group, a styrenyl group, a styrylvinyl group, etc., without limitation.

In the description, the carbon number of the amine group is not specifically limited, but may be 1 to 30. The amine group may include an alkyl amine group and an aryl amine group. Examples of the amine group include a methylamine group, a dimethylamine group, a phenylamine group, a diphenylamine group, a naphthylamine group, a 9-methyl-anthracenylamine group, a triphenylamine group, etc., without limitation. Meanwhile, the amine group of the description may not mean a fused ring type, but may mean a chain-type amine. That is, a ring-type amine group such as a pyrrole group, an imidazole group, a pyridine group, a bipyridine group, a pyrimidine group, a triazine group, an indole group, and a carbazole group is defined as a heteroaryl group in the description, and the amine group may mean only an amine group not forming a ring in the description.

In the description, alkyl groups in an alkylthiol group, alkylsulfoxy group, alkylaryl group, alkylamino group, alkyl boron group, alkyl silyl group, and alkyl amine group may be the same as the examples of the above-described alkyl group.

In the description, aryl groups in an aryloxy group, arylthio group, arylsulfoxy group, arylamino group, aryl boron group, aryl silyl group, and aryl amine group may be the same as the examples of the above-described aryl group.

In the description, a direct linkage may mean a single bond.

Meanwhile, in the description,

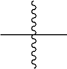

or “ [symbol] ” means a position to be connected.

Hereinafter, embodiments of the inventive concept will be explained referring to the drawings.

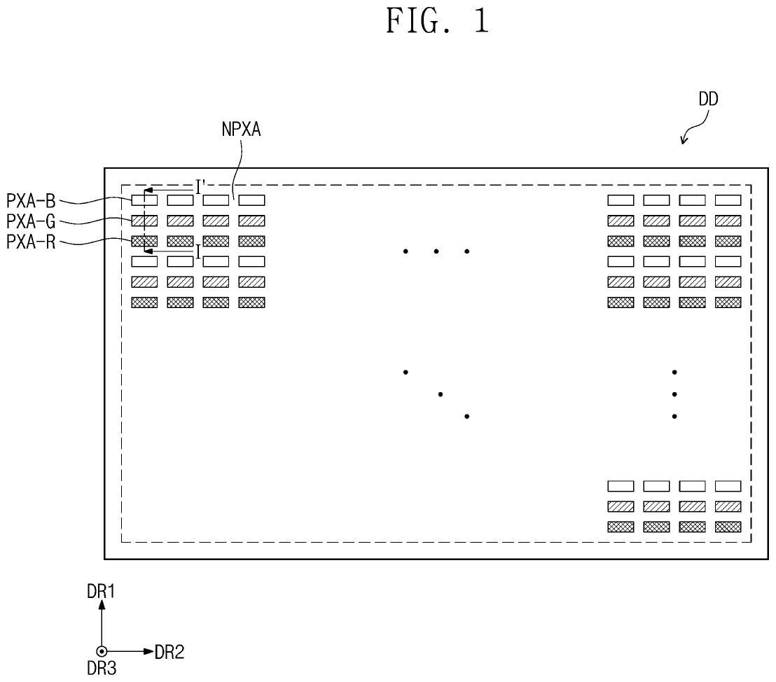

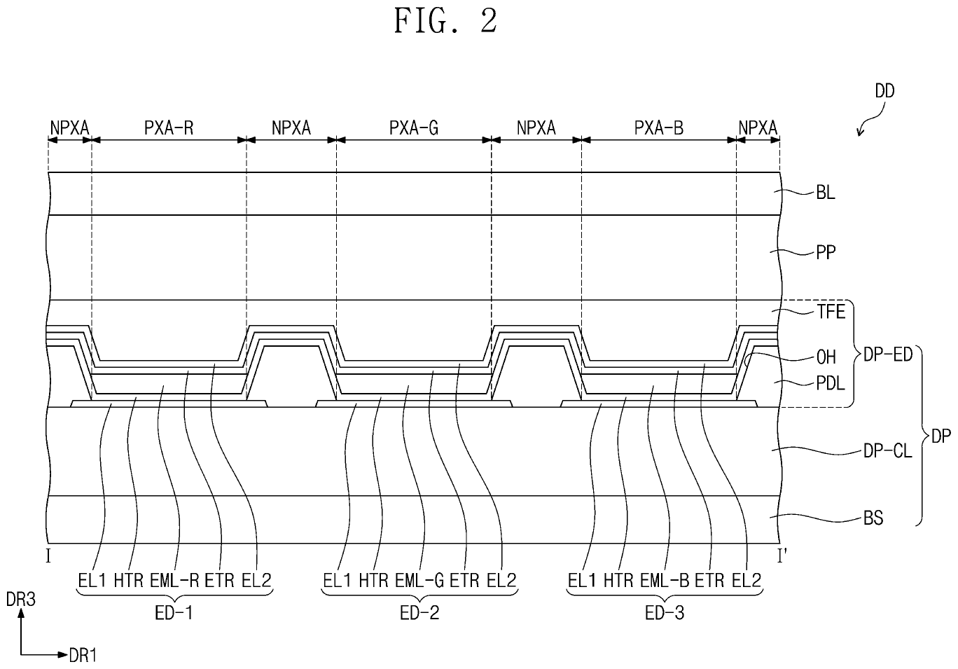

FIG. 1 is a plan view showing an embodiment of a display apparatus DD. FIG. 2 is a cross-sectional view of a display apparatus DD of an embodiment. FIG. 2 is a cross-sectional view showing a part corresponding to line I-I′ in FIG. 1.

The display apparatus DD may include a display panel DP and an optical layer PP disposed on the display panel DP. The display panel DP includes light emitting devices ED-1, ED-2 and ED-3. The display apparatus DD may include multiple light emitting devices ED-1, ED-2 and ED-3. The optical layer PP may be disposed on the display panel DP and control reflected light by external light at the display panel DP. The optical layer PP may include, for example, a polarization layer or a color filter layer. Meanwhile, different from the drawings, the optical layer PP may be omitted in the display apparatus DD of an embodiment.

On the optical layer PP, a base substrate BL may be disposed. The base substrate BL may be a member providing a base surface where the optical layer PP is disposed. The base substrate BL may be a glass substrate, a metal substrate, a plastic substrate, etc. However, an embodiment of the inventive concept is not limited thereto, and the base substrate BL may be an inorganic layer, an organic layer or a composite material layer. In addition, different from the drawings, the base substrate BL may be omitted in an embodiment.

The display apparatus DD according to an embodiment may further include a plugging layer (not shown). The plugging layer (not shown) may be disposed between a display device layer DP-ED and a base substrate BL. The plugging layer (not shown) may be an organic layer. The plugging layer (not shown) may include at least one among an acrylic resin, a silicon-based resin and an epoxy-based resin.

The display panel DP may include a base layer BS, a circuit layer DP-CL provided on the base layer BS and a display device layer DP-ED. The display device layer DP-ED may include a pixel definition layer PDL, light emitting devices ED-1, ED-2 and ED-3 disposed in the pixel definition layer PDL, and an encapsulating layer TFE disposed on the light emitting devices ED-1, ED-2 and ED-3.

The base layer BS may be a member providing a base surface where the display device layer DP-ED is disposed. The base layer BS may be a glass substrate, a metal substrate, a plastic substrate, etc. However, an embodiment of the inventive concept is not limited thereto, and the base layer BS may be an inorganic layer, an organic layer or a composite material layer.

In an embodiment, the circuit layer DP-CL is disposed on the base layer BS, and the circuit layer DP-CL may include multiple transistors (not shown). Each of the transistors (not shown) may include a control electrode, an input electrode, and an output electrode. For example, the circuit layer DP-CL may include switching transistors and driving transistors for driving the light emitting devices ED-1, ED-2 and ED-3 of the display device layer DP-ED.

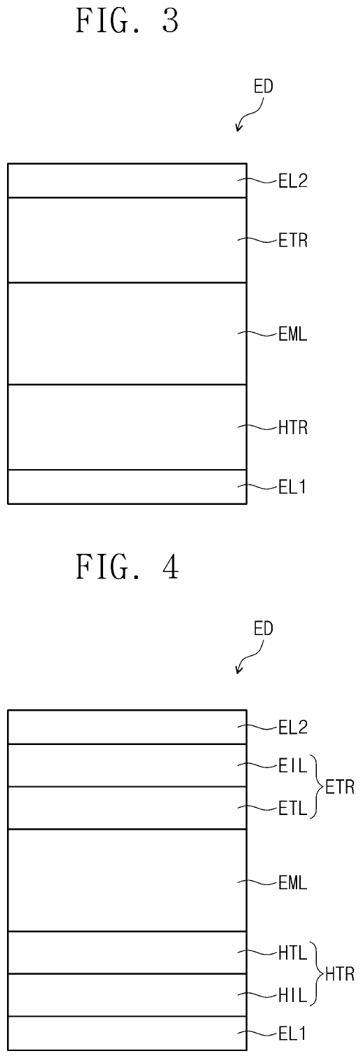

Each of the light emitting devices ED-1, ED-2 and ED-3 may have the structures of light emitting devices ED of embodiments according to FIG. 3 to FIG. 6, which will be explained later. Each of the light emitting devices ED-1, ED-2 and ED-3 may include a first electrode EL1, a hole transport region HTR, emission layers EML-R, EML-G and EML-B, an electron transport region ETR, and a second electrode EL2.

In FIG. 2, shown is an embodiment where the emission layers EML-R, EML-G and EML-B of light emitting devices ED-1, ED-2 and ED-3, which are in opening portions OH defined in a pixel definition layer PDL, are disposed, and a hole transport region HTR, an electron transport region ETR and a second electrode EL2 are provided as common layers in all light emitting devices ED-1, ED-2 and ED-3. However, an embodiment of the inventive concept is not limited thereto. Different from FIG. 2, in an embodiment, the hole transport region HTR and the electron transport region ETR may be patterned and provided in the opening portions OH defined in the pixel definition layer PDL. For example, in an embodiment, the hole transport region HTR, the emission layers EML-R, EML-G and EML-B, and the electron transport region ETR of the light emitting devices ED-1, ED-2 and ED-3 may be patterned by an inkjet printing method and provided.

An encapsulating layer TFE may cover the light emitting devices ED-1, ED-2 and ED-3. The encapsulating layer TFE may encapsulate a display device layer DP-ED. The encapsulating layer TFE may be a thin film encapsulating layer. The encapsulating layer TFE may be one layer or a stacked layer of multiple layers. The encapsulating layer TFE includes at least one insulating layer. The encapsulating layer TFE according to an embodiment may include at least one inorganic layer (hereinafter, encapsulating inorganic layer). In addition, the encapsulating layer TFE according to an embodiment may include at least one organic layer (hereinafter, encapsulating organic layer) and at least one encapsulating inorganic layer.

The encapsulating inorganic layer protects the display device layer DP-ED from moisture/oxygen, and the encapsulating organic layer protects the display device layer DP-ED from foreign materials such as dust particles. The encapsulating inorganic layer may include silicon nitride, silicon oxy nitride, silicon oxide, titanium oxide, or aluminum oxide, without specific limitation. The encapsulating organic layer may include an acrylic compound, an epoxy-based compound, etc. The encapsulating organic layer may include a photopolymerizable organic material, without specific limitation.

The encapsulating layer TFE may be disposed on the second electrode EL2 and may be disposed while filling the opening portion OH.

Referring to FIG. 1 and FIG. 2, the display apparatus DD may include a non-luminous area NPXA and luminous areas PXA-R, PXA-G and PXA-B. The luminous areas PXA-R, PXA-G and PXA-B may be areas emitting light produced from the light emitting devices ED-1, ED-2 and ED-3, respectively. The luminous areas PXA-R, PXA-G and PXA-B may be separated from each other on a plane.

The luminous areas PXA-R, PXA-G and PXA-B may be areas separated by the pixel definition layer PDL. The non-luminous areas NPXA may be areas between neighboring luminous areas PXA-R, PXA-G and PXA-B and may be areas corresponding to the pixel definition layer PDL. Meanwhile, in the disclosure, each of the luminous areas PXA-R, PXA-G and PXA-B may correspond to each pixel. The pixel definition layer PDL may divide the light emitting devices ED-1, ED-2 and ED-3. The emission layers EML-R, EML-G and EML-B of the light emitting devices ED-1, ED-2 and ED-3 may be disposed and divided in the opening portions OH defined in the pixel definition layer PDL.

The luminous areas PXA-R, PXA-G and PXA-B may be divided into multiple groups according to the color of light produced from the light emitting devices ED-1, ED-2 and ED-3. In the display apparatus DD of an embodiment, shown in FIG. 1 and FIG. 2, three luminous areas PXA-R, PXA-G and PXA-B emitting red light, green light and blue light are illustrated as an embodiment. For example, the display apparatus DD of an embodiment may include a red luminous area PXA-R, a green luminous area PXA-G and a blue luminous area PXA-B, which are separated from each other.

In the display apparatus DD according to an embodiment, multiple light emitting devices ED-1, ED-2 and ED-3 may emit light having different wavelength regions. For example, in an embodiment, the display apparatus DD may include a first light emitting device ED-1 emitting red light, a second light emitting device ED-2 emitting green light, and a third light emitting device ED-3 emitting blue light. That is, each of the red luminous area PXA-R, the green luminous area PXA-G, and the blue luminous area PXA-B of the display apparatus DD may correspond to the first light emitting device ED-1, the second light emitting device ED-2, and the third light emitting device ED-3.

However, an embodiment of the inventive concept is not limited thereto, and the first to third light emitting devices ED-1, ED-2 and ED-3 may emit light in the same wavelength region, or at least one thereof may emit light in a different wavelength region. For example, all the first to third light emitting devices ED-1, ED-2 and ED-3 may emit blue light.

The luminous areas PXA-R, PXA-G and PXA-B in the display apparatus DD according to an embodiment may be arranged in a stripe shape. Referring to FIG. 1, multiple red luminous areas PXA-R, multiple green luminous areas PXA-G and multiple blue luminous areas PXA-B may be arranged along a second direction axis DR2. In addition, the red luminous area PXA-R, the green luminous area PXA-G and the blue luminous area PXA-B may be arranged by turns along a first direction axis DR1.

In FIG. 1 and FIG. 2, the areas of the luminous areas PXA-R, PXA-G and PXA-B are shown similar, but an embodiment of the inventive concept is not limited thereto. The areas of the luminous areas PXA-R, PXA-G and PXA-B may be different from each other according to the wavelength region of light emitted. Meanwhile, the areas of the luminous areas PXA-R, PXA-G and PXA-B may mean areas on a plane defined by the first direction axis DR1 and the second direction axis DR2.

Meanwhile, the arrangement type of the luminous areas PXA-R, PXA-G and PXA-B is not limited to the configuration shown in FIG. 1, and the arrangement order of the red luminous areas PXA-R, the green luminous areas PXA-G and the blue luminous areas PXA-B may be provided in various combinations according to the properties of display quality required for the display apparatus DD. For example, the arrangement type of the luminous areas PXA-R, PXA-G and PXA-B may be a pentile (PENTILE™) arrangement type, or a diamond (Diamond Pixel™) arrangement type.

In addition, the areas of the luminous areas PXA-R, PXA-G and PXA-B may be different from each other. For example, in an embodiment, the area of the green luminous area PXA-G may be smaller than the area of the blue luminous area PXA-B, but an embodiment of the inventive concept is not limited thereto.

Hereinafter, FIG. 3 to FIG. 6 are cross-sectional views schematically showing light emitting devices according to embodiments. The light emitting device ED of an embodiment may include a first electrode EL1, a hole transport region HTR, an emission layer EML, an electron transport region ETR, and a second electrode EL2 stacked in order.

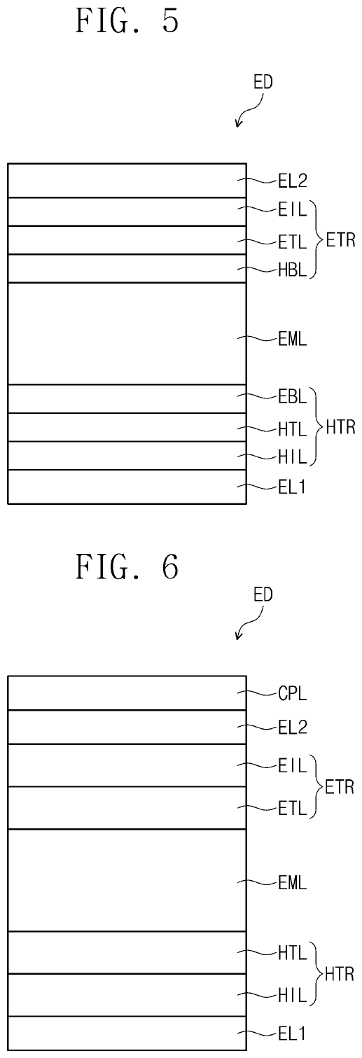

When compared with FIG. 3, FIG. 4 shows the cross-sectional view of a light emitting device ED of an embodiment, wherein a hole transport region HTR includes a hole injection layer HIL and a hole transport layer HTL, and an electron transport region ETR includes an electron injection layer EIL and an electron transport layer ETL. In addition, when compared with FIG. 3, FIG. 5 shows the cross-sectional view of a light emitting device ED of an embodiment, wherein a hole transport region HTR includes a hole injection layer HIL, a hole transport layer HTL, and an electron blocking layer EBL, and an electron transport region ETR includes an electron injection layer EIL, an electron transport layer ETL, and a hole blocking layer HBL. When compared with FIG. 4, FIG. 6 shows the cross-sectional view of a light emitting device ED of an embodiment, including a capping layer CPL disposed on the second electrode EL2.

In the light emitting element ED according to an embodiment, the first electrode EL1 has conductivity. The first electrode EL1 may be formed using a metal material, a metal alloy or a conductive compound. The first electrode EL1 may be an anode or a cathode. However, an embodiment of the inventive concept is not limited thereto. In addition, the first electrode EL1 may be a pixel electrode. The first electrode EL1 may be a transmissive electrode, a transflective electrode, or a reflective electrode. The first electrode EL1 may include at least one selected from Ag, Mg, Cu, Al, Pt, Pd, Au, Ni, Nd, Ir, Cr, Li, Ca, LiF, Mo, Ti, W, In, Sn, Zn, compounds of two or more selected therefrom, mixtures of two or more selected therefrom, or oxides thereof.

If the first electrode EL1 is the transmissive electrode, the first electrode EL1 may include a transparent metal oxide such as indium tin oxide (ITO), indium zinc oxide (IZO), zinc oxide (ZnO), and indium tin zinc oxide (ITZO). If the first electrode EL1 is the transflective electrode or the reflective electrode, the first electrode EL1 may include Ag, Mg, Cu, Al, Pt, Pd, Au, Ni, Nd, Ir, Cr, Li, Ca, LiF/Ca (stacked structure of LiF and Ca), LiF/Al (stacked structure of LiF and Al), Mo, Ti, W, compounds thereof, or mixtures thereof (for example, a mixture of Ag and Mg). Also, the first electrode EL1 may have a structure including multiple layers including a reflective layer or a transflective layer formed using the above materials, and a transmissive conductive layer formed using ITO, IZO, ZnO, or ITZO. For example, the first electrode EL1 may include a three-layer structure of ITO/Ag/ITO. However, an embodiment of the inventive concept is not limited thereto. The first electrode EL1 may include the above-described metal materials, combinations of two or more metal materials selected from the above-described metal materials, or oxides of the above-described metal materials. The thickness of the first electrode EL1 may be from about 700 Å to about 10,000 Å. For example, the thickness of the first electrode EL1 may be from about 1,000 Å to about 3,000 Å.

The hole transport region HTR is provided on the first electrode EL1. The hole transport region HTR may have a single layer formed of a single material, a single layer formed of a plurality of different materials, or a multilayer structure including a plurality of layers formed of a plurality of different materials.

The hole transport region HTR may include at least one of the hole injection layer HIL, the hole transport layer HTL, or the electron blocking layer EBL. In addition, although not illustrated, the hole transport region HTR may include a plurality of stacked hole transport layers.

In addition, unlike this, the hole transport region HTR may have a single layer structure of the hole injection layer HIL or the hole transport layer HTL, or may have a single layer structure formed of a hole injection material and a hole transport material. In an embodiment, the hole transport region HTR may have a single layer structure formed of a plurality of different materials, or a structure in which a hole injection layer HIL/hole transport layer HTL, a hole injection layer HIL/hole transport layer HTL/buffer layer (not shown), a hole injection layer HIL/buffer layer (not shown), or a hole transport layer HTL/buffer layer (not shown) are stacked in order from the first electrode EL1, but the embodiment of the inventive concept is not limited thereto.

The thickness of the hole transport region HTR may be, for example, from about 50 Å to about 15,000 Å. The hole transport region HTR may be formed using various methods such as a vacuum deposition method, a spin coating method, a cast method, a Langmuir-Blodgett (LB) method, an inkjet printing method, a laser printing method, and a laser induced thermal imaging (LITI) method.

The light emitting element ED of an embodiment may include the monoamine compound of an embodiment in the hole transport region HTR. In the light emitting element ED of an embodiment, the hole transport region HTR may include an electron injection layer EIL or a hole transport layer HTL, and the hole transport layer HTL may include the monoamine compound of an embodiment. The monoamine compound of an embodiment may be included in a layer, which is adjacent to the emission layer EML, among the layers included in the hole transport region HTR.

In an example embodiment, the monoamine compound may be represented by the following Formula 1:

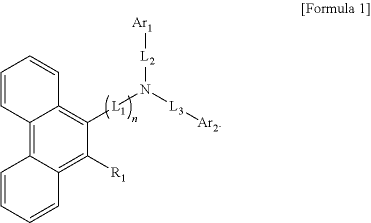

In Formula 1, L₁ may be a substituted or unsubstituted arylene group having 6 to 30 carbon atoms for forming a ring, or a substituted or unsubstituted heteroarylene group having 2 to 30 carbon atoms for forming a ring. L₁ may be a substituted or unsubstituted phenylene group. L₁ may be a substituted or unsubstituted divalent biphenyl group. L₁ may be a substituted or unsubstituted naphthylene group.

L₁ may be a substituted or unsubstituted arylene group having 6 to 15 carbon atoms for forming a ring.

L₁ may be an unsubstituted phenylene group. L₁ may be an m-phenylene group or a p-phenylene group. L₁ may be a monosubstituted phenylene group. For example, L₁ may be a phenylene group substituted with a phenyl group or a triphenylsilyl group.

L₁ may be an unsubstituted divalent biphenyl group. L₁ may be an unsubstituted naphthylene group.

Here, n is 1 or 2. In the case where n is 2, a plurality of L₁ may be the same or different.

L₂ and L₃ may be the same or different. L₂ and L₃ are each independently a direct linkage, a substituted or unsubstituted arylene group having 6 to 30 carbon atoms for forming a ring, or a substituted or unsubstituted heteroarylene group having 2 to 30 carbon atoms for forming a ring. L₂ and L₃ may each independently be a direct linkage, a substituted or unsubstituted phenylene group, or a substituted or unsubstituted divalent biphenyl group. L₂ and L₃ may each independently be a substituted or unsubstituted naphthylene group.

L₂ and L₃ may each independently be an unsubstituted divalent phenylene group. L₂ and L₃ may each independently be an m-phenylene group or a p-phenylene group. Each of L₂ and L₃ may be a monosubstituted phenylene group. Each of L₂ and L₃ may be a phenylene group substituted with an alkyl group having 1 to 10 carbon atoms or an aryl group having 6 to 30 carbon atoms. For example, each of L₂ and L₃ may be a phenylene group substituted with a methyl group or a phenyl group.

L₂ and L₃ may each independently be an unsubstituted divalent biphenyl group. L₂ and L₃ may each independently be an unsubstituted naphthylene group.

R₁ is an alkyl group having 1 to 10 carbon atoms, a substituted or unsubstituted aryl group having 6 to 30 carbon atoms for forming a ring, a substituted or unsubstituted heteroaryl group having 2 to 30 carbon atoms for forming a ring, or a substituted or unsubstituted aryl silyl group. R₁ may be a substituted or unsubstituted phenyl group, a substituted or unsubstituted naphthyl group, a substituted or unsubstituted biphenyl group, or a substituted or unsubstituted nitrogen-containing heteroaryl group. For example, R₁ may be a pyridinyl group or a triphenylsilyl group.

R₁ may be a monosubstituted phenyl group. R₁ may be a deuterium atom, a halogen atom, a cyano group, or a phenyl group substituted with an alkyl group having 1 to 10 carbon atoms. For example, R₁ may be a phenyl group substituted with a cyano group or an isopropyl group.

Ar₁ and Ar₂ may be the same or different. Ar₁ and Ar₂ are each independently a substituted or unsubstituted aryl group having 6 to 30 carbon atoms for forming a ring, or a substituted or unsubstituted heteroaryl group having 2 to 30 carbon atoms for forming a ring. Ar₁ and Ar₂ will be described in detail below.

In an example embodiment, in Formula 1, only the R₁ position in the illustrated phenanthryl group is substituted, and other positions are not substituted. In the case where a substituent is positioned at the other positions of the phenanthryl group, the energy levels of the HOMO and LUMO of the phenanthryl group may change due to the substituent, and hole transport properties may be deteriorated. In an example embodiment, in a monoamine compound in which only the R₁ position of the phenanthryl group is substituted, hole transport properties may be enhanced for use of the monoamine compound as a hole transport material.

In example embodiments, Formula 1 may be represented by the following Formula 2-1 or 2-2:

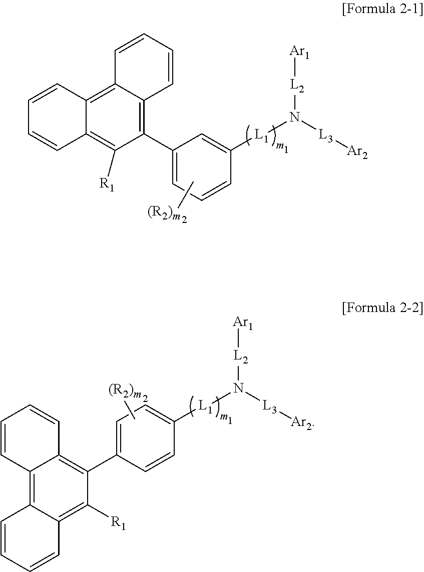

Here, m₁ may be 0 or 1, and m₂ may be an integer of 0 to 2.

R₂ may be a hydrogen atom, a deuterium atom, a substituted or unsubstituted alkyl group having 1 to 10 carbon atoms, a substituted or unsubstituted aryl group having 6 to 30 carbon atoms for forming a ring, a substituted or unsubstituted heteroaryl group having 2 to 30 carbon atoms for forming a ring, or a substituted or unsubstituted aryl silyl group. For example, R₂ may be a hydrogen atom, an unsubstituted phenyl group, or a triphenylsilyl group.

In the case where m₂ is 2, a plurality of R₂ may be the same or different. R₂ may be combined with an adjacent group to form a ring. For example, R₂ may be combined with an adjacent group to form a substituted or unsubstituted hydrocarbon ring, or a substituted or unsubstituted heterocycle. R₂ may be combined with an adjacent group to form a substituted or unsubstituted aromatic hydrocarbon ring.

In Formulae 2-1 and 2-2, the particular explanation on Ar₁, Ar₂, L₁, L₂, L₃, and R₁ is the same as the explanation referring to Formula 1 and will not be repeated.

Formula 2-1 may be represented by one of the following Formulae 2-1-1 to 2-1-3:

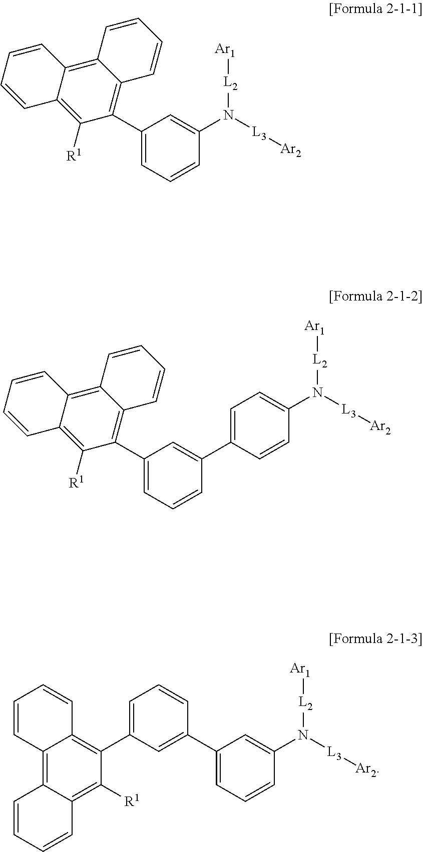

In Formulae 2-1-1 to 2-1-3, Ar₁, Ar₂, L₂, L₃, and R₁ are the same as defined in Formula 1.

In Formulae 2-1-1 to 2-1-3, R₁ may be a substituted or unsubstituted phenyl group, L₃ may be a substituted or unsubstituted phenylene group, and Ar₂ may be a substituted or unsubstituted naphthyl group. More specifically, the monoamine compound may be represented by the following Formula 2-1-1, R₁ may be a substituted or unsubstituted phenyl group, L₃ may be a substituted or unsubstituted phenylene group, and Ar₂ may be a substituted or unsubstituted naphthyl group. In case Ar₂ is a substituted or unsubstituted naphthyl group, L₃ may be connected at the carbon of position 1 of the naphthyl group.

In Formulae 2-1-1 to 2-1-3, L₂ may be a substituted or unsubstituted phenylene group, and Ar₁ may be a substituted or unsubstituted phenyl group. More specifically, the monoamine compound may be represented by the following Formula 2-1-1, R₁ may be a substituted or unsubstituted phenyl group, L₃ may be a substituted or unsubstituted phenylene group, Ar₂ may be a substituted or unsubstituted naphthyl group, L₂ may be a substituted or unsubstituted phenylene group, and Ar₁ may be a substituted or unsubstituted phenyl group.

In Formulae 2-1-1 to 2-1-3, L₂ may be a direct linkage, and Ar₁ may be a substituted or unsubstituted dibenzofuranyl group. More specifically, the monoamine compound may be represented by the following Formula 2-1-1, R₁ may be a substituted or unsubstituted phenyl group, L₃ may be a substituted or unsubstituted phenylene group, Ar₂ may be a substituted or unsubstituted naphthyl group, L₂ may be a direct linkage, and Ar₁ may be a substituted or unsubstituted dibenzofuranyl group.

Formula 2-2 may be represented by one of the following Formulae 2-2-1 to 2-2-3:

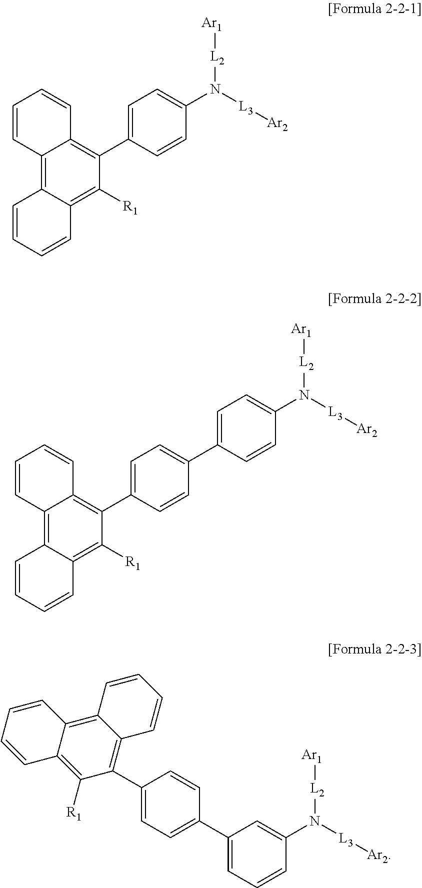

In Formulae 2-2-1 to 2-2-3, Ar₁, Ar₂, L₂, L₃, and R₁ are the same as defined in Formula 1.

In Formula 1, Ar₁ and Ar₂ may be each independently a substituted or unsubstituted phenyl group. Ar₁ and Ar₂ may each independently be a mono- or di-substituted phenyl group. Ar₁ and Ar₂ may each independently be a phenyl group substituted with a deuterium atom, a halogen atom, a cyano group, or an alkyl group having 1 to 10 carbon atoms. For example, Ar₁ and Ar₂ may each independently be a phenyl group substituted with a fluorine atom or a substituted or unsubstituted octyl group.

Ar₁ and Ar₂ may each independently be a substituted or unsubstituted biphenyl group. Ar₁ and Ar₂ may each independently be a substituted or unsubstituted terphenyl group. Ar₁ and Ar₂ may each independently be a substituted or unsubstituted naphthyl group. Ar₁ and Ar₂ may each independently be a substituted or unsubstituted phenanthryl group.

Ar₁ and Ar₂ may each independently be a substituted or unsubstituted fluorenyl group. Ar₁ and Ar₂ may each independently be a disubstituted fluorenyl group. For example, Ar₁ and Ar₂ may each independently be a fluorenyl group substituted with an aryl group.

Ar₁ and Ar₂ may each independently be a substituted or unsubstituted dibenzofuranyl group. Ar₁ and Ar₂ may each independently be a monosubstituted dibenzofuranyl group. Ar₁ and Ar₂ may each independently be a dibenzofuranyl group substituted with an alkyl group having 1 to 10 carbon atoms, a substituted or unsubstituted aryl group having 6 to 30 carbon atoms for forming a ring, or a substituted or unsubstituted heteroaryl group having 2 to 30 carbon atoms for forming a ring. For example, Ar₁ and Ar₂ may each independently be a dibenzofuranyl group substituted with a phenyl group or a cyclohexyl group.

Ar₁ and Ar₂ may each independently be a substituted or unsubstituted dibenzothiophenyl group. Ar₁ and Ar₂ may each independently be a substituted or unsubstituted benzonaphthofuranyl group. Ar₁ and Ar₂ may each independently be a substituted or unsubstituted benzonaphthothiophenyl group.

Ar₁ and Ar₂ may each independently be represented by the following Formula 3:

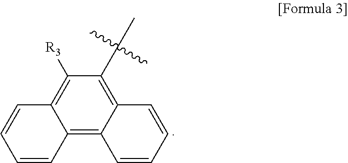

R₃ may be an alkyl group having 1 to 10 carbon atoms, a substituted or unsubstituted aryl group having 6 to 30 carbon atoms for forming a ring, a substituted or unsubstituted heteroaryl group having 2 to 30 carbon atoms for forming a ring, or a substituted or unsubstituted aryl silyl group, and

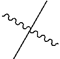

indicates bonding to an adjacent moiety.

In the case where Ar₁ and Ar₂ are each independently represented by Formula 3, in formula 1, L₂ and L₃ may each independently be a substituted or unsubstituted arylene group having 6 to 30 carbon atoms for forming a ring, or a substituted or unsubstituted heteroarylene group having 2 to 30 carbon atoms for forming a ring.

In the case where Ar₁ and Ar₂ are each independently represented by Formula 3, in formula 1, L₂ and L₃ may each independently be a substituted or unsubstituted phenylene group or a substituted or unsubstituted divalent biphenyl group. L₂ and L₃ may each independently be an unsubstituted phenylene group.

Ar₁ and Ar₂ may be represented by the following Formula 4:

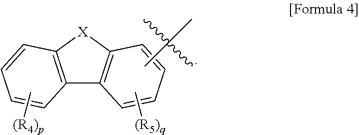

X may be O or S.

R₄ and R₅ may each independently be a hydrogen atom, a deuterium atom, a substituted or unsubstituted alkyl group having 1 to 10 carbon atoms, a substituted or unsubstituted aryl group having 6 to 30 carbon atoms for forming a ring, a substituted or unsubstituted heteroaryl group having 2 to 30 carbon atoms for forming a ring, or a substituted or unsubstituted aryl silyl group. R₄ and R₅ may each independently be a cycloalkyl group. For example, R₄ and R₅ may each independently be a cyclohexyl group.

“p” may be an integer of 0 to 4. “q” may be and integer of 0 to 3. In the case where “p” is an integer of 2 or more, a plurality of R₄ may be the same or different. In the case where “q” is an integer of 2 or more, a plurality of R₅ may be the same or different. In the case where “p” and “q” are each independently an integer of 2 or more, R₄ and R₅ may each independently be combined with an adjacent group to form a ring. For example, in the case where at least one of R₄ and R₅ forms an aromatic ring, Ar₁ and Ar₂ may be a heteroaryl group having four or five rings.

The monoamine compound represented by Formula 1 may be at least one selected from the monoamine compounds represented in the following Compound Group 1. However, an example embodiment is not limited thereto.

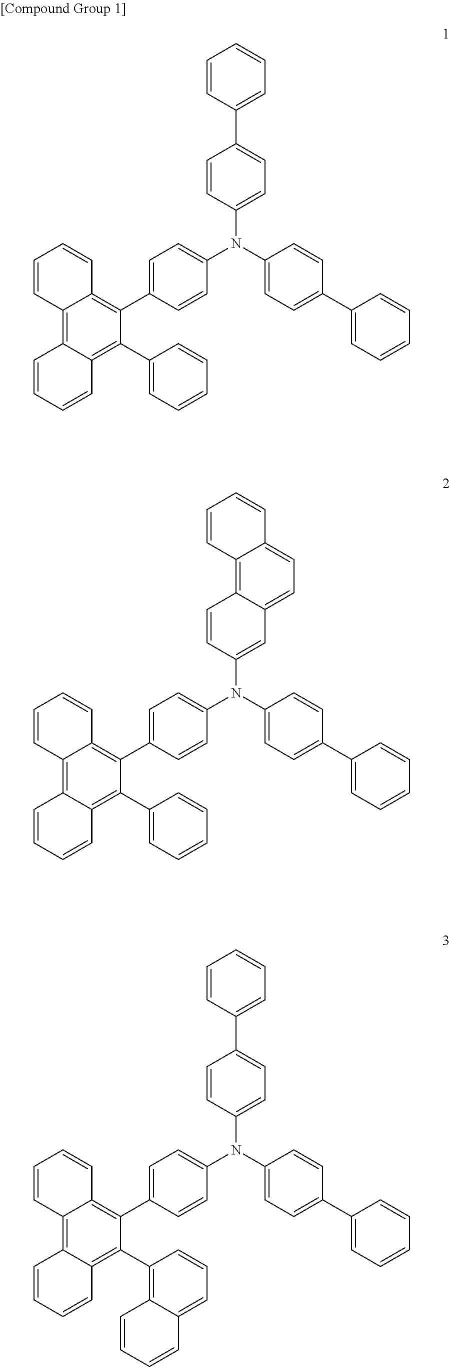 [SMILES: c1ccc(-c2ccc(N(c3ccc(-c4ccccc4)cc3)c3ccc(-c4c(-c5ccccc5)c5ccccc5c5ccccc45)cc3)cc2)cc1 ; c1ccc(-c2ccc(N(c3ccc(-c4c(-c5ccccc5)c5ccccc5c5ccccc45)cc3)c3ccc4c(ccc5ccccc54)c3)cc2)cc1 ; c1ccc(-c2ccc(N(c3ccc(-c4ccccc4)cc3)c3ccc(-c4c(-c5cccc6ccccc56)c5ccccc5c5ccccc45)cc3)cc2)cc1]

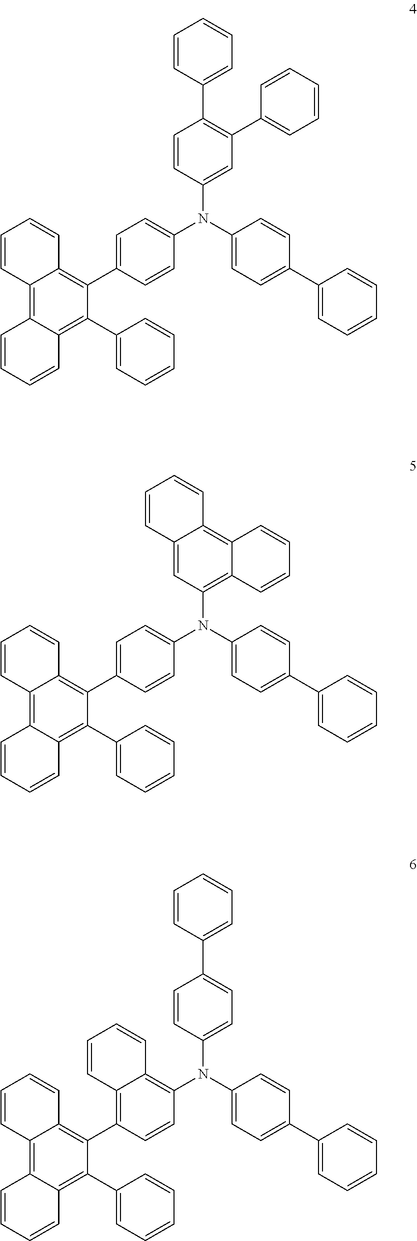 [SMILES: c1ccc(-c2ccc(N(c3ccc(-c4c(-c5ccccc5)c5ccccc5c5ccccc45)cc3)c3ccc(-c4ccccc4)c(-c4ccccc4)c3)cc2)cc1 ; c1ccc(-c2ccc(N(c3ccc(-c4c(-c5ccccc5)c5ccccc5c5ccccc45)cc3)c3cc4ccccc4c4ccccc34)cc2)cc1 ; c1ccc(-c2ccc(N(c3ccc(-c4ccccc4)cc3)c3ccc(-c4c(-c5ccccc5)c5ccccc5c5ccccc45)c4ccccc34)cc2)cc1]

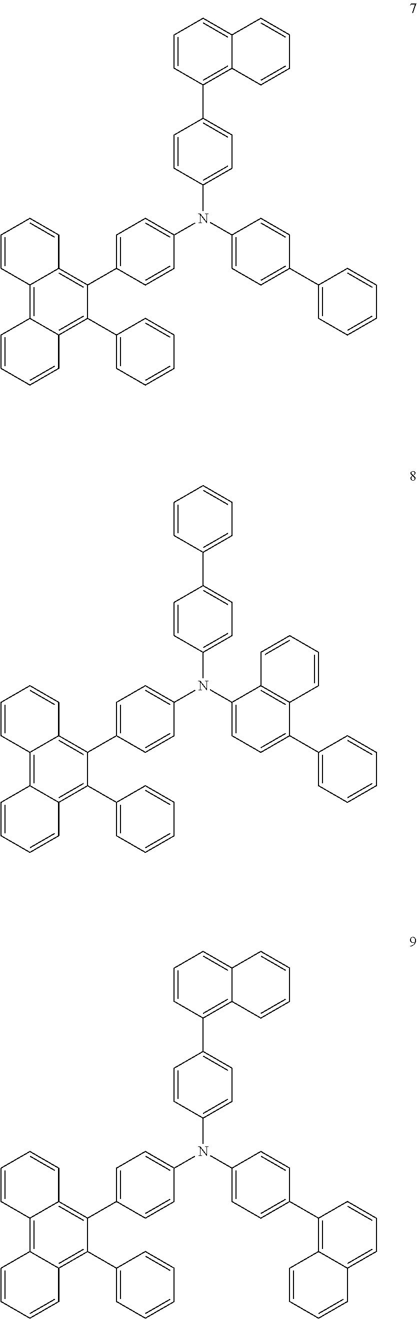 [SMILES: c1ccc(-c2ccc(N(c3ccc(-c4cccc5ccccc45)cc3)c3ccc(-c4c(-c5ccccc5)c5ccccc5c5ccccc45)cc3)cc2)cc1 ; c1ccc(-c2ccc(N(c3ccc(-c4c(-c5ccccc5)c5ccccc5c5ccccc45)cc3)c3ccc(-c4ccccc4)c4ccccc34)cc2)cc1 ; c1ccc(-c2c(-c3ccc(N(c4ccc(-c5cccc6ccccc56)cc4)c4ccc(-c5cccc6ccccc56)cc4)cc3)c3ccccc3c3ccccc23)cc1]

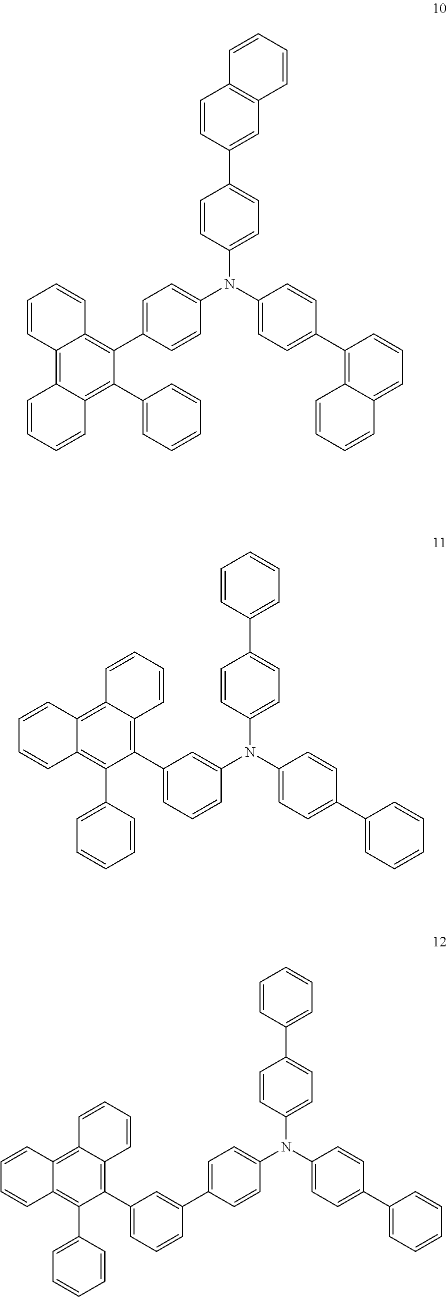 [SMILES: c1ccc(-c2c(-c3ccc(N(c4ccc(-c5ccc6ccccc6c5)cc4)c4ccc(-c5cccc6ccccc56)cc4)cc3)c3ccccc3c3ccccc23)cc1 ; c1ccc(-c2ccc(N(c3ccc(-c4ccccc4)cc3)c3cccc(-c4c(-c5ccccc5)c5ccccc5c5ccccc45)c3)cc2)cc1 ; c1ccc(-c2ccc(N(c3ccc(-c4ccccc4)cc3)c3ccc(-c4cccc(-c5c(-c6ccccc6)c6ccccc6c6ccccc56)c4)cc3)cc2)cc1]

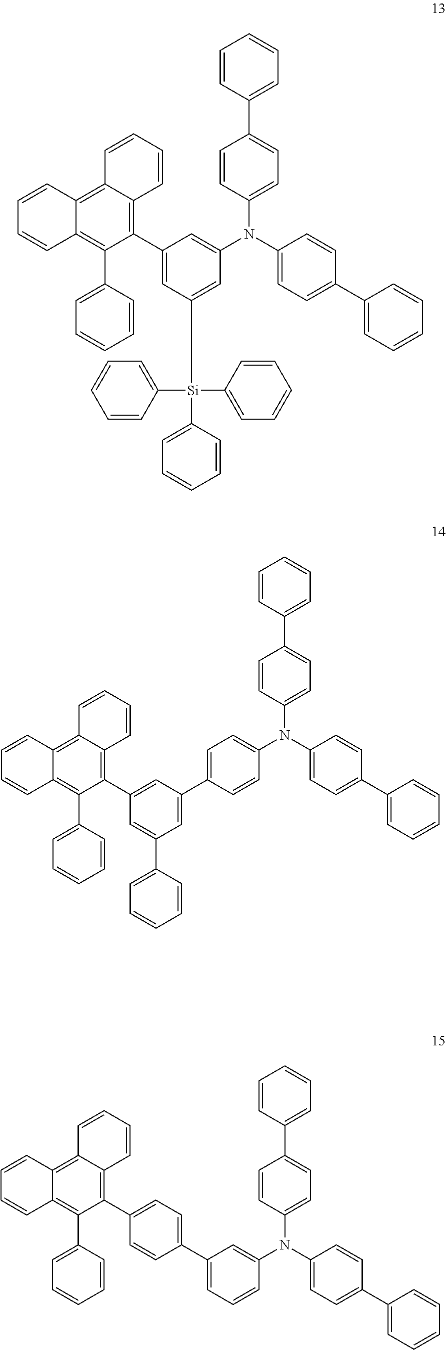 [SMILES: c1ccc(-c2ccc(N(c3ccc(-c4ccccc4)cc3)c3cc(-c4c(-c5ccccc5)c5ccccc5c5ccccc45)cc([Si](c4ccccc4)(c4ccccc4)c4ccccc4)c3)cc2)cc1 ; c1ccc(-c2ccc(N(c3ccc(-c4ccccc4)cc3)c3ccc(-c4cc(-c5ccccc5)cc(-c5c(-c6ccccc6)c6ccccc6c6ccccc56)c4)cc3)cc2)cc1 ; c1ccc(-c2ccc(N(c3ccc(-c4ccccc4)cc3)c3cccc(-c4ccc(-c5c(-c6ccccc6)c6ccccc6c6ccccc56)cc4)c3)cc2)cc1]

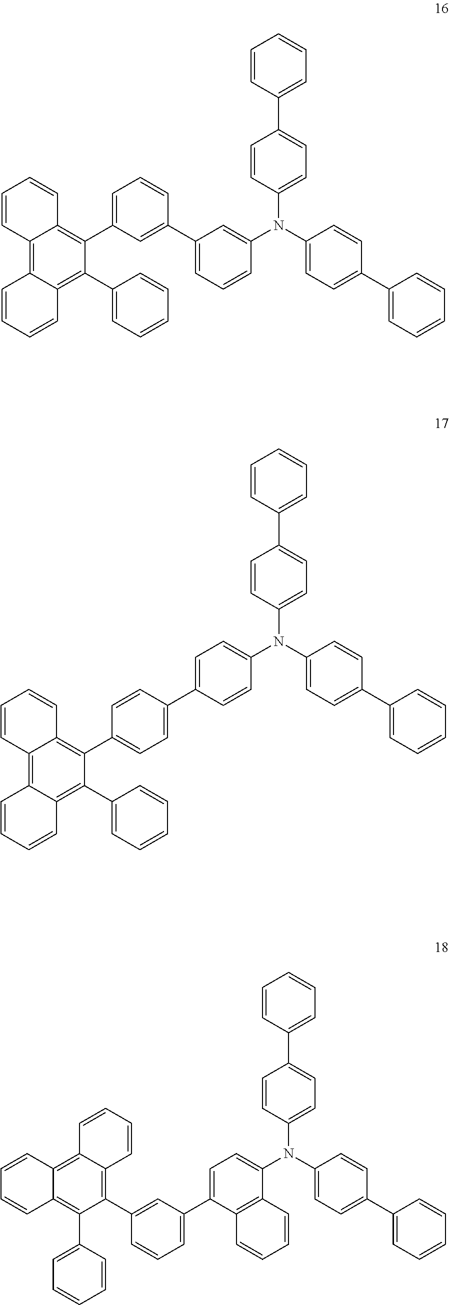 [SMILES: c1ccc(-c2ccc(N(c3ccc(-c4ccccc4)cc3)c3cccc(-c4cccc(-c5c(-c6ccccc6)c6ccccc6c6ccccc56)c4)c3)cc2)cc1 ; c1ccc(-c2ccc(N(c3ccc(-c4ccccc4)cc3)c3ccc(-c4ccc(-c5c(-c6ccccc6)c6ccccc6c6ccccc56)cc4)cc3)cc2)cc1 ; c1ccc(-c2ccc(N(c3ccc(-c4ccccc4)cc3)c3ccc(-c4cccc(-c5c(-c6ccccc6)c6ccccc6c6ccccc56)c4)c4ccccc34)cc2)cc1]

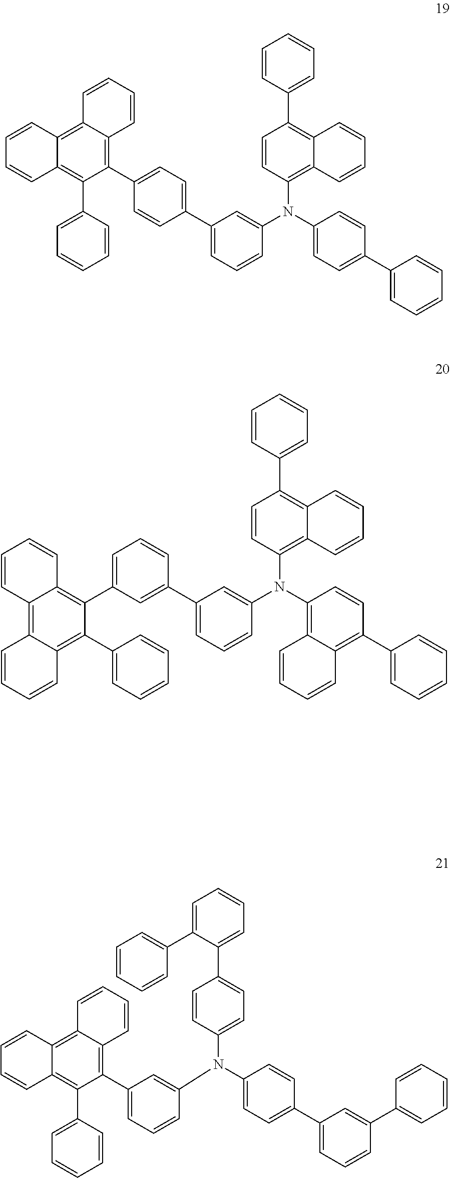 [SMILES: c1ccc(-c2ccc(N(c3cccc(-c4ccc(-c5c(-c6ccccc6)c6ccccc6c6ccccc56)cc4)c3)c3ccc(-c4ccccc4)c4ccccc34)cc2)cc1 ; c1ccc(-c2ccc(N(c3cccc(-c4cccc(-c5c(-c6ccccc6)c6ccccc6c6ccccc56)c4)c3)c3ccc(-c4ccccc4)c4ccccc34)c3ccccc23)cc1 ; c1ccc(-c2cccc(-c3ccc(N(c4ccc(-c5ccccc5-c5ccccc5)cc4)c4cccc(-c5c(-c6ccccc6)c6ccccc6c6ccccc56)c4)cc3)c2)cc1]

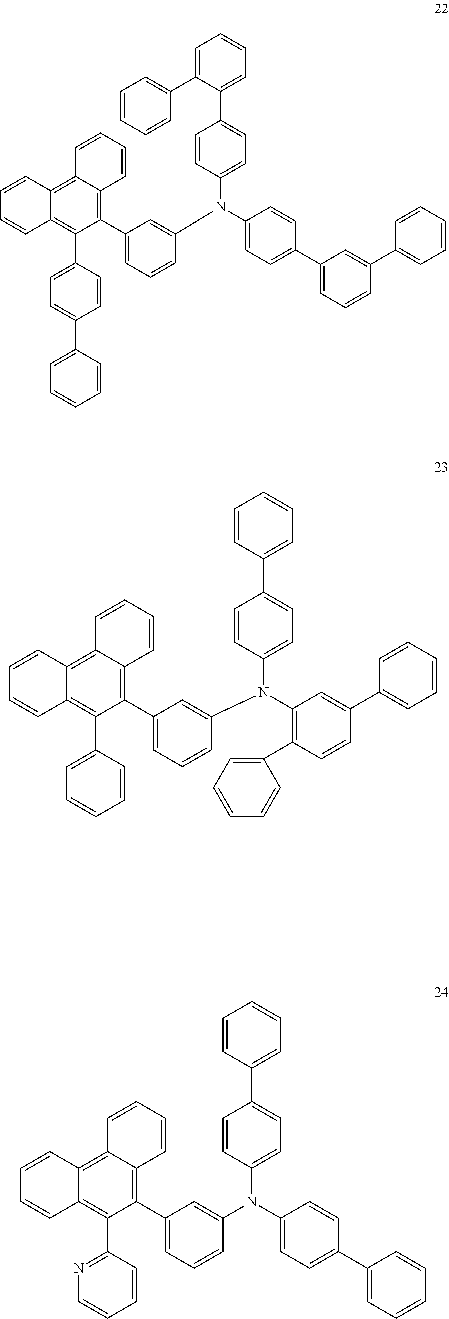 [SMILES: c1ccc(-c2ccc(-c3c(-c4cccc(N(c5ccc(-c6cccc(-c7ccccc7)c6)cc5)c5ccc(-c6ccccc6-c6ccccc6)cc5)c4)c4ccccc4c4ccccc34)cc2)cc1 ; c1ccc(-c2ccc(N(c3cccc(-c4c(-c5ccccc5)c5ccccc5c5ccccc45)c3)c3cc(-c4ccccc4)ccc3-c3ccccc3)cc2)cc1 ; c1ccc(-c2ccc(N(c3ccc(-c4ccccc4)cc3)c3cccc(-c4c(-c5ccccn5)c5ccccc5c5ccccc45)c3)cc2)cc1]

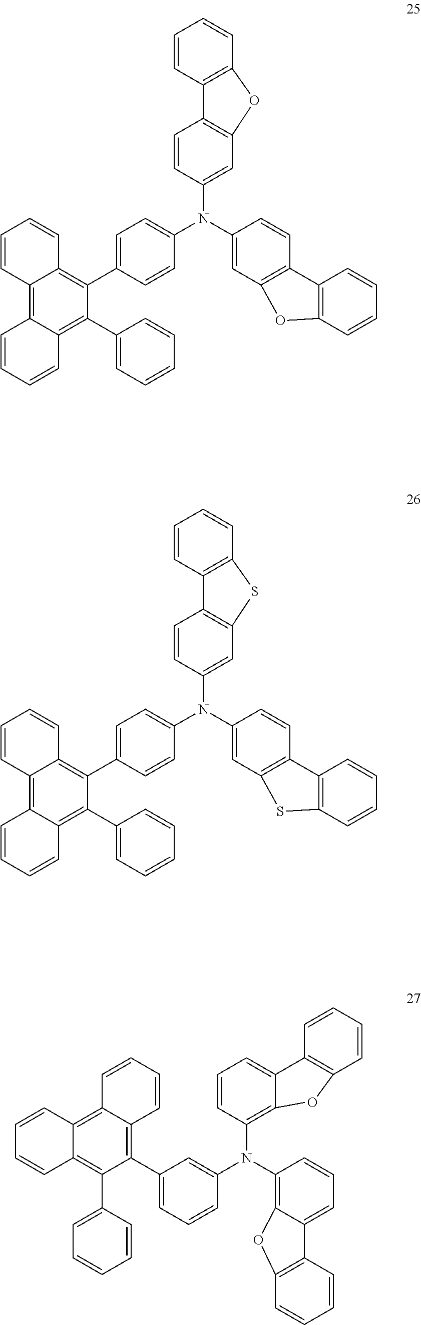 [SMILES: c1ccc(-c2c(-c3ccc(N(c4ccc5c(c4)oc4ccccc45)c4ccc5c(c4)oc4ccccc45)cc3)c3ccccc3c3ccccc23)cc1 ; c1ccc(-c2c(-c3ccc(N(c4ccc5c(c4)sc4ccccc45)c4ccc5c(c4)sc4ccccc45)cc3)c3ccccc3c3ccccc23)cc1 ; c1ccc(-c2c(-c3cccc(N(c4cccc5c4oc4ccccc45)c4cccc5c4oc4ccccc45)c3)c3ccccc3c3ccccc23)cc1]

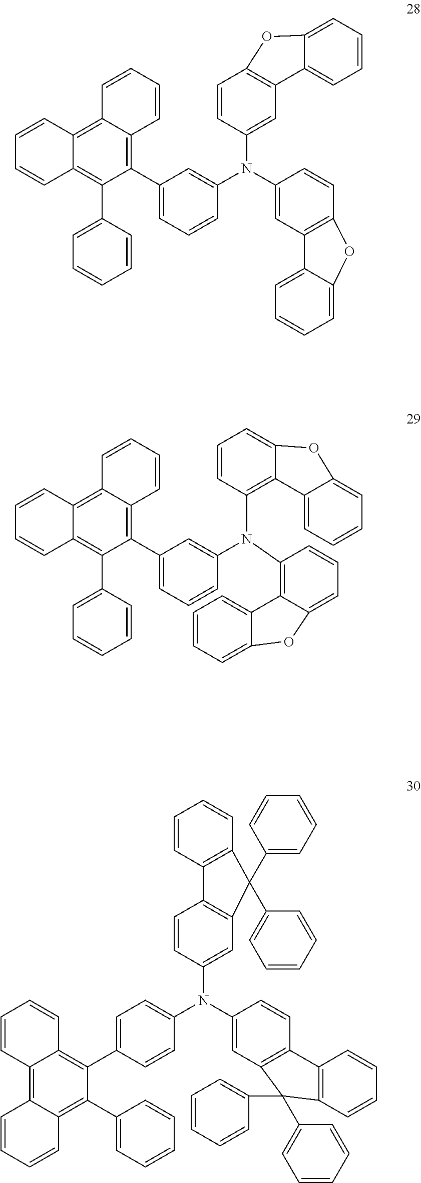 [SMILES: c1ccc(-c2c(-c3cccc(N(c4ccc5oc6ccccc6c5c4)c4ccc5oc6ccccc6c5c4)c3)c3ccccc3c3ccccc23)cc1 ; c1ccc(-c2c(-c3cccc(N(c4cccc5oc6ccccc6c45)c4cccc5oc6ccccc6c45)c3)c3ccccc3c3ccccc23)cc1 ; c1ccc(-c2c(-c3ccc(N(c4ccc5c(c4)C(c4ccccc4)(c4ccccc4)c4ccccc4-5)c4ccc5c(c4)C(c4ccccc4)(c4ccccc4)c4ccccc4-5)cc3)c3ccccc3c3ccccc23)cc1]

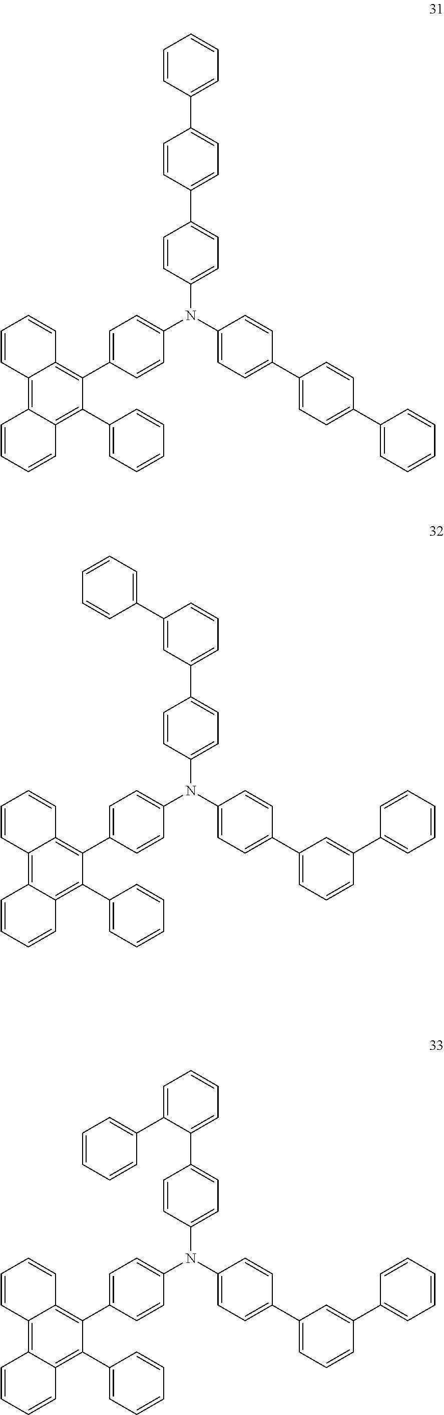 [SMILES: c1ccc(-c2ccc(-c3ccc(N(c4ccc(-c5ccc(-c6ccccc6)cc5)cc4)c4ccc(-c5c(-c6ccccc6)c6ccccc6c6ccccc56)cc4)cc3)cc2)cc1 ; c1ccc(-c2cccc(-c3ccc(N(c4ccc(-c5cccc(-c6ccccc6)c5)cc4)c4ccc(-c5c(-c6ccccc6)c6ccccc6c6ccccc56)cc4)cc3)c2)cc1 ; c1ccc(-c2cccc(-c3ccc(N(c4ccc(-c5ccccc5-c5ccccc5)cc4)c4ccc(-c5c(-c6ccccc6)c6ccccc6c6ccccc56)cc4)cc3)c2)cc1]

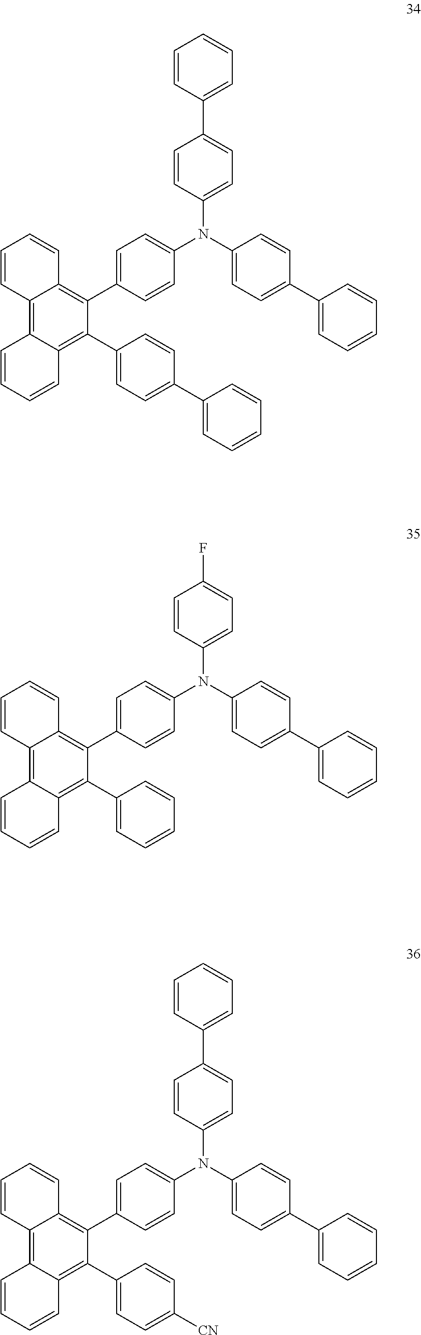 [SMILES: c1ccc(-c2ccc(-c3c(-c4ccc(N(c5ccc(-c6ccccc6)cc5)c5ccc(-c6ccccc6)cc5)cc4)c4ccccc4c4ccccc34)cc2)cc1 ; Fc1ccc(N(c2ccc(-c3ccccc3)cc2)c2ccc(-c3c(-c4ccccc4)c4ccccc4c4ccccc34)cc2)cc1 ; N#Cc1ccc(-c2c(-c3ccc(N(c4ccc(-c5ccccc5)cc4)c4ccc(-c5ccccc5)cc4)cc3)c3ccccc3c3ccccc23)cc1]

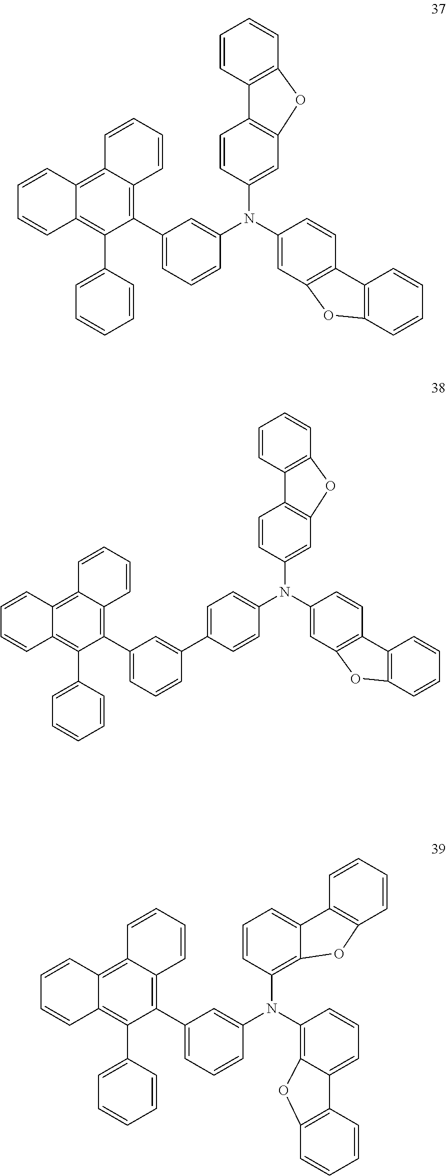 [SMILES: c1ccc(-c2c(-c3cccc(N(c4ccc5c(c4)oc4ccccc45)c4ccc5c(c4)oc4ccccc45)c3)c3ccccc3c3ccccc23)cc1 ; c1ccc(-c2c(-c3cccc(-c4ccc(N(c5ccc6c(c5)oc5ccccc56)c5ccc6c(c5)oc5ccccc56)cc4)c3)c3ccccc3c3ccccc23)cc1 ; c1ccc(-c2c(-c3cccc(N(c4cccc5c4oc4ccccc45)c4cccc5c4oc4ccccc45)c3)c3ccccc3c3ccccc23)cc1]

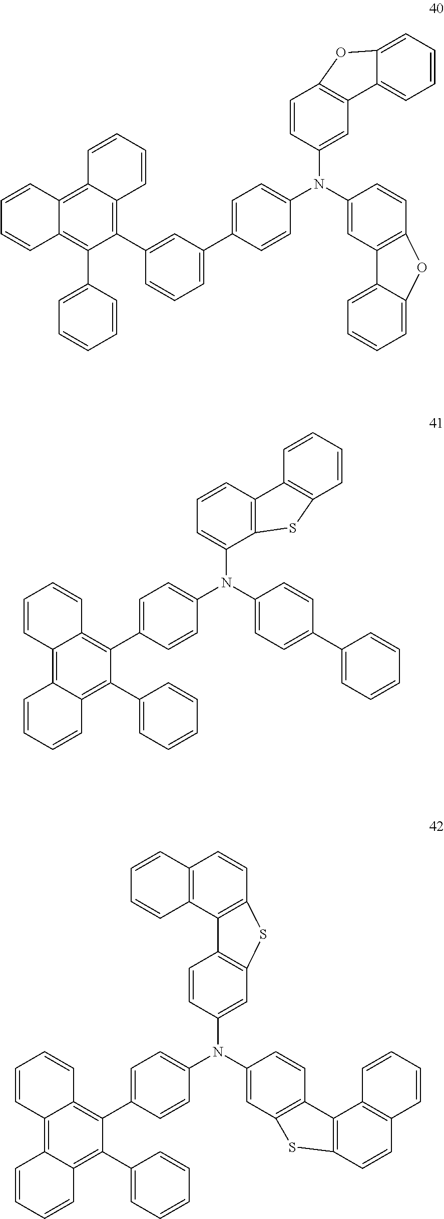 [SMILES: c1ccc(-c2c(-c3cccc(-c4ccc(N(c5ccc6oc7ccccc7c6c5)c5ccc6oc7ccccc7c6c5)cc4)c3)c3ccccc3c3ccccc23)cc1 ; c1ccc(-c2ccc(N(c3ccc(-c4c(-c5ccccc5)c5ccccc5c5ccccc45)cc3)c3cccc4c3sc3ccccc34)cc2)cc1 ; c1ccc(-c2c(-c3ccc(N(c4ccc5c(c4)sc4ccc6ccccc6c45)c4ccc5c(c4)sc4ccc6ccccc6c45)cc3)c3ccccc3c3ccccc23)cc1]

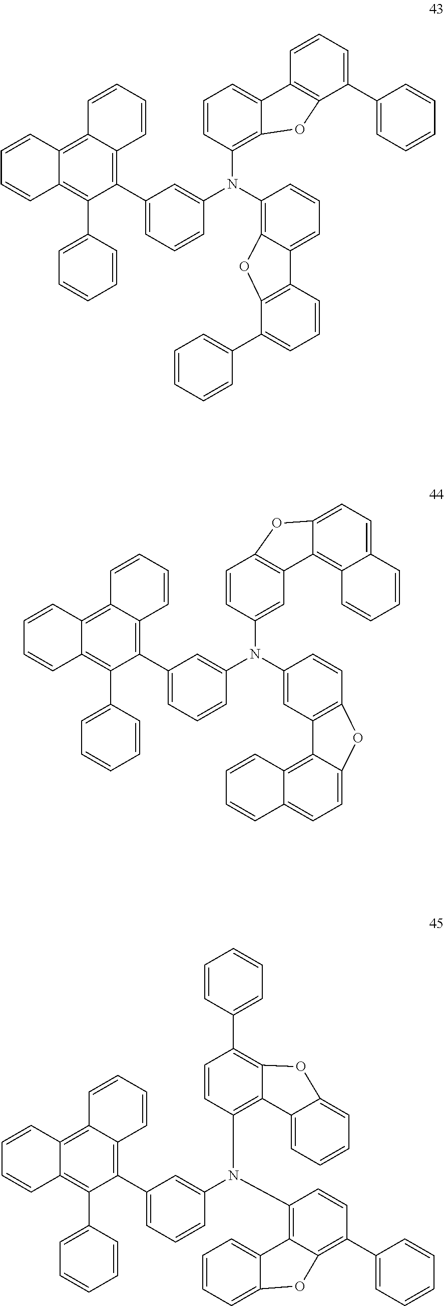 [SMILES: c1ccc(-c2c(-c3cccc(N(c4cccc5c4oc4c(-c6ccccc6)cccc45)c4cccc5c4oc4c(-c6ccccc6)cccc45)c3)c3ccccc3c3ccccc23)cc1 ; c1ccc(-c2c(-c3cccc(N(c4ccc5oc6ccc7ccccc7c6c5c4)c4ccc5oc6ccc7ccccc7c6c5c4)c3)c3ccccc3c3ccccc23)cc1 ; c1ccc(-c2c(-c3cccc(N(c4ccc(-c5ccccc5)c5oc6ccccc6c45)c4ccc(-c5ccccc5)c5oc6ccccc6c45)c3)c3ccccc3c3ccccc23)cc1]

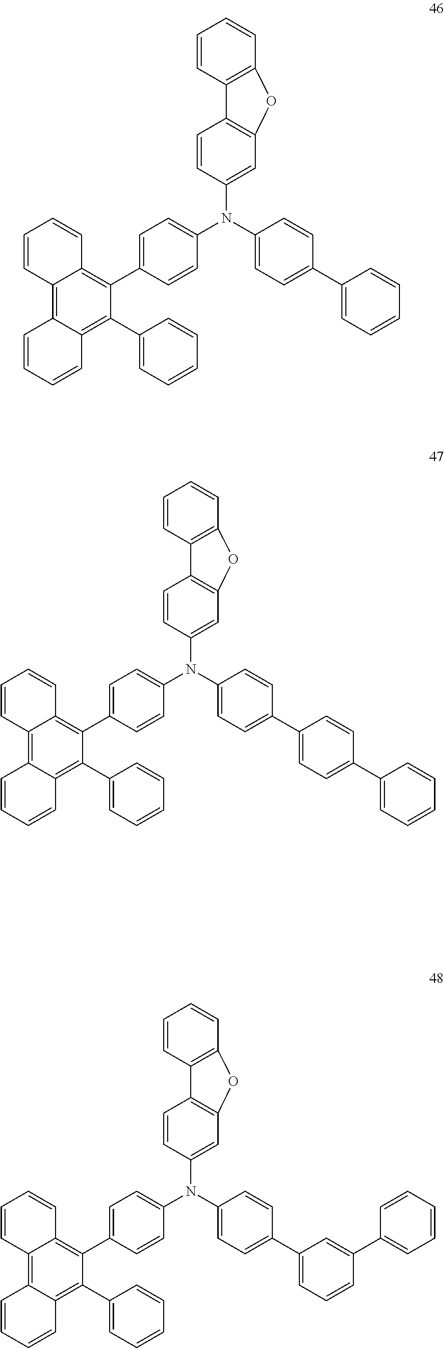 [SMILES: c1ccc(-c2ccc(N(c3ccc(-c4c(-c5ccccc5)c5ccccc5c5ccccc45)cc3)c3ccc4c(c3)oc3ccccc34)cc2)cc1 ; c1ccc(-c2ccc(-c3ccc(N(c4ccc(-c5c(-c6ccccc6)c6ccccc6c6ccccc56)cc4)c4ccc5c(c4)oc4ccccc45)cc3)cc2)cc1 ; c1ccc(-c2cccc(-c3ccc(N(c4ccc(-c5c(-c6ccccc6)c6ccccc6c6ccccc56)cc4)c4ccc5c(c4)oc4ccccc45)cc3)c2)cc1]

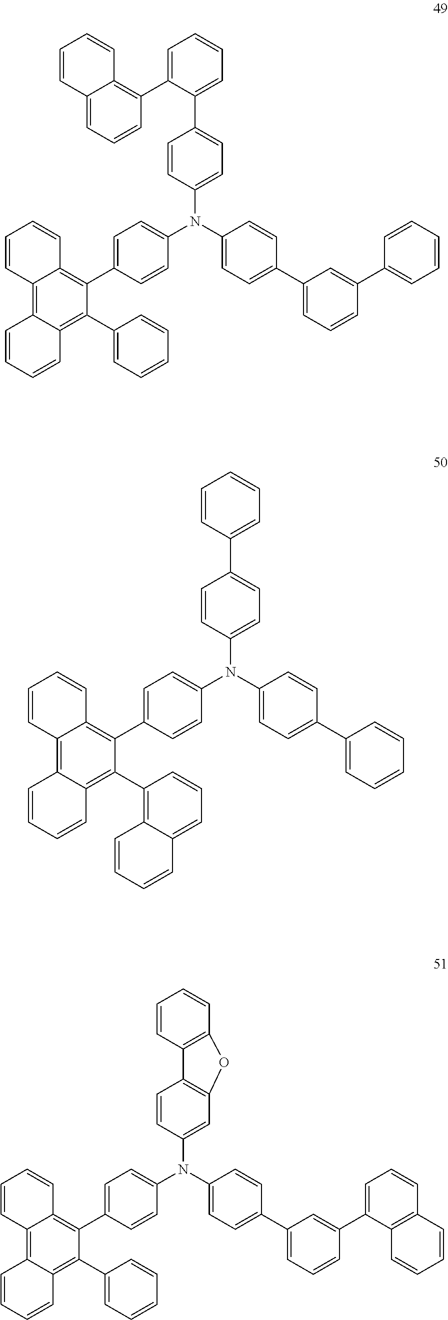 [SMILES: c1ccc(-c2cccc(-c3ccc(N(c4ccc(-c5ccccc5-c5cccc6ccccc56)cc4)c4ccc(-c5c(-c6ccccc6)c6ccccc6c6ccccc56)cc4)cc3)c2)cc1 ; c1ccc(-c2ccc(N(c3ccc(-c4ccccc4)cc3)c3ccc(-c4c(-c5cccc6ccccc56)c5ccccc5c5ccccc45)cc3)cc2)cc1 ; c1ccc(-c2c(-c3ccc(N(c4ccc(-c5cccc(-c6cccc7ccccc67)c5)cc4)c4ccc5c(c4)oc4ccccc45)cc3)c3ccccc3c3ccccc23)cc1]

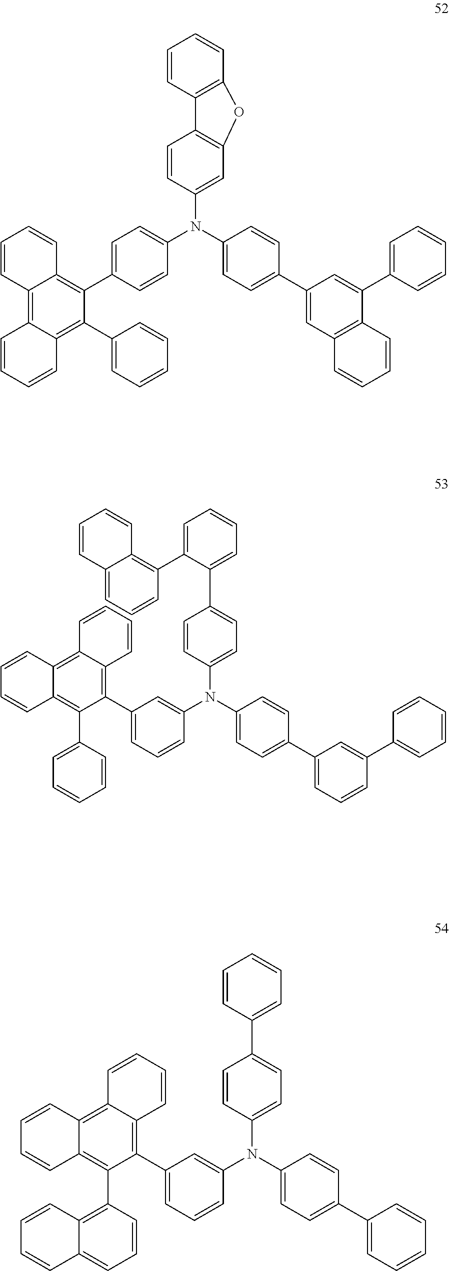 [SMILES: c1ccc(-c2cc(-c3ccc(N(c4ccc(-c5c(-c6ccccc6)c6ccccc6c6ccccc56)cc4)c4ccc5c(c4)oc4ccccc45)cc3)cc3ccccc23)cc1 ; c1ccc(-c2cccc(-c3ccc(N(c4ccc(-c5ccccc5-c5cccc6ccccc56)cc4)c4cccc(-c5c(-c6ccccc6)c6ccccc6c6ccccc56)c4)cc3)c2)cc1 ; c1ccc(-c2ccc(N(c3ccc(-c4ccccc4)cc3)c3cccc(-c4c(-c5cccc6ccccc56)c5ccccc5c5ccccc45)c3)cc2)cc1]

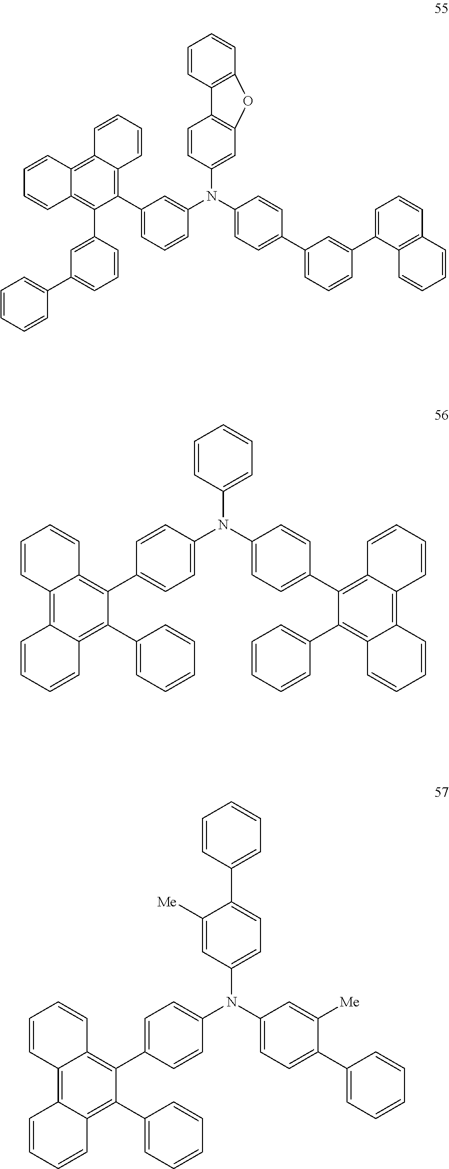 [SMILES: c1ccc(-c2cccc(-c3c(-c4cccc(N(c5ccc(-c6cccc(-c7cccc8ccccc78)c6)cc5)c5ccc6c(c5)oc5ccccc56)c4)c4ccccc4c4ccccc34)c2)cc1 ; c1ccc(-c2c(-c3ccc(N(c4ccccc4)c4ccc(-c5c(-c6ccccc6)c6ccccc6c6ccccc56)cc4)cc3)c3ccccc3c3ccccc23)cc1 ; Cc1cc(N(c2ccc(-c3c(-c4ccccc4)c4ccccc4c4ccccc34)cc2)c2ccc(-c3ccccc3)c(C)c2)ccc1-c1ccccc1]

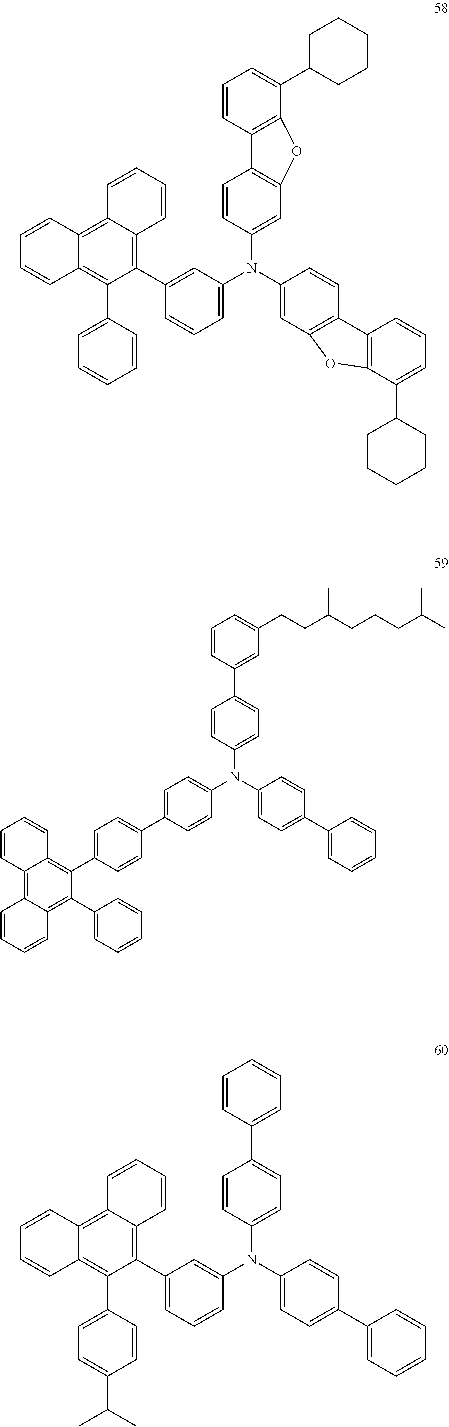 [SMILES: c1ccc(-c2c(-c3cccc(N(c4ccc5c(c4)oc4c(C6CCCCC6)cccc45)c4ccc5c(c4)oc4c(C6CCCCC6)cccc45)c3)c3ccccc3c3ccccc23)cc1 ; CC(C)CCCC(C)CCc1cccc(-c2ccc(N(c3ccc(-c4ccccc4)cc3)c3ccc(-c4ccc(-c5c(-c6ccccc6)c6ccccc6c6ccccc56)cc4)cc3)cc2)c1 ; CC(C)c1ccc(-c2c(-c3cccc(N(c4ccc(-c5ccccc5)cc4)c4ccc(-c5ccccc5)cc4)c3)c3ccccc3c3ccccc23)cc1]

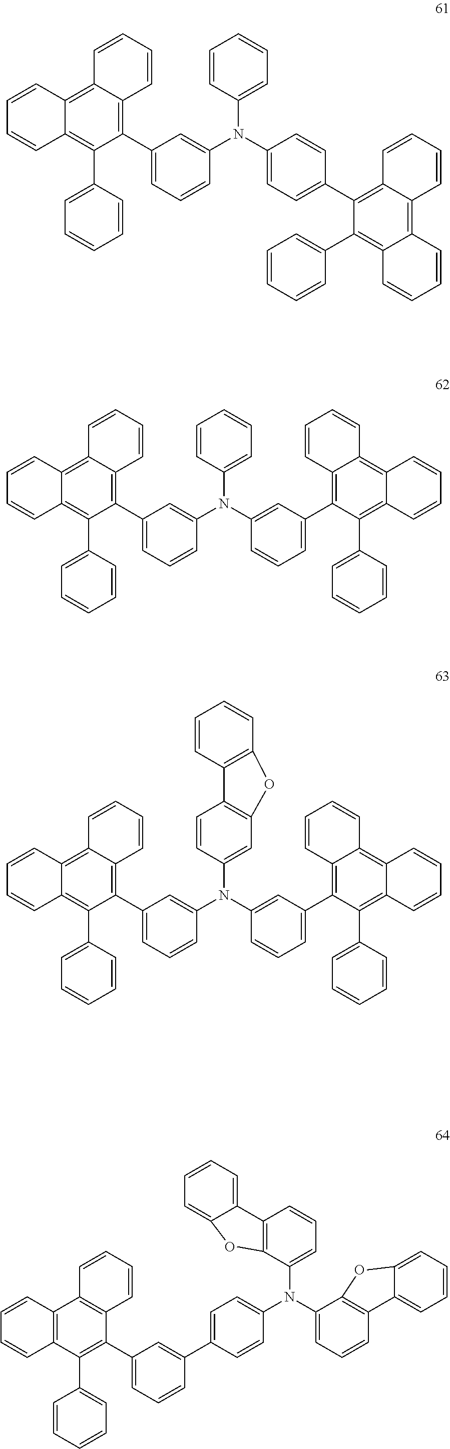 [SMILES: c1ccc(-c2c(-c3ccc(N(c4ccccc4)c4cccc(-c5c(-c6ccccc6)c6ccccc6c6ccccc56)c4)cc3)c3ccccc3c3ccccc23)cc1 ; c1ccc(-c2c(-c3cccc(N(c4ccccc4)c4cccc(-c5c(-c6ccccc6)c6ccccc6c6ccccc56)c4)c3)c3ccccc3c3ccccc23)cc1 ; c1ccc(-c2c(-c3cccc(N(c4cccc(-c5c(-c6ccccc6)c6ccccc6c6ccccc56)c4)c4ccc5c(c4)oc4ccccc45)c3)c3ccccc3c3ccccc23)cc1 ; c1ccc(-c2c(-c3cccc(-c4ccc(N(c5cccc6c5oc5ccccc56)c5cccc6c5oc5ccccc56)cc4)c3)c3ccccc3c3ccccc23)cc1]

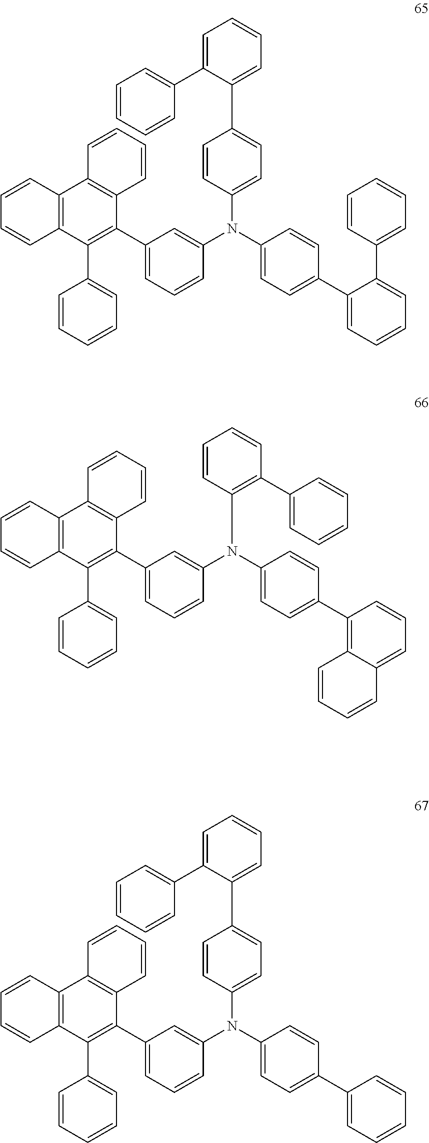 [SMILES: c1ccc(-c2ccccc2-c2ccc(N(c3ccc(-c4ccccc4-c4ccccc4)cc3)c3cccc(-c4c(-c5ccccc5)c5ccccc5c5ccccc45)c3)cc2)cc1 ; c1ccc(-c2ccccc2N(c2ccc(-c3cccc4ccccc34)cc2)c2cccc(-c3c(-c4ccccc4)c4ccccc4c4ccccc34)c2)cc1 ; c1ccc(-c2ccc(N(c3ccc(-c4ccccc4-c4ccccc4)cc3)c3cccc(-c4c(-c5ccccc5)c5ccccc5c5ccccc45)c3)cc2)cc1]

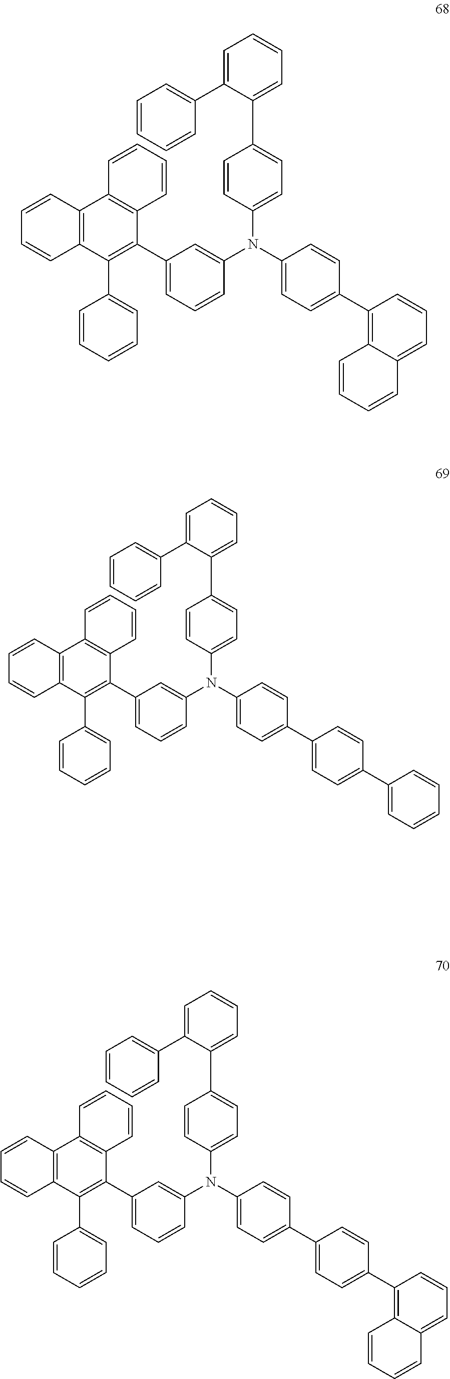 [SMILES: c1ccc(-c2ccccc2-c2ccc(N(c3ccc(-c4cccc5ccccc45)cc3)c3cccc(-c4c(-c5ccccc5)c5ccccc5c5ccccc45)c3)cc2)cc1 ; c1ccc(-c2ccc(-c3ccc(N(c4ccc(-c5ccccc5-c5ccccc5)cc4)c4cccc(-c5c(-c6ccccc6)c6ccccc6c6ccccc56)c4)cc3)cc2)cc1 ; c1ccc(-c2ccccc2-c2ccc(N(c3ccc(-c4ccc(-c5cccc6ccccc56)cc4)cc3)c3cccc(-c4c(-c5ccccc5)c5ccccc5c5ccccc45)c3)cc2)cc1]

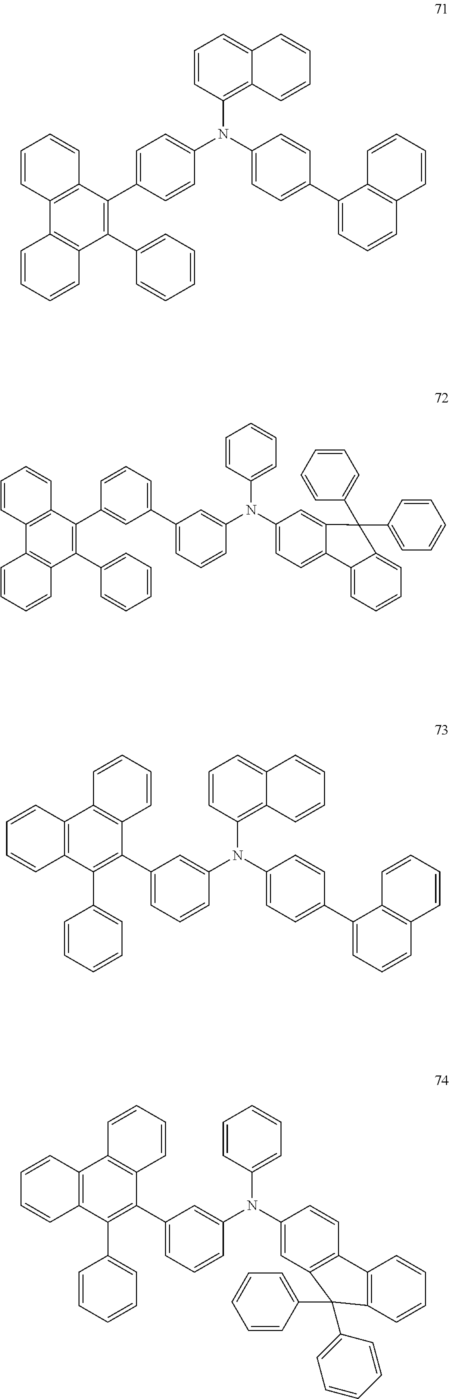 [SMILES: c1ccc(-c2c(-c3ccc(N(c4ccc(-c5cccc6ccccc56)cc4)c4cccc5ccccc45)cc3)c3ccccc3c3ccccc23)cc1 ; c1ccc(-c2c(-c3cccc(-c4cccc(N(c5ccccc5)c5ccc6c(c5)C(c5ccccc5)(c5ccccc5)c5ccccc5-6)c4)c3)c3ccccc3c3ccccc23)cc1 ; c1ccc(-c2c(-c3cccc(N(c4ccc(-c5cccc6ccccc56)cc4)c4cccc5ccccc45)c3)c3ccccc3c3ccccc23)cc1 ; c1ccc(-c2c(-c3cccc(N(c4ccccc4)c4ccc5c(c4)C(c4ccccc4)(c4ccccc4)c4ccccc4-5)c3)c3ccccc3c3ccccc23)cc1]

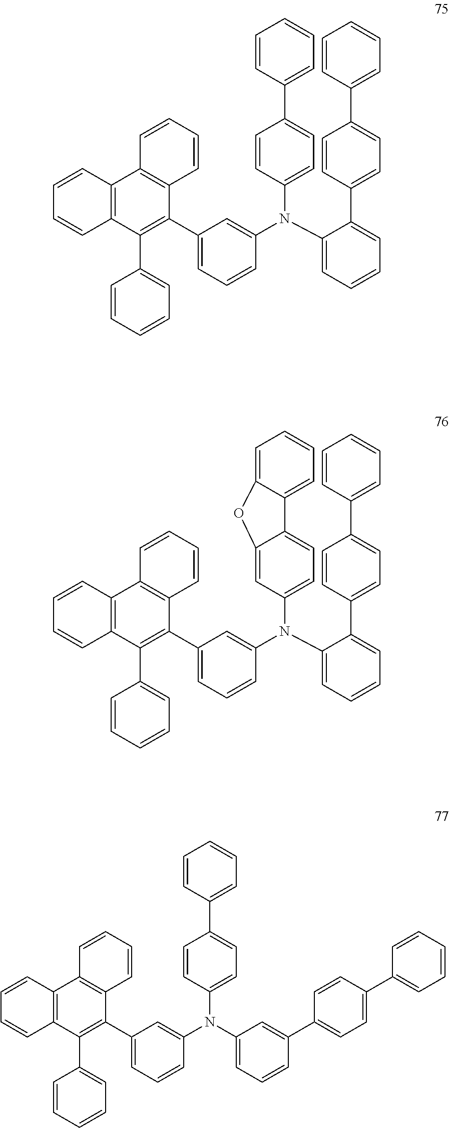 [SMILES: c1ccc(-c2ccc(-c3ccccc3N(c3ccc(-c4ccccc4)cc3)c3cccc(-c4c(-c5ccccc5)c5ccccc5c5ccccc45)c3)cc2)cc1 ; c1ccc(-c2ccc(-c3ccccc3N(c3cccc(-c4c(-c5ccccc5)c5ccccc5c5ccccc45)c3)c3ccc4c(c3)oc3ccccc34)cc2)cc1 ; c1ccc(-c2ccc(-c3cccc(N(c4ccc(-c5ccccc5)cc4)c4cccc(-c5c(-c6ccccc6)c6ccccc6c6ccccc56)c4)c3)cc2)cc1]

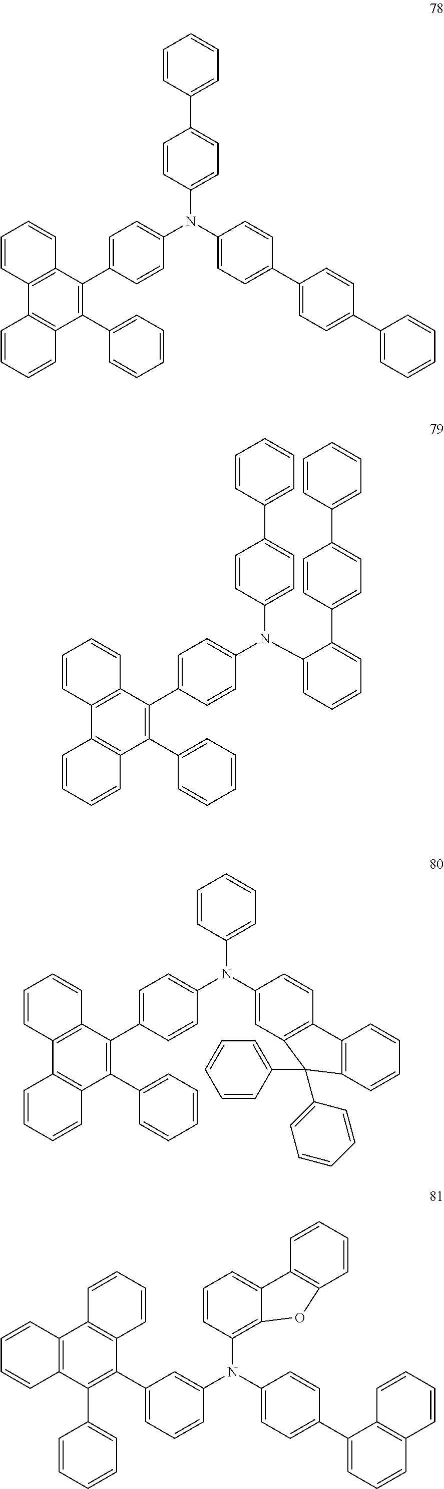 [SMILES: c1ccc(-c2ccc(-c3ccc(N(c4ccc(-c5ccccc5)cc4)c4ccc(-c5c(-c6ccccc6)c6ccccc6c6ccccc56)cc4)cc3)cc2)cc1 ; c1ccc(-c2ccc(-c3ccccc3N(c3ccc(-c4ccccc4)cc3)c3ccc(-c4c(-c5ccccc5)c5ccccc5c5ccccc45)cc3)cc2)cc1 ; c1ccc(-c2c(-c3ccc(N(c4ccccc4)c4ccc5c(c4)C(c4ccccc4)(c4ccccc4)c4ccccc4-5)cc3)c3ccccc3c3ccccc23)cc1 ; c1ccc(-c2c(-c3cccc(N(c4ccc(-c5cccc6ccccc56)cc4)c4cccc5c4oc4ccccc45)c3)c3ccccc3c3ccccc23)cc1]

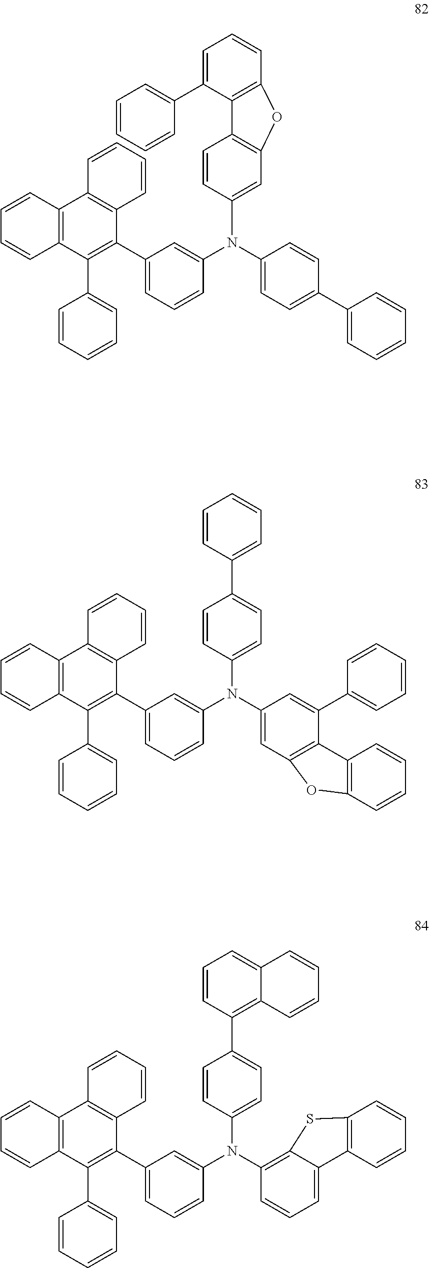 [SMILES: c1ccc(-c2ccc(N(c3cccc(-c4c(-c5ccccc5)c5ccccc5c5ccccc45)c3)c3ccc4c(c3)oc3cccc(-c5ccccc5)c34)cc2)cc1 ; c1ccc(-c2ccc(N(c3cccc(-c4c(-c5ccccc5)c5ccccc5c5ccccc45)c3)c3cc(-c4ccccc4)c4c(c3)oc3ccccc34)cc2)cc1 ; c1ccc(-c2c(-c3cccc(N(c4ccc(-c5cccc6ccccc56)cc4)c4cccc5c4sc4ccccc45)c3)c3ccccc3c3ccccc23)cc1]

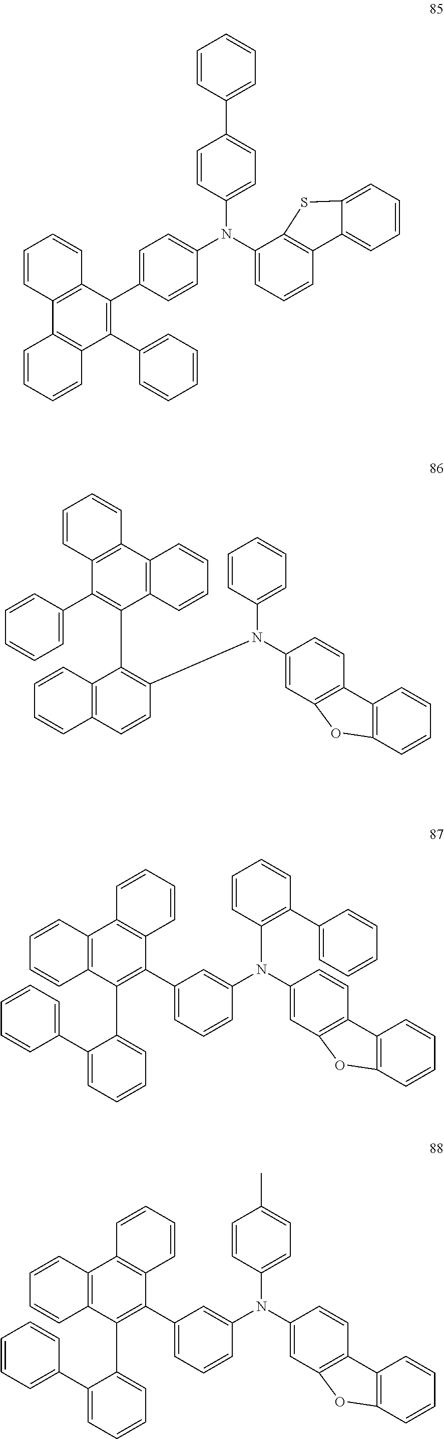 [SMILES: c1ccc(-c2ccc(N(c3ccc(-c4c(-c5ccccc5)c5ccccc5c5ccccc45)cc3)c3cccc4c3sc3ccccc34)cc2)cc1 ; c1ccc(-c2c(-c3c(N(c4ccccc4)c4ccc5c(c4)oc4ccccc45)ccc4ccccc34)c3ccccc3c3ccccc23)cc1 ; c1ccc(-c2ccccc2-c2c(-c3cccc(N(c4ccc5c(c4)oc4ccccc45)c4ccccc4-c4ccccc4)c3)c3ccccc3c3ccccc23)cc1 ; Cc1ccc(N(c2cccc(-c3c(-c4ccccc4-c4ccccc4)c4ccccc4c4ccccc34)c2)c2ccc3c(c2)oc2ccccc23)cc1]

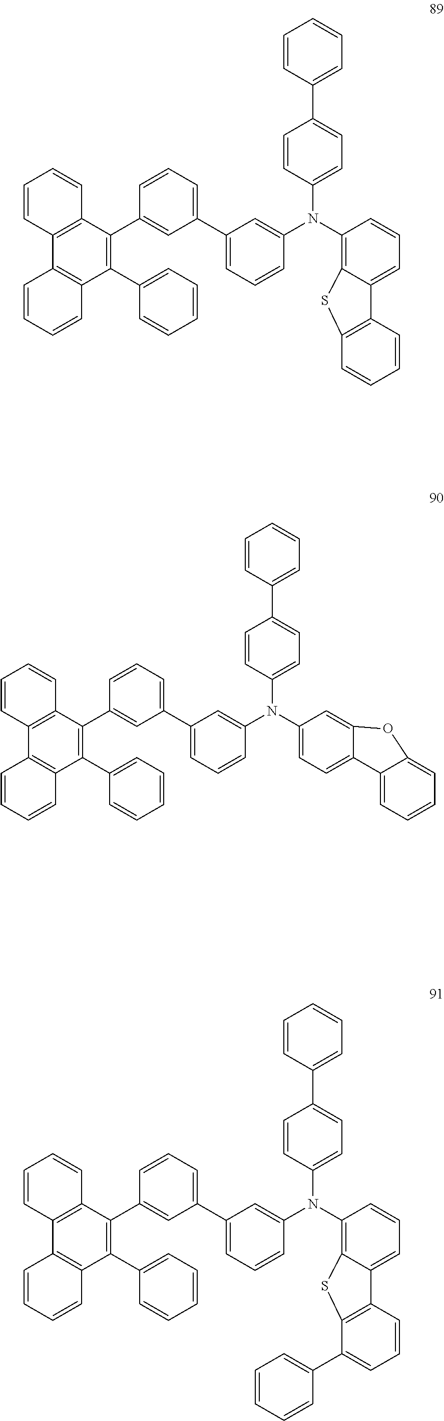 [SMILES: c1ccc(-c2ccc(N(c3cccc(-c4cccc(-c5c(-c6ccccc6)c6ccccc6c6ccccc56)c4)c3)c3cccc4c3sc3ccccc34)cc2)cc1 ; c1ccc(-c2ccc(N(c3cccc(-c4cccc(-c5c(-c6ccccc6)c6ccccc6c6ccccc56)c4)c3)c3ccc4c(c3)oc3ccccc34)cc2)cc1 ; c1ccc(-c2ccc(N(c3cccc(-c4cccc(-c5c(-c6ccccc6)c6ccccc6c6ccccc56)c4)c3)c3cccc4c3sc3c(-c5ccccc5)cccc34)cc2)cc1]

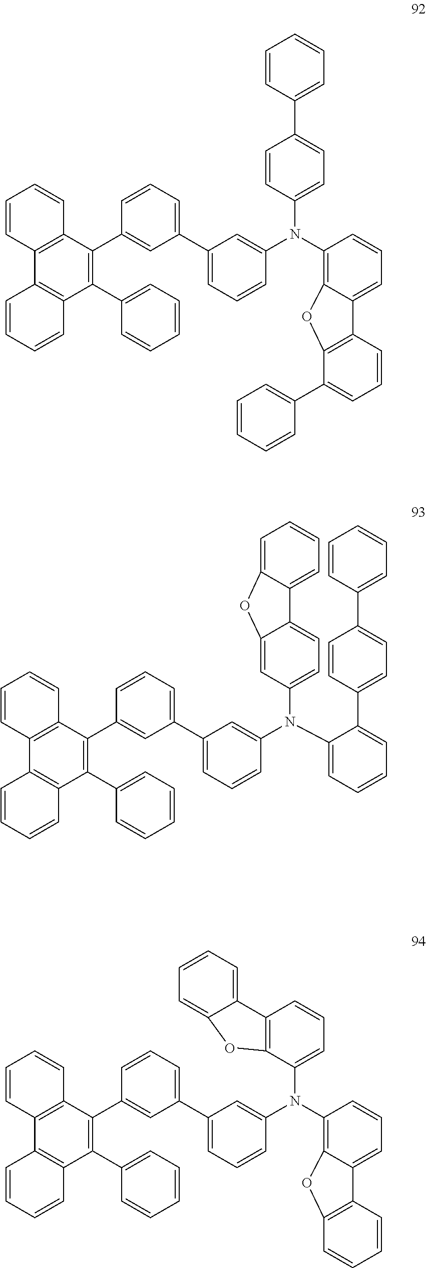 [SMILES: c1ccc(-c2ccc(N(c3cccc(-c4cccc(-c5c(-c6ccccc6)c6ccccc6c6ccccc56)c4)c3)c3cccc4c3oc3c(-c5ccccc5)cccc34)cc2)cc1 ; c1ccc(-c2ccc(-c3ccccc3N(c3cccc(-c4cccc(-c5c(-c6ccccc6)c6ccccc6c6ccccc56)c4)c3)c3ccc4c(c3)oc3ccccc34)cc2)cc1 ; c1ccc(-c2c(-c3cccc(-c4cccc(N(c5cccc6c5oc5ccccc56)c5cccc6c5oc5ccccc56)c4)c3)c3ccccc3c3ccccc23)cc1]

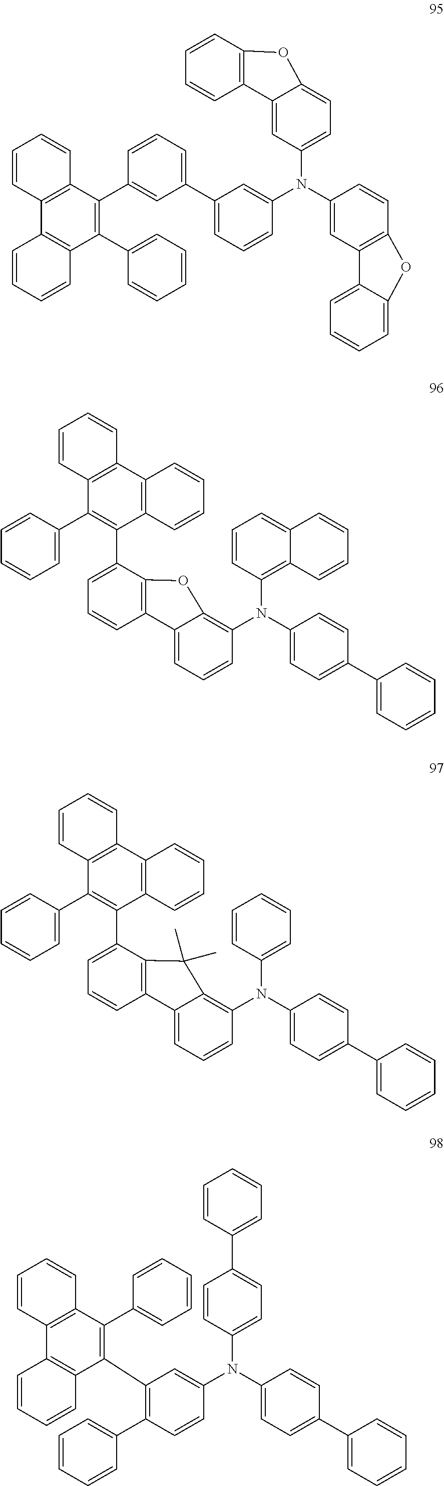 [SMILES: c1ccc(-c2c(-c3cccc(-c4cccc(N(c5ccc6oc7ccccc7c6c5)c5ccc6oc7ccccc7c6c5)c4)c3)c3ccccc3c3ccccc23)cc1 ; c1ccc(-c2ccc(N(c3cccc4ccccc34)c3cccc4c3oc3c(-c5c(-c6ccccc6)c6ccccc6c6ccccc56)cccc34)cc2)cc1 ; CC1(C)c2c(cccc2-c2c(-c3ccccc3)c3ccccc3c3ccccc23)-c2cccc(N(c3ccccc3)c3ccc(-c4ccccc4)cc3)c21 ; c1ccc(-c2ccc(N(c3ccc(-c4ccccc4)cc3)c3ccc(-c4ccccc4)c(-c4c(-c5ccccc5)c5ccccc5c5ccccc45)c3)cc2)cc1]

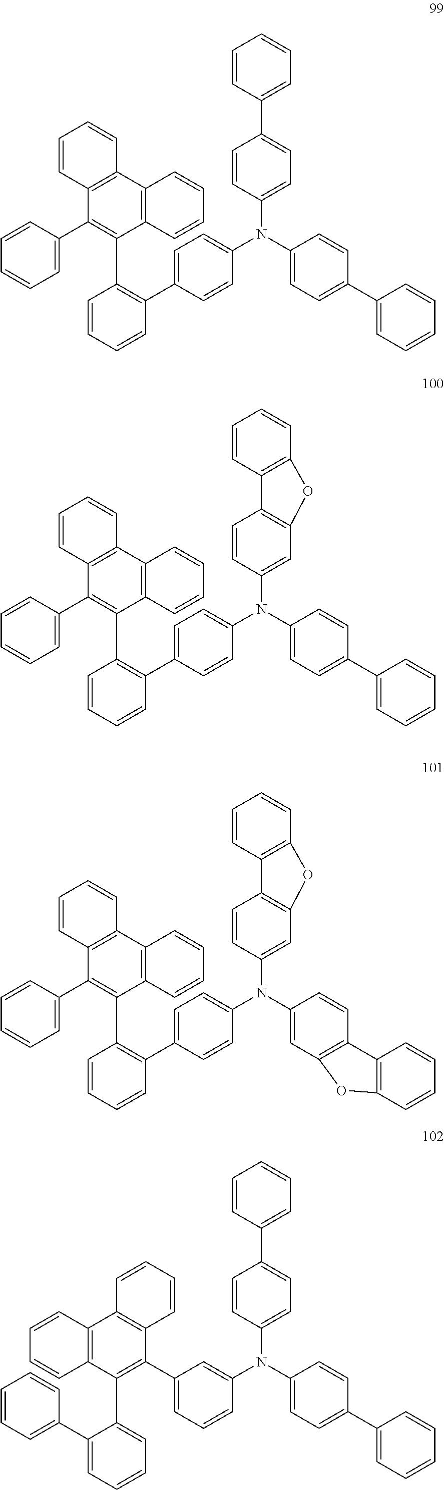 [SMILES: c1ccc(-c2ccc(N(c3ccc(-c4ccccc4)cc3)c3ccc(-c4ccccc4-c4c(-c5ccccc5)c5ccccc5c5ccccc45)cc3)cc2)cc1 ; c1ccc(-c2ccc(N(c3ccc(-c4ccccc4-c4c(-c5ccccc5)c5ccccc5c5ccccc45)cc3)c3ccc4c(c3)oc3ccccc34)cc2)cc1 ; c1ccc(-c2c(-c3ccccc3-c3ccc(N(c4ccc5c(c4)oc4ccccc45)c4ccc5c(c4)oc4ccccc45)cc3)c3ccccc3c3ccccc23)cc1 ; c1ccc(-c2ccc(N(c3ccc(-c4ccccc4)cc3)c3cccc(-c4c(-c5ccccc5-c5ccccc5)c5ccccc5c5ccccc45)c3)cc2)cc1]

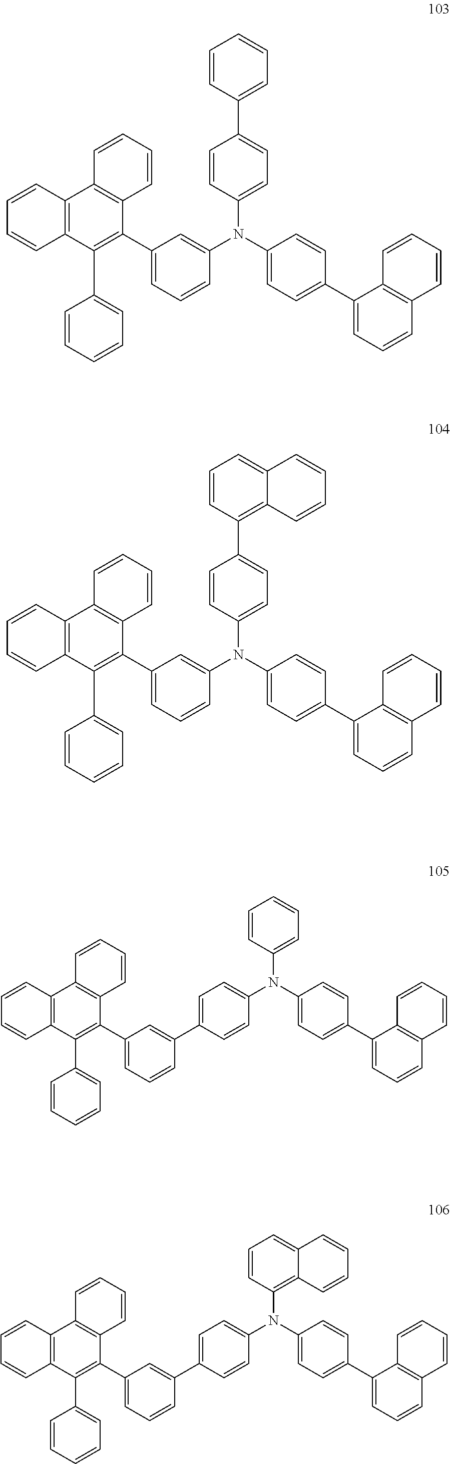 [SMILES: c1ccc(-c2ccc(N(c3ccc(-c4cccc5ccccc45)cc3)c3cccc(-c4c(-c5ccccc5)c5ccccc5c5ccccc45)c3)cc2)cc1 ; c1ccc(-c2c(-c3cccc(N(c4ccc(-c5cccc6ccccc56)cc4)c4ccc(-c5cccc6ccccc56)cc4)c3)c3ccccc3c3ccccc23)cc1 ; c1ccc(-c2c(-c3cccc(-c4ccc(N(c5ccccc5)c5ccc(-c6cccc7ccccc67)cc5)cc4)c3)c3ccccc3c3ccccc23)cc1 ; c1ccc(-c2c(-c3cccc(-c4ccc(N(c5ccc(-c6cccc7ccccc67)cc5)c5cccc6ccccc56)cc4)c3)c3ccccc3c3ccccc23)cc1]

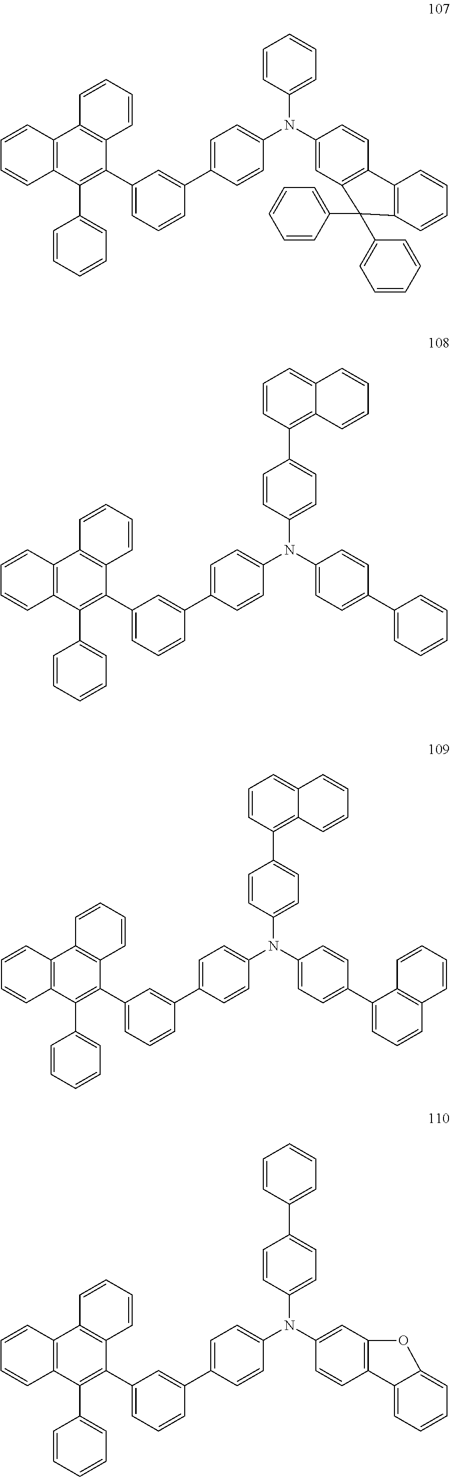 [SMILES: c1ccc(-c2c(-c3cccc(-c4ccc(N(c5ccccc5)c5ccc6c(c5)C(c5ccccc5)(c5ccccc5)c5ccccc5-6)cc4)c3)c3ccccc3c3ccccc23)cc1 ; c1ccc(-c2ccc(N(c3ccc(-c4cccc(-c5c(-c6ccccc6)c6ccccc6c6ccccc56)c4)cc3)c3ccc(-c4cccc5ccccc45)cc3)cc2)cc1 ; c1ccc(-c2c(-c3cccc(-c4ccc(N(c5ccc(-c6cccc7ccccc67)cc5)c5ccc(-c6cccc7ccccc67)cc5)cc4)c3)c3ccccc3c3ccccc23)cc1 ; c1ccc(-c2ccc(N(c3ccc(-c4cccc(-c5c(-c6ccccc6)c6ccccc6c6ccccc56)c4)cc3)c3ccc4c(c3)oc3ccccc34)cc2)cc1]

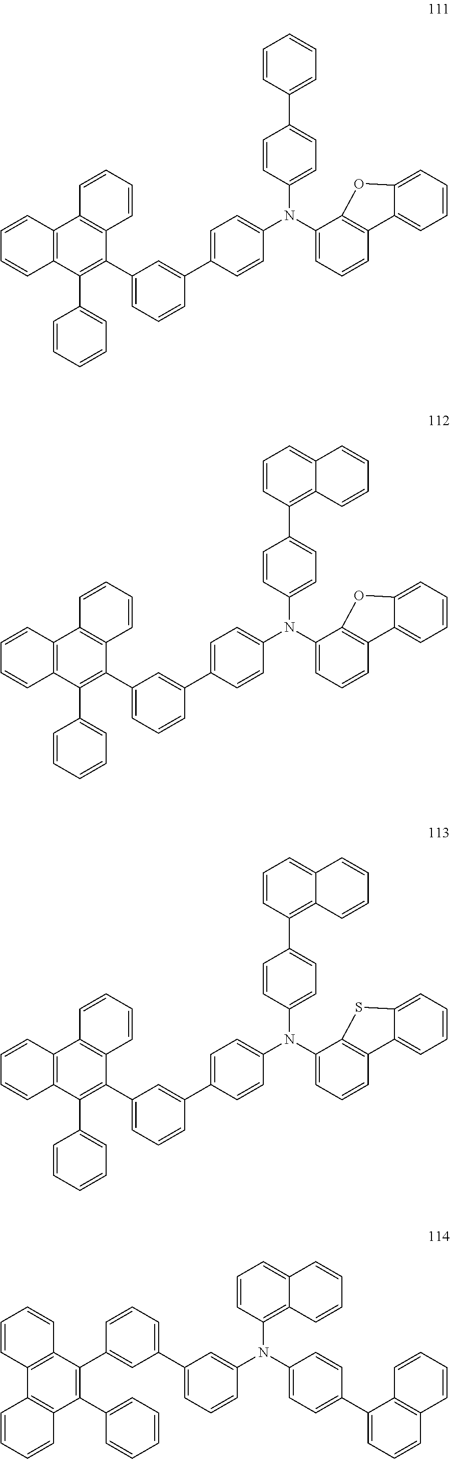 [SMILES: c1ccc(-c2ccc(N(c3ccc(-c4cccc(-c5c(-c6ccccc6)c6ccccc6c6ccccc56)c4)cc3)c3cccc4c3oc3ccccc34)cc2)cc1 ; c1ccc(-c2c(-c3cccc(-c4ccc(N(c5ccc(-c6cccc7ccccc67)cc5)c5cccc6c5oc5ccccc56)cc4)c3)c3ccccc3c3ccccc23)cc1 ; c1ccc(-c2c(-c3cccc(-c4ccc(N(c5ccc(-c6cccc7ccccc67)cc5)c5cccc6c5sc5ccccc56)cc4)c3)c3ccccc3c3ccccc23)cc1 ; c1ccc(-c2c(-c3cccc(-c4cccc(N(c5ccc(-c6cccc7ccccc67)cc5)c5cccc6ccccc56)c4)c3)c3ccccc3c3ccccc23)cc1]

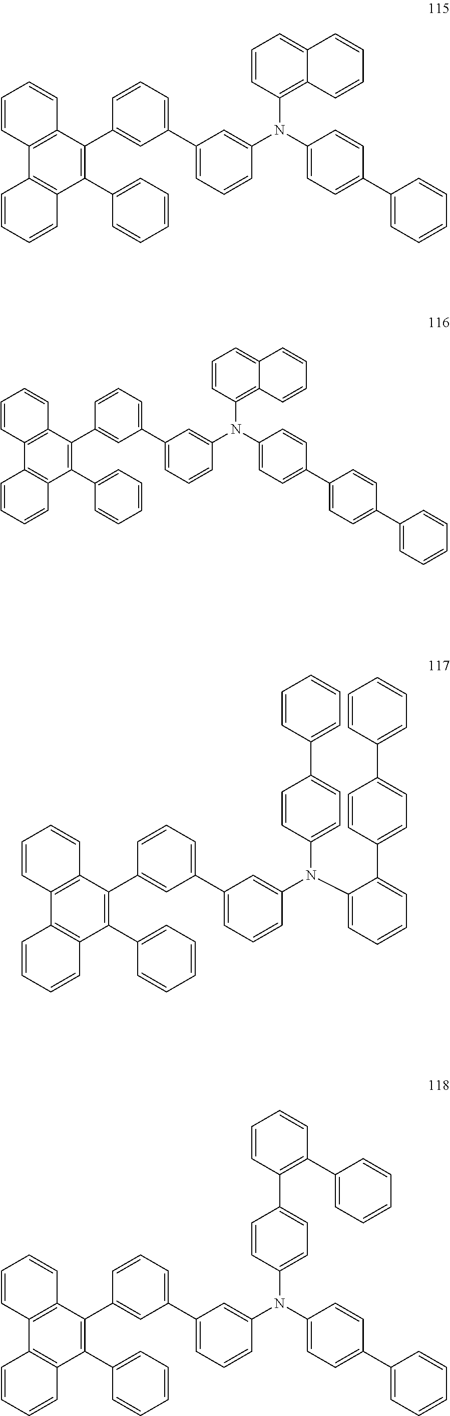 [SMILES: c1ccc(-c2ccc(N(c3cccc(-c4cccc(-c5c(-c6ccccc6)c6ccccc6c6ccccc56)c4)c3)c3cccc4ccccc34)cc2)cc1 ; c1ccc(-c2ccc(-c3ccc(N(c4cccc(-c5cccc(-c6c(-c7ccccc7)c7ccccc7c7ccccc67)c5)c4)c4cccc5ccccc45)cc3)cc2)cc1 ; c1ccc(-c2ccc(-c3ccccc3N(c3ccc(-c4ccccc4)cc3)c3cccc(-c4cccc(-c5c(-c6ccccc6)c6ccccc6c6ccccc56)c4)c3)cc2)cc1 ; c1ccc(-c2ccc(N(c3ccc(-c4ccccc4-c4ccccc4)cc3)c3cccc(-c4cccc(-c5c(-c6ccccc6)c6ccccc6c6ccccc56)c4)c3)cc2)cc1]

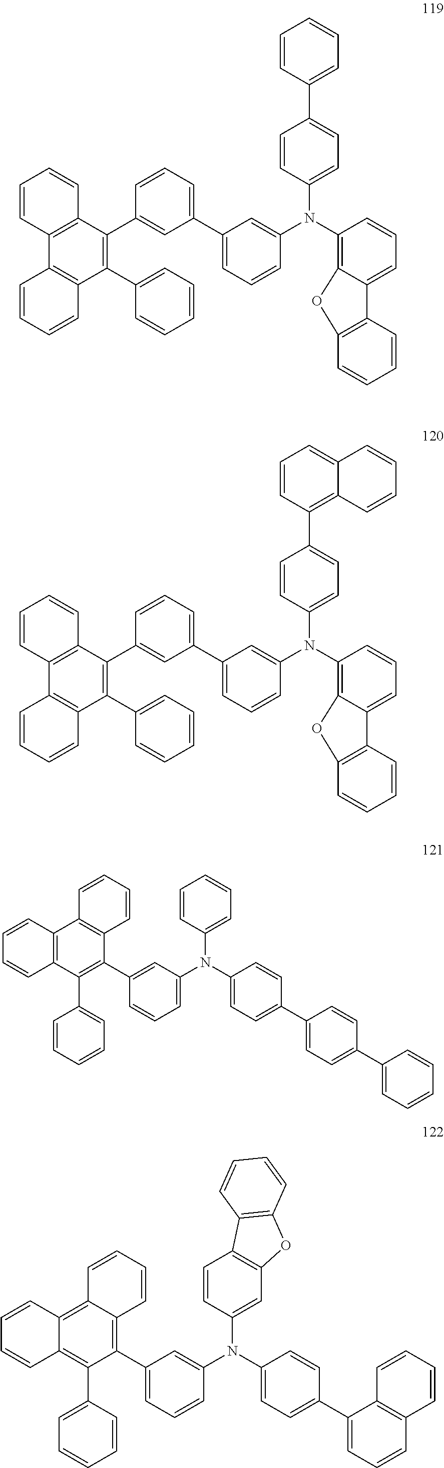 [SMILES: c1ccc(-c2ccc(N(c3cccc(-c4cccc(-c5c(-c6ccccc6)c6ccccc6c6ccccc56)c4)c3)c3cccc4c3oc3ccccc34)cc2)cc1 ; c1ccc(-c2c(-c3cccc(-c4cccc(N(c5ccc(-c6cccc7ccccc67)cc5)c5cccc6c5oc5ccccc56)c4)c3)c3ccccc3c3ccccc23)cc1 ; c1ccc(-c2ccc(-c3ccc(N(c4ccccc4)c4cccc(-c5c(-c6ccccc6)c6ccccc6c6ccccc56)c4)cc3)cc2)cc1 ; c1ccc(-c2c(-c3cccc(N(c4ccc(-c5cccc6ccccc56)cc4)c4ccc5c(c4)oc4ccccc45)c3)c3ccccc3c3ccccc23)cc1]

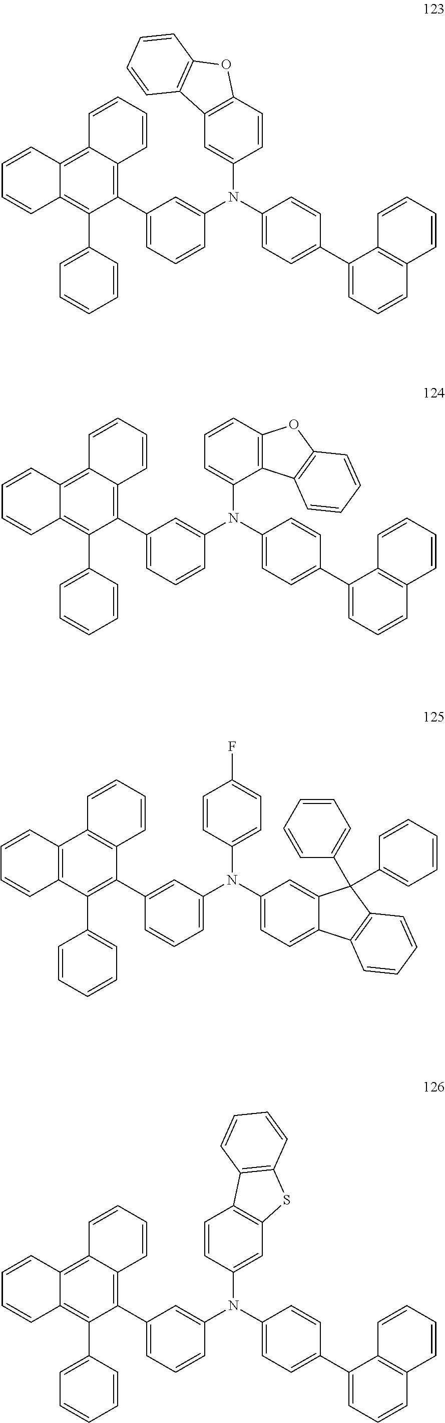 [SMILES: c1ccc(-c2c(-c3cccc(N(c4ccc(-c5cccc6ccccc56)cc4)c4ccc5oc6ccccc6c5c4)c3)c3ccccc3c3ccccc23)cc1 ; c1ccc(-c2c(-c3cccc(N(c4ccc(-c5cccc6ccccc56)cc4)c4cccc5oc6ccccc6c45)c3)c3ccccc3c3ccccc23)cc1 ; Fc1ccc(N(c2cccc(-c3c(-c4ccccc4)c4ccccc4c4ccccc34)c2)c2ccc3c(c2)C(c2ccccc2)(c2ccccc2)c2ccccc2-3)cc1 ; c1ccc(-c2c(-c3cccc(N(c4ccc(-c5cccc6ccccc56)cc4)c4ccc5c(c4)sc4ccccc45)c3)c3ccccc3c3ccccc23)cc1]

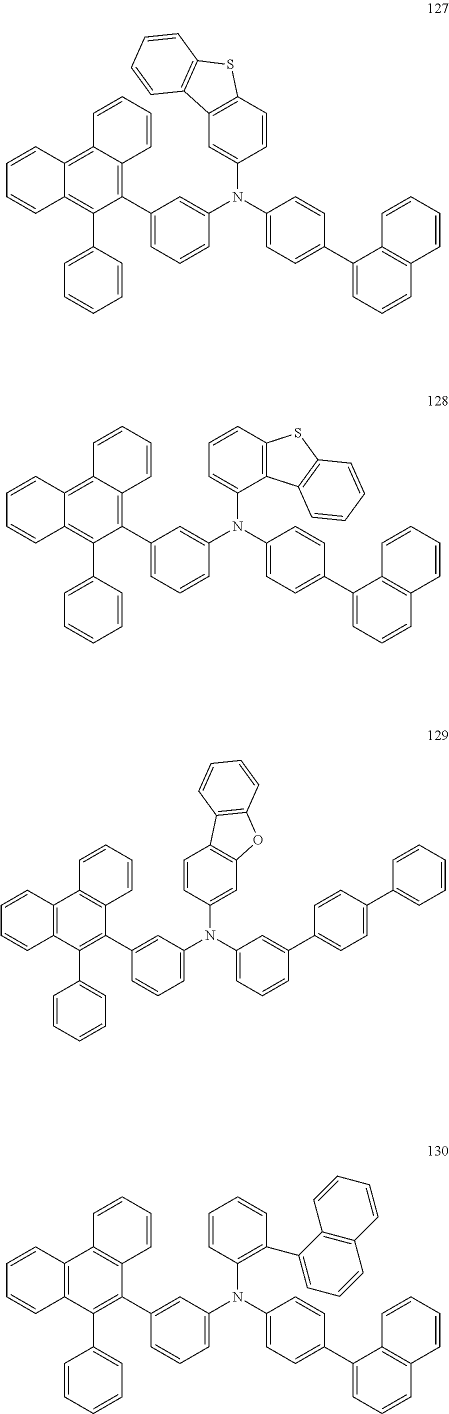 [SMILES: c1ccc(-c2c(-c3cccc(N(c4ccc(-c5cccc6ccccc56)cc4)c4ccc5sc6ccccc6c5c4)c3)c3ccccc3c3ccccc23)cc1 ; c1ccc(-c2c(-c3cccc(N(c4ccc(-c5cccc6ccccc56)cc4)c4cccc5sc6ccccc6c45)c3)c3ccccc3c3ccccc23)cc1 ; c1ccc(-c2ccc(-c3cccc(N(c4cccc(-c5c(-c6ccccc6)c6ccccc6c6ccccc56)c4)c4ccc5c(c4)oc4ccccc45)c3)cc2)cc1 ; c1ccc(-c2c(-c3cccc(N(c4ccc(-c5cccc6ccccc56)cc4)c4ccccc4-c4cccc5ccccc45)c3)c3ccccc3c3ccccc23)cc1]

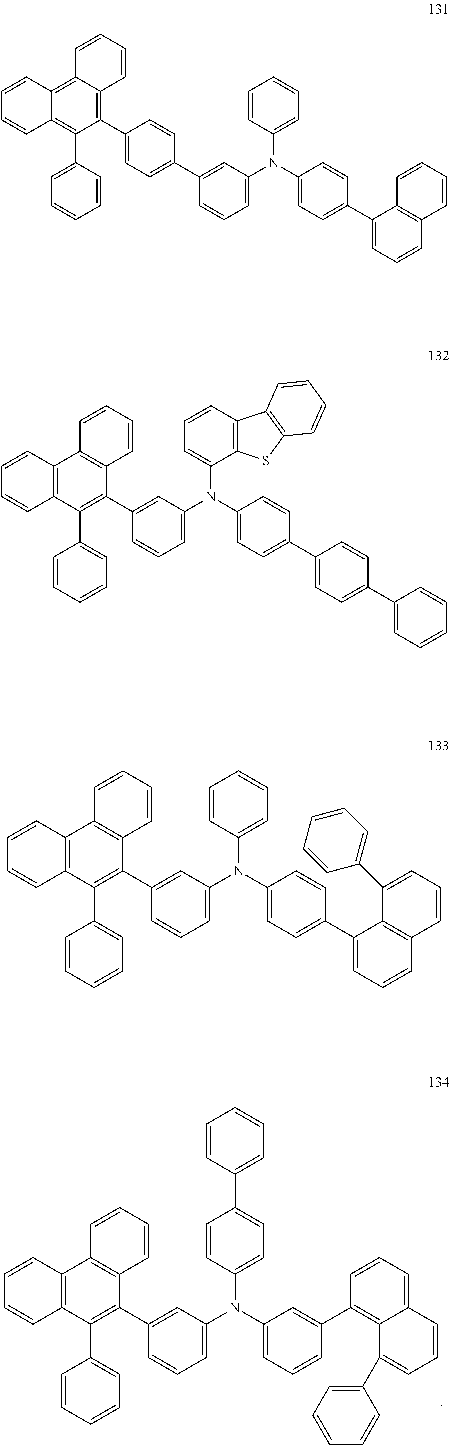 [SMILES: c1ccc(-c2c(-c3ccc(-c4cccc(N(c5ccccc5)c5ccc(-c6cccc7ccccc67)cc5)c4)cc3)c3ccccc3c3ccccc23)cc1 ; c1ccc(-c2ccc(-c3ccc(N(c4cccc(-c5c(-c6ccccc6)c6ccccc6c6ccccc56)c4)c4cccc5c4sc4ccccc45)cc3)cc2)cc1 ; c1ccc(-c2c(-c3cccc(N(c4ccccc4)c4ccc(-c5cccc6cccc(-c7ccccc7)c56)cc4)c3)c3ccccc3c3ccccc23)cc1 ; c1ccc(-c2ccc(N(c3cccc(-c4c(-c5ccccc5)c5ccccc5c5ccccc45)c3)c3cccc(-c4cccc5cccc(-c6ccccc6)c45)c3)cc2)cc1]

The monoamine compound according to an example embodiment may have a relatively large volume. Accordingly, when the monoamine compound represented by Formula 1 is applied to a light emitting element, high emission efficiency may be secured.

In particular, in an example embodiment, the monoamine compound represented by Formula 1 has a phenanthryl group connected with an amine group via an arylene linker, and a substituent having a large volume such as an aryl group is positioned at an adjacent position to a position where the phenanthryl group is connected with the arylene linker. Accordingly, steric repulsion may arise between the substituent and the arylene linker, the bonding angle between the phenanthryl group and the arylene linker may increase, and the volume occupied by the phenanthryl group may increase. Therefore, the monoamine compound according to an example embodiment may decrease interaction between phenanthryl groups. The decrease of the interaction between the phenanthryl groups may result in the decrease of electron mobility. In the case where the monoamine compound according to an example embodiment is disposed in a hole transport layer HTL adjacent to an emission layer EML, the diffusion of electrons from the emission layer to a hole transport region HTR may be restrained, and high emission efficiency of a light emitting element may be secured.

The hole transport region HTR in the light emitting element ED of an embodiment may further include a compound represented by Formula H-1 below:

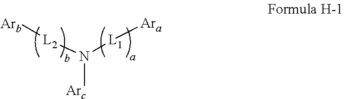

In Formula H-1 above, L₁ and L₂ may be each independently a direct linkage, a substituted or unsubstituted arylene group having 6 to 30 ring-forming carbon atoms, or a substituted or unsubstituted heteroarylene group having 2 to 30 ring-forming carbon atoms. a and b may be each independently an integer of 0 to 10. Meanwhile, when a or b is an integer of 2 or greater, a plurality of L₁'s and L₂'s may be each independently a substituted or unsubstituted arylene group having 6 to 30 ring-forming carbon atoms, or a substituted or unsubstituted heteroarylene group having 2 to 30 ring-forming carbon atoms.

In Formula H-1, Arₐ and Ar_{b} may be each independently a substituted or unsubstituted aryl group having 6 to 30 ring-forming carbon atoms, or a substituted or unsubstituted heteroaryl group having 2 to 30 ring-forming carbon atoms. In addition, in Formula H-1, Ar_{c} may be a substituted or unsubstituted aryl group having 6 to 30 ring-forming carbon atoms.

The compound represented by Formula H-1 above may be a monoamine compound. Alternatively, the compound represented by Formula H-1 above may be a diamine compound in which at least one among Arₐ to Ar_{c} contains an amine group as a substituent. In addition, the compound represented by Formula H-1 above may be a carbazole-based compound containing a substituted or unsubstituted carbazole group in at least one of Arₐ or Ar_{b}, or a fluorene-based compound containing a substituted or unsubstituted fluorene group in at least one of Arₐ or Ar_{b}.

The compound represented by Formula H-1 may be represented by any one among the compounds of Compound Group H below. However, the compounds listed in Compound Group H below are examples, and the compounds represented by Formula H-1 are not limited to those represented by Compound Group H below:

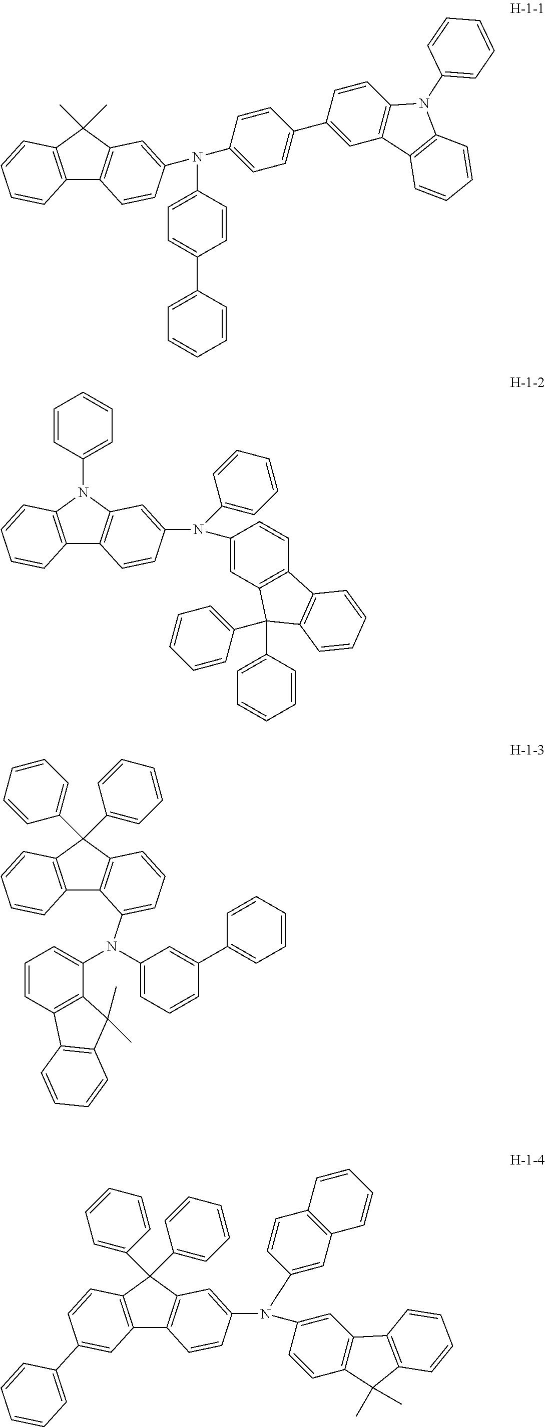 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(N(c2ccc3c(c2)C(c2ccccc2)(c2ccccc2)c2ccccc2-3)c2ccc3c4ccccc4n(-c4ccccc4)c3c2)cc1 ; CC1(C)c2ccccc2-c2cccc(N(c3cccc(-c4ccccc4)c3)c3cccc4c3-c3ccccc3C4(c3ccccc3)c3ccccc3)c21 ; CC1(C)c2ccccc2-c2cc(N(c3ccc4c(c3)C(c3ccccc3)(c3ccccc3)c3ccc(-c5ccccc5)cc3-4)c3ccc4ccccc4c3)ccc21]

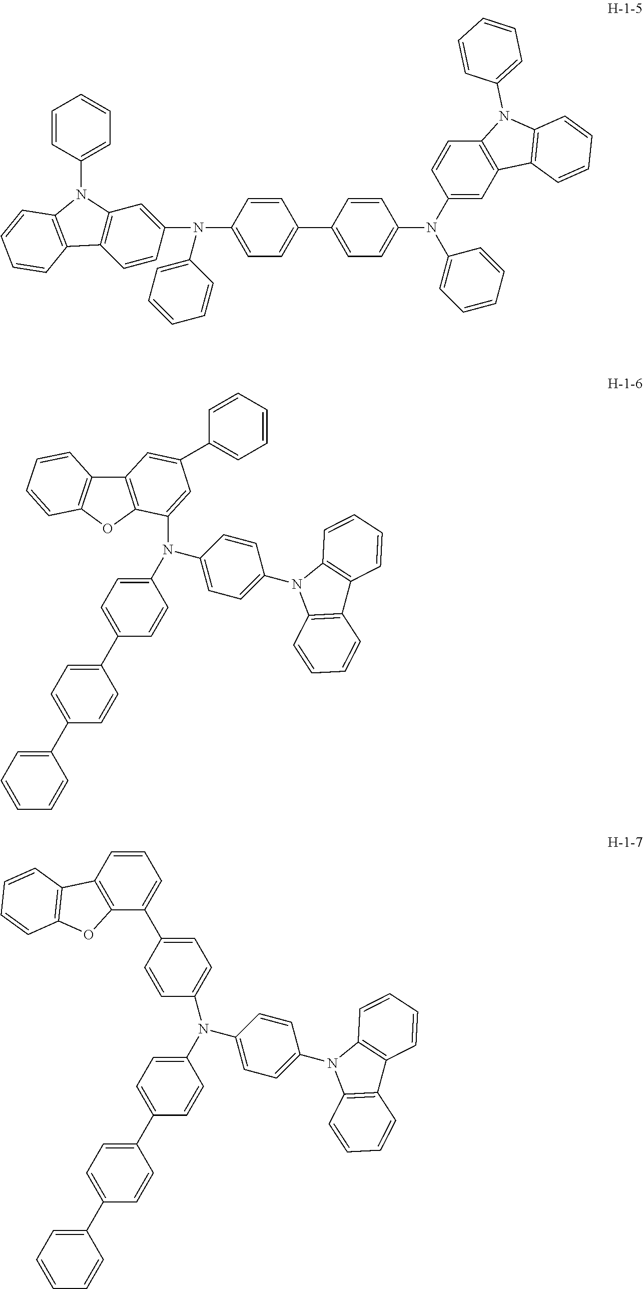 [SMILES: c1ccc(N(c2ccc(-c3ccc(N(c4ccccc4)c4ccc5c6ccccc6n(-c6ccccc6)c5c4)cc3)cc2)c2ccc3c(c2)c2ccccc2n3-c2ccccc2)cc1 ; c1ccc(-c2ccc(-c3ccc(N(c4ccc(-n5c6ccccc6c6ccccc65)cc4)c4cc(-c5ccccc5)cc5c4oc4ccccc45)cc3)cc2)cc1 ; c1ccc(-c2ccc(-c3ccc(N(c4ccc(-c5cccc6c5oc5ccccc56)cc4)c4ccc(-n5c6ccccc6c6ccccc65)cc4)cc3)cc2)cc1]

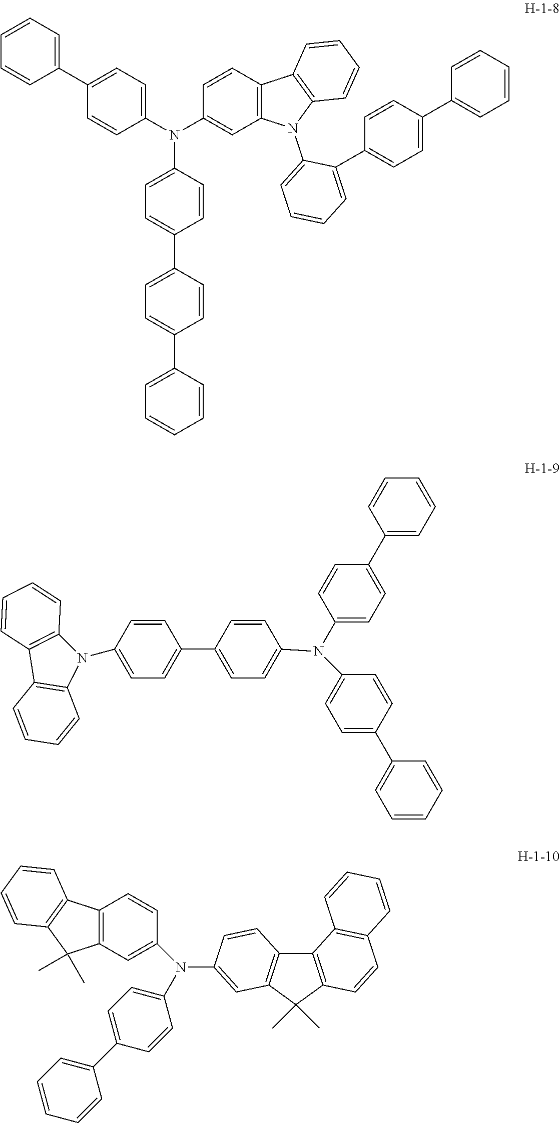 [SMILES: c1ccc(-c2ccc(-c3ccc(N(c4ccc(-c5ccccc5)cc4)c4ccc5c6ccccc6n(-c6ccccc6-c6ccc(-c7ccccc7)cc6)c5c4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1 ; CC1(C)c2ccccc2-c2ccc(N(c3ccc(-c4ccccc4)cc3)c3ccc4c(c3)C(C)(C)c3ccc5ccccc5c3-4)cc21]

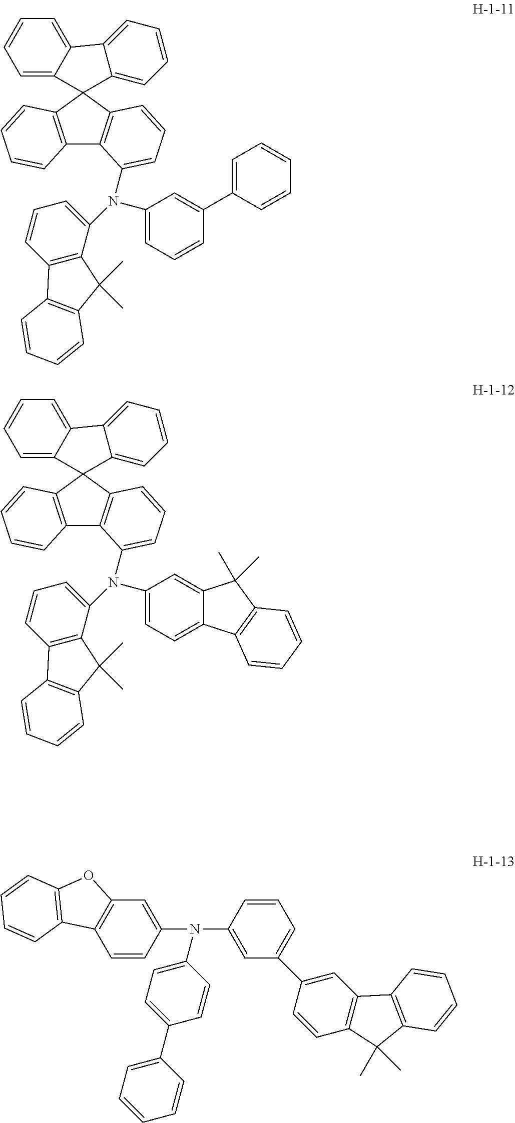

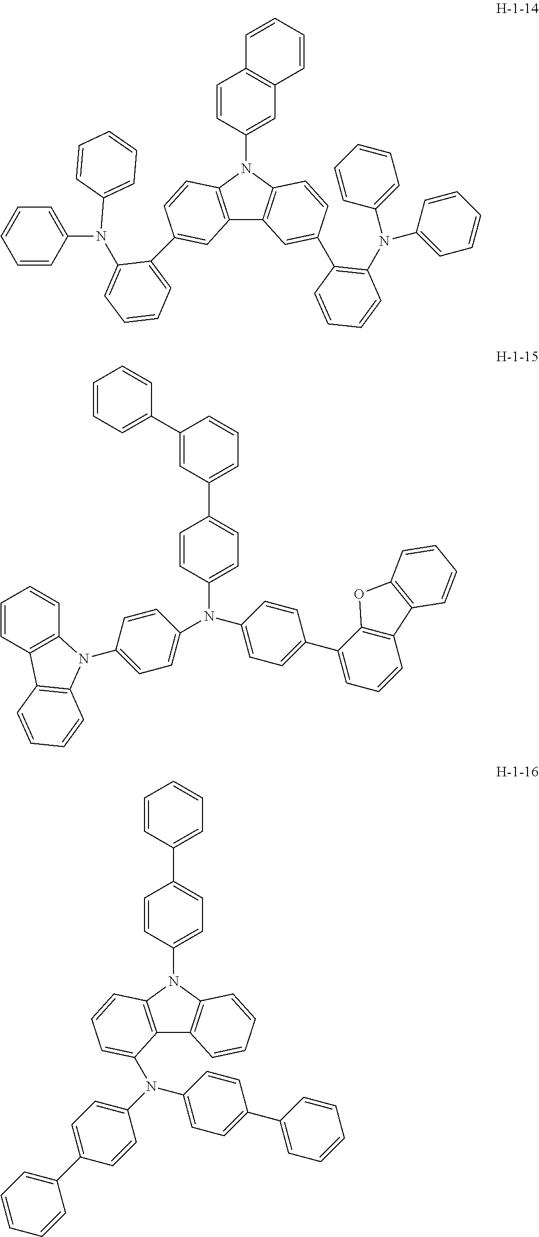

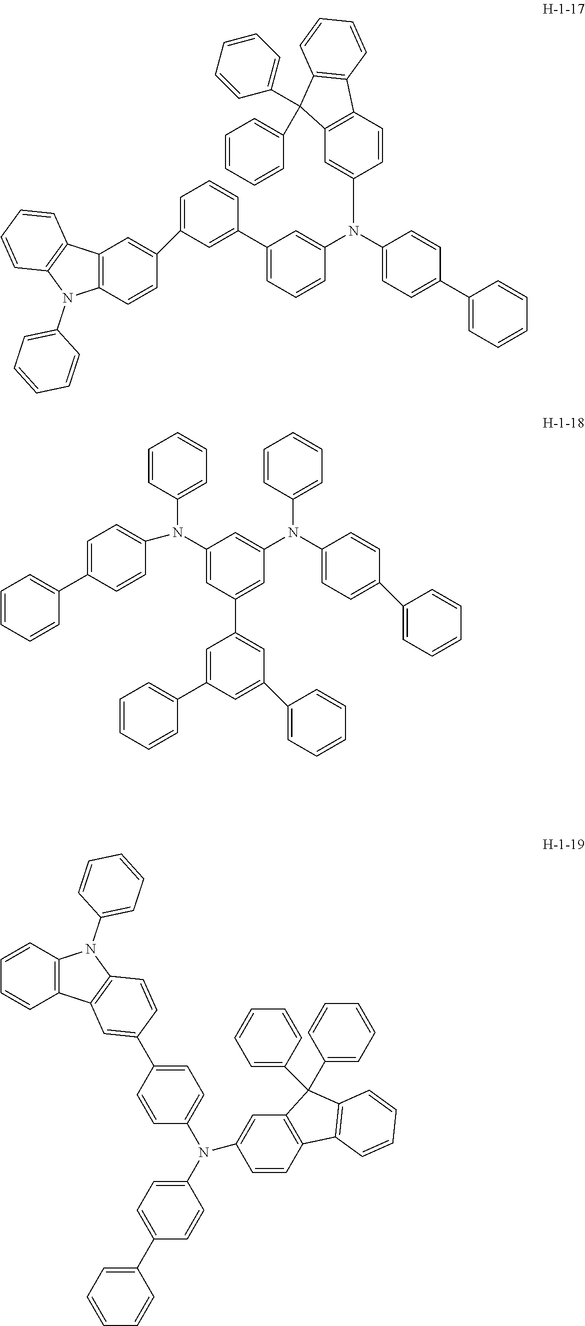 [SMILES: c1ccc(-c2ccc(N(c3cccc(-c4cccc(-c5ccc6c(c5)c5ccccc5n6-c5ccccc5)c4)c3)c3ccc4c(c3)C(c3ccccc3)(c3ccccc3)c3ccccc3-4)cc2)cc1 ; c1ccc(-c2ccc(N(c3ccccc3)c3cc(-c4cc(-c5ccccc5)cc(-c5ccccc5)c4)cc(N(c4ccccc4)c4ccc(-c5ccccc5)cc4)c3)cc2)cc1 ; c1ccc(-c2ccc(N(c3ccc(-c4ccc5c(c4)c4ccccc4n5-c4ccccc4)cc3)c3ccc4c(c3)C(c3ccccc3)(c3ccccc3)c3ccccc3-4)cc2)cc1]

In addition, the hole transport region HTR may further include a known hole transport material.

For example, the hole transport region HTR may include a phthalocyanine compound such as copper phthalocyanine; N¹,N¹′-([1,1′-biphenyl]-4,4′-diyl)bis(N¹-phenyl-N⁴,N⁴-di-m-tolylbenzene-1,4-diamine) (DNTPD), 4,4′,4″-[tris(3-methylphenyl)phenylamino] triphenylamine (m-MTDATA), 4,4′4″-tris(N,N-diphenylamino)triphenylamine (TDATA), 4,4′,4″-tris[N(1-naphthyl)-N-phenylamino]-triphenylamine (1-TNATA), 4,4′,4″-tris[N(2-naphthyl)-N-phenylamino]-triphenylamine (2-TNATA), poly(3,4-ethylenedioxythiophene)/poly(4-styrenesulfonate) (PEDOT/PSS), polyaniline/dodecylbenzenesulfonic acid (PANI/DBSA), polyaniline/camphor sulfonic acid (PANI/CSA), polyaniline/poly(4-styrenesulfonate) (PANI/PSS), N,N′-di(naphthalene-I-yl)-N,N′-diphenyl-benzidine (NPB or NPD), triphenylamine-containing polyetherketone (TPAPEK), 4-isopropyl-4′-methyldiphenyliodonium [tetrakis(pentafluorophenyl)borate], dipyrazino[2,3-f: 2′,3′-h]quinoxaline-2,3,6,7,10,11-hexacarbonitrile (HATCN), etc.

The hole transport region HTR may include a carbazole-based derivative such as N-phenyl carbazole or polyvinyl carbazole, a fluorene-based derivative, a triphenyl amine-based derivative such as 4,4′,4″-tris(N-carbazolyl)triphenylamine (TCTA), or N,N′-bis(3-methylphenyl)-N,N′-diphenyl-[1,1′-biphenyl]-4,4′-diamine (TPD), N,N′-di(naphthalene-I-yl)-N,N′-diphenyl-benzidine (NPB), 4,4′-cyclohexylidene bis[N,N-bis(4-methylphenyl)benzenamine](TAPC), 4,4′-bis[N,N′-(3-tolyl)amino]-3,3′-dimethylbiphenyl (HMTPD), 1,3-bis(N-carbazolyl)benzene (mCP), etc.

In addition, the hole transport region HTR may further include 9-(4-tert-butylphenyl)-3,6-bis(triphenylsilyl)-9H-carbazole (CzSi), 9-phenyl-9H-3,9′-bicarbazole (CCP), 1,3-bis(1,8-dimethyl-9H-carbazol-9-yl)benzene (mDCP), etc.

The hole transport region HTR may include the above-described compounds of the hole transport region in at least one of a hole injection layer HIL, a hole transport layer HTL, or an electron blocking layer EBL.

The thickness of the hole transport region HTR may be from about 100 Å to about 10,000 Å, for example, from about 100 Å to about 5,000 Å. When the hole transport region HTR includes the hole injection layer HIL, the hole injection layer HIL may have, for example, a thickness of about 30 Å to about 1,000 Å. When the hole transport region HTR includes the hole transport layer HTL, the hole transport layer HTL may have a thickness of about 250 Å to about 1,000 Å. For example, when the hole transport region HTR includes the electron blocking layer EBL, the electron blocking layer EBL may have a thickness of about 10 Å to about 1,000 Å. If the thicknesses of the hole transport region HTR, the hole injection layer HIL, the hole transport layer HTL and the electron blocking layer EBL satisfy the above-described ranges, satisfactory hole transport properties may be achieved without a substantial increase in driving voltage.

The hole transport region HTR may further include a charge generating material to increase conductivity in addition to the above-described materials. The charge generating material may be dispersed uniformly or non-uniformly in the hole transport region HTR. The charge generating material may be, for example, a p-dopant. The p-dopant may include at least one of a halogenated metal compound, a quinone derivative, a metal oxide, or a cyano group-containing compound, but the embodiment of the inventive concept is not limited thereto. For example, the p-dopant may include a metal halide compound such as CuI or RbI, a quinone derivative such as tetracyanoquinodimethane (TCNQ) or 2,3,5,6-tetrafluoro-7,7,8,8-tetracyanoquinodimethane (F4-TCNQ), a metal oxide such as tungsten oxide or molybdenum oxide, a cyano group-containing compound such as dipyrazino[2,3-f: 2′,3′-h]quinoxaline-2,3,6,7,10,11-hexacarbonitrile (HATCN) or 4-[[2,3-bis[cyano-(4-cyano-2,3,5,6-tetrafluorophenyl)methylidene]cyclopropylidene]-cyanomethyl]-2,3,5,6-tetrafluorobenzonitrile (NDP9), etc., but the embodiment of the inventive concept is not limited thereto.

As described above, the hole transport region HTR may further include a buffer layer (not shown) in addition to the hole injection layer HIL, the hole transport layer HTL, and the electron blocking layer EBL. The buffer layer (not shown) may compensate for a resonance distance according to the wavelength of light emitted from the emission layer EML and may thus increase light emission efficiency. A material that may be included in the hole transport region HTR may be used as a material to be included in the buffer layer (not shown).

The emission layer EML is provided on the hole transport region HTR. The emission layer EML may have a thickness of, for example, about 100 Å to about 1,000 Å or about 100 Å to about 300 Å. The emission layer EML may have a single layer formed of a single material, a single layer formed of a plurality of different materials, or a multilayer structure having a plurality of layers formed of a plurality of different materials.

The emission layer EML in the light emitting element ED of an embodiment may emit blue light. The light emitting element ED of an embodiment may include the above-described amine compound of an embodiment in the hole transport region HTR, thereby exhibiting high efficiency and long service life characteristics in the blue light emitting region. However, the embodiment of the inventive concept is not limited thereto.

In an embodiment, the emission layer EML may include multiple compounds.

The emission layer EML of an embodiment may include at least one among a compound represented by Formula HT-1, a compound represented by Formula ET-1 and a compound represented by Formula D-1.

In an embodiment, the emission layer EML may include a compound represented by Formula HT-1. In an embodiment, the HT-1 compound may be used as a hole transport host material in an emission layer EML.

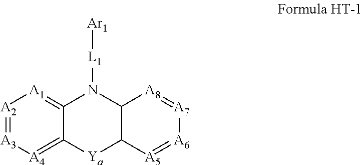

In Formula HT-1, A₁ to A₈ may be each independently N or CR₄₁. For example, all A₁ to A₈ may be CR₄₁. Otherwise, any one among A₁ to A₈ may be N, and the remainder may be CR₄₁.

In Formula HT-1, L₁ may be a direct linkage, a substituted or unsubstituted arylene group of 6 to 30 ring-forming carbon atoms, or a substituted or unsubstituted heteroarylene group of 2 to 30 ring-forming carbon atoms. For example, L₁ may be a direct linkage, a substituted or unsubstituted phenylene group, a substituted or unsubstituted divalent biphenyl group, a substituted or unsubstituted divalent carbazole group, or the like, but an embodiment of the inventive concept is not limited thereto.

In Formula HT-1, Yₐ may be a direct linkage, CR₄₂R₄₃, or SiR₄₄R₄₅. That is, it may mean that two benzene rings connected with the nitrogen atom of Formula HT-1 may be connected via a direct linkage,

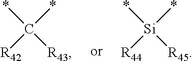

In Formula HT-1, if Yₐ is the direct linkage, the substituent represented by Formula HT-1 may include a carbazole moiety.

In Formula HT-1, Ar₁ may be a substituted or unsubstituted aryl group of 6 to 30 ring-forming carbon atoms, or a substituted or unsubstituted heteroaryl group of 2 to 30 ring-forming carbon atoms. For example, Ar₁ may be a substituted or unsubstituted carbazole group, a substituted or unsubstituted dibenzofuran group, a substituted or unsubstituted dibenzothiophene group, a substituted or unsubstituted biphenyl group, or the like, but an embodiment of the inventive concept is not limited thereto.

In Formula HT-1, R₄₁ to R₄₅ may be each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbona toms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 60 ring-forming carbon atoms. Otherwise, each of R₄₁ to R₄₅ may be combined with an adjacent group to form a ring. For example, R₄₁ to R₄₅ may be each independently a hydrogen atom or a deuterium atom. R₄₁ to R₄₅ may be each independently an unsubstituted methyl group or an unsubstituted phenyl group.

In an embodiment, the compound represented by Formula HT-1 may be represented by any one among the compounds represented in Compound Group 2. An emission layer EML may include at least one among the compounds represented in Compound Group 2 as a hole transport host material.

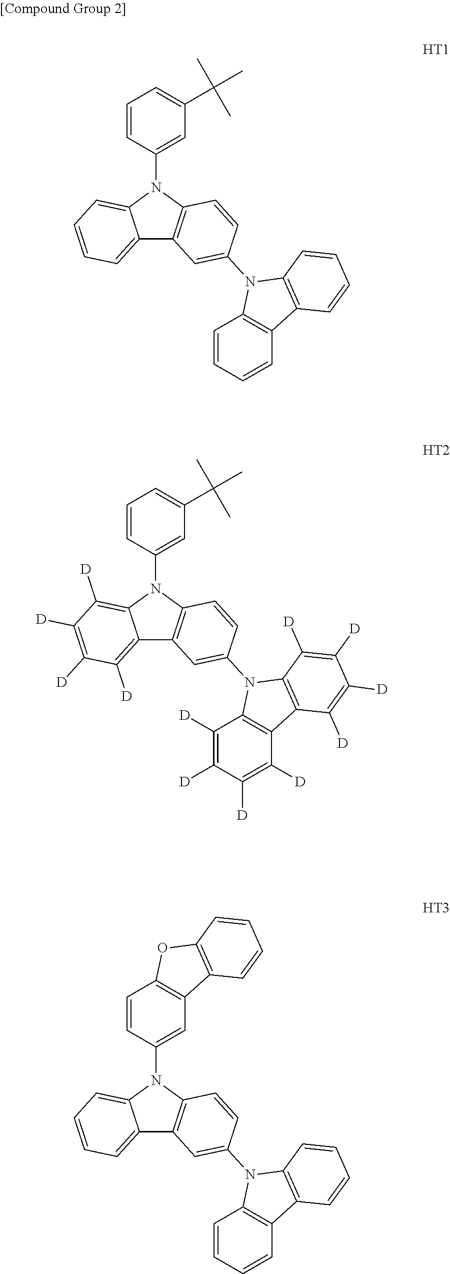 [SMILES: CC(C)(C)c1cccc(-n2c3ccccc3c3cc(-n4c5ccccc5c5ccccc54)ccc32)c1 ; [2H]c1c([2H])c([2H])c2c(c1[2H])c1cc(-n3c4c([2H])c([2H])c([2H])c([2H])c4c4c([2H])c([2H])c([2H])c([2H])c43)ccc1n2-c1cccc(C(C)(C)C)c1 ; c1ccc2c(c1)oc1ccc(-n3c4ccccc4c4cc(-n5c6ccccc6c6ccccc65)ccc43)cc12]

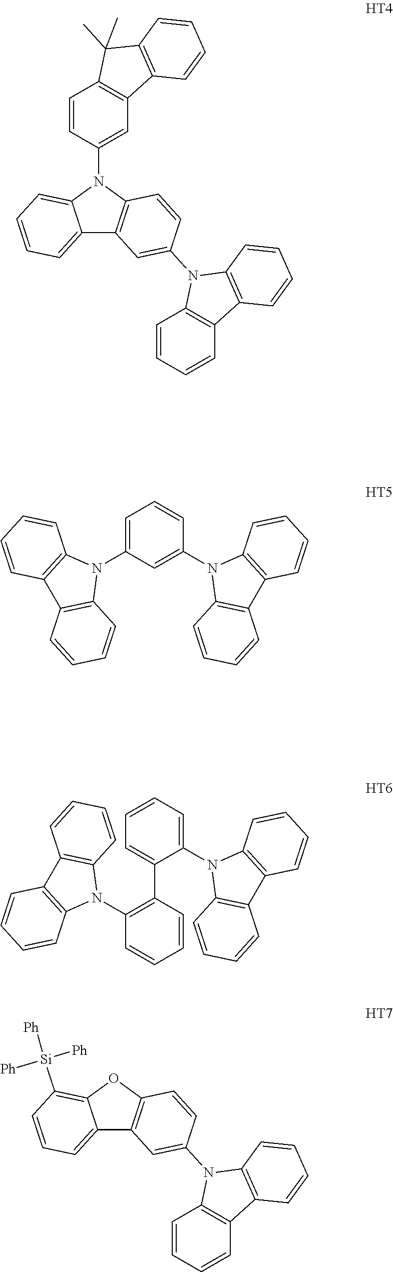 [SMILES: CC1(C)c2ccccc2-c2cc(-n3c4ccccc4c4cc(-n5c6ccccc6c6ccccc65)ccc43)ccc21 ; c1cc(-n2c3ccccc3c3ccccc32)cc(-n2c3ccccc3c3ccccc32)c1 ; c1ccc(-n2c3ccccc3c3ccccc32)c(-c2ccccc2-n2c3ccccc3c3ccccc32)c1 ; c1ccc([Si](c2ccccc2)(c2ccccc2)c2cccc3c2oc2ccc(-n4c5ccccc5c5ccccc54)cc23)cc1]

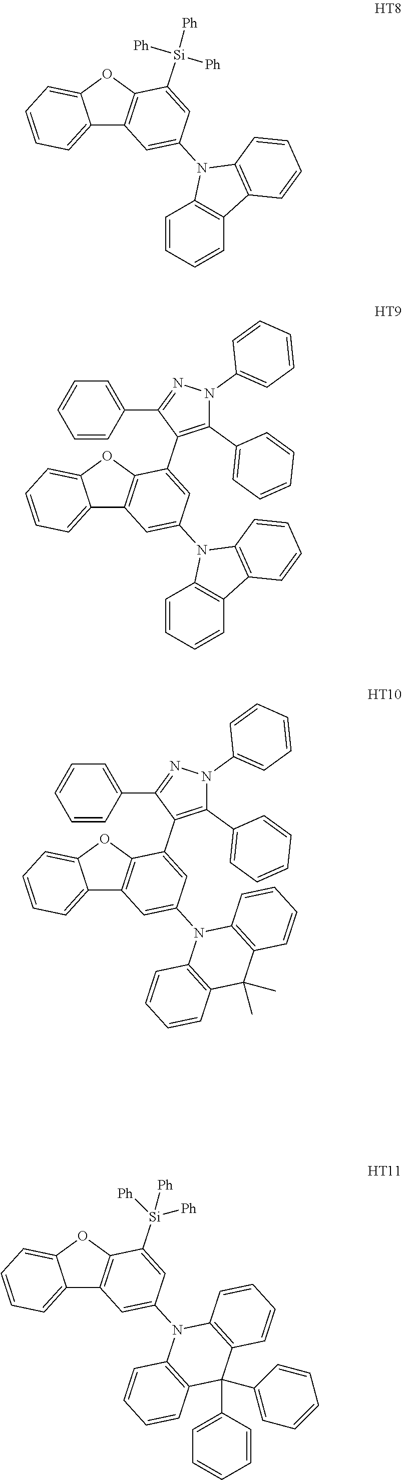 [SMILES: c1ccc([Si](c2ccccc2)(c2ccccc2)c2cc(-n3c4ccccc4c4ccccc43)cc3c2oc2ccccc23)cc1 ; c1ccc(-c2nn(-c3ccccc3)c(-c3ccccc3)c2-c2cc(-n3c4ccccc4c4ccccc43)cc3c2oc2ccccc23)cc1 ; CC1(C)c2ccccc2N(c2cc(-c3c(-c4ccccc4)nn(-c4ccccc4)c3-c3ccccc3)c3oc4ccccc4c3c2)c2ccccc21 ; c1ccc(C2(c3ccccc3)c3ccccc3N(c3cc([Si](c4ccccc4)(c4ccccc4)c4ccccc4)c4oc5ccccc5c4c3)c3ccccc32)cc1]

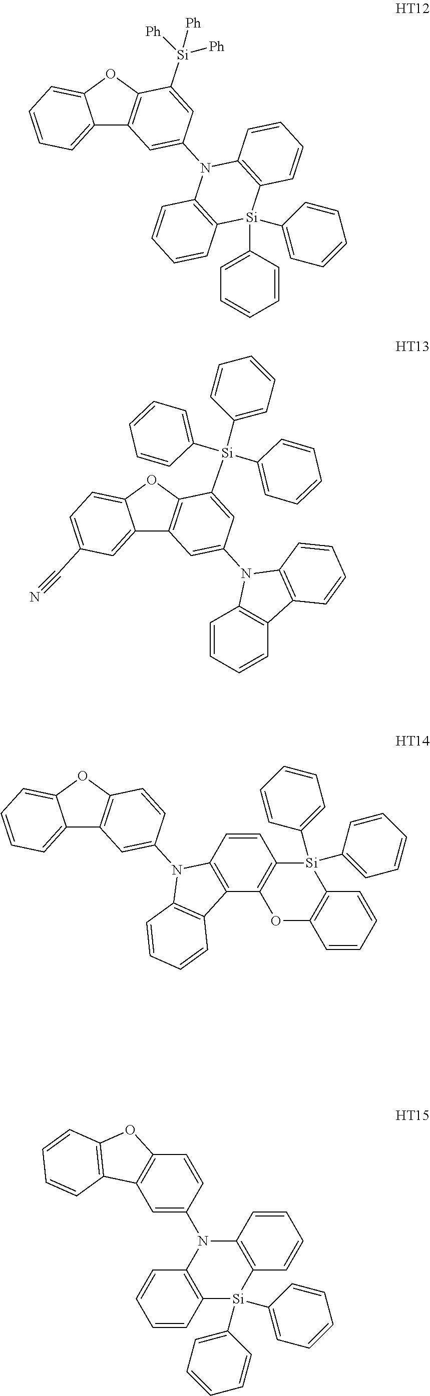

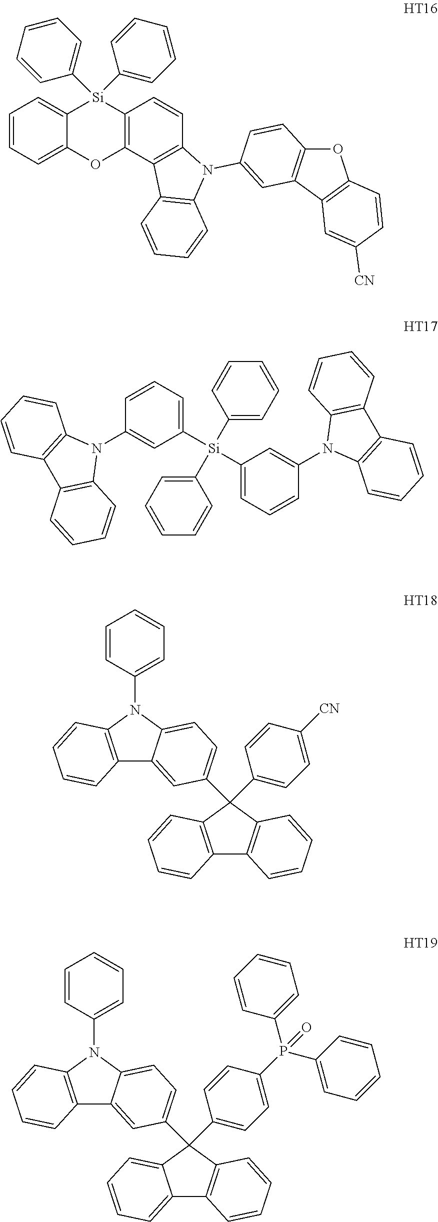 [SMILES: N#Cc1ccc2oc3ccc(-n4c5ccccc5c5c6c(ccc54)[Si](c4ccccc4)(c4ccccc4)c4ccccc4O6)cc3c2c1 ; c1ccc([Si](c2ccccc2)(c2cccc(-n3c4ccccc4c4ccccc43)c2)c2cccc(-n3c4ccccc4c4ccccc43)c2)cc1 ; N#Cc1ccc(C2(c3ccc4c(c3)c3ccccc3n4-c3ccccc3)c3ccccc3-c3ccccc32)cc1 ; O=P(c1ccccc1)(c1ccccc1)c1ccc(C2(c3ccc4c(c3)c3ccccc3n4-c3ccccc3)c3ccccc3-c3ccccc32)cc1]

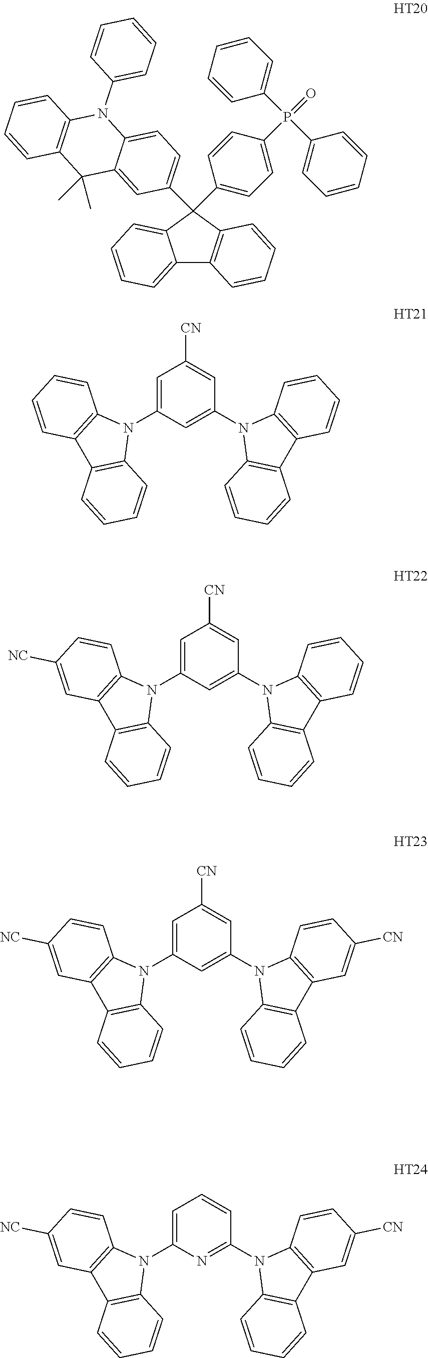 [SMILES: CC1(C)c2ccccc2N(c2ccccc2)c2ccc(C3(c4ccc(P(=O)(c5ccccc5)c5ccccc5)cc4)c4ccccc4-c4ccccc43)cc21 ; N#Cc1cc(-n2c3ccccc3c3ccccc32)cc(-n2c3ccccc3c3ccccc32)c1 ; N#Cc1cc(-n2c3ccccc3c3ccccc32)cc(-n2c3ccccc3c3cc(C#N)ccc32)c1 ; N#Cc1cc(-n2c3ccccc3c3cc(C#N)ccc32)cc(-n2c3ccccc3c3cc(C#N)ccc32)c1 ; N#Cc1ccc2c(c1)c1ccccc1n2-c1cccc(-n2c3ccccc3c3cc(C#N)ccc32)n1]

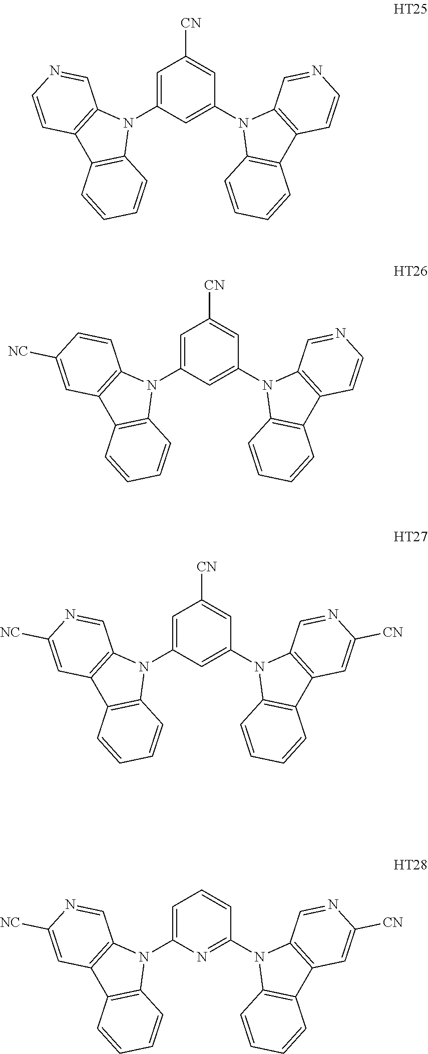 [SMILES: N#Cc1cc(-n2c3ccccc3c3ccncc32)cc(-n2c3ccccc3c3ccncc32)c1 ; N#Cc1cc(-n2c3ccccc3c3cc(C#N)ccc32)cc(-n2c3ccccc3c3ccncc32)c1 ; N#Cc1cc(-n2c3ccccc3c3cc(C#N)ncc32)cc(-n2c3ccccc3c3cc(C#N)ncc32)c1 ; N#Cc1cc2c3ccccc3n(-c3cccc(-n4c5ccccc5c5cc(C#N)ncc54)n3)c2cn1]

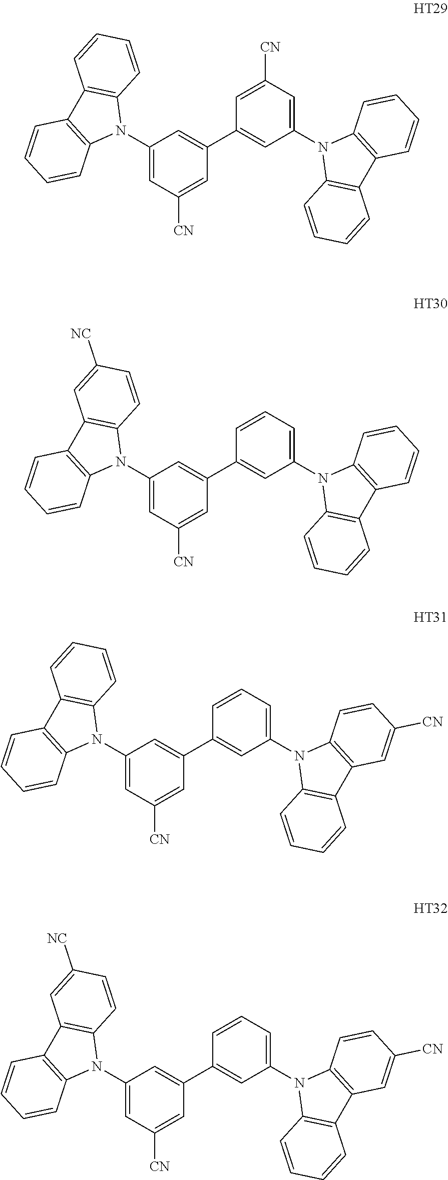 [SMILES: N#Cc1cc(-c2cc(C#N)cc(-n3c4ccccc4c4ccccc43)c2)cc(-n2c3ccccc3c3ccccc32)c1 ; N#Cc1cc(-c2cccc(-n3c4ccccc4c4ccccc43)c2)cc(-n2c3ccccc3c3cc(C#N)ccc32)c1 ; N#Cc1cc(-c2cccc(-n3c4ccccc4c4cc(C#N)ccc43)c2)cc(-n2c3ccccc3c3ccccc32)c1 ; N#Cc1cc(-c2cccc(-n3c4ccccc4c4cc(C#N)ccc43)c2)cc(-n2c3ccccc3c3cc(C#N)ccc32)c1]

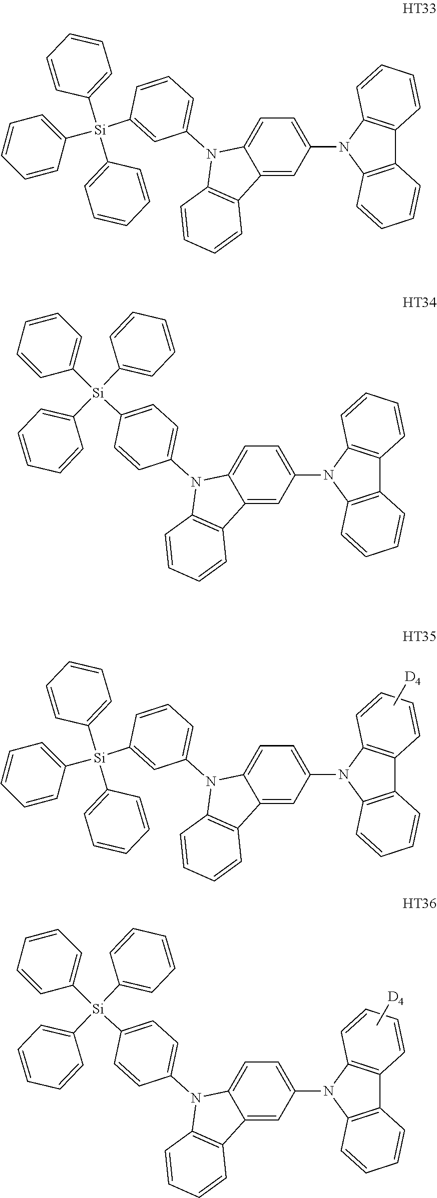

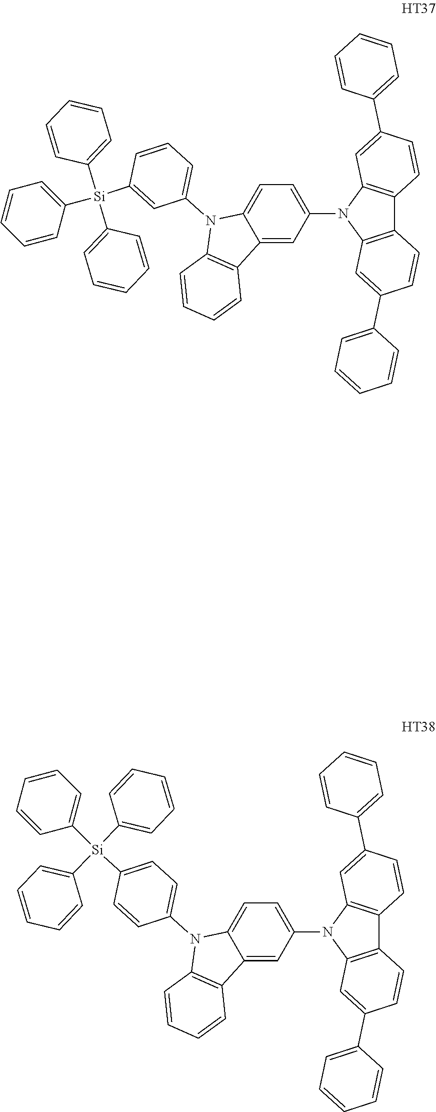 [SMILES: c1ccc(-c2ccc3c4ccc(-c5ccccc5)cc4n(-c4ccc5c(c4)c4ccccc4n5-c4cccc([Si](c5ccccc5)(c5ccccc5)c5ccccc5)c4)c3c2)cc1 ; c1ccc(-c2ccc3c4ccc(-c5ccccc5)cc4n(-c4ccc5c(c4)c4ccccc4n5-c4ccc([Si](c5ccccc5)(c5ccccc5)c5ccccc5)cc4)c3c2)cc1]

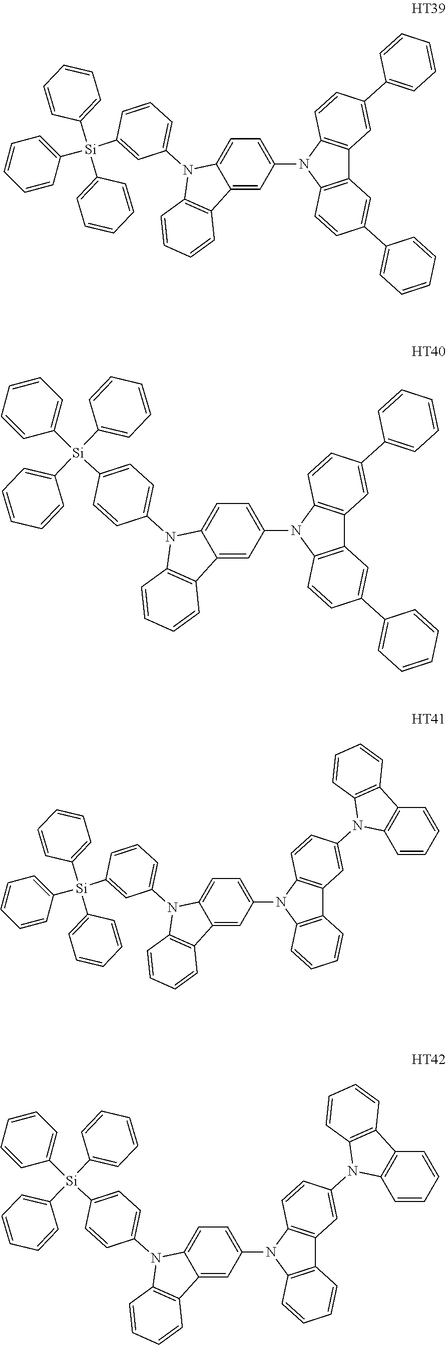 [SMILES: c1ccc(-c2ccc3c(c2)c2cc(-c4ccccc4)ccc2n3-c2ccc3c(c2)c2ccccc2n3-c2cccc([Si](c3ccccc3)(c3ccccc3)c3ccccc3)c2)cc1 ; c1ccc(-c2ccc3c(c2)c2cc(-c4ccccc4)ccc2n3-c2ccc3c(c2)c2ccccc2n3-c2ccc([Si](c3ccccc3)(c3ccccc3)c3ccccc3)cc2)cc1 ; c1ccc([Si](c2ccccc2)(c2ccccc2)c2cccc(-n3c4ccccc4c4cc(-n5c6ccccc6c6cc(-n7c8ccccc8c8ccccc87)ccc65)ccc43)c2)cc1 ; c1ccc([Si](c2ccccc2)(c2ccccc2)c2ccc(-n3c4ccccc4c4cc(-n5c6ccccc6c6cc(-n7c8ccccc8c8ccccc87)ccc65)ccc43)cc2)cc1]

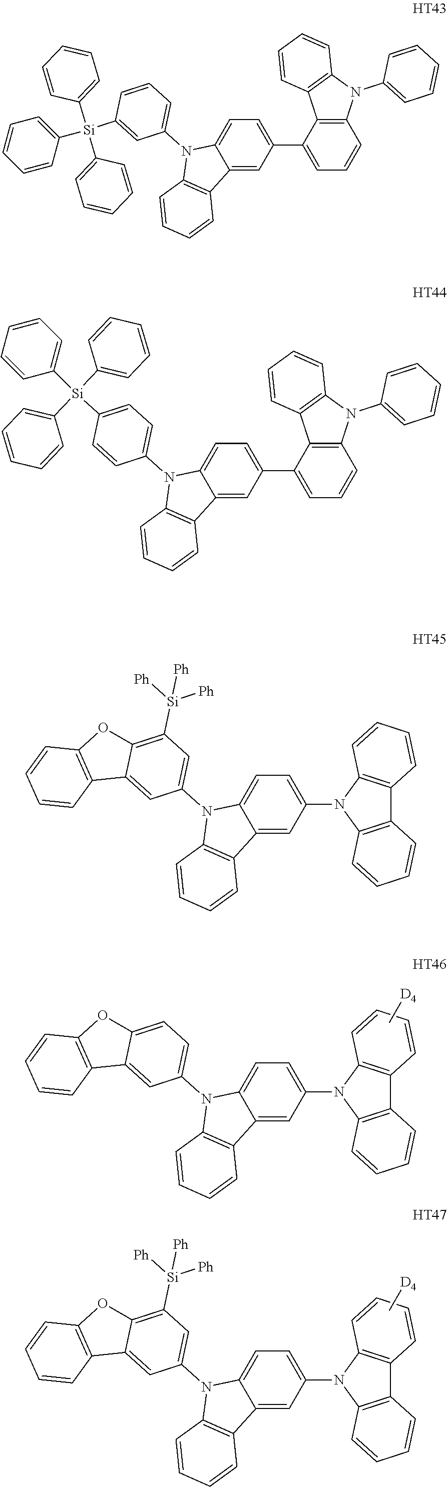

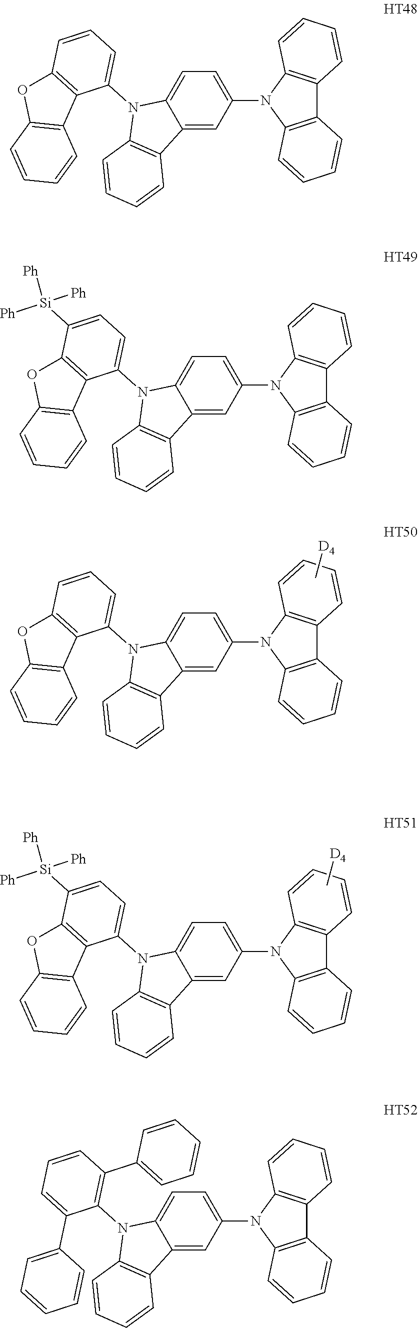

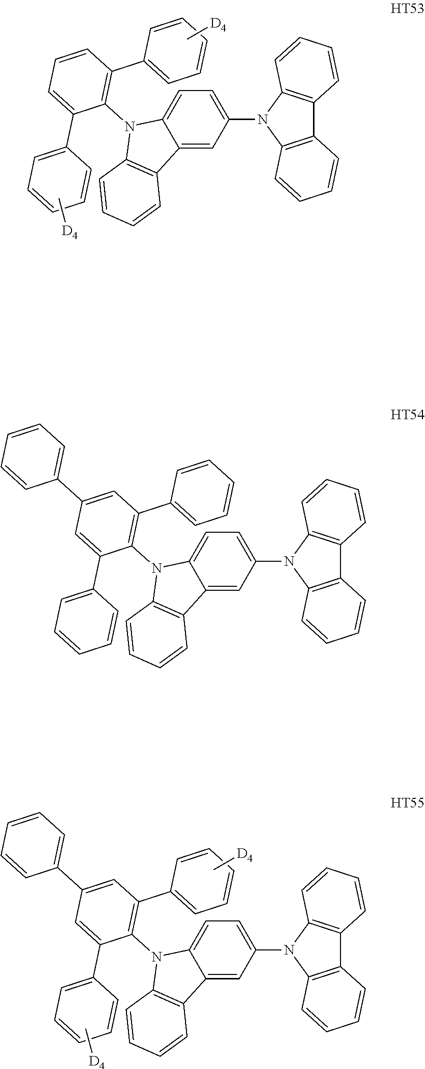

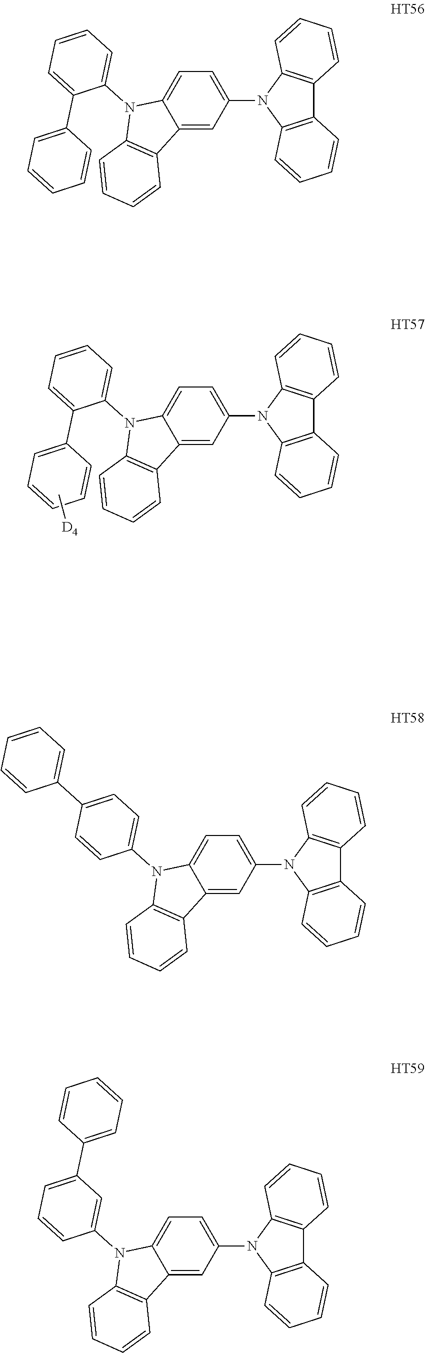

In the particular compounds suggested in Compound Group 2, “D” means a deuterium atom, and “Ph” means an unsubstituted phenyl group. For example, in the particular compounds suggested in Compound Group 2, “Ph” may be an unsubstituted phenyl group.

In an embodiment, the emission layer EML may include a compound represented by Formula ET-1 below. For example, the ET-1 compound may be used as an electron transport host material in the emission layer EML.

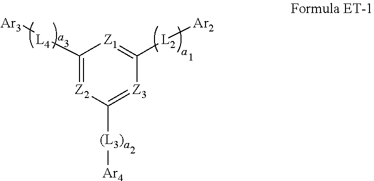

In Formula ET-1, at least one among Z₁ to Z₃ may be N, and the remainder may be CRₐ₃, and Rₐ₃ may be a hydrogen atom, a deuterium atom, a substituted or unsubstituted alkyl group of 1 to 20 carbon atoms, a substituted or unsubstituted aryl group of 6 to 60 ring-forming carbon atoms, or a substituted or unsubstituted heteroaryl group of 2 to 60 ring-forming carbon atoms.

a₁ to a₃ are each independently an integer of 0 to 10.

L₂ to L₄ may be each independently a direct linkage, a substituted or unsubstituted arylene group of 6 to 30 ring-forming carbon atoms, or a substituted or unsubstituted heteroarylene group of 2 to 30 ring-forming carbon atoms. Meanwhile, if each of a₁ to a₃ is an integer of 2 or more, L₂ to L₄ may be each independently a substituted or unsubstituted arylene group of 6 to 30 ring-forming carbon atoms, or a substituted or unsubstituted heteroarylene group of 2 to 30 ring-forming carbon atoms.

Ar₂ to Ar₄ may be each independently a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. For example, Ar₂ to Ar₄ may be substituted or unsubstituted phenyl groups or substituted or unsubstituted carbazole group.

The ET-1 compound may be represented by any one among the compounds in Compound Group 3. The light emitting element ED of an embodiment may include any one among the compounds in Compound Group 3.

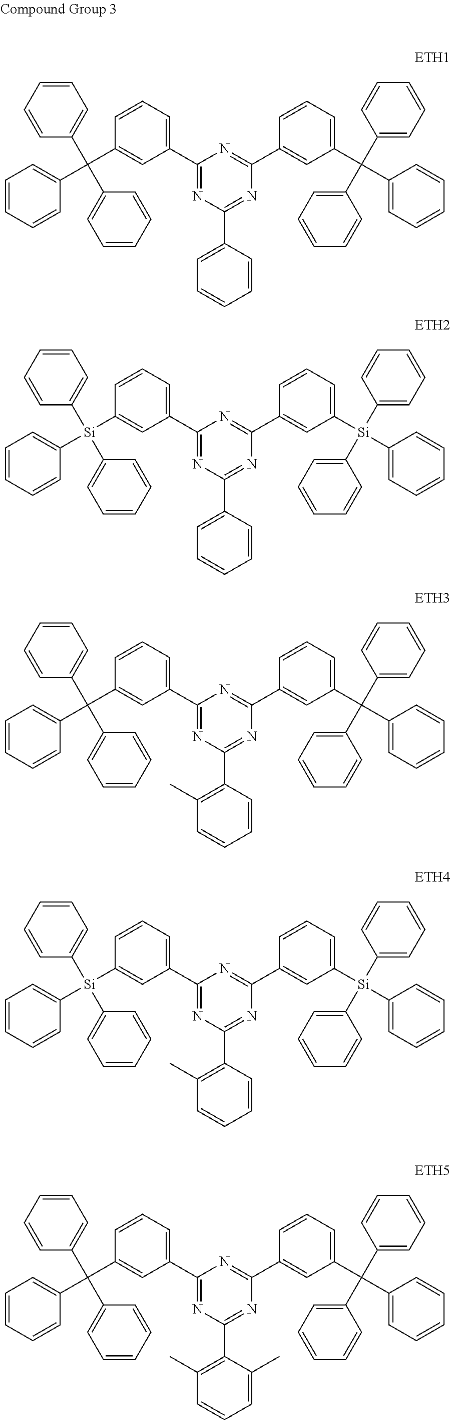

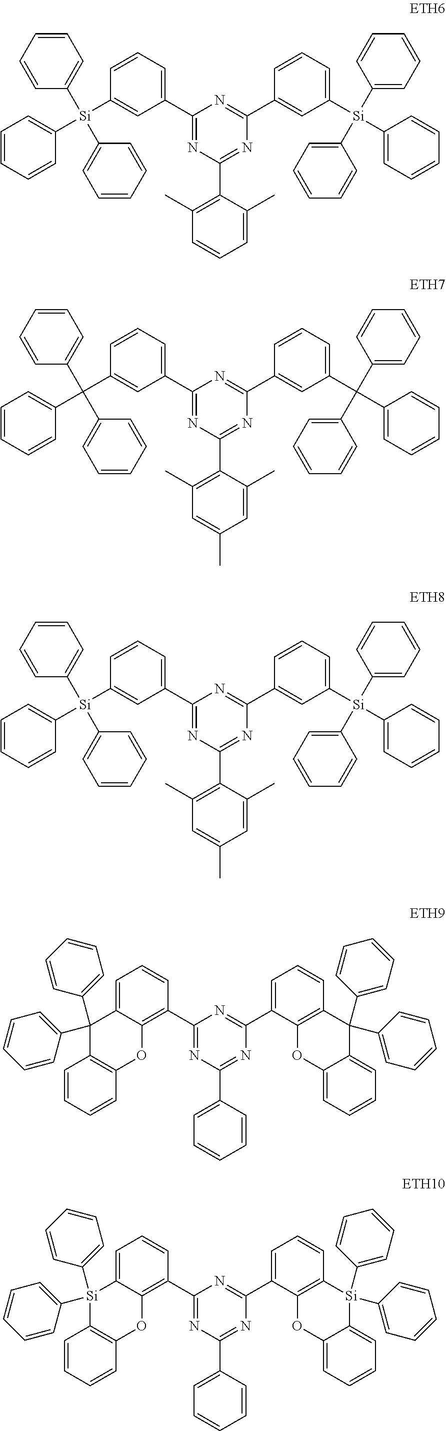

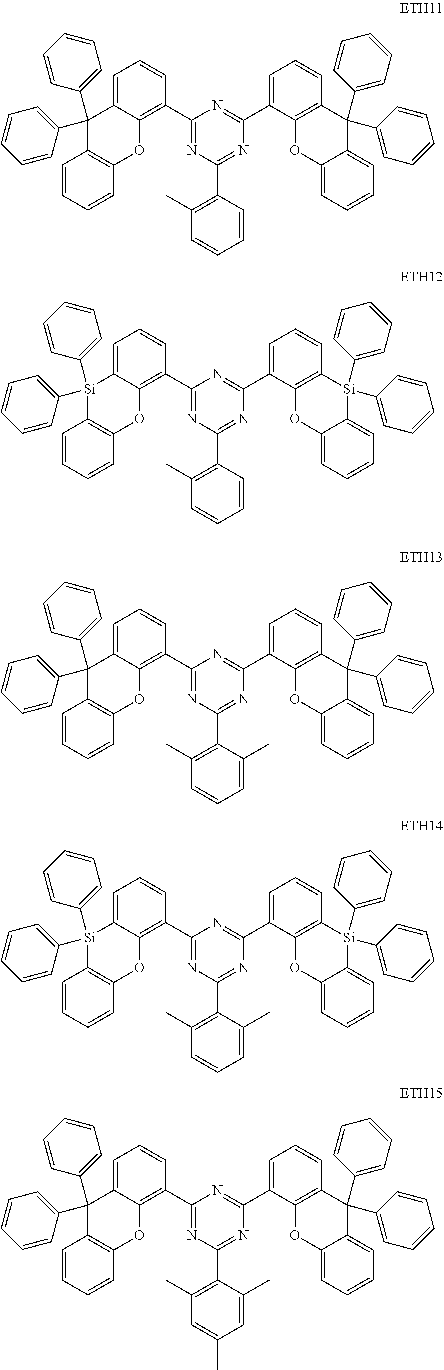

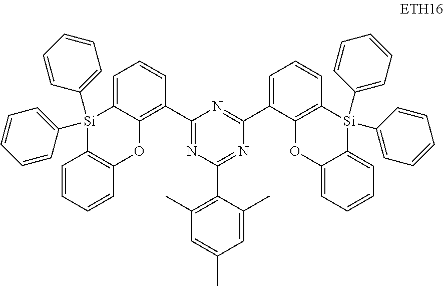 [SMILES: Cc1cc(C)c(-c2nc(-c3cccc4c3Oc3ccccc3[Si]4(c3ccccc3)c3ccccc3)nc(-c3cccc4c3Oc3ccccc3[Si]4(c3ccccc3)c3ccccc3)n2)c(C)c1]

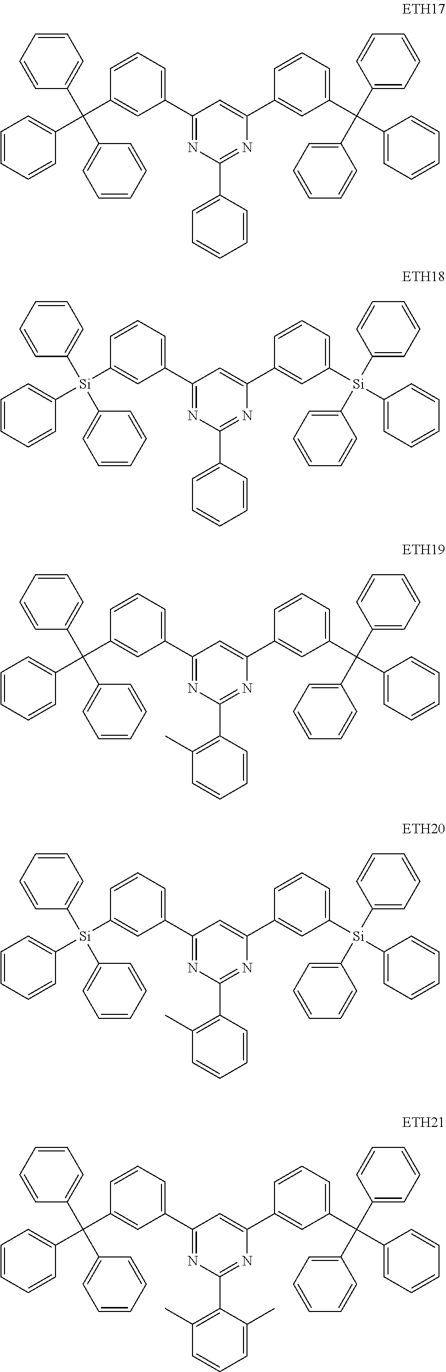

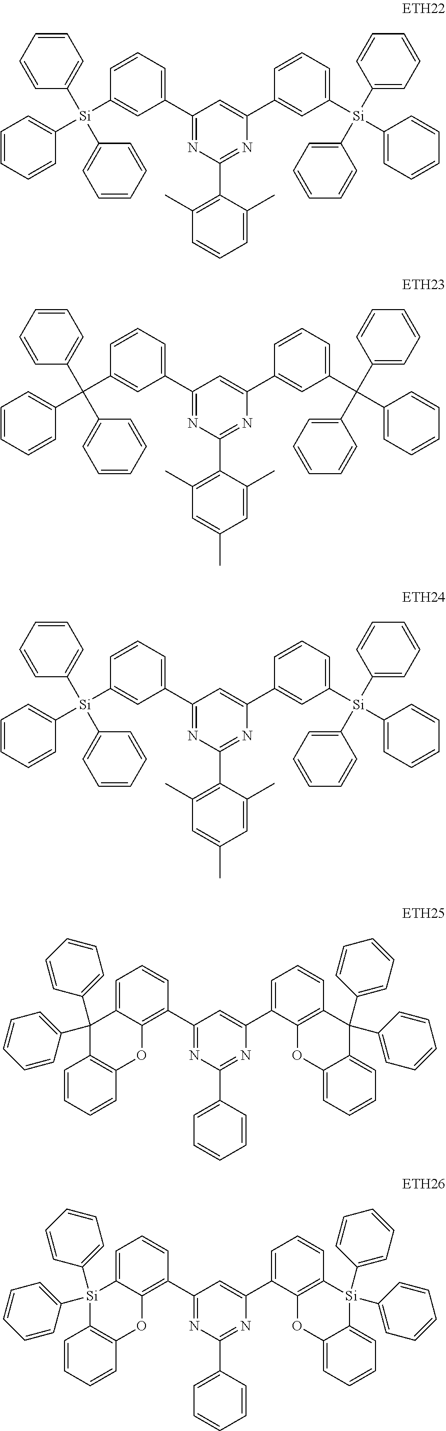

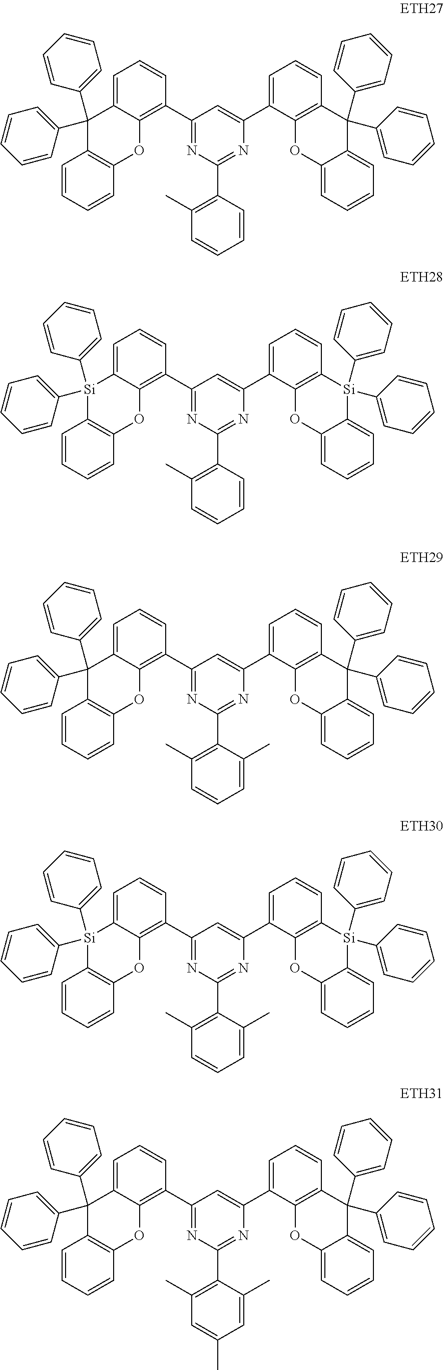

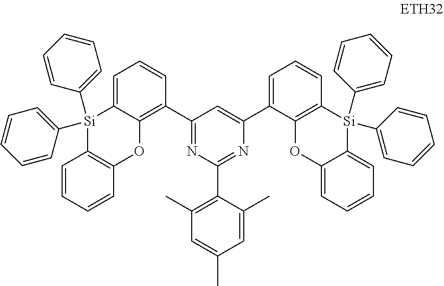 [SMILES: Cc1cc(C)c(-c2nc(-c3cccc4c3Oc3ccccc3[Si]4(c3ccccc3)c3ccccc3)cc(-c3cccc4c3Oc3ccccc3[Si]4(c3ccccc3)c3ccccc3)n2)c(C)c1]

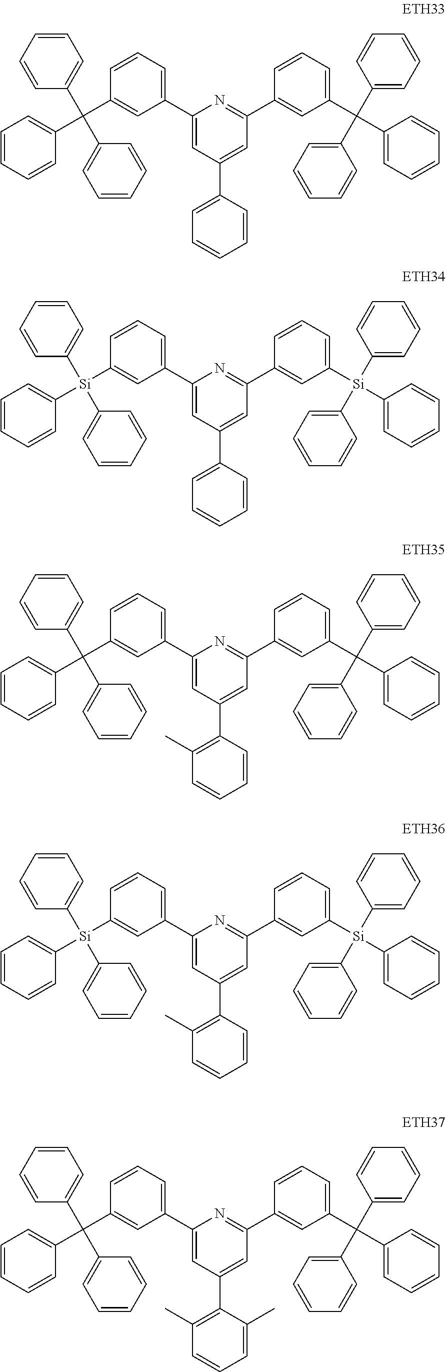

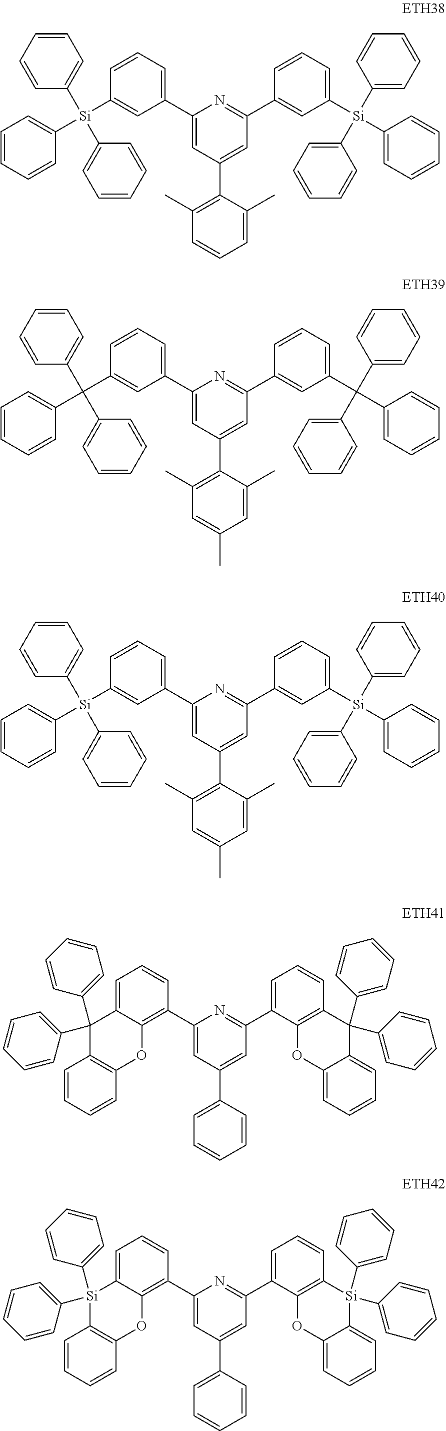

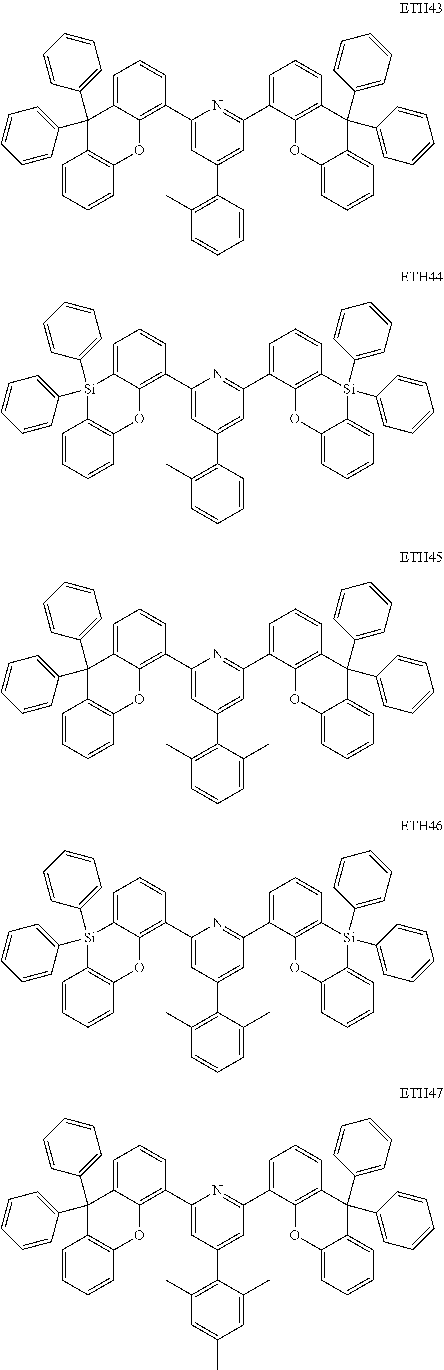

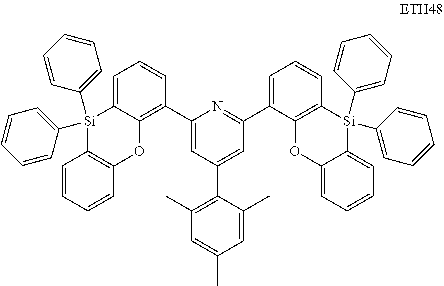 [SMILES: Cc1cc(C)c(-c2cc(-c3cccc4c3Oc3ccccc3[Si]4(c3ccccc3)c3ccccc3)nc(-c3cccc4c3Oc3ccccc3[Si]4(c3ccccc3)c3ccccc3)c2)c(C)c1]

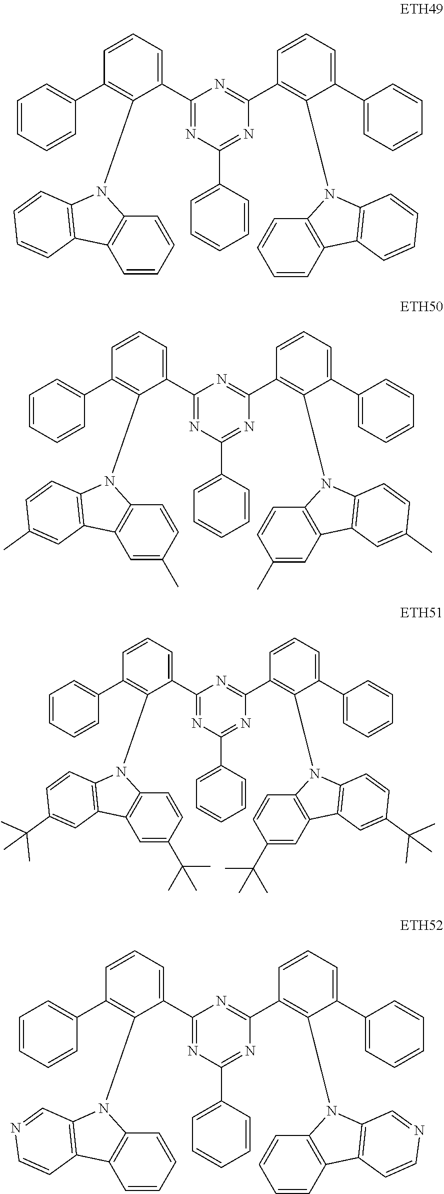

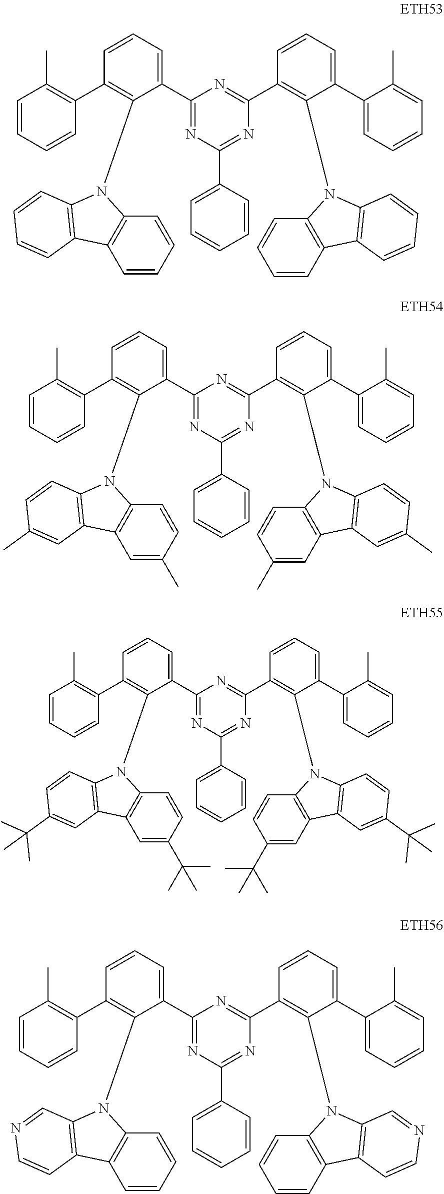

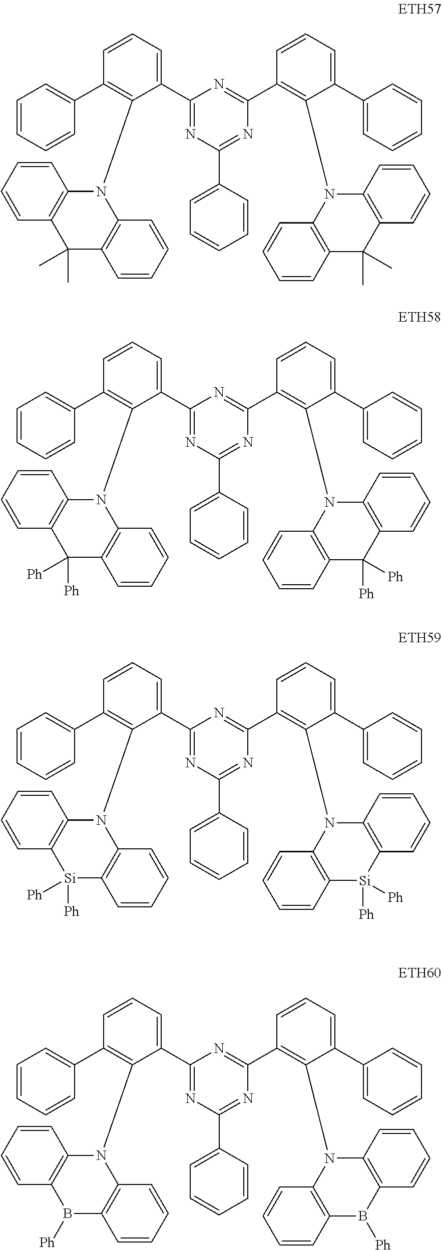

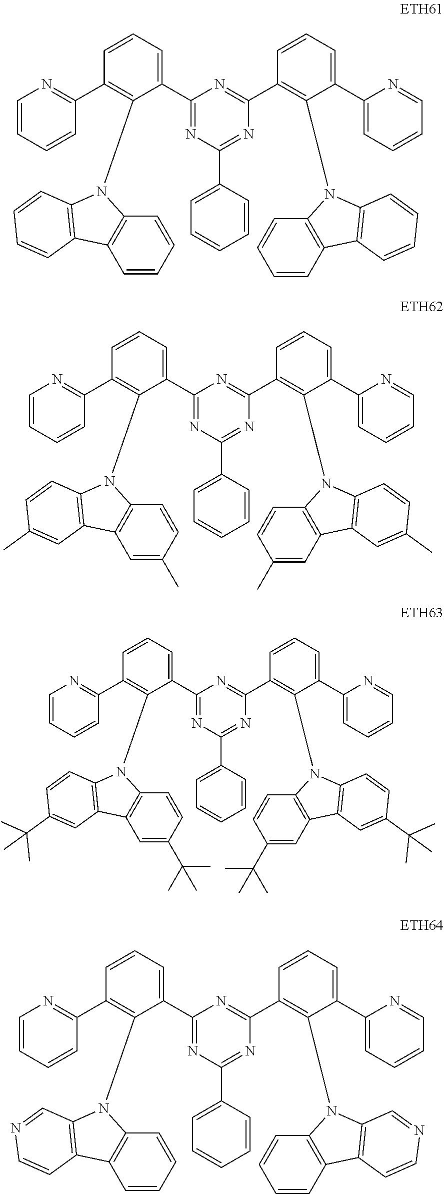

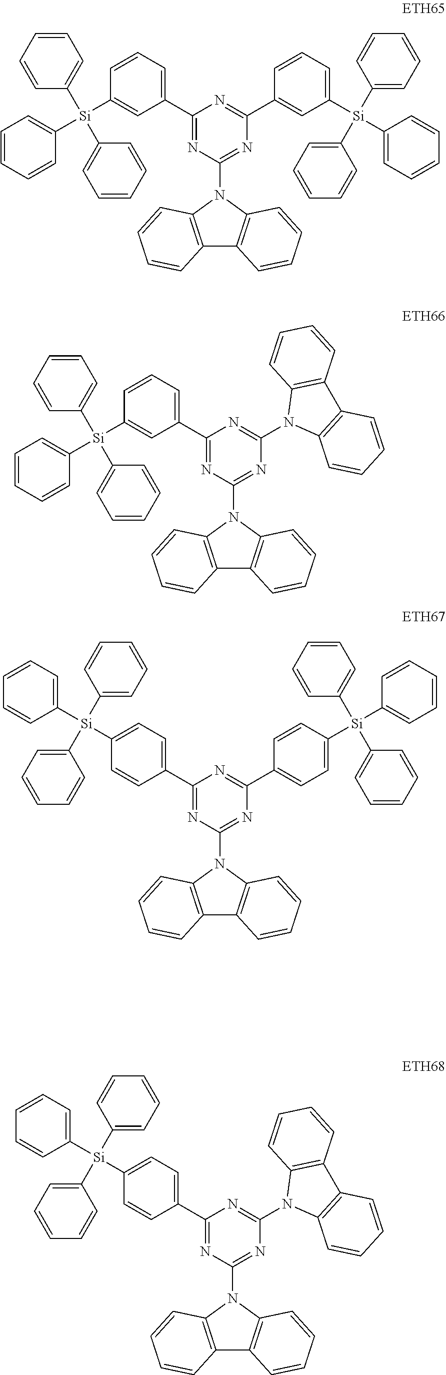 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5ccccc54)n3)c2)cc1 ; c1ccc([Si](c2ccccc2)(c2ccccc2)c2cccc(-c3nc(-n4c5ccccc5c5ccccc54)nc(-n4c5ccccc5c5ccccc54)n3)c2)cc1 ; c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5ccccc54)n3)cc2)cc1 ; c1ccc([Si](c2ccccc2)(c2ccccc2)c2ccc(-c3nc(-n4c5ccccc5c5ccccc54)nc(-n4c5ccccc5c5ccccc54)n3)cc2)cc1]

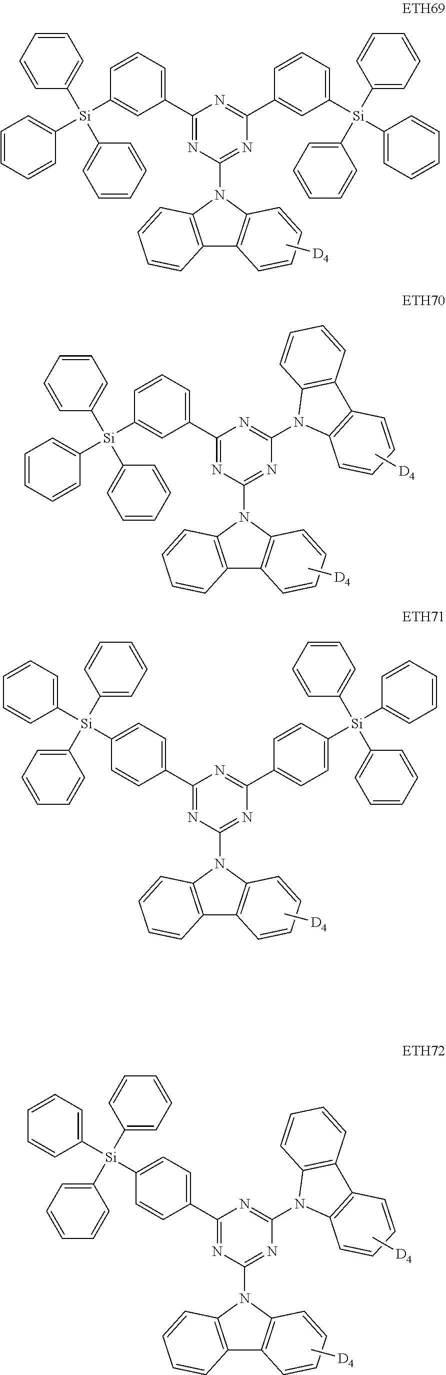

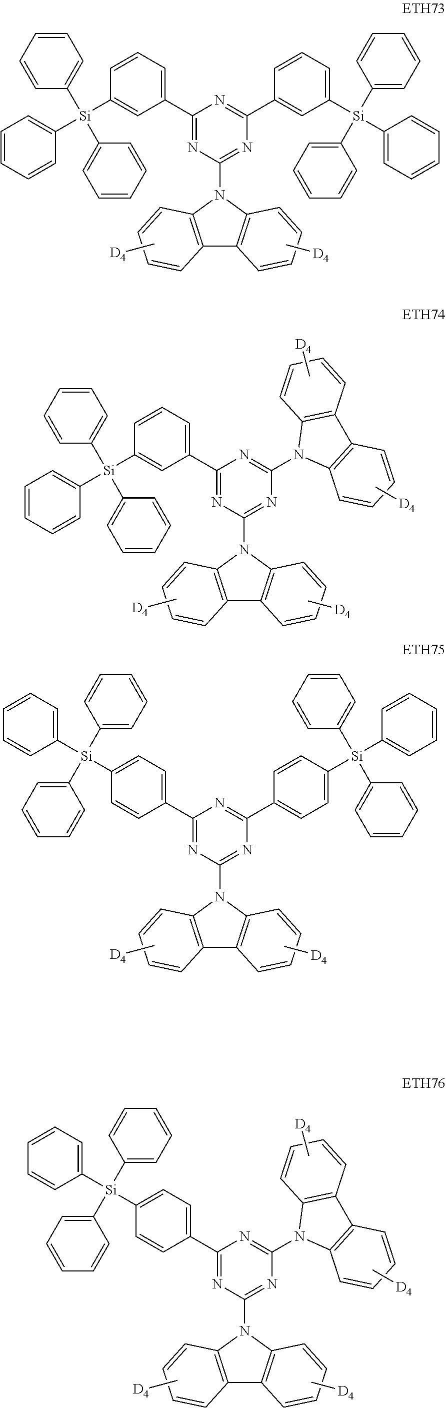

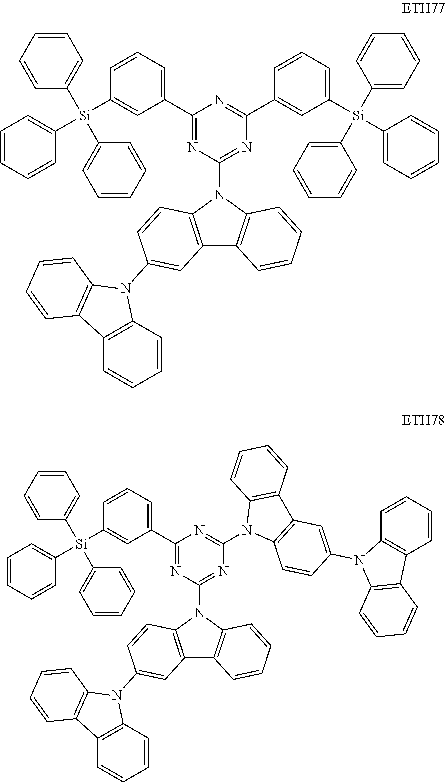 [SMILES: c1ccc([Si](c2ccccc2)(c2ccccc2)c2cccc(-c3nc(-c4cccc([Si](c5ccccc5)(c5ccccc5)c5ccccc5)c4)nc(-n4c5ccccc5c5cc(-n6c7ccccc7c7ccccc76)ccc54)n3)c2)cc1 ; c1ccc([Si](c2ccccc2)(c2ccccc2)c2cccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)c2)cc1]

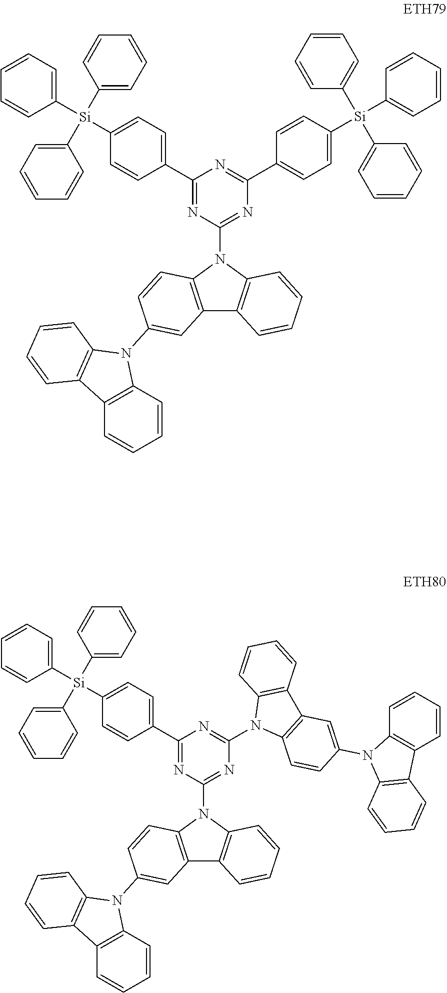 [SMILES: c1ccc([Si](c2ccccc2)(c2ccccc2)c2ccc(-c3nc(-c4ccc([Si](c5ccccc5)(c5ccccc5)c5ccccc5)cc4)nc(-n4c5ccccc5c5cc(-n6c7ccccc7c7ccccc76)ccc54)n3)cc2)cc1 ; c1ccc([Si](c2ccccc2)(c2ccccc2)c2ccc(-c3nc(-n4c5ccccc5c5cc(-n6c7ccccc7c7ccccc76)ccc54)nc(-n4c5ccccc5c5cc(-n6c7ccccc7c7ccccc76)ccc54)n3)cc2)cc1]

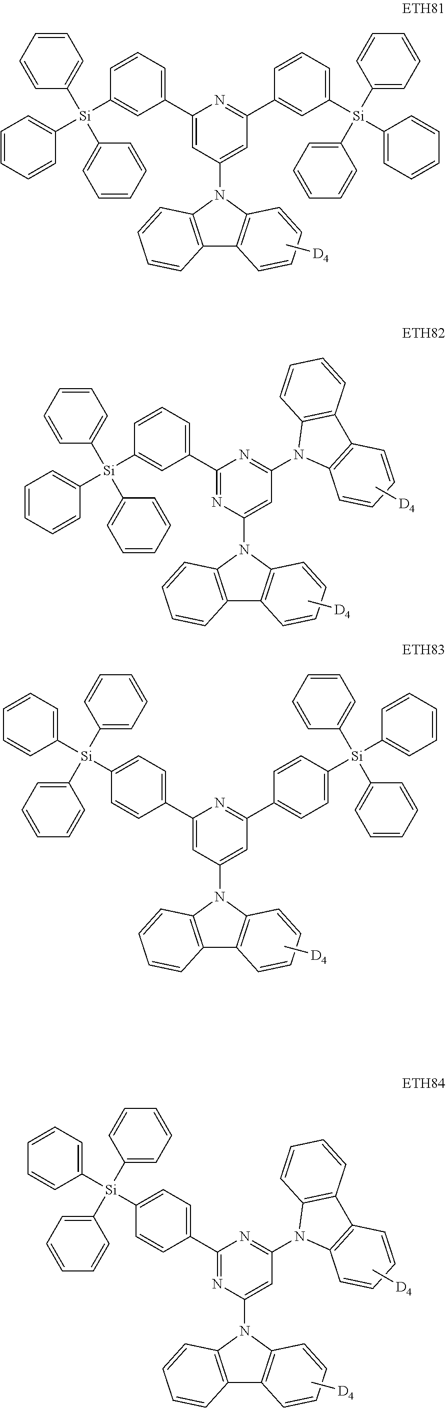

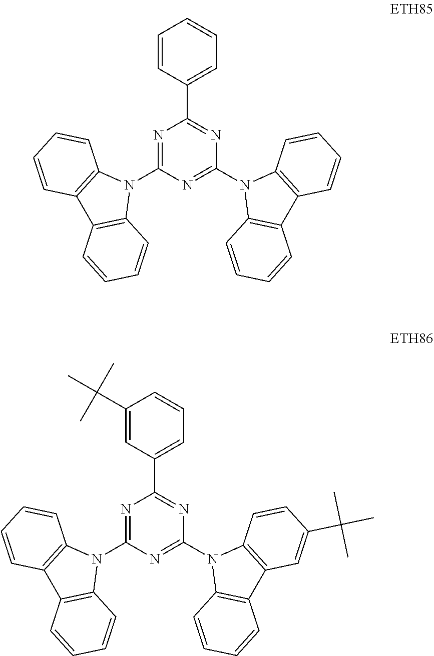 [SMILES: c1ccc(-c2nc(-n3c4ccccc4c4ccccc43)nc(-n3c4ccccc4c4ccccc43)n2)cc1 ; CC(C)(C)c1cccc(-c2nc(-n3c4ccccc4c4ccccc43)nc(-n3c4ccccc4c4cc(C(C)(C)C)ccc43)n2)c1]

In the particular compounds suggested in Compound Group 3, “D” means a deuterium atom, and “Ph” means an unsubstituted phenyl group.

The emission layer EML may include the compound represented by Formula HT-1 and the compound represented by Formula ET-1, and the Formula HT-1 compound and the Formula ET-1 compound may form exciplex. In the emission layer EML, exciplex may be formed by a hole transport host and an electron transport host. In this case, the triplet energy of the exciplex formed by the hole transport host and the electron transport host may correspond to a difference between the lowest unoccupied molecular orbital (LUMO) energy level of the electron transport host and the highest occupied molecular orbital (HOMO) energy level of the hole transport host.

For example, the absolute value of the triplet energy level (T1) of the exciplex formed by the hole transport host and the electron transport host may be about 2.4 eV to about 3.0 eV. In addition, the triplet energy of the exciplex may be a smaller value than the energy gap of each host material. The exciplex may have a triplet energy of about 3.0 eV or less, that is the energy gap between the hole transport host and the electron transport host.

In an embodiment, the emission layer EML may include a D-1 compound in addition to the HT-1 compound and the ET-1 compound. The D-1 compound may be used as a phosphorescence sensitizer of an emission layer EML. Since energy may transfer from the D-1 compound to the dopant compound, light emission may arise.

For example, the emission layer EML may include an organometallic complex including platinum (Pt) as a central metal atom and ligands bonded to the central metal atom, as the D-1 compound. In the light emitting device ED of an embodiment, the emission layer EML may include a compound represented by Formula D-1.

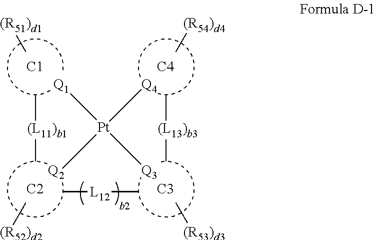

In Formula D-1, Q₁ to Q₄ may be each independently C or N.

In Formula D-1, C1 to 04 may be each independently a substituted or unsubstituted hydrocarbon ring of 5 to 30 ring-forming carbon atoms, or a substituted or unsubstituted heterocycle of 2 to 30 ring-forming carbon atoms.

In Formula D-1, L₁₁ to L₁₃ may be each independently a direct linkage,

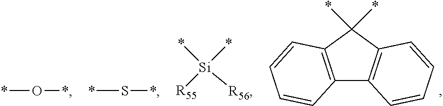

a substituted or unsubstituted divalent alkyl group of 1 to 20 carbon atoms, a substituted or unsubstituted arylene group of 6 to 30 ring-forming carbon atoms, or a substituted or unsubstituted heteroarylene group of 2 to 30 ring-forming carbon atoms. In L₁₁ to L₁₃, “ [symbol] ” means a part connected with C1 to C4.

In Formula D-1, b1 to b3 may be each independently 0 or 1. If b1 is 0, C1 and C2 may be unconnected. If b2 is 0, C2 and C3 may be unconnected. If b3 is 0, C3 and C4 may be unconnected.

In Formula D-1, R₅₁ to R₅₆ may be each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group of 1 to 20 carbon atoms, a substituted or unsubstituted alkenyl group of 2 to 20 carbon atoms, a substituted or unsubstituted aryl group of 6 to 60 ring-forming carbon atoms, or a substituted or unsubstituted heteroaryl group of 2 to 60 ring-forming carbon atoms. Otherwise, each of R₅₁ to R₅₆ may be combined with an adjacent group to form a ring. R₅₁ to R₅₆ may be each independently a substituted or unsubstituted methyl group, or a substituted or unsubstituted t-butyl group.

In Formula D-1, d1 to d4 are each independently an integer of 0 to 4. In Formula D-1, if d1 to d4 are 0, the compound may be unsubstituted with R₅₁ to R₅₄, respectively. A case where d1 to d4 are 4, and R₅₁ to R₅₄ are hydrogen atoms, may be the same as a case where d1 to d4 are 0. If d1 to d4 are integers of 2 or more, each of multiple R₅₁ to R₅₄ may be all the same, or at least one among multiple R₅₁ to R₅₄ may be different.

In Formula D-1, C1 to C4 may be each independently a substituted or unsubstituted hydrocarbon ring or a substituted or unsubstituted heterocycle, represented by any one among C-1 to C-4 below.

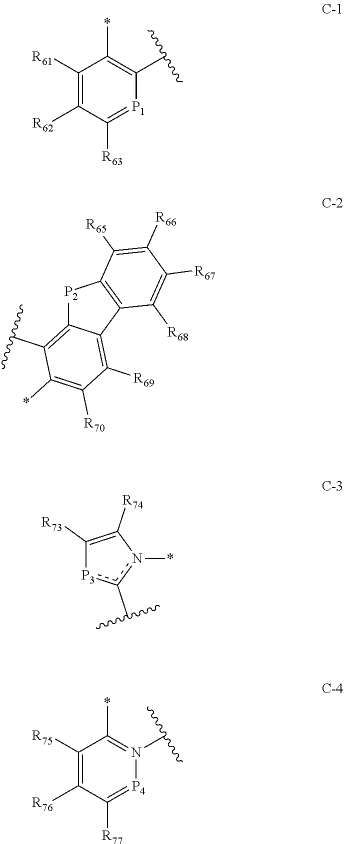

In C-1 to C-4, P₁ may be [symbol] or CR₆₄, P₂ may be [symbol] or NR₇₁, P₃ may be [symbol] or NR₇₂, and P₄ may be [symbol] or CR₇₈. R₆₁ to R₆₈ may be each independently a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In addition, in C-1 to C-4,

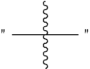

is a part connected with a central metal atom of Pt, and “ [symbol] ” corresponds to a part connected with an adjacent ring group (C1 to C4) or linker (L₁₁ to L₁₃).

The emission layer EML may include the HT-1 compound and the FT-1 compound. In the emission layer EML, the HT-1 compound and the FT-1 compound may form exiplex, and in the exiplex, energy transfer to the dopant compound may arise, and light emission may arise.

In addition, the emission layer EML may include the HT-1 compound, the FT-1 compound and the D-1 compound. In the emission layer EML, the HT-1 compound and the ET-1 compound may form exiplex, and in the exiplex, energy transfer to the D-1 compound may arise, and light emission may arise. In an embodiment, the D-1 compound may be a sensitizer. In the light emitting device ED of an embodiment, the D-1 compound included in the emission layer EML may act as a sensitizer and may play the role of transferring energy from a host to the dopant compound. That is, the D-1 compound that plays the role of an auxiliary dopant may accelerate energy transfer to the dopant compound that is a light emitting dopant and increase the light emitting ratio of the dopant compound. Accordingly, the emission efficiency of the emission layer EML of an embodiment may be improved. In addition, if the energy transfer to the dopant compound increases, excitons formed in the emission layer EML may not be accumulated but rapidly emit light, and the deterioration of a device may be reduced. Accordingly, the lifetime of the light emitting device ED of an embodiment may increase.

In an embodiment, the compound represented by Formula D-1 may be represented by at least one among the compounds represented in Compound Group 4. The emission layer EML may include at least one among the compounds represented in Compound Group 4 as a sensitizer material.

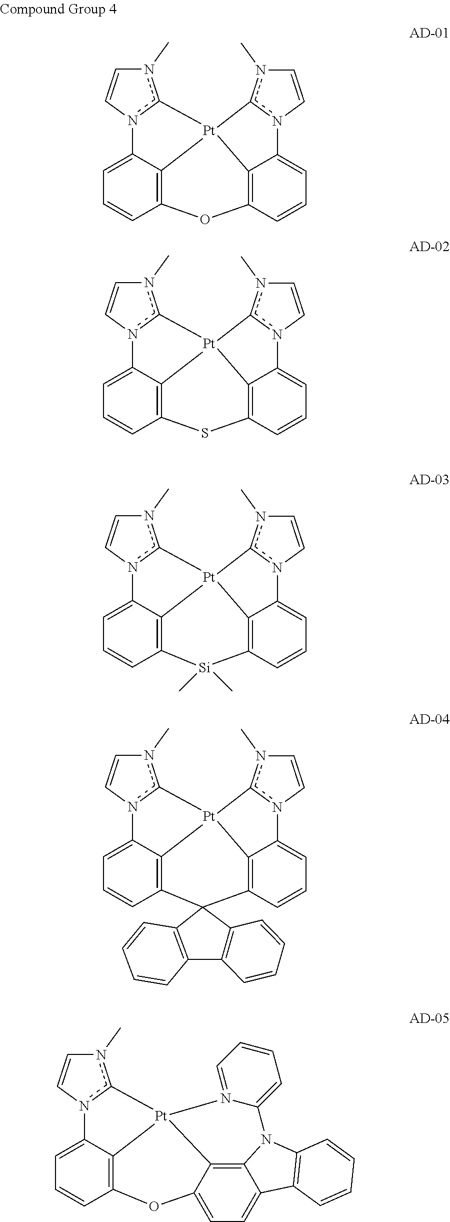

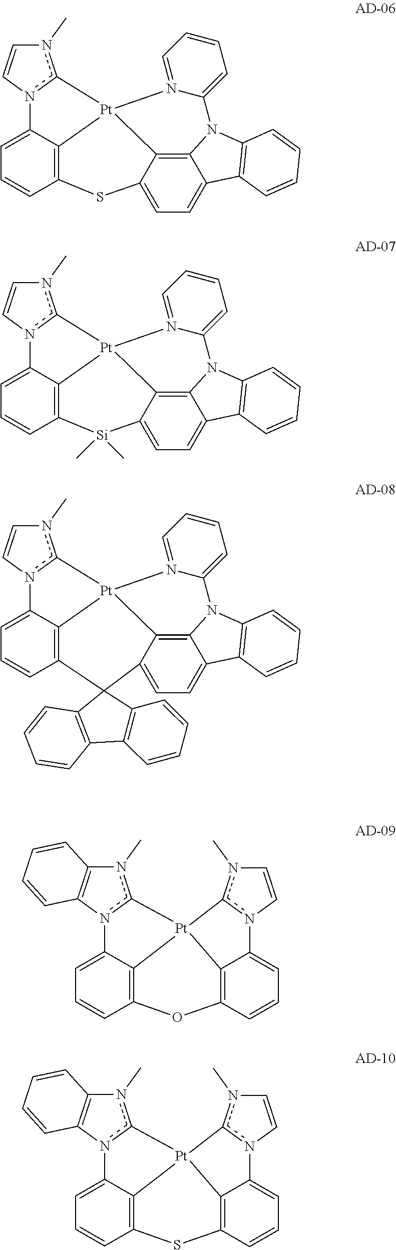

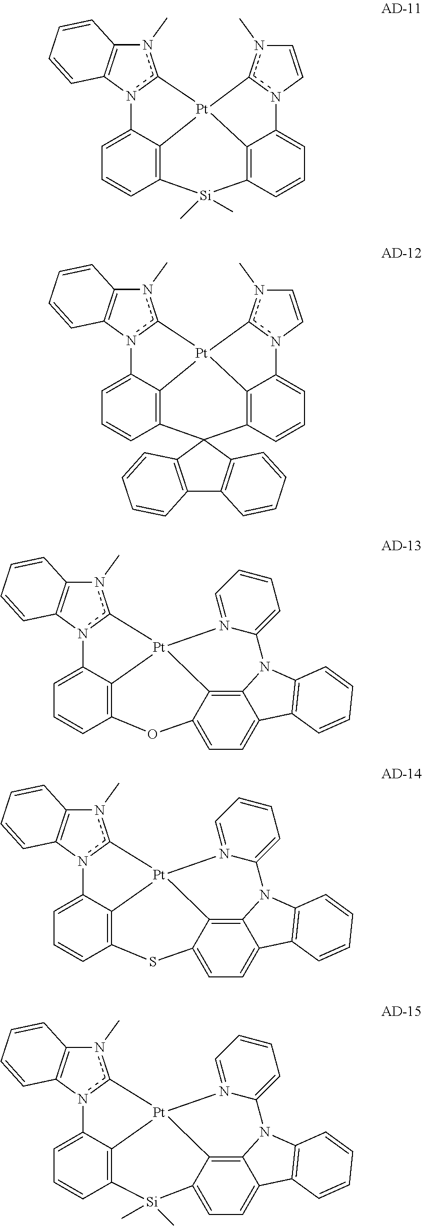

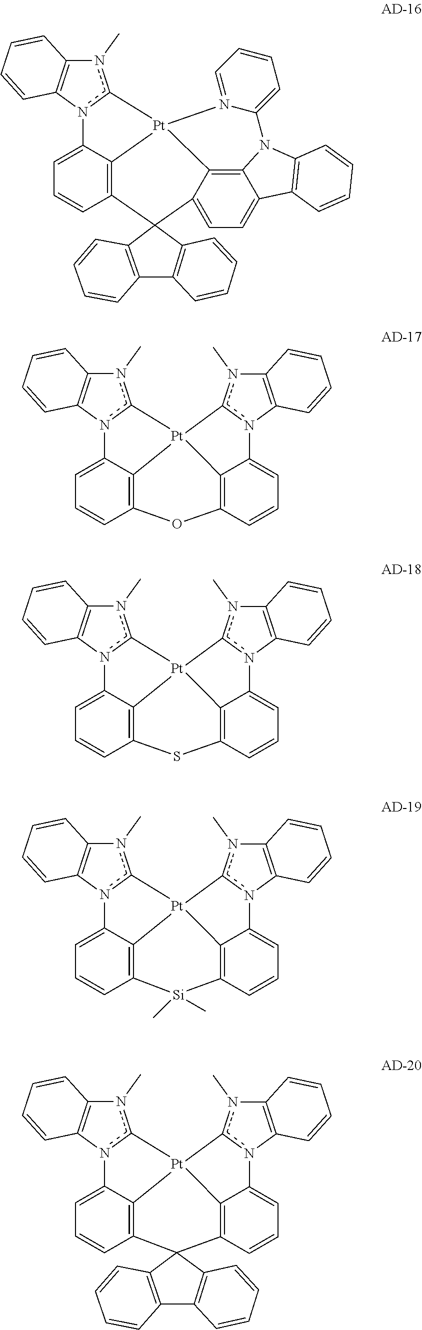

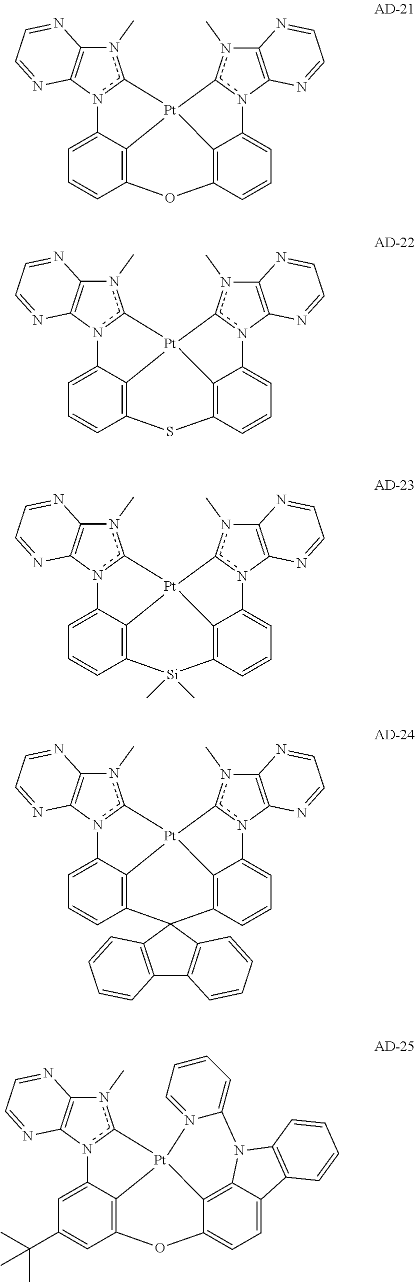

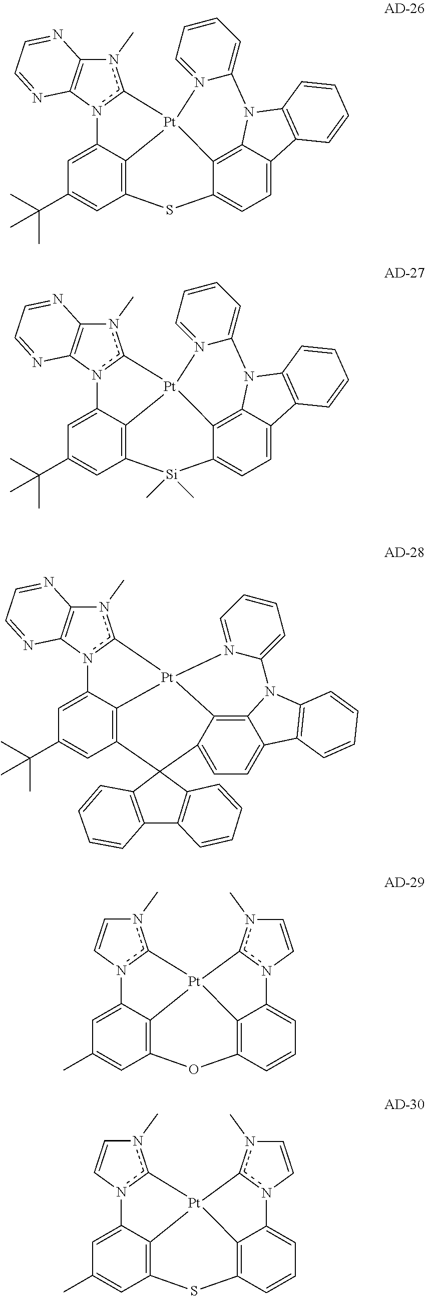

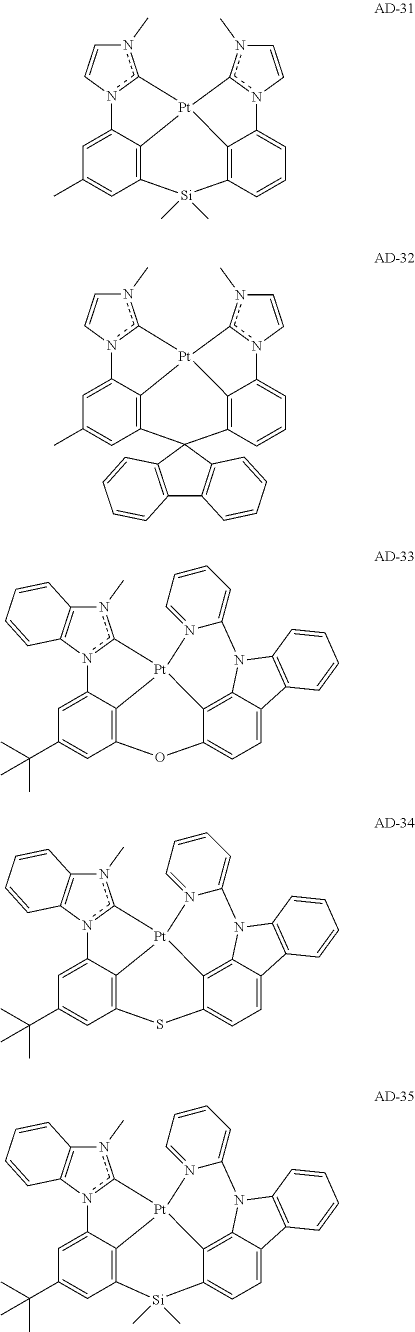

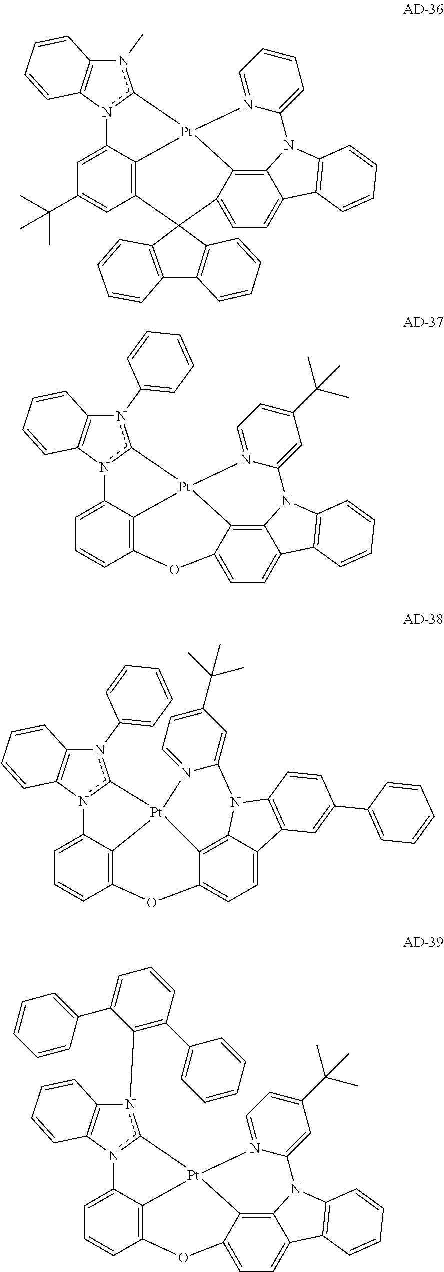

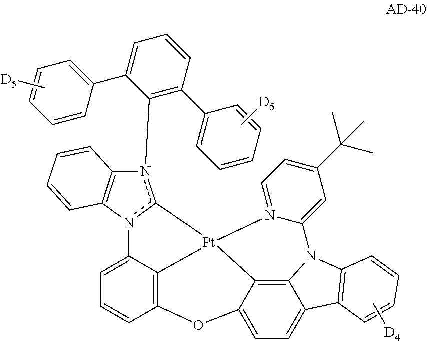

Meanwhile, the light emitting device ED of an embodiment may include multiple emission layers. Multiple emission layers may be stacked in order and provided, and for example a light emitting device ED including multiple emission layers may emit white light. The light emitting device including multiple emission layers may be a light emitting device of a tandem structure. In addition, if the light emitting device ED includes multiple emission layers, at least one emission layer EML may include all of the HT-1 compound, the ET-1 compound and the D-1 compound as described above.

In the light emitting device ED of an embodiment, the emission layer EML may further include anthracene derivatives, pyrene derivatives, fluoranthene derivatives, chrysene derivatives, dihydrobenzanthracene derivatives, or triphenylene derivatives. Particularly, the emission layer EML may include anthracene derivatives or pyrene derivatives.

In the light emitting devices ED of embodiments, shown in FIG. 3 to FIG. 6, the emission layer EML may further include known hosts and dopants in addition to the above-described host and dopant. For example, the emission layer EML may include a compound represented by Formula E-1 below. The compound represented by Formula E-1 below may be used as a fluorescence host material.

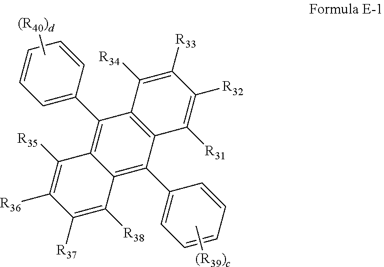

In Formula E-1, R₃₁ to R₄₀ may be each independently a hydrogen atom, a deuterium atom, a halogen atom, a substituted or unsubstituted silyl group, a substituted or unsubstituted thiol group, a substituted or unsubstituted oxy group, a substituted or unsubstituted alkyl group of 1 to 10 carbon atoms, a substituted or unsubstituted alkenyl group of 2 to 10 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring. Meanwhile, R₃₁ to R₄₀ may be combined with an adjacent group to form a saturated hydrocarbon ring, an unsaturated hydrocarbon ring, a saturated heterocycle or an unsaturated heterocycle.

In Formula E-1, “c” and “d” may be each independently an integer of 0 to 5.

Formula E-1 may be represented by any one among Compound E1 to

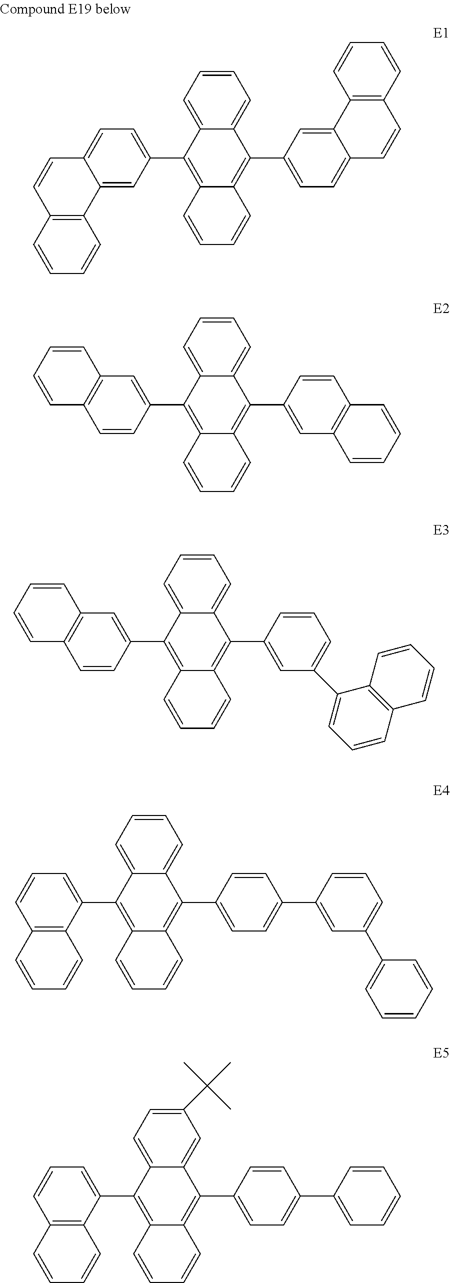

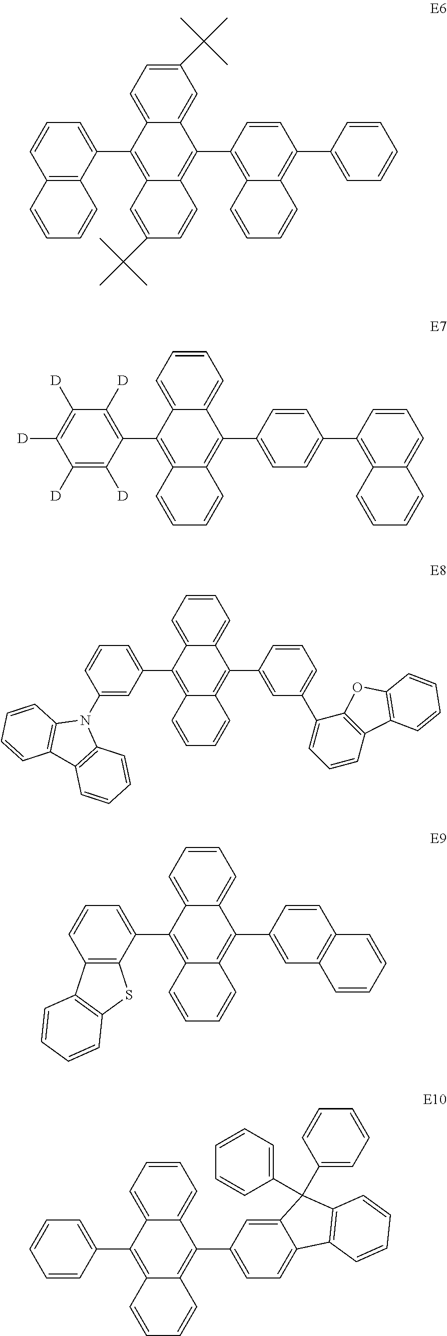

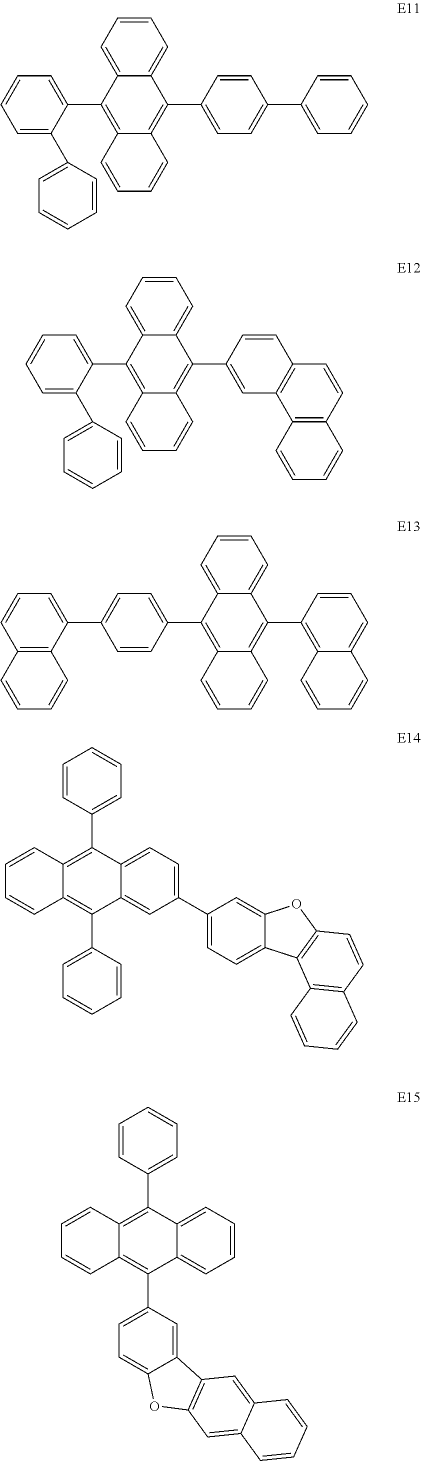

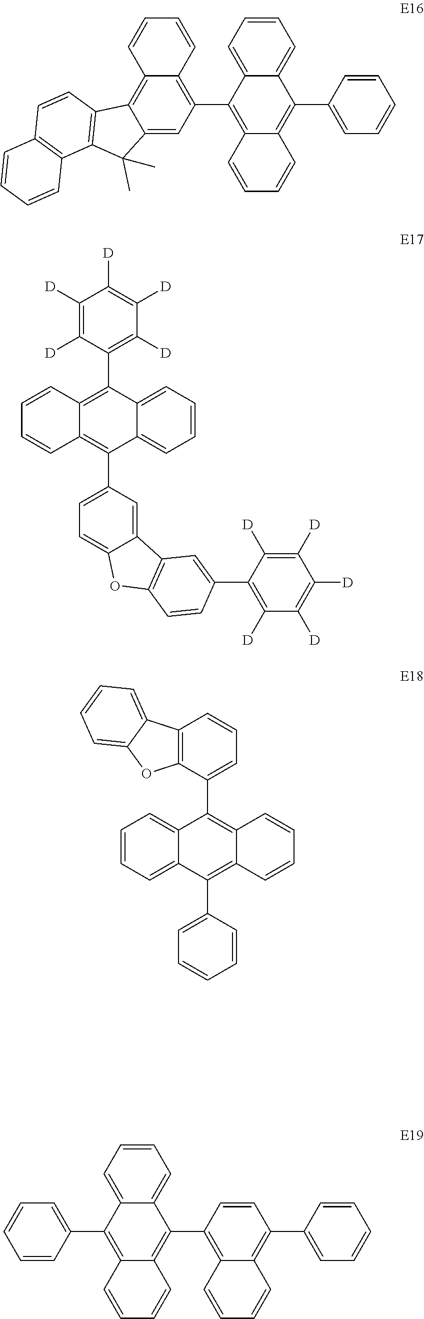

In an embodiment, the emission layer EML may include a compound represented by Formula E-2a or Formula E-2b below. The compound represented by Formula E-2a or Formula E-2b may be used as a phosphorescence host material.

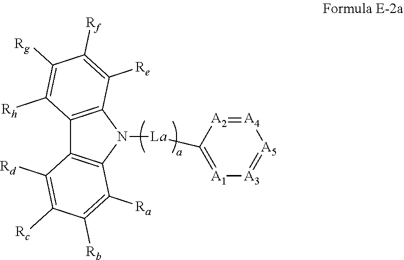

In Formula E-2a, “a” may be an integer of 0 to 10, Lₐ may be a direct linkage, a substituted or unsubstituted arylene group of 6 to 30 ring-forming carbon atoms, or a substituted or unsubstituted heteroarylene group of 2 to 30 ring-forming carbon atoms. Meanwhile, if “a” is an integer of 2 or more, multiple Lₐ may be each independently a substituted or unsubstituted arylene group of 6 to 30 ring-forming carbon atoms, or a substituted or unsubstituted heteroarylene group of 2 to 30 ring-forming carbon atoms.

In addition, in Formula E-2a, A₁ to A₅ may be each independently N or CRi. Rₐ to R₁ may be each independently a hydrogen atom, a deuterium atom, a substituted or unsubstituted amine group, a substituted or unsubstituted thiol group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Rₐ to R₁ may be combined with an adjacent group to form a hydrocarbon ring or a heterocycle including N, O, S, etc. as a ring-forming atom.

Meanwhile, in Formula E-2a, two or three selected from A₁ to A₅ may be N, and the remainder may be CRI.

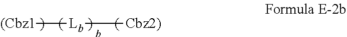

In Formula E-2b, Cbz1 and Cbz2 may be each independently an unsubstituted carbazole group, or a carbazole group substituted with an aryl group of 6 to 30 ring-forming carbon atoms. L_{b} may be a direct linkage, a substituted or unsubstituted arylene group of 6 to 30 ring-forming carbon atoms, or a substituted or unsubstituted heteroarylene group of 2 to 30 ring-forming carbon atoms. “b” is an integer of 0 to 10, and if “b” is an integer of 2 or more, multiple L_{b} may be each independently a substituted or unsubstituted arylene group of 6 to 30 ring-forming carbon atoms, or a substituted or unsubstituted heteroarylene group of 2 to 30 ring-forming carbon atoms.

The compound represented by Formula E-2a or Formula E-2b may be represented by any one among the compounds in Compound Group E-2 below. However, the compounds shown in Compound Group E-2 are only illustrations, and the compound represented by Formula E-2a or Formula E-2b is not limited to the compounds represented in Compound Group E-2.

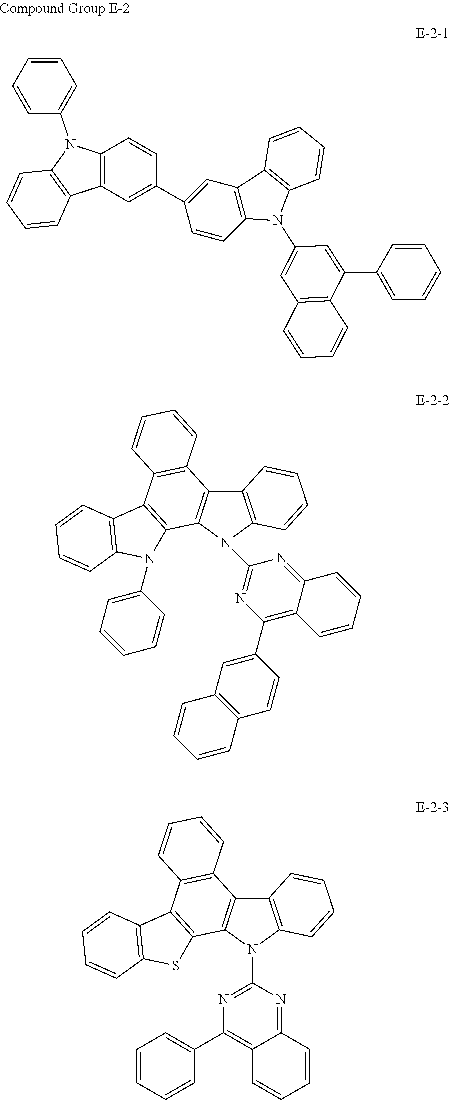 [SMILES: c1ccc(-c2cc(-n3c4ccccc4c4cc(-c5ccc6c(c5)c5ccccc5n6-c5ccccc5)ccc43)cc3ccccc23)cc1 ; c1ccc(-n2c3ccccc3c3c4ccccc4c4c5ccccc5n(-c5nc(-c6ccc7ccccc7c6)c6ccccc6n5)c4c32)cc1 ; c1ccc(-c2nc(-n3c4ccccc4c4c5ccccc5c5c6ccccc6sc5c43)nc3ccccc23)cc1]

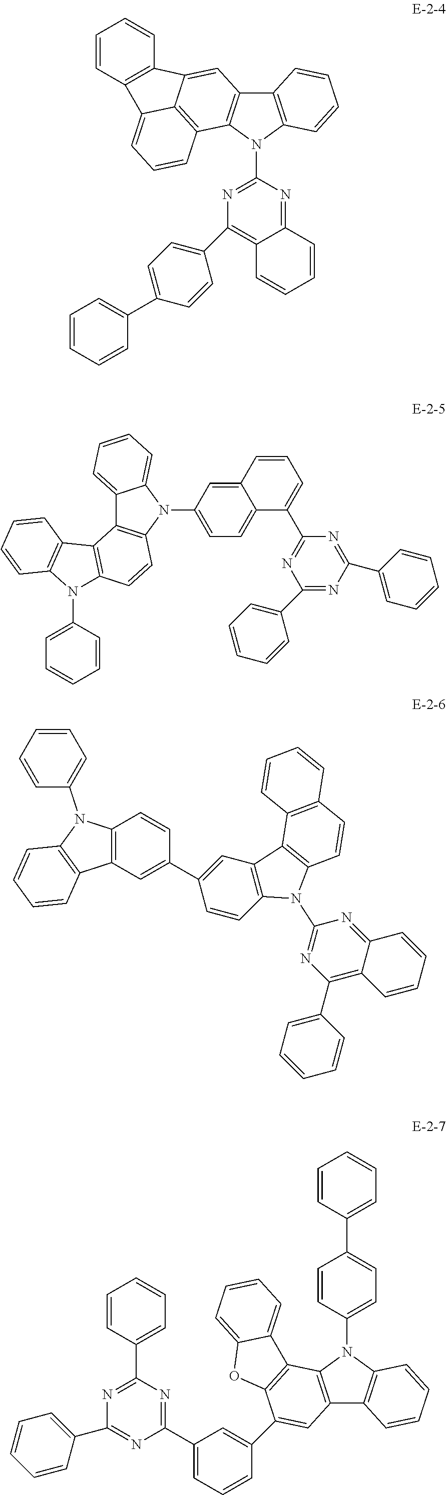

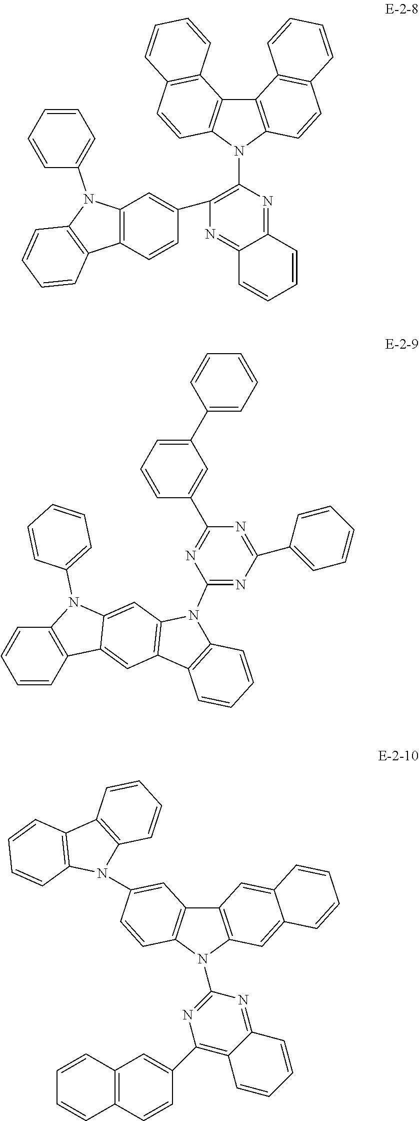 [SMILES: c1ccc(-n2c3ccccc3c3ccc(-c4nc5ccccc5nc4-n4c5ccc6ccccc6c5c5c6ccccc6ccc54)cc32)cc1 ; c1ccc(-c2cccc(-c3nc(-c4ccccc4)nc(-n4c5ccccc5c5cc6c7ccccc7n(-c7ccccc7)c6cc54)n3)c2)cc1 ; c1ccc2cc(-c3nc(-n4c5ccc(-n6c7ccccc7c7ccccc76)cc5c5cc6ccccc6cc54)nc4ccccc34)ccc2c1]

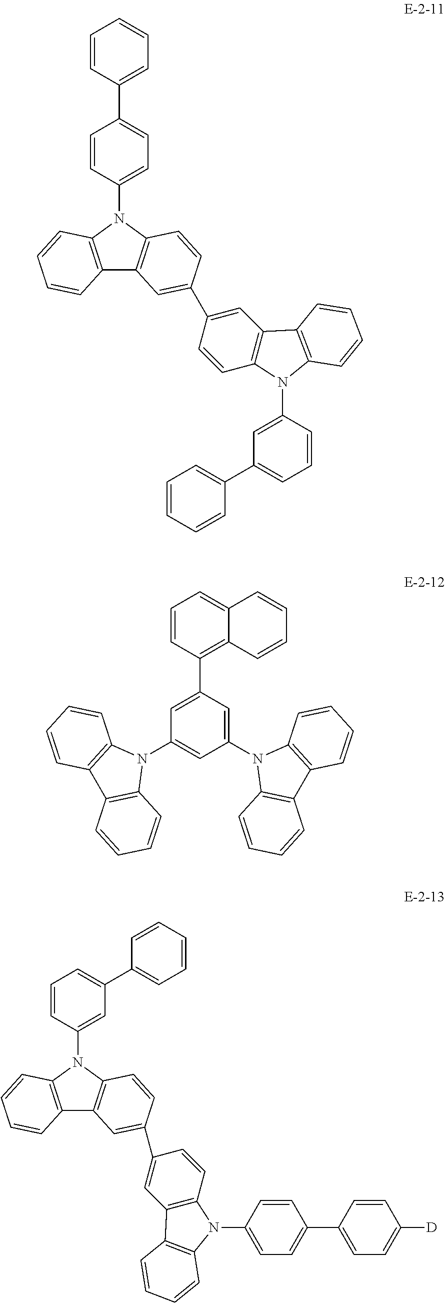 [SMILES: c1ccc(-c2ccc(-n3c4ccccc4c4cc(-c5ccc6c(c5)c5ccccc5n6-c5cccc(-c6ccccc6)c5)ccc43)cc2)cc1 ; c1ccc2c(-c3cc(-n4c5ccccc5c5ccccc54)cc(-n4c5ccccc5c5ccccc54)c3)cccc2c1 ; [2H]c1ccc(-c2ccc(-n3c4ccccc4c4cc(-c5ccc6c(c5)c5ccccc5n6-c5cccc(-c6ccccc6)c5)ccc43)cc2)cc1]

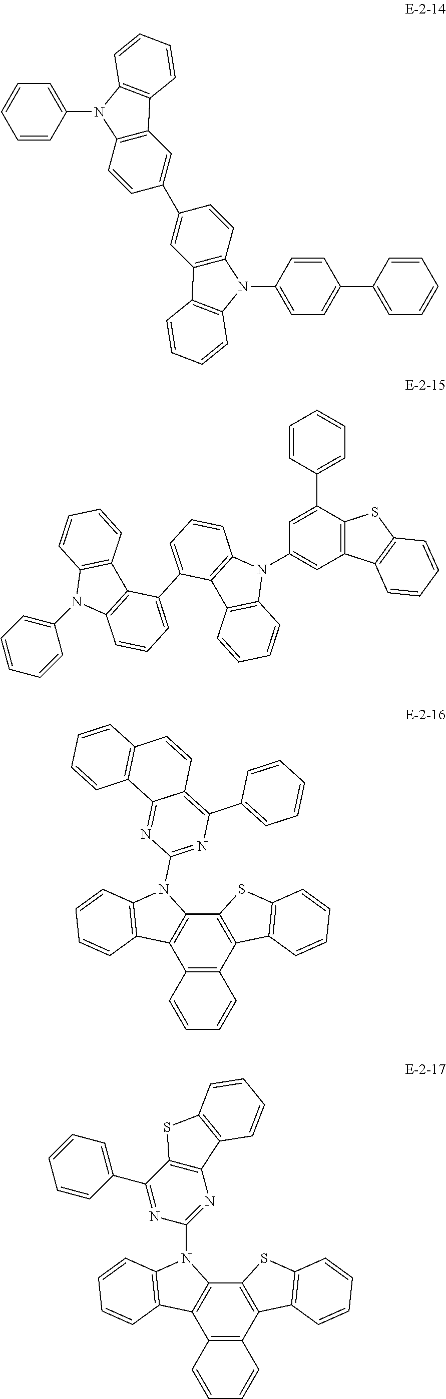 [SMILES: c1ccc(-c2ccc(-n3c4ccccc4c4cc(-c5ccc6c(c5)c5ccccc5n6-c5ccccc5)ccc43)cc2)cc1 ; c1ccc(-c2cc(-n3c4ccccc4c4c(-c5cccc6c5c5ccccc5n6-c5ccccc5)cccc43)cc3c2sc2ccccc23)cc1 ; c1ccc(-c2nc(-n3c4ccccc4c4c5ccccc5c5c6ccccc6sc5c43)nc3c2ccc2ccccc23)cc1 ; c1ccc(-c2nc(-n3c4ccccc4c4c5ccccc5c5c6ccccc6sc5c43)nc3c2sc2ccccc23)cc1]

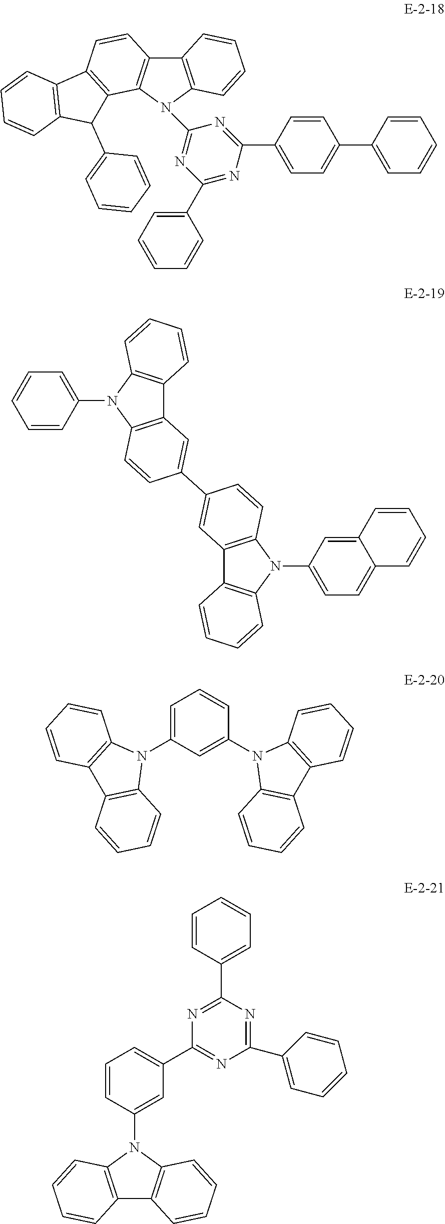 [SMILES: c1ccc(-c2ccc(-c3nc(-c4ccccc4)nc(-n4c5ccccc5c5ccc6c(c54)C(c4ccccc4)c4ccccc4-6)n3)cc2)cc1 ; c1ccc(-n2c3ccccc3c3cc(-c4ccc5c(c4)c4ccccc4n5-c4ccc5ccccc5c4)ccc32)cc1 ; c1cc(-n2c3ccccc3c3ccccc32)cc(-n2c3ccccc3c3ccccc32)c1 ; c1ccc(-c2nc(-c3ccccc3)nc(-c3cccc(-n4c5ccccc5c5ccccc54)c3)n2)cc1]

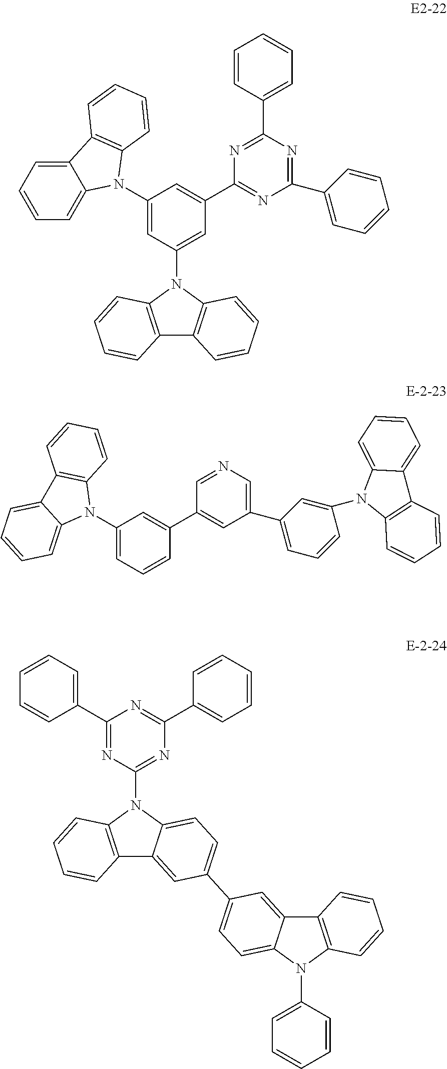 [SMILES: c1ccc(-c2nc(-c3ccccc3)nc(-c3cc(-n4c5ccccc5c5ccccc54)cc(-n4c5ccccc5c5ccccc54)c3)n2)cc1 ; c1cc(-c2cncc(-c3cccc(-n4c5ccccc5c5ccccc54)c3)c2)cc(-n2c3ccccc3c3ccccc32)c1 ; c1ccc(-c2nc(-c3ccccc3)nc(-n3c4ccccc4c4cc(-c5ccc6c(c5)c5ccccc5n6-c5ccccc5)ccc43)n2)cc1]

The emission layer EML may further include a common material well-known in the art as a host material. For example, the emission layer EML may include as a host material, at least one of bis(4-(9H-carbazol-9-yl)phenyl) diphenylsilane (BCPDS), (4-(1-(4-(diphenylamino)phenyl)cyclohexyl)phenyl)diphenyl-phosphine oxide (POPCPA), bis[2-(diphenylphosphino)phenyl]ether oxide (DPEPO), 4,4′-bis(N-carbazolyl)-1,1′-biphenyl (CBP), 1,3-bis(carbazol-9-yl)benzene (mCP), 2,8-bis(diphenyl phosphoryl)dibenzo[b,d]furan (PPF), 4,4′,4″-tris(carbazol-9-yl)-triphenylamine (TCTA), or 1,3,5-tris(1-phenyl-1H-benzo[d]imidazole-2-yl)benzene (TPBi). However, an embodiment of the inventive concept is not limited thereto. For example, tris(8-hydroxyquinolino)aluminum (Alq₃), 9,10-di(naphthalene-2-yl)anthracene (ADN), 2-tert-butyl-9,10-di(naphth-2-yl)anthracene (TBADN), distyrylarylene (DSA), 4,4′-bis(9-carbazolyl)-2,2′-dimethyl-biphenyl (CDBP), 2-methyl-9,10-bis(naphthalen-2-yl)anthracene (MADN), hexaphenyl cyclotriphosphazene (CP1), 1,4-bis(triphenylsilyl)benzene (UGH2), hexaphenylcyclotrisiloxane (DPSiO₃), octaphenylcyclotetra siloxane (DPSiO₄), etc. may be used as the host material.

The emission layer EML may include a compound represented by Formula M-a or Formula M-b below. The compound represented by Formula M-a or Formula M-b below may be used as a phosphorescent dopant material. In addition, the compound represented by Formula M-a or Formula M-b in an embodiment may be used as an auxiliary dopant material.

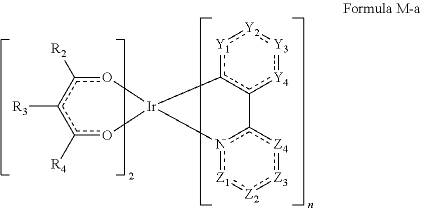

In Formula M-a, Y₁ to Y₄, and Z₁ to Z₄ may be each independently CR₁ or N, and R₁ to R₄ may be each independently a hydrogen atom, a deuterium atom, a substituted or unsubstituted amine group, a substituted or unsubstituted thiol group, a substituted or unsubstituted oxy group, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. In Formula M-a, “m” is 0 or 1, and “n” is 2 or 3. In Formula M-a, if “m” is 0, “n” is 3, and if “m” is 1, “n” is 2.

The compound represented by Formula M-a may be used as a phosphorescence dopant.

The compound represented by Formula M-a may be represented by any one among Compounds M-a₁ to M-a₂₅ below. However, Compounds M-a₁ to M-a₂₅ are illustrations, and the compound represented by Formula M-a is not limited to the compounds represented by Compounds M-a₁ to M-a₂₅ below.

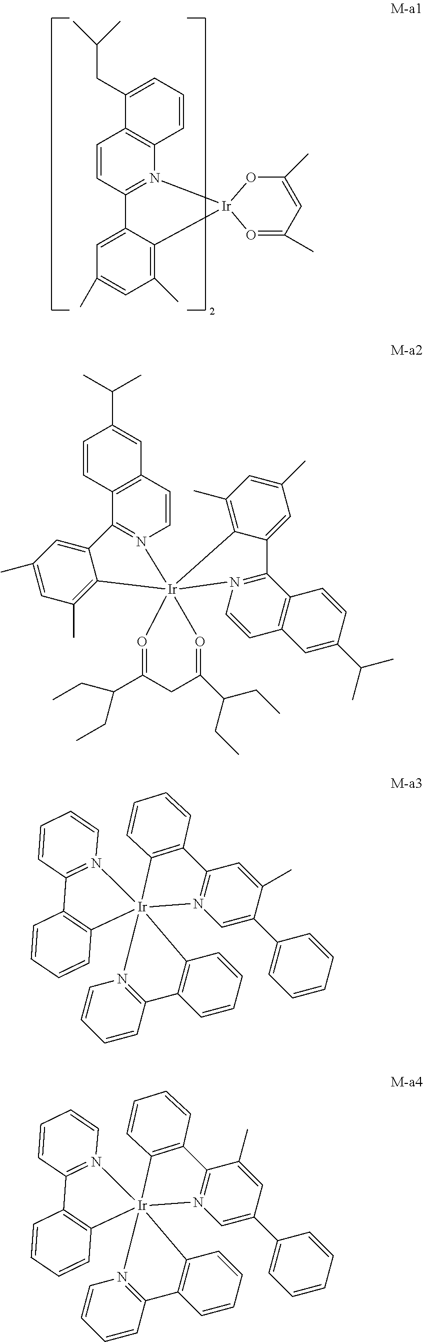

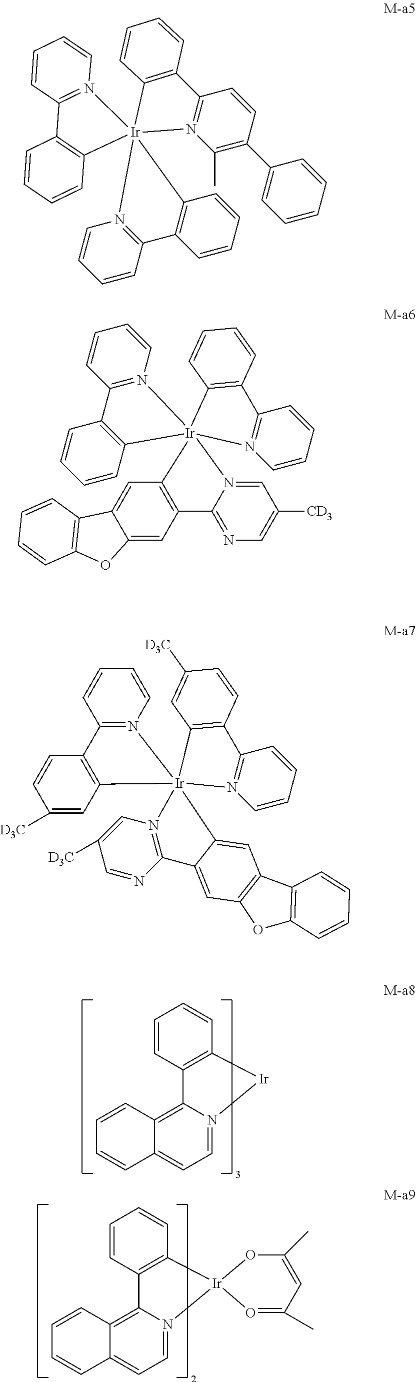

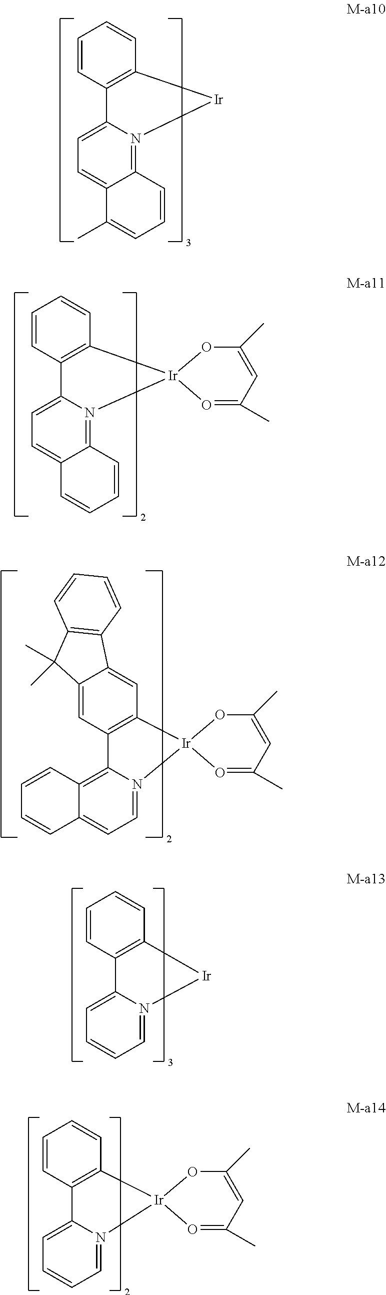

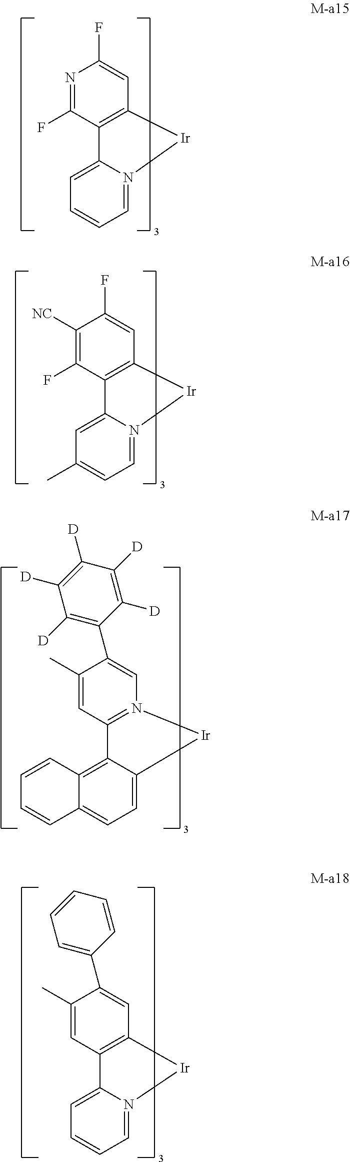

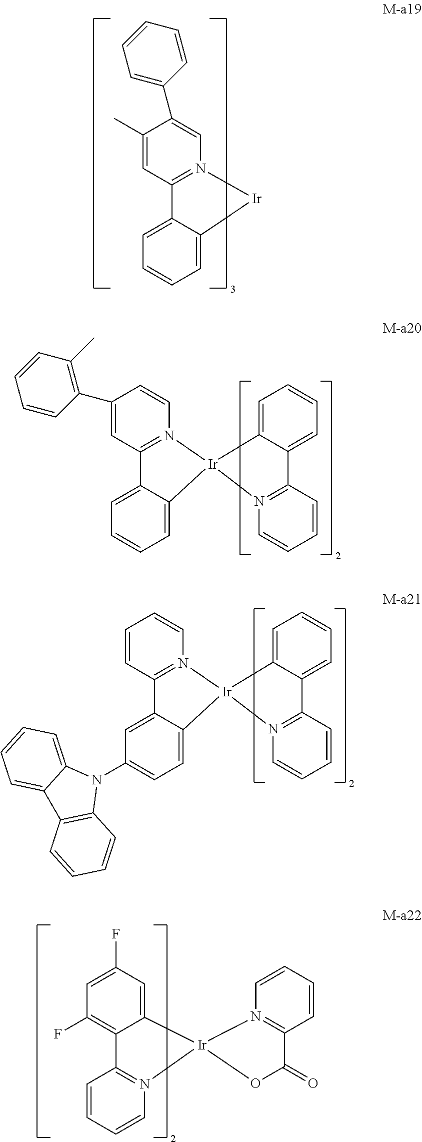

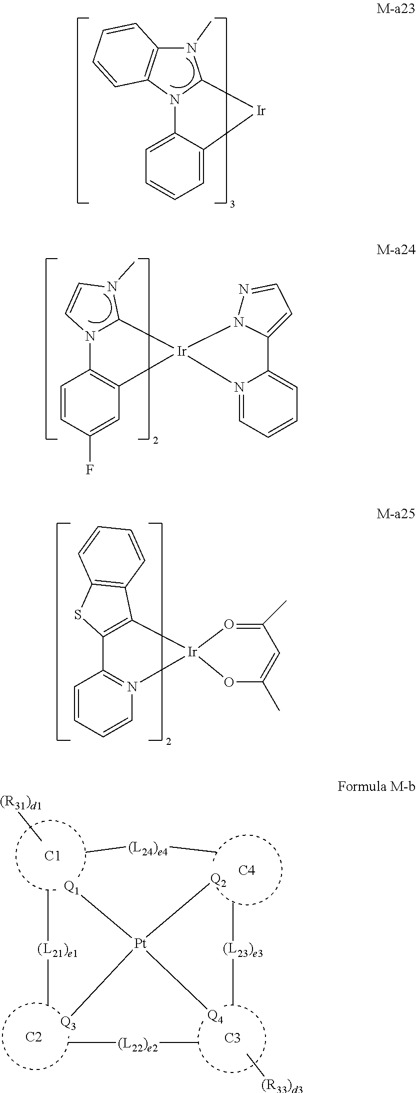

In Formula M-b, Q₁ to Q₄ are each independently C or N, and C1 to 04 are each independently a substituted or unsubstituted hydrocarbon ring having 5 to 30 ring-forming carbon atoms, or a substituted or unsubstituted heterocycle having 2 to 30 ring-forming carbon atoms. L₂₁ to L₂₄ are each independently a direct linkage,

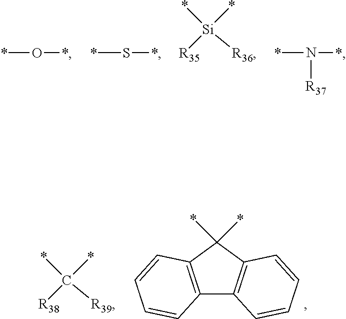

a substituted or unsubstituted divalent alkyl group having 1 to 20 carbon atoms, a substituted or unsubstituted arylene group having 6 to 30 ring-forming carbon atoms, or a substituted or unsubstituted heteroarylene group having 2 to 30 ring-forming carbon atoms, and e1 to e4 are each independently 0 or 1. R₃₁ to R₃₉ are each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms, or are bonded to an adjacent group to form a ring, and d1 to d4 are each independently an integer of 0 to 4.

The compound represented by Formula M-b may be used as a blue phosphorescent dopant or a green phosphorescent dopant. In addition, the compound represented by Formula M-b may be further included as an auxiliary dopant in the emission layer EML in an embodiment.

The compound represented by Formula M-b may be represented by any one among Compound M-b-1 to Compound M-b-11 below. However, the following compounds are examples, and the compounds represented by Formula M-b are not limited to Compounds M-b-1 to Compound M-b-11 below:

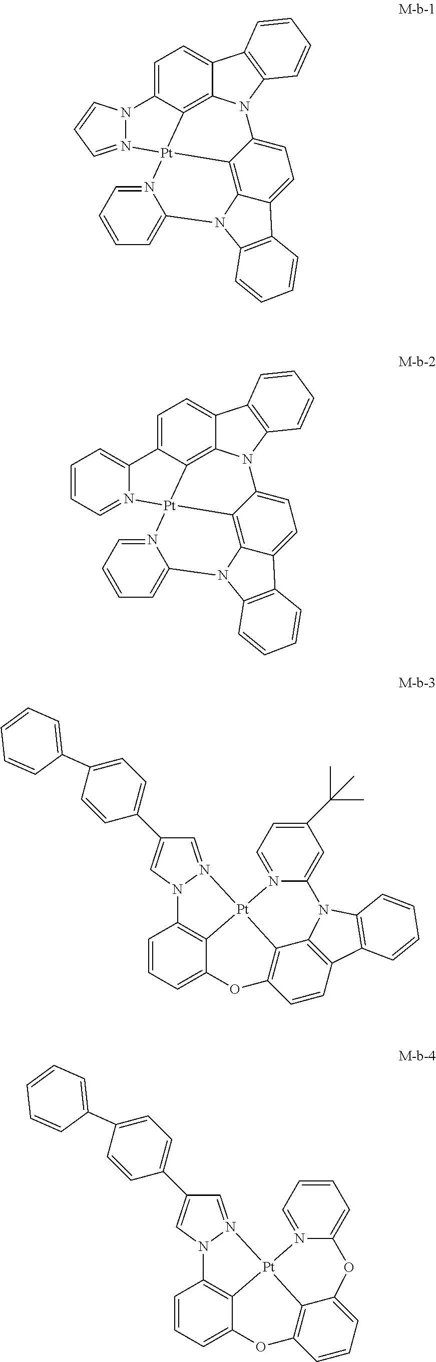

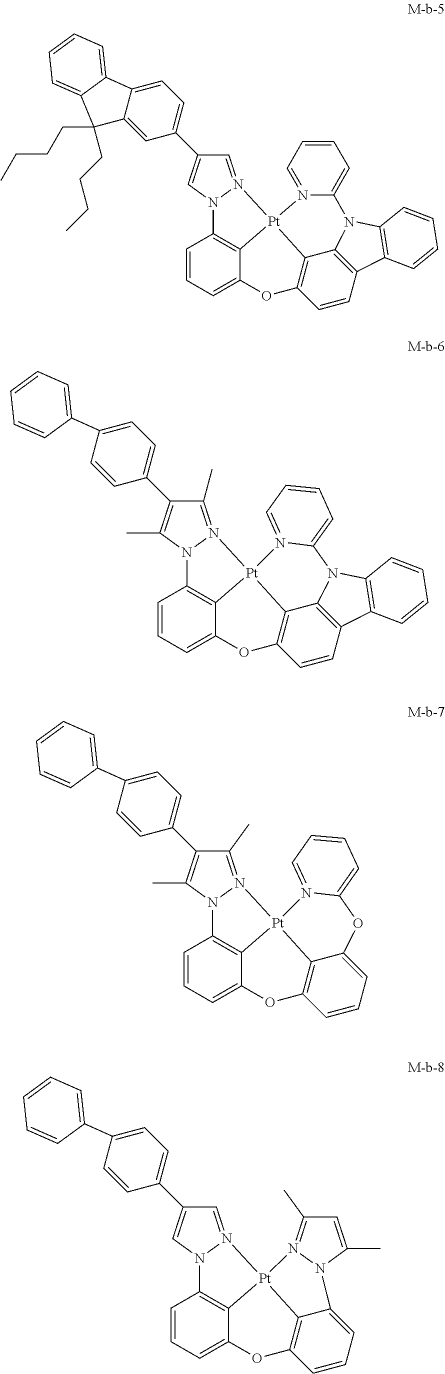

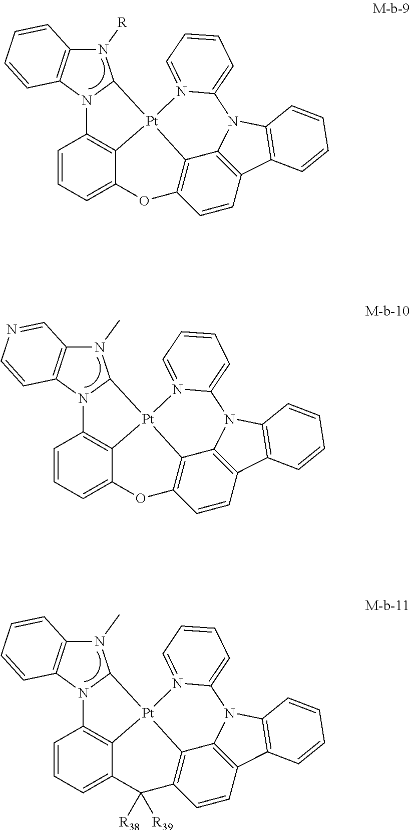

In the compounds, R, R₃₈, and R₃₉ may be each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 ring-forming carbon atoms, or a substituted or unsubstituted heteroaryl group having 2 to 30 ring-forming carbon atoms.

The emission layer EML may further include any one among Formula F-a to Formula F-c below. The compounds represented by Formula F-a to Formula F-c may be used as fluorescence dopant materials.

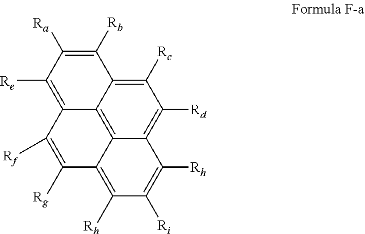

In Formula F-a, two selected from Rₐ to Rⱼ may be each independently substituted with [symbol] . The remainder not substituted with [symbol] among Rₐ to Rⱼ may be each independently a hydrogen atom, a deuterium atom, a halogen atom, a cyano group, a substituted or unsubstituted amine group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In [symbol] , Ar₁ and Ar₂ may be each independently a substituted or unsubstituted aryl group of 6 to 30 ring-forming carbon atoms, or a substituted or unsubstituted heteroaryl group of 2 to 30 ring-forming carbon atoms. For example, at least one among Ar₁ and Ar₂ may be a heteroaryl group including O or S as a ring-forming atom.

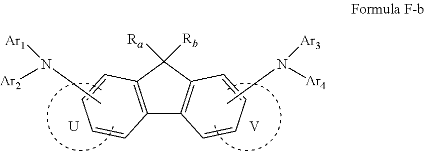

In Formula F-b, Rₐ and R_{b} may be each independently a hydrogen atom, a deuterium atom, a substituted or unsubstituted alkyl group of 1 to 20 carbon atoms, a substituted or unsubstituted alkenyl group of 2 to 20 carbon atoms, a substituted or unsubstituted aryl group of 6 to 30 ring-forming carbon atoms, or a substituted or unsubstituted heteroaryl group of 2 to 30 ring-forming carbon atoms, or may be combined with an adjacent group to form a ring. Ar₁ to Ar₄ may be each independently a substituted or unsubstituted aryl group of 6 to 30 ring-forming carbon atoms, or a substituted or unsubstituted heteroaryl group of 2 to 30 ring-forming carbon atoms.

In Formula F-b, U and V may be each independently a substituted or unsubstituted hydrocarbon ring of 5 to 30 ring-forming carbon atoms, or a substituted or unsubstituted heterocycle of 2 to 30 ring-forming carbon atoms. At least one among Ar₁ to Ar₄ may be a heteroaryl group including O or S as a ring-forming atom.

In Formula F-b, the number of rings represented by U and V may be each independently 0 or 1. For example, in Formula F-b, if the number of U or V is 1, one ring forms a fused ring at the designated part by U or V, and if the number of U or V is 0, a ring is not present at the designated part by U or V. Particularly, if the number of U is 0, and the number of V is 1, or if the number of U is 1, and the number of V is 0, a fused ring having the fluorene core of Formula F-b may be a ring compound with four rings. In addition, if the number of both U and V is 0, the fused ring of Formula F-b may be a ring compound with three rings. In addition, if the number of both U and V is 1, a fused ring having the fluorene core of Formula F-b may be a ring compound with five rings.

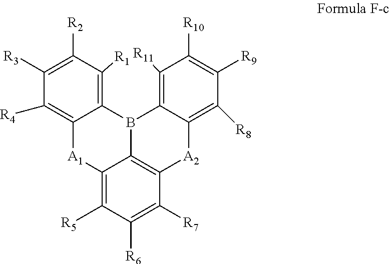

In Formula F-c, A₁ and A₂ may be each independently O, S, Se, or NRₘ, and Rₘ may be a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms. R₁ to R₁₁ are each independently a hydrogen atom, a deuterium atom, a halogen atom, a cyano group, a substituted or unsubstituted amine group, a substituted or unsubstituted boryl group, a substituted or unsubstituted oxy group, a substituted or unsubstituted thiol group, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms, or combined with an adjacent group to form a ring.

In Formula F-c, A₁ and A₂ may be each independently combined with the substituents of an adjacent ring to form a fused ring. For example, if A₁ and A₂ may be each independently NRₘ, A₁ may be combined with R₄ or R₅ to form a ring. In addition, A₂ may be combined with R₇ or Rₐ to form a ring.

In an embodiment, the emission layer EML may include as a known dopant material, styryl derivatives (for example, 1,4-bis[2-(3-N-ethylcarbazoryl)vinyl]benzene (BCzVB), 4-(di-p-tolylamino)-4′-[(di-p-tolylamino)styryl]stilbene (DPAVB), N-(4-((E)-2-(6-((E)-4-(diphenylamino)styryl)naphthalen-2-yl)vinyl)phenyl)-N-phenylbenzenamine (N-BDAVBi), and 4,4′-bis[2-(4-(N,N-diphenylamino)phenyl)vinyl]biphenyl (DPAVBi)), perylene and the derivatives thereof (for example, 2,5,8,11-tetra-t-butylperylene (TBP)), pyrene and the derivatives thereof (for example, 1,1-dipyrene, 1,4-dipyrenylbenzene, and 1,4-bis(N,N-diphenylamino)pyrene), etc.

The emission layer EML may include a known phosphorescence dopant material. For example, the phosphorescence dopant may use a metal complex including iridium (Ir), platinum (Pt), osmium (Os), gold (Au), titanium (Ti), zirconium (Zr), hafnium (Hf), europium (Eu), terbium (Tb) or thulium (Tm). Particularly, iridium(III) bis(4,6-difluorophenylpyridinato-N,C2′)picolinate (Firpic), bis(2,4-difluorophenylpyridinato)-tetrakis(1-pyrazolyl)borate iridium(III) (Fir6), or platinum octaethyl porphyrin (PtOEP) may be used as the phosphorescence dopant. However, an embodiment of the inventive concept is not limited thereto.

The emission layer EML may include a quantum dot material. The core of the quantum dot may be selected from a II-VI group compound, a III-VI group compound, a 1-III-VI group compound, a III-V group compound, a III-II-V group compound, a IV-VI group compound, a IV group element, a IV group compound, and combinations thereof.

The II-VI group compound may be selected from the group consisting of: a binary compound selected from the group consisting of CdSe, CdTe, CdS, ZnS, ZnSe, ZnTe, ZnO, HgS, HgSe, HgTe, MgSe, MgS, and mixtures thereof; a ternary compound selected from the group consisting of CdSeS, CdSeTe, CdSTe, ZnSeS, ZnSeTe, ZnSTe, HgSeS, HgSeTe, HgSTe, CdZnS, CdZnSe, CdZnTe, CdHgS, CdHgSe, CdHgTe, HgZnS, HgZnSe, HgZnTe, MgZnSe, MgZnS, and mixtures thereof; and a quaternary compound selected from the group consisting of HgZnTeS, CdZnSeS, CdZnSeTe, CdZnSTe, CdHgSeS, CdHgSeTe, CdHgSTe, HgZnSeS, HgZnSeTe, HgZnSTe, and mixtures thereof.

The III-VI group compound may include a binary compound such as In₂S₃, and In₂Se₃, a ternary compound such as InGaS₃, and InGaSe₃, or arbitrary combinations thereof.

The I-III-VI group compound may be selected from a ternary compound selected from the group consisting of AgInS, AgInS₂, CuInS, CuInS₂, AgGaS₂, CuGaS₂, CuGaO₂, AgGaO₂, AgAlO₂ and mixtures thereof, or a quaternary compound such as AgInGaS₂, and CuInGaS₂.

The III-V group compound may be selected from the group consisting of a binary compound selected from the group consisting of GaN, GaP, GaAs, GaSb, AlN, AlP, AlAs, AlSb, InN, InP, InAs, InSb, and mixtures thereof, a ternary compound selected from the group consisting of GaNP, GaNAs, GaNSb, GaPAs, GaPSb, AlNP, AlNAs, AlNSb, AlPAs, AlPSb, InGaP, InAlP, InNP, InNAs, InNSb, InPAs, InPSb, and mixtures thereof, and a quaternary compound selected from the group consisting of GaAlNP, GaAlNAs, GaAlNSb, GaAlPAs, GaAlPSb, GaInNP, GaInNAs, GalnNSb, GaInPAs, GalnPSb, InAlNP, InAlNAs, InAlNSb, InAlPAs, InAlPSb, and mixtures thereof. Meanwhile, the III-V group compound may further include a II group metal. For example, InZnP, etc. may be selected as a III-II-V group compound.

The IV-VI group compound may be selected from the group consisting of a binary compound selected from the group consisting of SnS, SnSe, SnTe, PbS, PbSe, PbTe, and mixtures thereof, a ternary compound selected from the group consisting of SnSeS, SnSeTe, SnSTe, PbSeS, PbSeTe, PbSTe, SnPbS, SnPbSe, SnPbTe, and mixtures thereof, and a quaternary compound selected from the group consisting of SnPbSSe, SnPbSeTe, SnPbSTe, and mixtures thereof. The IV group element may be selected from the group consisting of Si, Ge, and a mixture thereof. The IV group compound may be a binary compound selected from the group consisting of SiC, SiGe, and a mixture thereof.

In this case, the binary compound, the ternary compound or the quaternary compound may be present at uniform concentration in a particle or may be present at a partially different concentration distribution state in the same particle. In addition, a core/shell structure in which one quantum dot wraps another quantum dot may be possible. The interface of the core and the shell may have a concentration gradient in which the concentration of an element present in the shell is decreased toward the center.

In some embodiments, the quantum dot may have the above-described core-shell structure including a core including a nanocrystal and a shell wrapping the core. The shell of the quantum dot may play the role of a protection layer for preventing the chemical deformation of the core to maintain semiconductor properties and/or a charging layer for imparting the quantum dot with electrophoretic properties.

The shell may have a single layer or a multilayer. Examples of the shell of the quantum dot may include a metal or non-metal oxide, a semiconductor compound, or combinations thereof.

For example, the metal or non-metal oxide may include a binary compound such as SiO₂, Al₂O₃, TiO₂, ZnO, MnO, Mn₂O₃, Mn₃O₄, CuO, FeO, Fe₂O₃, Fe₃O₄, CoO, Co₃O₄ and NiO, or a ternary compound such as MgAl₂O₄, CoFe₂O₄, NiFe₂O₄ and CoMn₂O₄, but an embodiment of the inventive concept is not limited thereto.

Also, the semiconductor compound may include CdS, CdSe, CdTe, ZnS, ZnSe, ZnTe, ZnSeS, ZnTeS, GaAs, GaP, GaSb, HgS, HgSe, HgTe, InAs, InP, InGaP, InSb, AlAs, AlP, AlSb, etc., but an embodiment of the inventive concept is not limited thereto.

The quantum dot may have a full width of half maximum (FWHM) of emission wavelength spectrum of about 45 nm or less, preferably, about 40 nm or less, more preferably, about 30 nm or less. Within this range, color purity or color reproducibility may be improved. In addition, light emitted via such quantum dot is emitted in all directions, and light view angle properties may be improved.

In addition, the shape of the quantum dot may be generally used shapes in the art, without specific limitation. More particularly, the shape of spherical, pyramidal, multi-arm, or cubic nanoparticle, nanotube, nanowire, nanofiber, nanoplate particle, etc. may be used.

The quantum dot may control the color of light emitted according to the particle size, and accordingly, the quantum dot may have various emission colors such as blue, red and green.

In the light emitting devices ED of embodiments, as shown in FIG. 3 to FIG. 6, the electron transport region ETR is provided on the emission layer EML. The electron transport region ETR may include at least one of an electron blocking layer HBL, an electron transport layer ETL or an electron injection layer EIL. However, an embodiment of the inventive concept is not limited thereto.

The electron transport region ETR may have a single layer formed using a single material, a single layer formed using multiple different materials, or a multilayer structure having multiple layers formed using multiple different materials.

For example, the electron transport region ETR may have a single layer structure of an electron injection layer EIL or an electron transport layer ETL, or a single layer structure formed using an electron injection material and an electron transport material. Further, the electron transport region ETR may have a single layer structure formed using multiple different materials, or a structure stacked from the emission layer EML of electron transport layer ETL/electron injection layer EIL, or hole blocking layer HBL/electron transport layer ETL/electron injection layer EIL, without limitation. The thickness of the electron transport region ETR may be, for example, from about 1,000 Å to about 1,500 Å.

The electron transport region ETR may be formed using various methods such as a vacuum deposition method, a spin coating method, a cast method, a Langmuir-Blodgett (LB) method, an inkjet printing method, a laser printing method, and a laser induced thermal imaging (LITI) method.

The electron transport region ETR may include a compound represented by Formula ET-2 below.

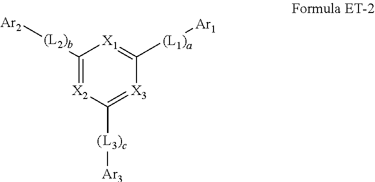

In Formula ET-2, at least one among X₁ to X₃ is N, and the remainder are CRₐ. Rₐ may be a hydrogen atom, a deuterium atom, a substituted or unsubstituted alkyl of 1 to 20 carbon atoms, a substituted or unsubstituted aryl group of 6 to 30 ring-forming carbon atoms, or a substituted or unsubstituted heteroaryl group of 2 to 30 ring-forming carbon atoms. Ar₁ to Ar₃ may be each independently a hydrogen atom, a deuterium atom, a substituted or unsubstituted alkyl group of 1 to 20 carbon atoms, a substituted or unsubstituted aryl group of 6 to 30 ring-forming carbon atoms, or a substituted or unsubstituted heteroaryl group of 2 to 30 ring-forming carbon atoms.

In Formula ET-2, “a” to “c” may be each independently an integer of 0 to 10. In Formula ET-2, L₁ to L₃ may be each independently a direct linkage, a substituted or unsubstituted arylene group of 6 to 30 ring-forming carbon atoms, or a substituted or unsubstituted heteroarylene group of 2 to 30 ring-forming carbon atoms. Meanwhile, if “a” to “c” are integers of 2 or more, L₁ to L₃ may be each independently a substituted or unsubstituted arylene group of 6 to 30 ring-forming carbon atoms, or a substituted or unsubstituted heteroarylene group of 2 to 30 ring-forming carbon atoms.

The electron transport region ETR may include an anthracene-based compound. However, an embodiment of the inventive concept is not limited thereto, and the electron transport region ETR may include, for example, tris(8-hydroxyquinolinato)aluminum (Alq₃), 1,3,5-tri[(3-pyridyl)-phen-3-yl]benzene, 2,4,6-tris(3′-(pyridin-3-yl)biphenyl-3-yl)-1,3,5-triazine, 2-(4-(N-phenylbenzoimidazolyl-1-yl)phenyl)-9,10-dinaphthylanthracene, 1,3,5-tri(1-phenyl-1H-benzo[d]imidazol-2-yl)benzene (TPBi), 2,9-dimethyl-4,7-diphenyl-1,10-phenanthroline (BCP), 4,7-diphenyl-1,10-phenanthroline (Bphen), 3-(4-biphenylyl)-4-phenyl-5-tert-butylphenyl-1,2,4-triazole (TAZ), 4-(naphthalen-1-yl)-3,5-diphenyl-4H-1,2,4-triazole (NTAZ), 2-(4-biphenylyl)-5-(4-tert-butylphenyl)-1,3,4-oxadiazole (tBu-PBD), bis(2-methyl-8-quinolinolato-N1,O8)-(1,1′-biphenyl-4-olato)aluminum (BAlq), berylliumbis(benzoquinolin-10-olate (Bebq₂), 9,10-di(naphthalene-2-yl)anthracene (ADN), 1,3-bis[3,5-di(pyridin-3-yl)phenyl]benzene (BmPyPhB), and mixtures thereof, without limitation.

The electron transport region ETR may include at least one among Compounds ET1 to ET36 below.

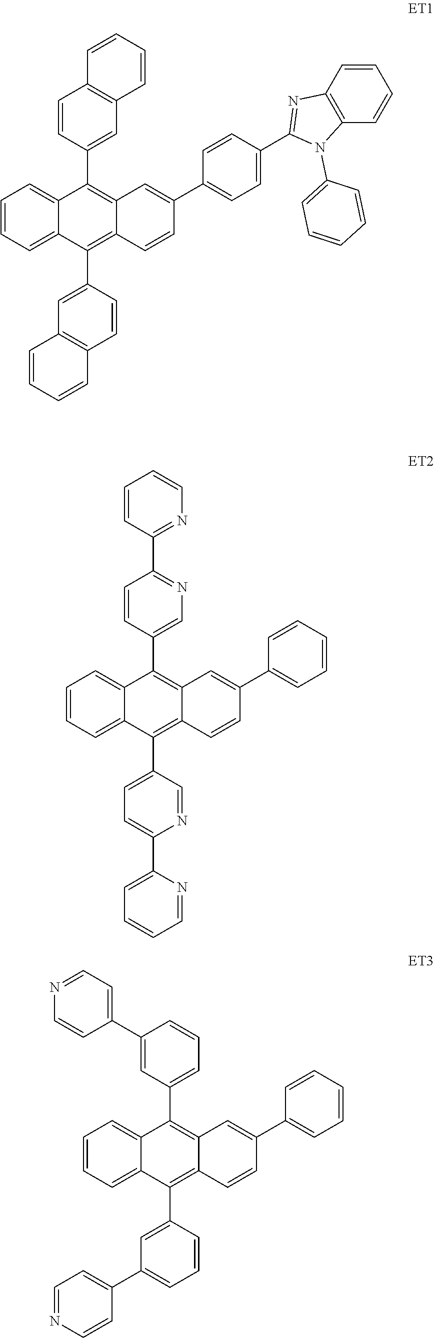 [SMILES: c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; c1ccc(-c2ccc3c(-c4ccc(-c5ccccn5)nc4)c4ccccc4c(-c4ccc(-c5ccccn5)nc4)c3c2)cc1 ; c1ccc(-c2ccc3c(-c4cccc(-c5ccncc5)c4)c4ccccc4c(-c4cccc(-c5ccncc5)c4)c3c2)cc1]

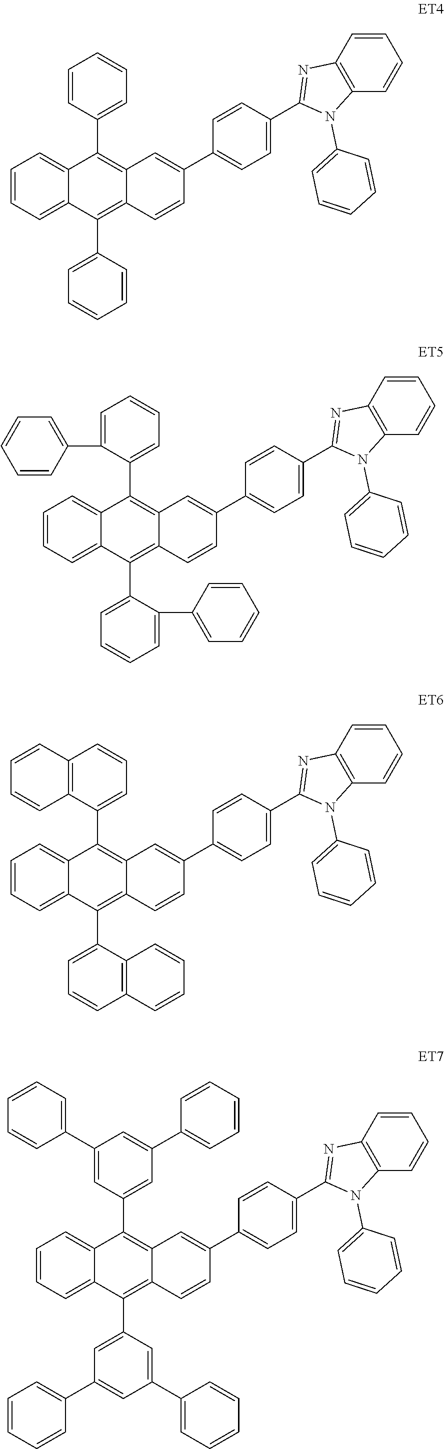 [SMILES: c1ccc(-c2c3ccccc3c(-c3ccccc3)c3cc(-c4ccc(-c5nc6ccccc6n5-c5ccccc5)cc4)ccc23)cc1 ; c1ccc(-c2ccccc2-c2c3ccccc3c(-c3ccccc3-c3ccccc3)c3cc(-c4ccc(-c5nc6ccccc6n5-c5ccccc5)cc4)ccc23)cc1 ; c1ccc(-n2c(-c3ccc(-c4ccc5c(-c6cccc7ccccc67)c6ccccc6c(-c6cccc7ccccc67)c5c4)cc3)nc3ccccc32)cc1 ; c1ccc(-c2cc(-c3ccccc3)cc(-c3c4ccccc4c(-c4cc(-c5ccccc5)cc(-c5ccccc5)c4)c4cc(-c5ccc(-c6nc7ccccc7n6-c6ccccc6)cc5)ccc34)c2)cc1]

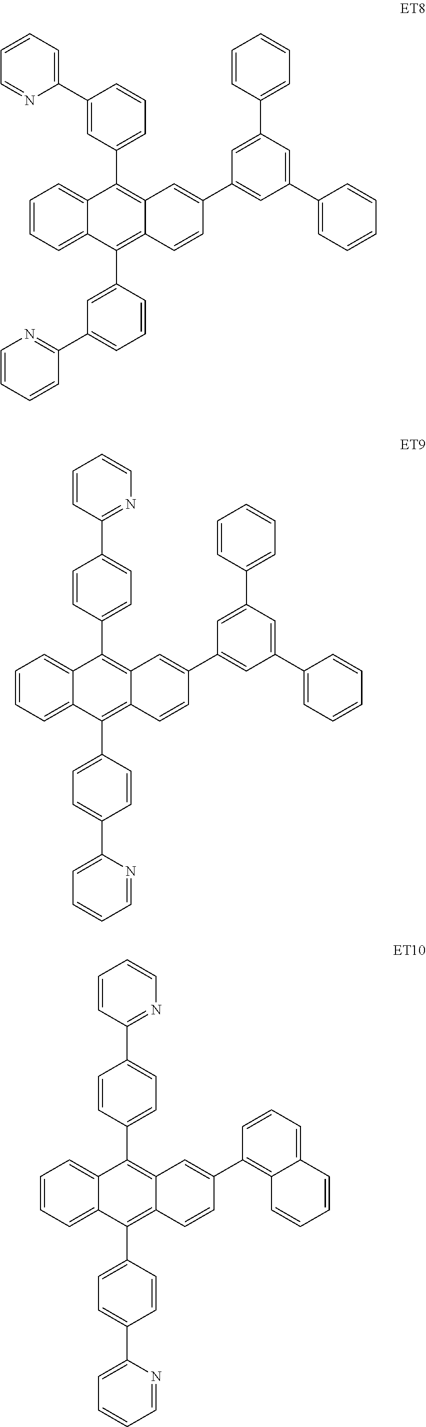

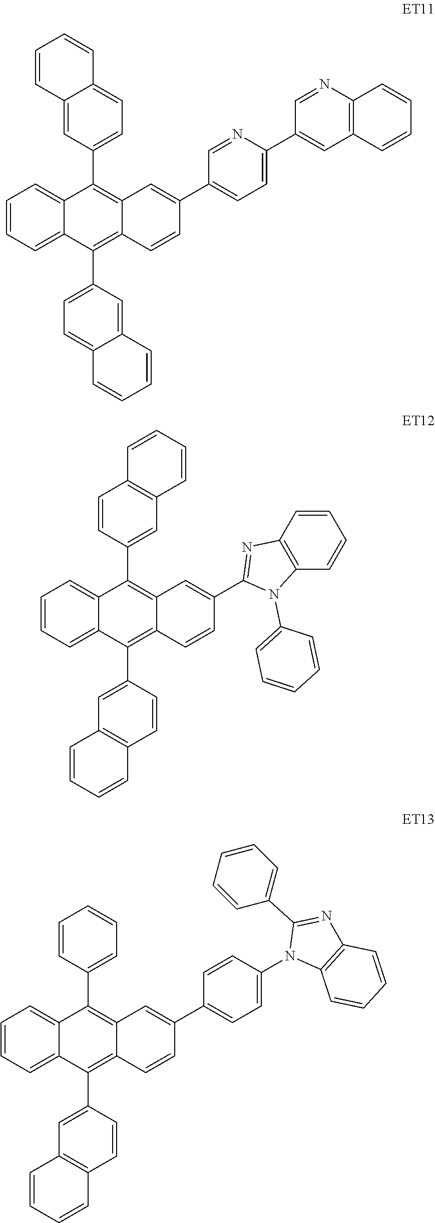

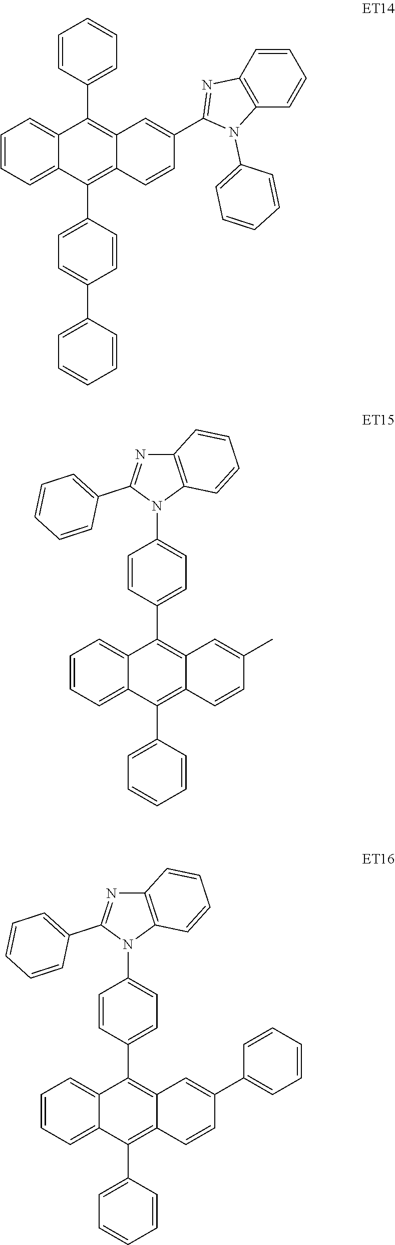 [SMILES: c1ccc(-c2ccc(-c3c4ccccc4c(-c4ccccc4)c4cc(-c5nc6ccccc6n5-c5ccccc5)ccc34)cc2)cc1 ; Cc1ccc2c(-c3ccccc3)c3ccccc3c(-c3ccc(-n4c(-c5ccccc5)nc5ccccc54)cc3)c2c1 ; c1ccc(-c2ccc3c(-c4ccccc4)c4ccccc4c(-c4ccc(-n5c(-c6ccccc6)nc6ccccc65)cc4)c3c2)cc1]

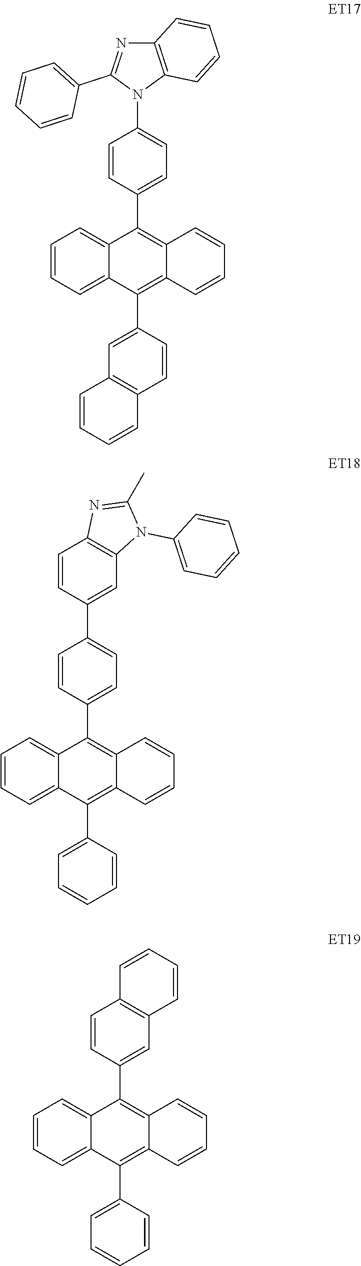

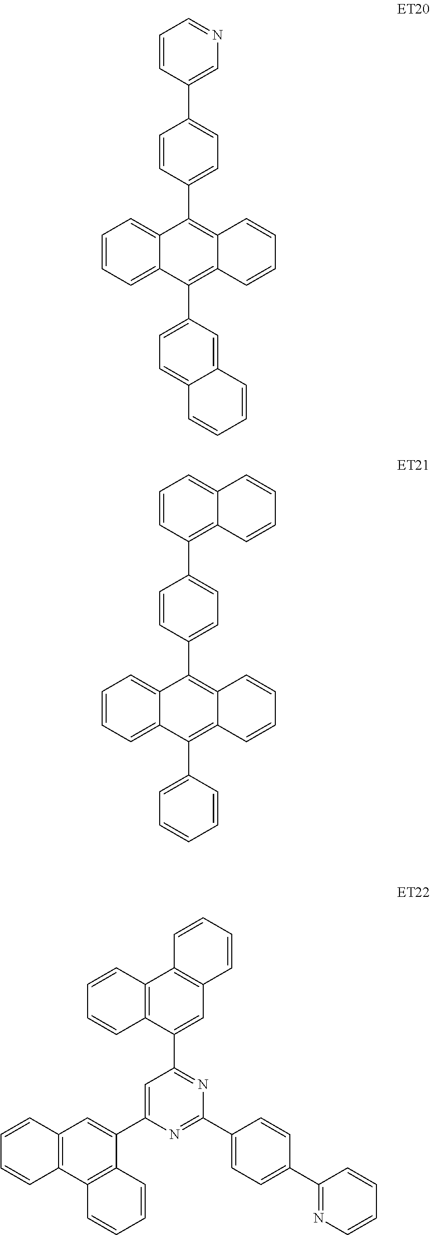

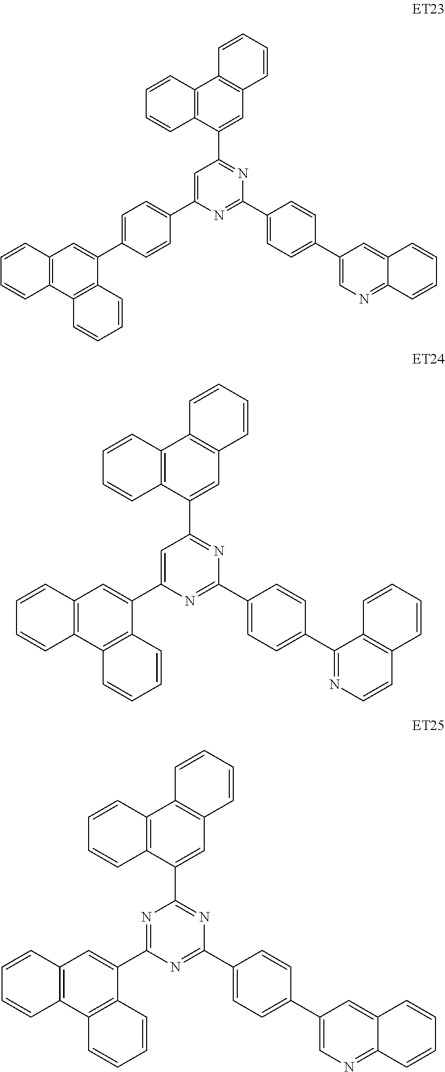 [SMILES: c1ccc2ncc(-c3ccc(-c4nc(-c5ccc(-c6cc7ccccc7c7ccccc67)cc5)cc(-c5cc6ccccc6c6ccccc56)n4)cc3)cc2c1 ; c1ccc2c(-c3ccc(-c4nc(-c5cc6ccccc6c6ccccc56)cc(-c5cc6ccccc6c6ccccc56)n4)cc3)nccc2c1 ; c1ccc2ncc(-c3ccc(-c4nc(-c5cc6ccccc6c6ccccc56)nc(-c5cc6ccccc6c6ccccc56)n4)cc3)cc2c1]

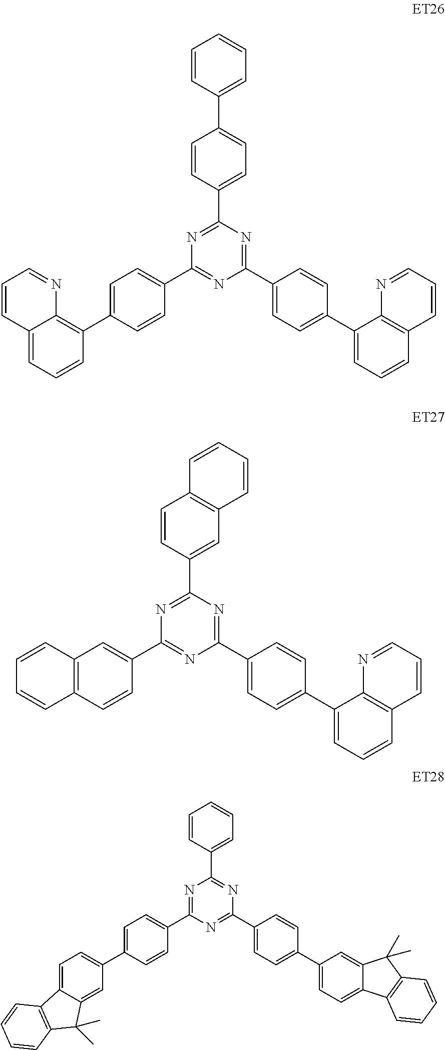 [SMILES: c1ccc(-c2ccc(-c3nc(-c4ccc(-c5cccc6cccnc56)cc4)nc(-c4ccc(-c5cccc6cccnc56)cc4)n3)cc2)cc1 ; c1ccc2cc(-c3nc(-c4ccc(-c5cccc6cccnc56)cc4)nc(-c4ccc5ccccc5c4)n3)ccc2c1 ; CC1(C)c2ccccc2-c2ccc(-c3ccc(-c4nc(-c5ccccc5)nc(-c5ccc(-c6ccc7c(c6)C(C)(C)c6ccccc6-7)cc5)n4)cc3)cc21]

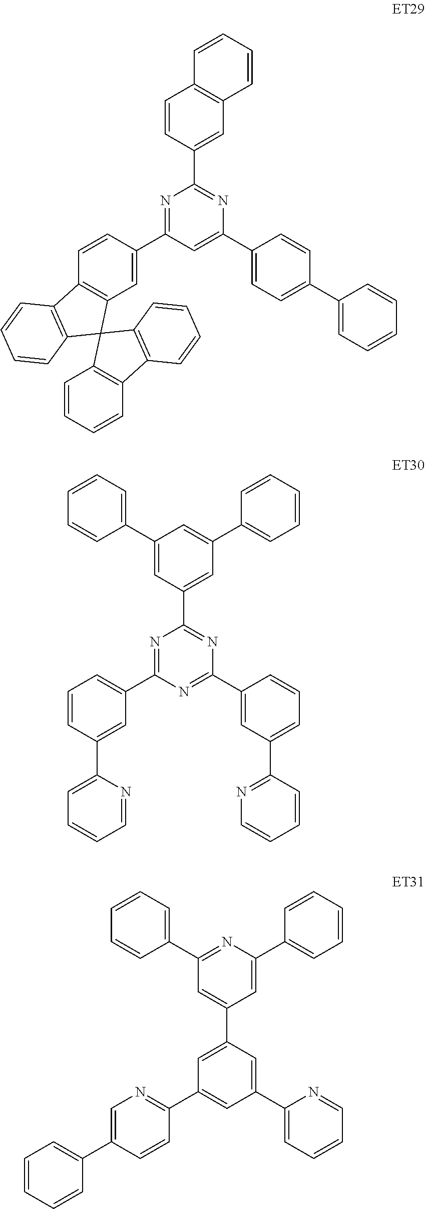

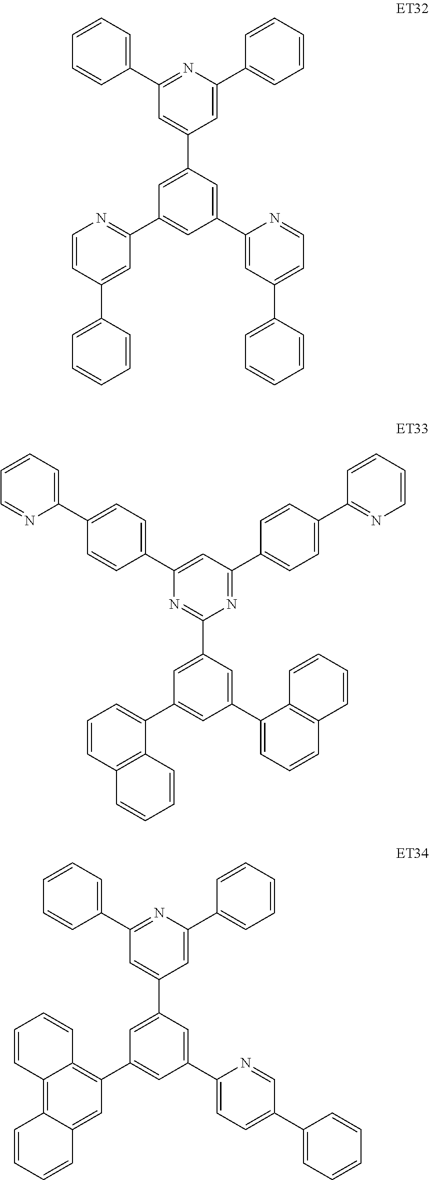 [SMILES: c1ccc(-c2ccnc(-c3cc(-c4cc(-c5ccccc5)nc(-c5ccccc5)c4)cc(-c4cc(-c5ccccc5)ccn4)c3)c2)cc1 ; c1ccc(-c2ccc(-c3cc(-c4ccc(-c5ccccn5)cc4)nc(-c4cc(-c5cccc6ccccc56)cc(-c5cccc6ccccc56)c4)n3)cc2)nc1 ; c1ccc(-c2ccc(-c3cc(-c4cc(-c5ccccc5)nc(-c5ccccc5)c4)cc(-c4cc5ccccc5c5ccccc45)c3)nc2)cc1]

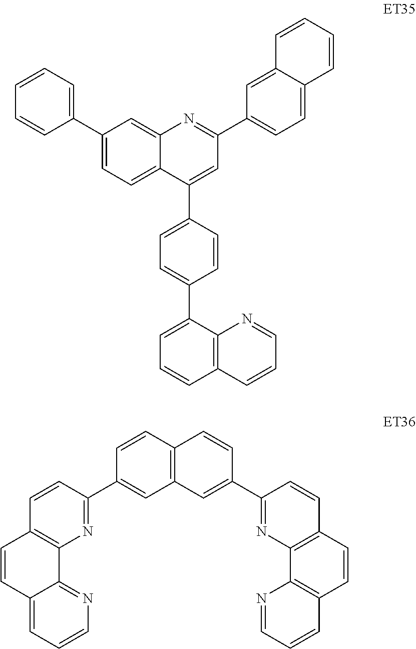 [SMILES: c1ccc(-c2ccc3c(-c4ccc(-c5cccc6cccnc56)cc4)cc(-c4ccc5ccccc5c4)nc3c2)cc1 ; c1cnc2c(c1)ccc1ccc(-c3ccc4ccc(-c5ccc6ccc7cccnc7c6n5)cc4c3)nc12]

In addition, the electron transport region ETR may include a metal halide such as LiF, NaCl, CsF, RbCl, RbI, CuI and KI, a metal in lanthanoides such as Yb, or a co-depositing material of the metal halide and the metal in lanthanoides. For example, the electron transport region ETR may include KI:Yb, RbI:Yb, LiF:Yb, etc., as the co-depositing material. Meanwhile, the electron transport region ETR may use a metal oxide such as Li₂O and BaO, or 8-hydroxy-lithium quinolate (Liq). However, an embodiment of the inventive concept is not limited thereto. The electron transport region ETR also may be formed using a mixture material of an electron transport material and an insulating organo metal salt. The organo metal salt may be a material having an energy band gap of about 4 eV or more. Particularly, the organo metal salt may include, for example, metal acetates, metal benzoates, metal acetoacetates, metal acetylacetonates, or metal stearates.

The electron transport region ETR may include at least one of 2,9-dimethyl-4,7-diphenyl-1,10-phenanthroline (BCP), diphenyl(4-(triphenylsilyl)phenyl)phosphine oxide (TSPO1) or 4,7-diphenyl-1,10-phenanthroline (Bphen) in addition to the aforementioned materials. However, an embodiment of the inventive concept is not limited thereto.

The electron transport region ETR may include the compounds of the electron transport region in at least one among an electron injection layer EIL, an electron transport layer ETL, and a hole blocking layer HBL.

If the electron transport region ETR includes the electron transport layer ETL, the thickness of the electron transport layer ETL may be from about 100 Å to about 1,000 Å, for example, from about 150 Å to about 500 Å. If the thickness of the electron transport layer ETL satisfies the above-described range, satisfactory electron transport properties may be obtained without substantial increase of a driving voltage.

If the electron transport region ETR includes the electron injection layer EIL, the thickness of the electron injection layer EIL may be from about 1 Å to about 100 Å, and from about 3 Å to about 90 Å. If the thickness of the electron injection layer EIL satisfies the above described range, satisfactory electron injection properties may be obtained without inducing substantial increase of a driving voltage.

The second electrode EL2 is provided on the electron transport region ETR. The second electrode EL2 may be a common electrode. The second electrode EL2 may be a cathode or an anode, but an embodiment of the inventive concept is not limited thereto. For example, if the first electrode EL1 is an anode, the second cathode EL2 may be a cathode, and if the first electrode EL1 is a cathode, the second electrode EL2 may be an anode.

The second electrode EL2 may be a transmissive electrode, a transflective electrode or a reflective electrode. If the second electrode EL2 is the transmissive electrode, the second electrode EL2 may include a transparent metal oxide, for example, ITO, IZO, ZnO, ITZO, etc.

If the second electrode EL2 is the transflective electrode or the reflective electrode, the second electrode EL2 may include Ag, Mg, Cu, Al, Pt, Pd, Au, Ni, Nd, Ir, Cr, L₁, Ca, LiF/Ca, LiF/Al, Mo, Ti, Yb, W, compounds including thereof, or mixtures thereof (for example, AgMg, AgYb, or MgYb). Otherwise, the second electrode EL2 may have a multilayered structure including a reflective layer or a transflective layer formed using the above-described materials and a transparent conductive layer formed using ITO, IZO, ZnO, ITZO, etc. For example, the second electrode EL2 may include the aforementioned metal materials, combinations of two or more metal materials selected from the aforementioned metal materials, or oxides of the aforementioned metal materials.

Though not shown, the second electrode EL2 may be connected with an auxiliary electrode. If the second electrode EL2 is connected with the auxiliary electrode, the resistance of the second electrode EL2 may decrease.

Meanwhile, on the second electrode EL2 in the light emitting device ED of an embodiment, a capping layer CPL may be further disposed. The capping layer CPL may include a multilayer or a single layer.

In an embodiment, the capping layer CPL may be an organic layer or an inorganic layer. For example, if the capping layer CPL includes an inorganic material, the inorganic material may include an alkali metal compound such as LiF, an alkaline earth metal compound such as MgF₂, SiON, SiNx, SiOy, etc.

For example, if the capping layer CPL includes an organic material, the organic material may include α-NPD, NPB, TPD, m-MTDATA, Alq₃, CuPc, N4,N4,N4′,N4′-tetra(biphenyl-4-yl) biphenyl-4,4′-diamine (TPD15), 4,4′,4″-tris(carbazol sol-9-yl)triphenylamine (TCTA), etc., or includes an epoxy resin, or acrylate such as methacrylate. In addition, a capping layer CPL may include at least one among Compounds P₁ to P₅ below, but an embodiment of the inventive concept is not limited thereto.

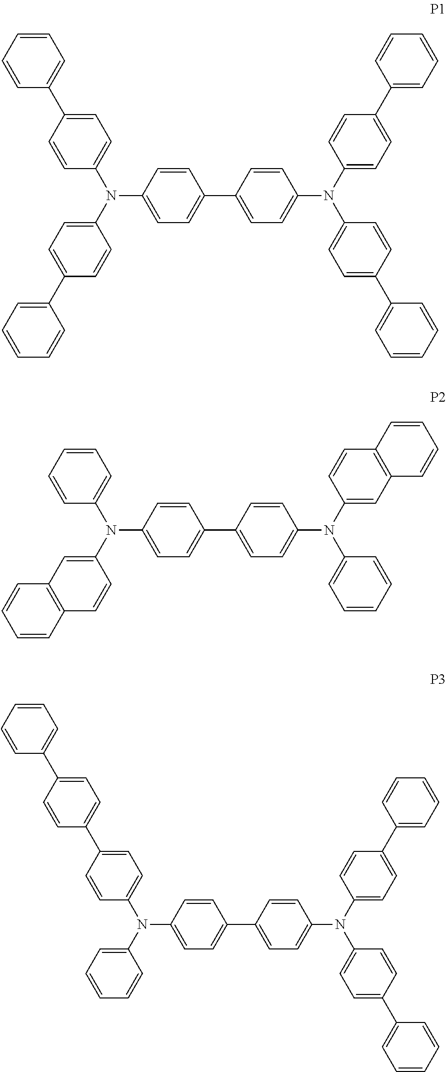 [SMILES: c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; c1ccc(N(c2ccc(-c3ccc(N(c4ccccc4)c4ccc5ccccc5c4)cc3)cc2)c2ccc3ccccc3c2)cc1 ; c1ccc(-c2ccc(-c3ccc(N(c4ccccc4)c4ccc(-c5ccc(N(c6ccc(-c7ccccc7)cc6)c6ccc(-c7ccccc7)cc6)cc5)cc4)cc3)cc2)cc1]

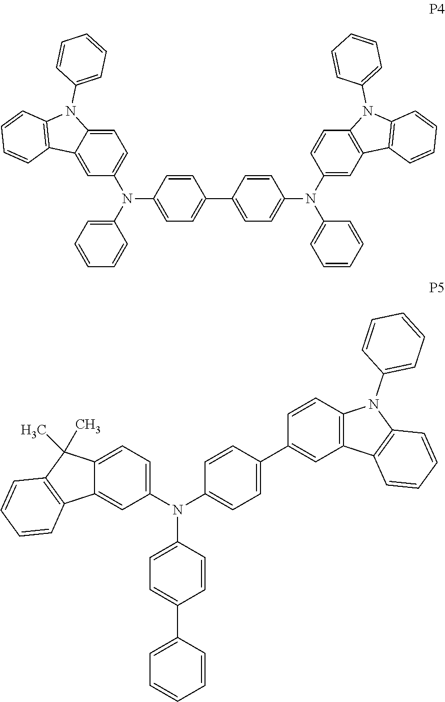 [SMILES: c1ccc(N(c2ccc(-c3ccc(N(c4ccccc4)c4ccc5c(c4)c4ccccc4n5-c4ccccc4)cc3)cc2)c2ccc3c(c2)c2ccccc2n3-c2ccccc2)cc1 ; CC1(C)c2ccccc2-c2cc(N(c3ccc(-c4ccccc4)cc3)c3ccc(-c4ccc5c(c4)c4ccccc4n5-c4ccccc4)cc3)ccc21]

Meanwhile, the refractive index of the capping layer CPL may be about 1.6 or more. Particularly, the refractive index of the capping layer CPL with respect to light in a wavelength range of about 550 nm to about 660 nm may be about 1.6 or more.

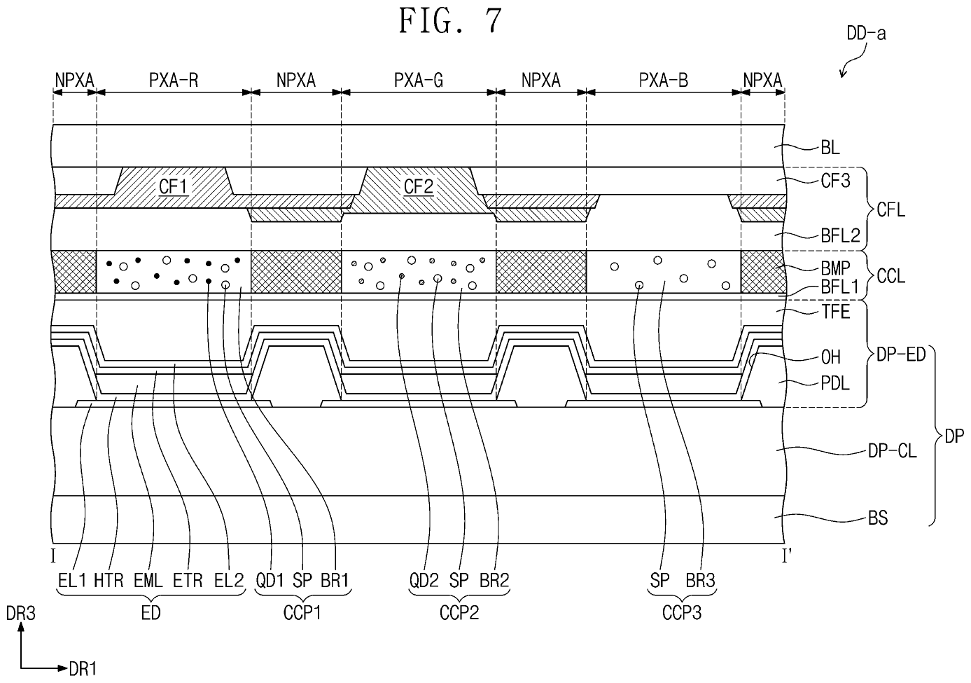

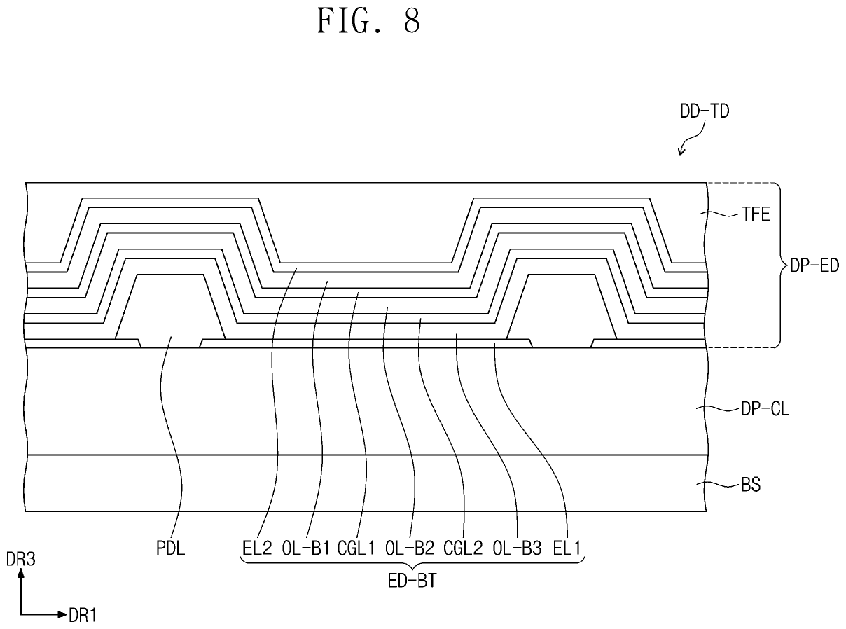

FIG. 7 and FIG. 8 are cross-sectional views on display apparatuses according to embodiments of the inventive concept, respectively. In the explanation on the display apparatuses of embodiments referring to FIG. 7 and FIG. 8, the overlapping parts with the explanation on FIG. 1 to FIG. 6 will not be explained again, and the different features will be explained chiefly.

Referring to FIG. 7, the display apparatus DD according to an embodiment may include a display panel DP including a display device layer DP-ED, a light controlling layer CCL disposed on the display panel DP and a color filter layer CFL.

In an embodiment shown in FIG. 7, the display panel DP includes a base layer BS, a circuit layer DP-CL provided on the base layer BS and a display device layer DP-ED, and the display device layer DP-ED may include a light emitting device ED.

The light emitting device ED may include a first electrode EL1, a hole transport region HTR disposed on the first electrode EL1, an emission layer EML disposed on the hole transport region HTR, an electron transport region ETR disposed on the emission layer EML, and a second electrode EL2 disposed on the electron transport region ETR. Meanwhile, the same structures as the light emitting devices of FIG. 3 to FIG. 6 may be applied to the structure of the light emitting device ED shown in FIG. 7.

Referring to FIG. 7, the emission layer EML may be disposed in an opening part OH defined in a pixel definition layer PDL. For example, the emission layer EML divided by the pixel definition layer PDL and correspondingly provided to each of luminous areas PXA-R, PXA-G and PXA-B may emit light in the same wavelength region. In the display apparatus DD of an embodiment, the emission layer EML may emit blue light. Meanwhile, different from the drawings, in an embodiment, the emission layer EML may be provided as a common layer for all luminous areas PXA-R, PXA-G and PXA-B.

The light controlling layer CCL may be disposed on the display panel DP. The light controlling layer CCL may include a light converter. The light converter may be a quantum dot or a phosphor. The light converter may transform the wavelength of light provided and then emit. That is, the light controlling layer CCL may be a layer including a quantum dot or a layer including a phosphor.

The light controlling layer CCL may include multiple light controlling parts CCP1, CCP2 and CCP3. The light controlling parts CCP1, CCP2 and CCP3 may be separated from one another.

Referring to FIG. 7, a partition pattern BMP may be disposed between the separated light controlling parts CCP1, CCP2 and CCP3, but an embodiment of the inventive concept is not limited thereto. In FIG. 7, the partition pattern BMP is shown not to be overlapped with the light controlling parts CCP1, CCP2 and CCP3, but at least a portion of the edge of the light controlling parts CCP1, CCP2 and CCP3 may be overlapped with the partition pattern BMP.

The light controlling layer CCL may include a first light controlling part CCP1 including a first quantum dot QD1 converting first color light provided from the light emitting device ED into second color light, a second light controlling part CCP2 including a second quantum dot QD2 converting first color light into third color light, and a third light controlling part CCP3 transmitting first color light.

In an embodiment, the first light controlling part CCP1 may provide red light which is the second color light, and the second light controlling part CCP2 may provide green light which is the third color light. The third color controlling part CCP3 may transmit and provide blue light which is the first color light provided from the light emitting device ED. For example, the first quantum dot QD1 may be a red quantum dot, and the second quantum dot QD2 may be a green quantum dot. On the quantum dots QD1 and QD2, the same contents as those described above may be applied.

In addition, the light controlling layer CCL may further include a scatterer SP. The first light controlling part CCP1 may include the first quantum dot QD1 and the scatterer SP, the second light controlling part CCP2 may include the second quantum dot QD2 and the scatterer SP, and the third light controlling part CCP3 may not include a quantum dot but include the scatterer SP.

The scatterer SP may be an inorganic particle. For example, the scatterer SP may include at least one among TiO₂, ZnO, Al₂O₃, SiO₂, and hollow silica. The scatterer SP may include at least one among TiO₂, ZnO, Al₂O₃, SiO₂, and hollow silica, or may be a mixture of two or more materials selected among TiO₂, ZnO, Al₂O₃, SiO₂, and hollow silica.

Each of the first light controlling part CCP1, the second light controlling part CCP2, and the third light controlling part CCP3 may include base resins BR1, BR2 and BR3 dispersing the quantum dots QD1 and QD2 and the scatterer SP. In an embodiment, the first light controlling part CCP1 may include the first quantum dot QD1 and the scatterer SP dispersed in the first base resin BR1, the second light controlling part CCP2 may include the second quantum dot QD2 and the scatterer SP dispersed in the second base resin BR2, and the third light controlling part CCP3 may include the scatterer particle SP dispersed in the third base resin BR3. The base resins BR1, BR2 and BR3 are mediums in which the quantum dots QD1 and QD2 and the scatterer SP are dispersed, and may be composed of various resin compositions which may be generally referred to as a binder. For example, the base resins BR1, BR2 and BR3 may be acrylic resins, urethane-based resins, silicone-based resins, epoxy-based resins, etc. The base resins BR1, BR2 and BR3 may be transparent resins. In an embodiment, the first base resin BR1, the second base resin BR2 and the third base resin BR3 may be the same or different from each other.

The light controlling layer CCL may include a barrier layer BFL1. The barrier layer BFL1 may play the role of blocking the penetration of moisture and/or oxygen (hereinafter, will be referred to as “humidity/oxygen”). The barrier layer BFL1 may be disposed between the light controlling parts CCP1, CCP2 and CCP3 and the encapsulating layer TFE to block the exposure of the light controlling parts CCP1, CCP2 and CCP3 to humidity/oxygen. Meanwhile, the barrier layer BFL1 may cover the light controlling parts CCP1, CCP2 and CCP3. In addition, a color filter layer CFL, which will be explained later, may include a barrier layer BFL2 disposed on the light controlling parts CCP1, CCP2 and CCP3.

The barrier layers BFL1 and BFL2 may include at least one inorganic layer. That is, the barrier layers BFL1 and BFL2 may be formed by including an inorganic material. For example, the barrier layers BFL1 and BFL2 may be formed by including silicon nitride, aluminum nitride, zirconium nitride, titanium nitride, hafnium nitride, tantalum nitride, silicon oxide, aluminum oxide, titanium oxide, tin oxide, cerium oxide and silicon oxynitride or a metal thin film securing light transmittance. Meanwhile, the barrier layers BFL1 and BFL2 may further include an organic layer. The barrier layers BFL1 and BFL2 may be composed of a single layer of multiple layers.

In the display apparatus DD of an embodiment, the color filter layer CFL may be disposed on the light controlling layer CCL. For example, the color filter layer CFL may be disposed directly on the light controlling layer CCL. In this case, the barrier layer BFL2 may be omitted.

The color filter layer CFL may include filters CF1, CF2 and CF3. The color filter layer CFL may include a first filter CF1 transmitting second color light, a second filter CF2 transmitting third color light, and a third filter CF3 transmitting first color light. For example, the first filter CF1 may be a red filter, the second filter CF2 may be a green filter, and the third filter CF3 may be a blue filter. Each of the filters CF1, CF2 and CF3 may include a polymer photosensitive resin and a pigment or dye. The first filter CF1 may include a red pigment or dye, the second filter CF2 may include a green pigment or dye, and the third filter CF3 may include a blue pigment or dye. Meanwhile, an embodiment of the inventive concept is not limited thereto, and the third filter CF3 may not include the pigment or dye. The third filter CF3 may include a polymer photosensitive resin and not include a pigment or dye. The third filter CF3 may be transparent. The third filter CF3 may be formed using a transparent photosensitive resin.

In addition, in an embodiment, the first filter CF1 and the second filter CF2 may be yellow filters. The first filter CF1 and the second filter CF2 may be provided in one body without distinction.

Although not shown, the color filter layer CFL may further include the light blocking part. The light blocking part may be a black matrix. The light blocking part may be formed by including an organic light blocking material or an inorganic light blocking material including a black pigment or black dye. The light blocking part may prevent light leakage phenomenon and divide the boundaries among adjacent filters CF1, CF2 and CF3. In addition, in an embodiment, the light blocking part may be formed as a blue filter.

The first to third filters CF1, CF2 and CF3 may be disposed corresponding to a red luminous area PXA-R, green luminous area PXA-G, and blue luminous area PXA-B, respectively.

On the color filter layer CFL, a base substrate BL may be disposed. The base substrate BL may be a member providing a base surface on which the color filter layer CFL, the light controlling layer CCL, etc. are disposed. The base substrate BL may be a glass substrate, a metal substrate, a plastic substrate, etc. However, an embodiment of the inventive concept is not limited thereto, and the base substrate BL may be an inorganic layer, an organic layer or a composite material layer. In addition, different from the drawing, the base substrate BL may be omitted in an embodiment.

FIG. 8 is a cross-sectional view showing a portion of the display apparatus according to an embodiment. In a display apparatus DD-TD of an embodiment, the light emitting device ED-BT may include multiple light emitting structures OL-B1, OL-B2 and OL-B3. The light emitting device ED-BT may include oppositely disposed first electrode EL1 and second electrode EL2, and the multiple light emitting structures OL-B1, OL-B2 and OL-B3 stacked in order in a thickness direction and provided between the first electrode EL1 and the second electrode EL2. Each of the light emitting structures OL-B1, OL-B2 and OL-B3 may include an emission layer EML (FIG. 7), and a hole transport region HTR and an electron transport region ETR disposed with the emission layer EML (FIG. 7) therebetween.

That is, the light emitting device ED-BT included in the display apparatus DD-TD of an embodiment may be a light emitting device of a tandem structure including multiple emission layers.

In an embodiment shown in FIG. 8, light emitted from the light emitting structures OL-B1, OL-B2 and OL-B3 may be all blue light. However, an embodiment of the inventive concept is not limited thereto, and the wavelength regions of light emitted from the light emitting structures OL-B1, OL-B2 and OL-B3 may be different from each other. For example, the light emitting device ED-BT including the multiple light emitting structures OL-B1, OL-B2 and OL-B3 emitting light in different wavelength regions may emit white light.

Between neighboring light emitting structures OL-B1, OL-B2 and OL-B3, charge generating layers CGL1 and CGL2 may be disposed. The charge generating layers CGL1 and CGL2 may include a p-type charge generating layer and/or an n-type charge generating layer.

In at least one among the light emitting structures OL-B1, OL-B2 and OL-B3, included in the display apparatus DD-TD of an embodiment, the monoamine compound of an embodiment may be included. That is, at least one among multiple hole transport region included in the light emitting device ED-BT may include the monoamine compound of an embodiment.

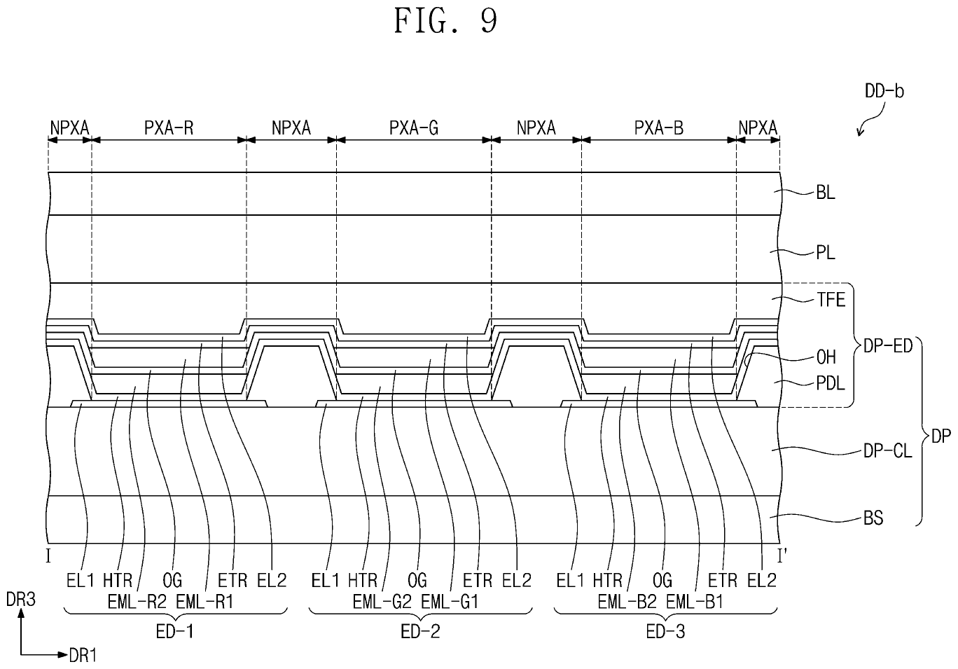

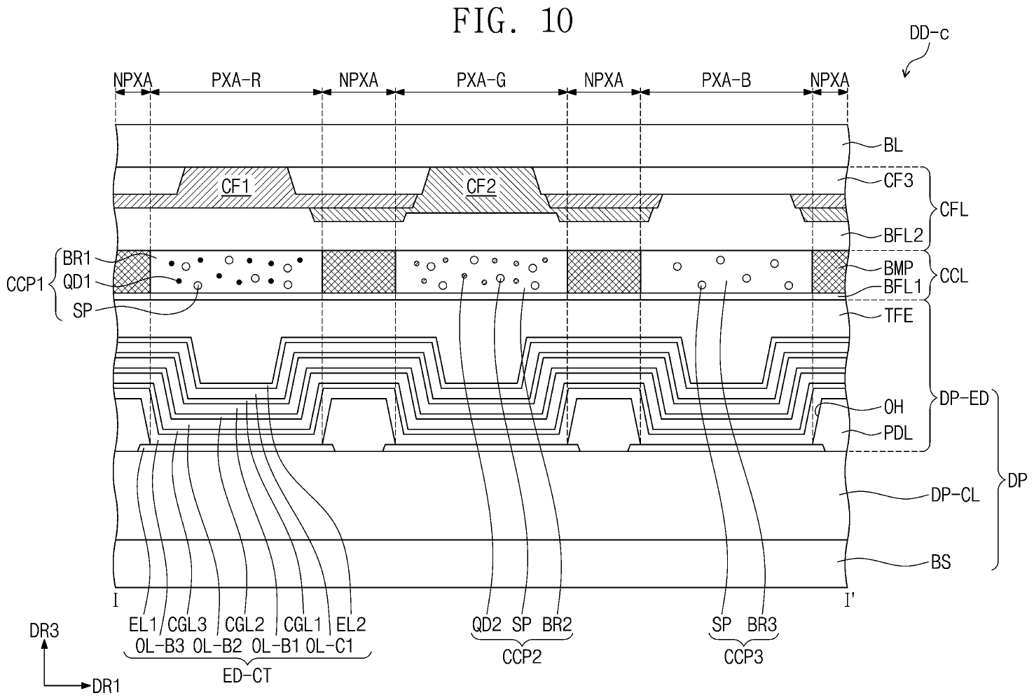

FIG. 9 is a cross-sectional view showing a display apparatus according to an embodiment of the inventive concept. FIG. 10 is a cross-sectional view showing a display apparatus according to an embodiment of the inventive concept.

Referring to FIG. 9, a display apparatus DD-b according to an embodiment may include light emitting devices ED-1, ED-2 and ED-3, formed by stacking two emission layers. Compared to the display apparatus DD of an embodiment, shown in FIG. 2, an embodiment shown in FIG. 9 is different in that first to third light emitting devices ED-1, ED-2 and ED-3 include two emission layers stacked in a thickness direction, each. In the first to third light emitting devices ED-1, ED-2 and ED-3, two emission layers may emit light in the same wavelength region.

The first light emitting device ED-1 may include a first red emission layer EML-R1 and a second red emission layer EML-R₂. The second light emitting device ED-2 may include a first green emission layer EML-G1 and a second green emission layer EML-G2. In addition, the third light emitting device ED-3 may include a first blue emission layer EML-B1 and a second blue emission layer EML-B2. Between the first red emission layer EML-R1 and the second red emission layer EML-R₂, between the first green emission layer EML-G1 and the second green emission layer EML-G2, and between the first blue emission layer EML-B1 and the second blue emission layer EML-B2, an emission auxiliary part OG may be disposed.

The emission auxiliary part OG may include a single layer or a multilayer. The emission auxiliary part OG may include a charge generating layer. More particularly, the emission auxiliary part OG may include an electron transport region, a charge generating layer, and a hole transport region stacked in order. The emission auxiliary part OG may be provided as a common layer in all of the first to third light emitting devices ED-1, ED-2 and ED-3. However, an embodiment of the inventive concept is not limited thereto, and the emission auxiliary part OG may be patterned and provided in an opening part OH defined in a pixel definition layer PDL.

The first red emission layer EML-R1, the first green emission layer EML-G1 and the first blue emission layer EML-B1 may be disposed between the hole transport region HTR and the emission auxiliary part OG. The second red emission layer EML-R₂, the second green emission layer EML-G2 and the second blue emission layer EML-B2 may be disposed between the emission auxiliary part OG and the electron transport region ETR.

That is, the first light emitting device ED-1 may include a first electrode EL1, a hole transport region HTR, a second red emission layer EML-R2, an emission auxiliary part OG, a first red emission layer EML-R₁, an electron transport region ETR, and a second electrode EL2, stacked in order. The second light emitting device ED-2 may include a first electrode EL1, a hole transport region HTR, a second green emission layer EML-G2, an emission auxiliary part OG, a first green emission layer EML-G1, an electron transport region ETR, and a second electrode EL2, stacked in order. The third light emitting device ED-3 may include a first electrode EL1, a hole transport region HTR, a second blue emission layer EML-B2, an emission auxiliary part OG, a first blue emission layer EML-B1, an electron transport region ETR, and a second electrode EL2, stacked in order.

Meanwhile, an optical auxiliary layer PL may be disposed on a display device layer DP-ED. The optical auxiliary layer PL may include a polarization layer. The optical auxiliary layer PL may be disposed on a display panel DP and may control reflected light at the display panel DP by external light. Different from the drawings, the optical auxiliary layer PL may be omitted from the display apparatus according to an embodiment.

At least one emission layer included in the display apparatus DD-b of an embodiment, shown in FIG. 9 may include the monoamine compound of an embodiment. For example, in an embodiment, hole transport region HTR may include the monoamine compound of an embodiment.

Different from FIG. 8 and FIG. 9, a display apparatus DD-c in FIG. 10 is shown to include four light emitting structures OL-B1, OL-B2, OL-B3 and OL-C1. A light emitting device ED-CT may include oppositely disposed first electrode EL1 and second electrode EL2, and first to fourth light emitting structures OL-B1, OL-B2, OL-B3 and OL-C1 stacked in order in a thickness direction between the first electrode EL1 and the second electrode EL2. Between the first to fourth light emitting structures OL-B1, OL-B2, OL-B3 and OL-C1, charge generating layers CGL1, CGL2 and CGL3 may be disposed. Among the four light emitting structures, the first to third light emitting structures OL-B1, OL-B2 and OL-B3 emit blue light, and the fourth light emitting structure OL-C1 may emit green light. However, an embodiment of the inventive concept is not limited thereto, and the first to fourth light emitting structures OL-B1, OL-B2, OL-B3 and OL-C1 may emit different wavelengths of light.

Charge generating layers CGL1, CGL2 and CGL3 disposed among neighboring light emitting structures OL-B1, OL-B2, OL-B3 and OL-C1 may include a p-type charge generating layer and/or an n-type charge generating layer.

In at least one among the light emitting structures OL-B1, OL-B2, OL-B3 and OL-C1, included in the display apparatus DD-c of an embodiment, the monoamine compound of an embodiment may be included. For example, in an embodiment, at least one among the first to third light emitting structures OL-B1, OL-B2, and OL-B3 may include the monoamine compound of an embodiment.

In an embodiment, an electronic apparatus may include a display apparatus including multiple light emitting devices and a control part controlling the display apparatus. The electronic apparatus of an embodiment may be an apparatus activated according to electrical signals. The electronic apparatus may include display apparatuses of various embodiments. For example, the electronic apparatus may include televisions, monitors, large-size display apparatuses such as outside billboards, personal computers, laptop computers, personal digital terminals, display apparatuses for automobiles, game consoles, portable electronic devices, medium- and small-size display apparatuses such as cameras.

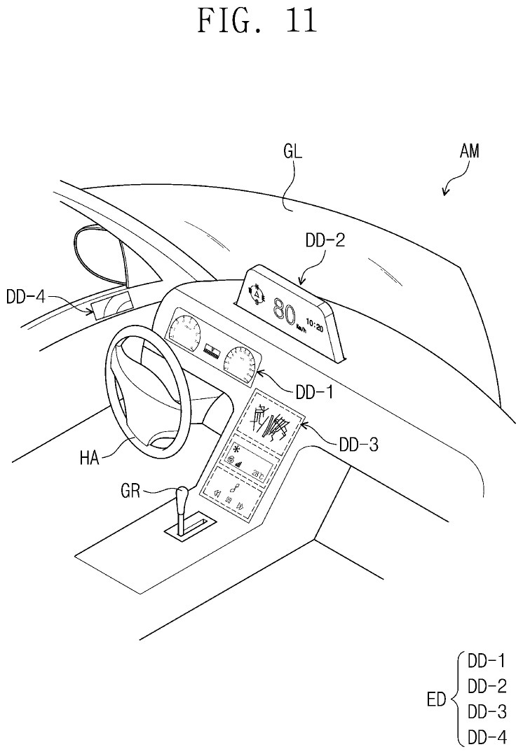

FIG. 11 is a perspective view schematically showing an electronic apparatus including the display apparatus according to an embodiment. In FIG. 11, an electronic apparatus including display apparatuses for automobiles is shown as an illustration.

Referring to FIG. 11, an electronic apparatus ED of an embodiment may include display apparatuses DD-1, DD-2, DD-3 and DD-4 for an automobile AM. In FIG. 5, first to fourth display apparatuses DD-1, DD-2, DD-3 and DD-4 are shown as display apparatuses for an automobile AM, disposed in the automobile AM. In FIG. 1, an automobile is shown but is an illustration, and the first to fourth display apparatuses DD-1, DD-2, DD-3 and DD-4 may be disposed in various transport means such as bicycles, motorcycles, trains, ships and airplanes. At least one among the first to fourth display apparatuses DD-1, DD-2, DD-3 and DD-4 may include the configuration of the display apparatuses DD, DD-a, DD-b and DD-c, explained above referring to FIG. 1, FIG. 2, and FIG. 7 to FIG. 10.

In an embodiment, at least one among the first to fourth display apparatuses DD-1, DD-2, DD-3 and DD-4 may include the light emitting device ED explained referring to FIG. 3 to FIG. 6. The first to fourth display apparatuses DD-1, DD-2, DD-3 and DD-4 may each independently include multiple light emitting devices ED, and each of the light emitting devices ED may include a first electrode EL1, a hole transport region HTL disposed on the first electrode EL1, an emission layer EML disposed on the hole transport region HTL, an electron transport region ETL disposed on the emission layer EML and a second electrode EL2 disposed on the electron transport region ETL. In addition, the emission layer EML may include the above-explained polycyclic compound of an embodiment, represented by Formula 1. Accordingly, the electronic apparatus ED of an embodiment may show improved quality of images.

Referring to FIG. 11, the automobile AM may include a steering wheel HA for the operation of the automobile AM and a gear GR, and a front window GL may be disposed to face a driver.

A first display apparatus DD-1 may be disposed in a first region overlapping with the steering wheel HA. For example, the first display apparatus DD-1 may be a digital cluster displaying the first information of the automobile AM. The first information may include a graduation showing the number of revolution of an engine (i.e., revolutions per minute (RPM)), and images showing a fuel state. First graduation and second graduation may be represented by digital images.

A second display apparatus DD-2 may be disposed in a second region facing a driver's seat and overlapping with the front window GL. The driver's seat may be a seat where the steering wheel HA is disposed. For example, the second display apparatus DD-2 may be a head up display (HUD) showing the second information of the automobile AM. The second display apparatus DD-2 may be optically clear. The second information may include digital numbers DN showing the running speed of the automobile AM and may further include information including the current time.

A third display apparatus DD-3 may be disposed in a third region adjacent to the gear GR. For example, the third display apparatus DD-3 may be a center information display (CID) for an automobile, disposed between a driver's seat and a passenger seat and showing third information. The passenger seat may be a seat separated from the driver's seat with the gear GR therebetween. The third information may include information on road conditions (for example, navigation information), on playing music or radio, on playing a dynamic image, on the temperature in the automobile AM, or the like.

A fourth display apparatus DD-4 may be disposed in a fourth region separated from the steering wheel HA and the gear GR and adjacent to the side of the automobile AM. For example, the fourth display apparatus DD-4 may be a digital wing mirror displaying fourth information. The fourth display apparatus DD-4 may display the external image of the automobile AM, taken by a camera module CM disposed at the outside of the automobile AM. The fourth information may include the external image of the automobile AM.

The above-described first to fourth information is for illustration, and the first to fourth display apparatuses DD-1, DD-2, DD-3 and DD-4 may further display information on the inside and outside of the automobile. The first to fourth information may include different information from each other. However, an embodiment of the inventive concept is not limited thereto, and a portion of the first to fourth information may include the same information.

The light emitting element according to an example embodiment includes a monoamine compound represented by Formula 1, which may help provide high emission efficiency. The monoamine compound represented by Formula 1 may have a relatively large volume. In an example embodiment, in the monoamine compound represented by Formula 1, a phenanthryl group is connected with an amine group via an arylene linker, and a substituent having a large volume such as an aryl group is positioned at an adjacent position to a position where the phenanthryl group is connected with the arylene linker. Accordingly, steric repulsion may arise between the substituent and the arylene linker, the bonding angle between the phenanthryl group and the arylene linker may increase, and the volume occupied by the phenanthryl group may increase. Therefore, a monoamine compound according to an example embodiment may decrease interaction between phenanthryl groups. The decrease of the interaction between the phenanthryl groups may result in the decrease of electron mobility. In the case where the monoamine compound according to an example embodiment is disposed in a hole transport layer HTL which is adjacent to an emission layer EML, the diffusion of electrons from the emission layer to a hole transport region HTR may be restrained, and high emission efficiency of a light emitting element may be secured.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Compounds according to example embodiments may be synthesized, for example, as follows. However, an example embodiment is not limited thereto.

### Synthetic Examples

### 1. Synthesis of Compound 1

Compound 1 which is a compound according to an example embodiment may be synthesized by the following reaction.

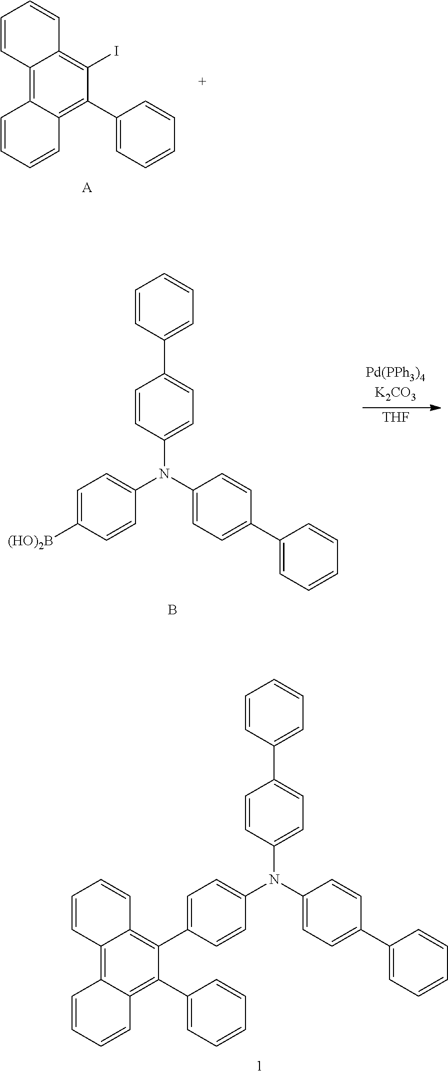

Under an argon (Ar) atmosphere, 4.30 g of Compound A, 9.98 g of Compound B, 654 mg of tetrakis(triphenylphosphine)palladium(0) (Pd(PPh₃)₄), and 6.91 g of potassium carbonate (K₂CO₃) were added to a solvent of THF (200 ml)/water (50 ml) and deaerated. The reaction mixture was stirred and refluxed for 8 hours. After that, the reactant was cooled, extracted with chloroform, and washed with a saturated saline solution. The organic layer thus obtained was dried with anhydrous sodium sulfate, filtered, and concentrated, and residues were separated by column chromatography to obtain 5.29 g (yield 72%) of Compound 1 as a white solid. The molecular weight of the compound measured by FAB-MS was 649. In addition, the chemical shift values of the compound measured by ¹H-NMR were 8.93 (m, 2H), 7.83 (m, 1H), 7.76-7.55 (12H), 7.51 (ddd, 1H, J=1, 7, 8 Hz), 7.47-7.34 (7H), 7.34-7.26 (2H), 7.26-7.19 (2H), 7.18-7.09 (6H), 7.05 (ddd, 2H, J=2, 2, 9 Hz). From the results, the white solid compound was identified as Compound 1.

### 2. Synthesis of Compound 12

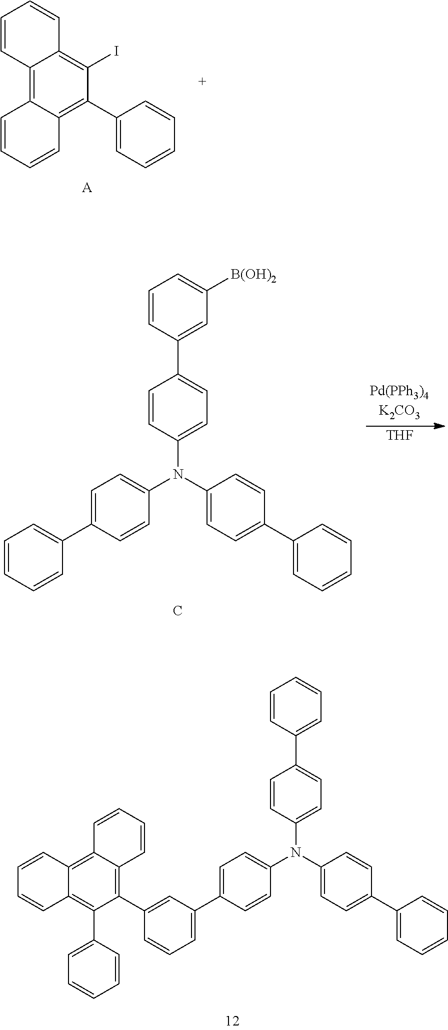

Under an argon (Ar) atmosphere, 3.78 g of Compound A, 10.3 g of Compound C, 575 mg of tetrakis(triphenylphosphine)palladium(0) (Pd(PPh₃)₄), and 6.34 g of potassium carbonate (K₂CO₃) were added to a solvent of THF (200 ml)/water (50 ml) and deaerated. The reaction mixture was stirred and refluxed for 8 hours.

After that, the reactant was cooled, extracted with chloroform, and washed with a saturated saline solution. The organic layer thus obtained was dried with anhydrous sodium sulfate, filtered, and concentrated, and residues were separated by column chromatography to obtain 4.55 g (yield 63%) of Compound 12 as a white solid. The molecular weight of the compound measured by FAB-MS was 725. In addition, the chemical shift values of the compound measured by ¹H-NMR were 8.93 (d, 2H, J=8 Hz), 7.74-7.55 (12H), 7.55-7.46 (6H), 7.46-7.12 (19H). From the results, the white solid compound was identified as Compound 12.

### 3. Synthesis of Compound 38

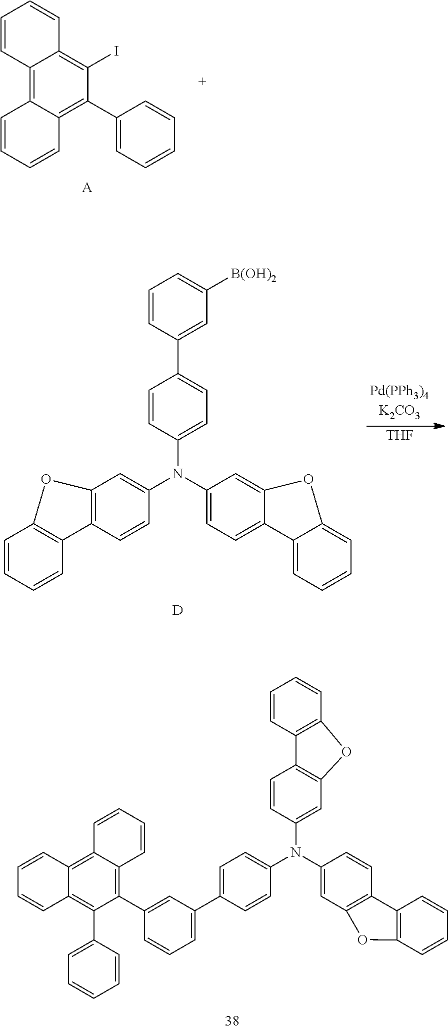

Under an argon (Ar) atmosphere, 2.51 g of Compound A, 7.19 g of Compound D, 381 mg of tetrakis(triphenylphosphine)palladium(0) (Pd(PPh₃)₄), and 5.31 g of potassium carbonate (K₂CO₃) were added to a solvent of THF (200 ml)/water (50 ml) and deaerated. The reaction mixture was stirred and refluxed for 8 hours.

After that, the reactant was cooled, extracted with chloroform, and washed with a saturated saline solution. The organic layer thus obtained was dried with anhydrous sodium sulfate, filtered, and concentrated, and residues were separated by column chromatography to obtain 3.38 g (yield 68%) of Compound 38 as a white solid. The molecular weight of the compound measured by FAB-MS was 753. In addition, the chemical shift values of the compound measured by ¹H-NMR were 8.87 (m, 2H), 7.96 (m, 2H), 7.92 (d, 2H, J=8H), 7,75 (m, 1H), 7.71-7.50 (7H), 7.50-7.36 (5H), 7.36-7.10 (16H). From the results, the white solid compound was identified as Compound 38.

### 4. Synthesis of Compound 11 (Synthesis of Compound F)

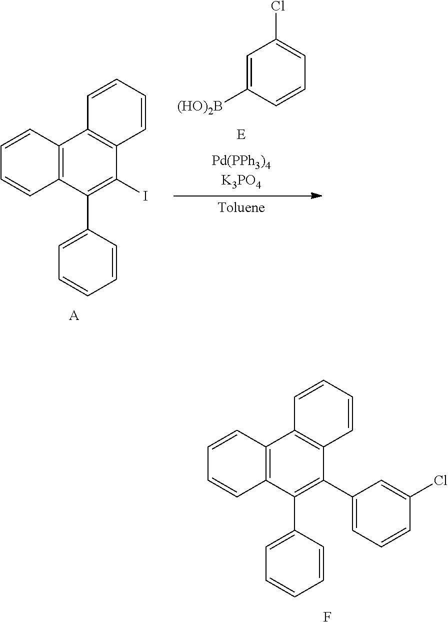

Under an argon (Ar) atmosphere, 10.0 g of Compound A, 29.2 g of Compound E, 2.22 g of tetrakis(triphenylphosphine)palladium(0) (Pd(PPh₃)₄), and 431 mg of palladium acetate (Pd(OAc)₂) were added to a solvent of toluene (300 ml)/ethyl alcohol (130 ml)/aqueous solution of 2 M tripotassium orthophosphate (K₃PO₄) (64 ml), and deaerated. The reaction mixture was stirred and refluxed for 24 hours. After that, the reaction product was cooled, extracted with chloroform, and washed with a saturated saline solution.

The organic layer thus obtained was dried with anhydrous sodium sulfate, filtered, and concentrated, and residues were separated by column chromatography to obtain 16.1 g (yield 69%) of Compound F as a white solid.

### (Synthesis of Compound 11)

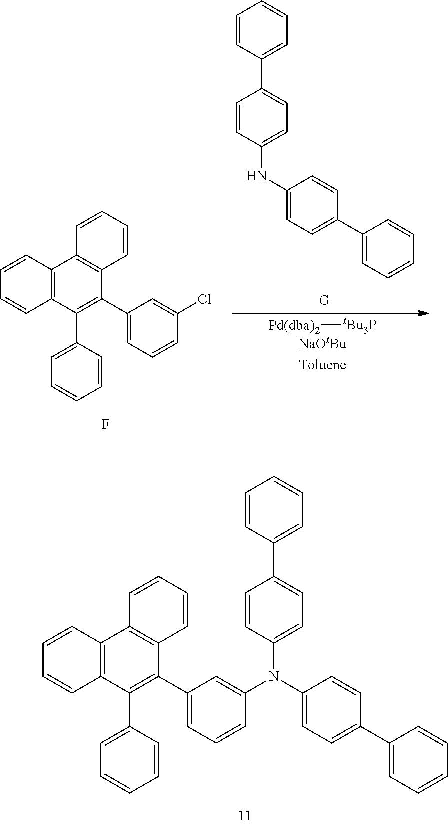

Under an argon (Ar) atmosphere, 4.01 g of Compound F, 3.89 g of Compound G, 1.06 g of sodium t-butoxide, 316 mg of bis(dibenzylideneacetone)palladium(0) (Pd(dba)₂), and 1.47 ml of 1.5 M toluene solution of tri-t-butyl phosphine (^{t}Bu₃P) were added to 150 ml of toluene, and deaerated. The reaction mixture was stirred and refluxed for 24 hours. After that, the reaction product was cooled, extracted with chloroform, and washed with a saturated saline solution.

The organic layer thus obtained was dried with anhydrous sodium sulfate, filtered, and concentrated, and residues were separated by column chromatography to obtain 6.01 g (yield 84%) of Compound 11 as a white solid. The molecular weight of the compound measured by FAB-MS was 649. In addition, the chemical shift values of the compound measured by ¹H-NMR were 8.88 (d, 2H, J=10 Hz), 7.76-7.43 (21H), 7.43-7.17 (5H), 7.09 (ddd, 4H, J=2, 2, 8 Hz), 7.04-6.90 (3H). From the results, the white solid compound was identified as Compound 11.

### 5. Synthesis of Compound 16

### (Synthesis of Compound H)

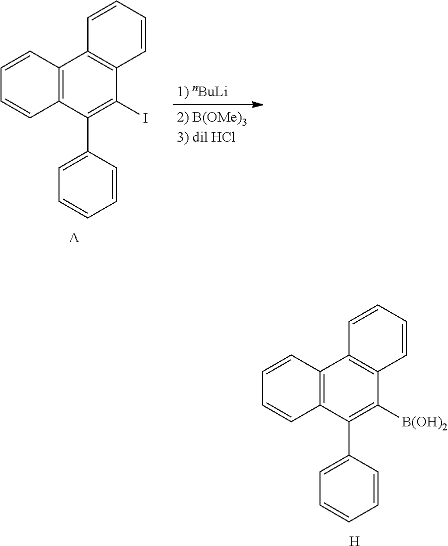

Under an argon (Ar) atmosphere, 20.0 g of Compound A was stirred in 300 ml of THF at −78° C. for 10 minutes, and 21.7 ml of n-butyllithium (n-BuLi) with 1.6 M concentration was slowly added thereto dropwisely using a dropping funnel, followed by additionally stirring for 30 minutes. Then, 7.05 ml of trimethyl borate (B(OMe)₃) was slowly added thereto dropwisely using a dropping funnel and additionally stirred at room temperature for 3 hours. After that, 300 ml of 1 M HCl solution was added and extracted once. Then, the product thus obtained was additionally extracted three times using water and toluene. The organic layer thus obtained was dried with anhydrous sodium sulfate, filtered, and concentrated, and residues were separated by column chromatography to obtain 4.78 g (yield 30%) of Compound H as a white solid.

### (Synthesis of Compound J)

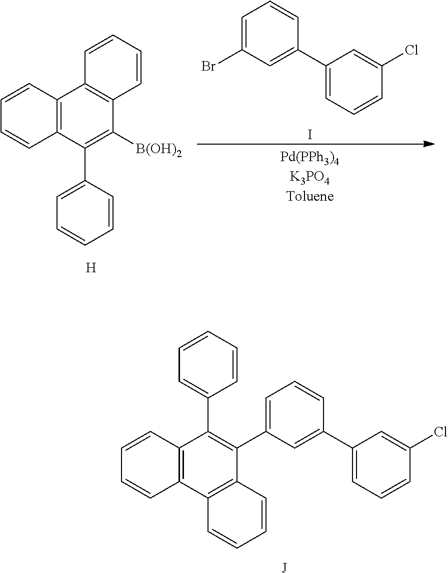

Under an argon (Ar) atmosphere, 4.50 g of Compound H, 4.04 g of Compound I, 9.61 g of tripotassium orthophosphate (K₃PO₄), and 872 mg of tetrakis(triphenylphosphine)palladium(0) (Pd(PPh₃)₄), were added to a solvent of toluene (150 ml)/ethyl alcohol (10 ml)/water (5 ml), and deaerated. The reaction mixture was stirred and refluxed for 24 hours. After that, the reaction product was cooled, extracted with chloroform, and washed with a saturated saline solution. The organic layer thus obtained was dried with anhydrous sodium sulfate, filtered, and concentrated, and residues were separated by column chromatography to obtain 2.80 g (yield 42%) of Compound J as a white solid.

### (Synthesis of Compound 16)

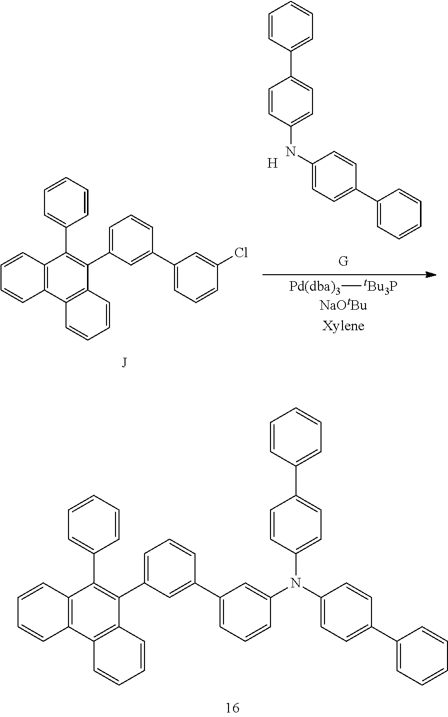

Under an argon (Ar) atmosphere, 2.45 g of Compound J, 1.79 g of Compound G, 1.06 g of sodium t-butoxide, 152 mg of tris(dibenzylideneacetone)dipalladium(0) (Pd₂(dba)₃), and 0.21 ml of 1.6 M toluene solution of tri-t-butyl phosphine (^{t}Bu₃P) were added to 150 ml of xylene, and deaerated. The reaction mixture was stirred and refluxed for 8 hours. After that, the reaction product was cooled, extracted with chloroform, and washed with a saturated saline solution. The organic layer thus obtained was dried with anhydrous sodium sulfate, filtered, and concentrated, and residues were separated by column chromatography to obtain 3.11 g (yield 70%) of Compound 16 as a white solid. The molecular weight of the compound measured by FAB-MS was 725. In addition, the chemical shift values of the compound measured by ¹H-NMR were 8.92 (d, 2H, J=8 Hz), 7.75-7.56 (11H), 7.56-6.95 (26H). From the results, the white solid compound was identified as Compound 16.

### 5. Synthesis of Compound 73

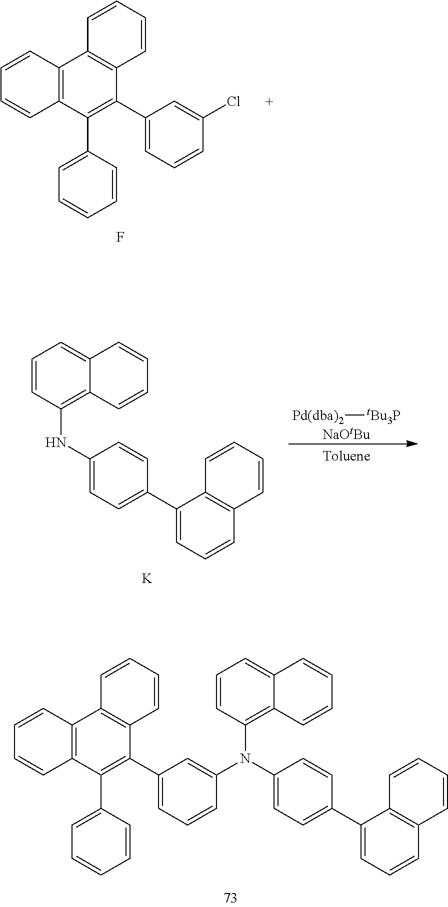

Compound F was synthesized by the same synthetic method of Compound F in the synthetic method of Compound 11. Then, under an argon (Ar) atmosphere, 5.00 g of Compound F, 5.21 g of Compound K, 1.32 g of sodium t-butoxide, 390 mg of bis(dibenzylideneacetone)palladium(0) (Pd(dba)₂), and 1.83 ml of 1.5 M toluene solution of tri-t-butyl phosphine (^{t}Bu₃P) were added to 150 ml of toluene, and deaerated. The reaction mixture was stirred and refluxed for 24 hours. After that, the reaction product was cooled, extracted with chloroform, and washed with a saturated saline solution.

The organic layer thus obtained was dried with anhydrous sodium sulfate, filtered, and concentrated, and residues were separated by column chromatography to obtain 6.65 g (yield 72%) of Compound 73 as a white solid. The molecular weight of the compound measured by FAB-MS was 673. In addition, the chemical shift values of the compound measured by ¹H-NMR were 8.88 (d, 2H, J=9 Hz), 8.05-7.77 (6H), 7.72-7.60 (3H), 7.60-7.27 (16H), 7.24 (m, 1H), 7.20-7.08 (3H), 6.97 (m, 2H), 6.92-6.82 (2H).

From the results, the white solid compound was identified as Compound 16.

### 7. Synthesis of Compound 103

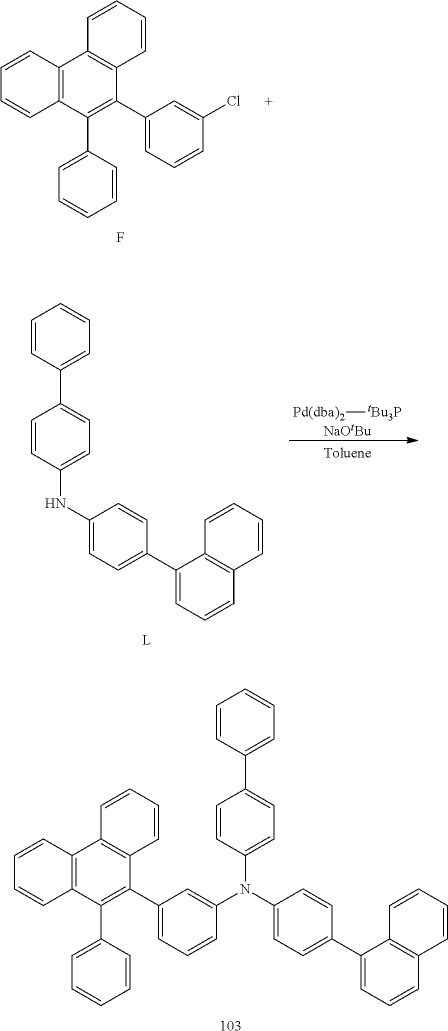

Compound F was synthesized by the same synthetic method of Compound F in the synthetic method of Compound 11. Then, under an argon (Ar) atmosphere, 4.20 g of Compound F, 4.70 g of Compound L, 1.11 g of sodium t-butoxide, 331 mg of bis(dibenzylideneacetone)palladium(0) (Pd(dba)₂), and 1.53 ml of 1.5 M toluene solution of tri-t-butyl phosphine (^{t}Bu₃P) were added to 150 ml of toluene, and deaerated. The reaction mixture was stirred and refluxed for 24 hours. After that, the reaction product was cooled, extracted with chloroform, and washed with a saturated saline solution.

The organic layer thus obtained was dried with anhydrous sodium sulfate, filtered, and concentrated, and residues were separated by column chromatography to obtain 6.93 g (yield 86%) of Compound 103 as a white solid. The molecular weight of the compound measured by FAB-MS was 699. In addition, the chemical shift values of the compound measured by ¹H-NMR were 8.89 (d, 2H, J=8 Hz), 8.00 (d, 1H, J=8 Hz), 7.92 (dd, 1H, J=2, 8 Hz), 7.86 (d, 1H, J=8 Hz), 7.78-7.33 (21H), 7.33-7.20 (4H), 7.20-7.03 (6H), 6.99 (ddd, 1H, J=1, 1, 8 Hz). From the results, the white solid compound was identified as Compound 103.

8. Synthesis of Compound 104

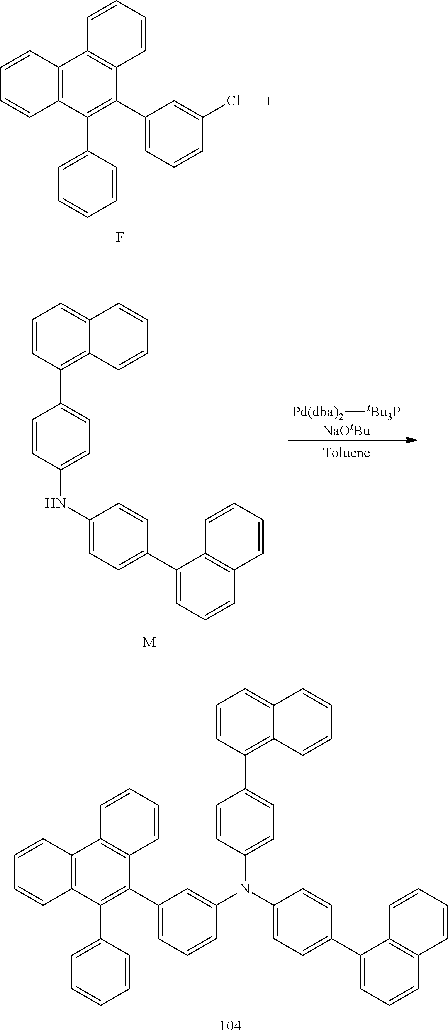

Compound F was synthesized by the same synthetic method of Compound F in the synthetic method of Compound 11. Then, under an argon (Ar) atmosphere, 5.00 g of Compound F, 6.35 g of Compound M, 1.32 g of sodium t-butoxide, 390 mg of bis(dibenzylideneacetone)palladium(0) (Pd(dba)₂), and 1.83 ml of 1.5 M toluene solution of tri-t-butyl phosphine (^{t}Bu₃P) were added to 150 ml of toluene, and deaerated. The reaction mixture was stirred and refluxed for 24 hours. After that, the reaction product was cooled, extracted with chloroform, and washed with a saturated saline solution.

The organic layer thus obtained was dried with anhydrous sodium sulfate, filtered, and concentrated, and residues were separated by column chromatography to obtain 8.53 g (yield 83%) of Compound 104 as a white solid. The molecular weight of the compound measured by FAB-MS was 699. In addition, the chemical shift values of the compound measured by ¹H-NMR were 8.90 (d, 2H, J=9 Hz), 8.02 (d, 2H, J=9 Hz), 7.92 (d, 2H, J=8 Hz), 7.86 (d, 2H, J=8 Hz), 7.78-7.62 (3H), 7.62-7.40 (16H), 7.40-7.09 (11H), 7.00 (m, 1H). From the results, the white solid compound was identified as Compound 104.

### 9. Synthesis of Compound 122

### (Synthesis of Compound P)

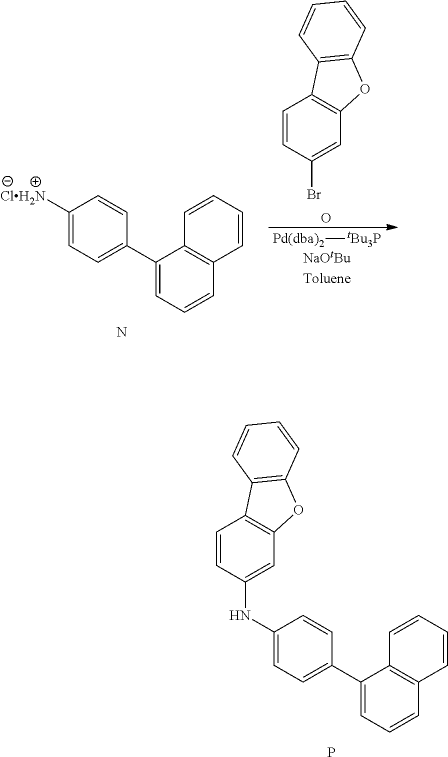

Under an argon (Ar) atmosphere, 7.25 g (28.3 mmol) of Compound N, 7.00 g (28.3 mmol) of Compound 0, 326 mg (0.566 mmol) of bis(dibenzylideneacetone)palladium(0) (Pd(dba)₂), 0.687 ml (1.13 mmol) of a 1.65 M tri-t-butylphosphine (^{t}Bu₃P) solution, and 2.72 g (28.3 mmol) of sodium t-butoxide were added to 200 ml of toluene, and deaerated. The reaction mixture was stirred at about 90° C. for about 4 hours. After that, the reaction product was cooled at room temperature, and treated with a filtration column, and the reaction product thus filtered was concentrated. The concentrated reaction product was recrystallized with toluene-ethanol to obtain 7.30 g (18.9 mmol, yield 67%) of Compound P.

### (Synthesis of Compound 122)

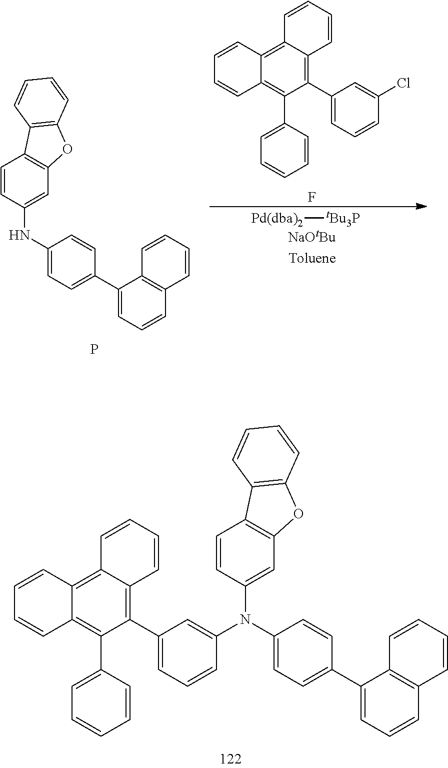

Under an argon (Ar) atmosphere, 6.80 g (17.6 mmol) of Compound P, 6.44 g (17.6 mmol) of Compound F, 406 mg (0.706 mmol) of bis(dibenzylideneacetone)palladium(O) (Pd(dba)₂), 0.856 ml (1.41 mmol) of a 1.65 M tri-t-butylphosphine (^{t}Bu₃P) solution, and 2.54 g (26.3 mmol) of sodium t-butoxide were added to 200 ml of toluene, and deaerated. The reaction mixture was heated and refluxed while heating and stirring for about 20 hours. After that, the reaction product was cooled at room temperature, and treated with a filtration column, and the reaction product thus filtered was concentrate. The concentrated reaction product was recrystallized with toluene-ethanol to obtain 8.52 g (12.0 mmol, yield 68%) of Compound 122. The molecular weight of the compound measured by FAB-MS was 713. In addition, the chemical shift values of the compound measured by ¹H-NMR were 8.89 (d, 2H, J=9 Hz), 8.05-7.82 (5H), 7.79-7.05 (27H), 6.99 (ddd, 1H, J=1.2 Hz, 1.2 Hz, 7.4 Hz). From the results, the compound thus obtained was identified as Compound 122.

### Device Manufacturing Examples

Hereinafter, device manufacture and evaluation of emission efficiency properties were conducted twice with respect to devices having different configuration.

### Device Manufacturing Examples 1

Light emitting elements according to Examples 1 to 3 were manufactured using Compounds 1, 12 and 38 as hole transport materials.

### Example Compounds

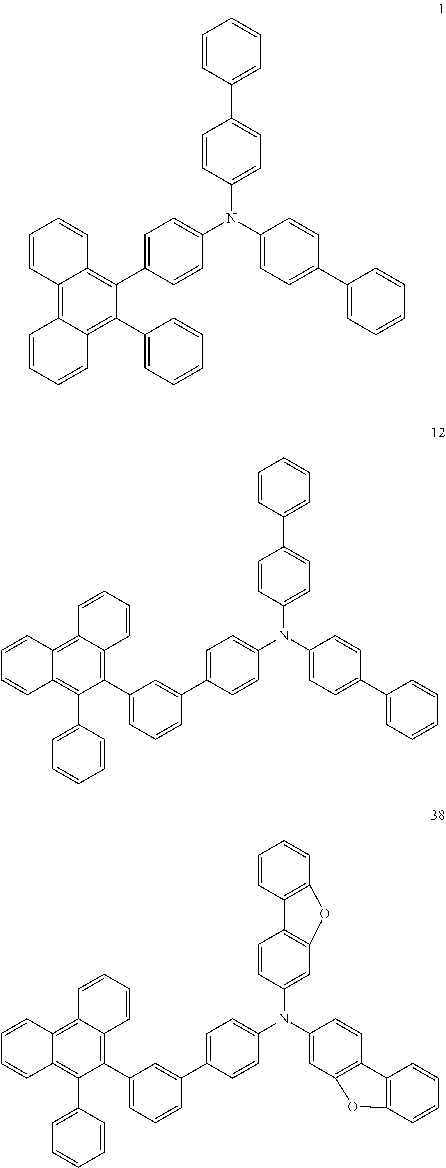 [SMILES: c1ccc(-c2ccc(N(c3ccc(-c4ccccc4)cc3)c3ccc(-c4c(-c5ccccc5)c5ccccc5c5ccccc45)cc3)cc2)cc1 ; c1ccc(-c2ccc(N(c3ccc(-c4ccccc4)cc3)c3ccc(-c4cccc(-c5c(-c6ccccc6)c6ccccc6c6ccccc56)c4)cc3)cc2)cc1 ; c1ccc(-c2c(-c3cccc(-c4ccc(N(c5ccc6c(c5)oc5ccccc56)c5ccc6c(c5)oc5ccccc56)cc4)c3)c3ccccc3c3ccccc23)cc1]

Light emitting elements according to Comparative Examples 1 to 6 were manufactured using Comparative Compounds c1 to c6 as hole transport materials.

### Comparative Compounds

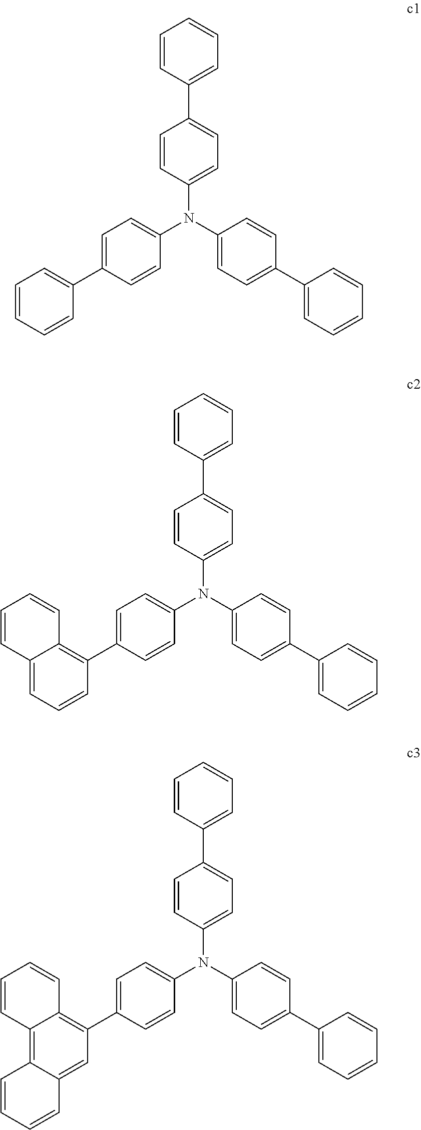 [SMILES: c1ccc(-c2ccc(N(c3ccc(-c4ccccc4)cc3)c3ccc(-c4ccccc4)cc3)cc2)cc1 ; c1ccc(-c2ccc(N(c3ccc(-c4ccccc4)cc3)c3ccc(-c4cccc5ccccc45)cc3)cc2)cc1 ; c1ccc(-c2ccc(N(c3ccc(-c4ccccc4)cc3)c3ccc(-c4cc5ccccc5c5ccccc45)cc3)cc2)cc1]

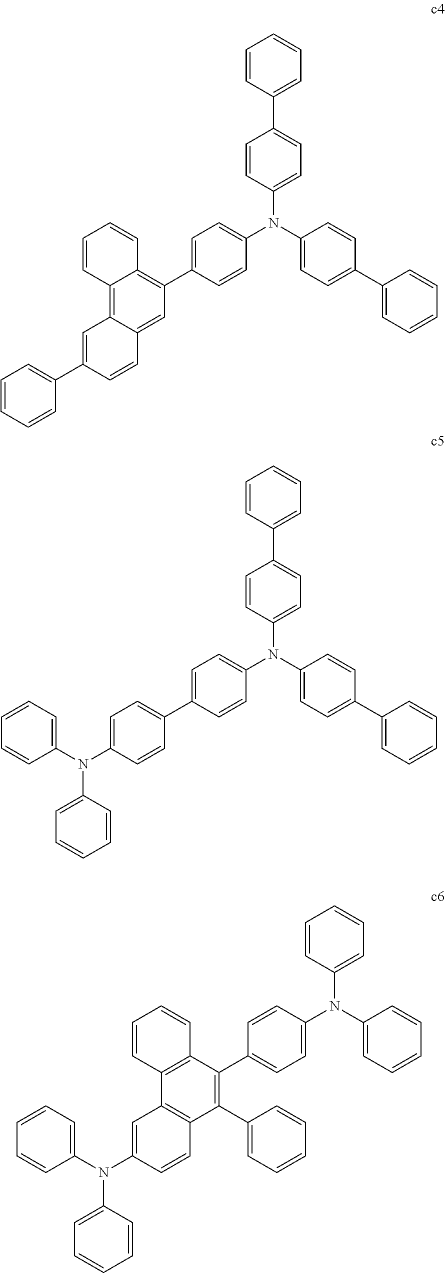 [SMILES: c1ccc(-c2ccc(N(c3ccc(-c4ccccc4)cc3)c3ccc(-c4cc5ccc(-c6ccccc6)cc5c5ccccc45)cc3)cc2)cc1 ; c1ccc(-c2ccc(N(c3ccc(-c4ccccc4)cc3)c3ccc(-c4ccc(N(c5ccccc5)c5ccccc5)cc4)cc3)cc2)cc1 ; c1ccc(-c2c(-c3ccc(N(c4ccccc4)c4ccccc4)cc3)c3ccccc3c3cc(N(c4ccccc4)c4ccccc4)ccc23)cc1]

Light emitting elements according to Examples 1 to 3 and Comparative Examples 1 to 6 were manufactured by forming a first electrode using ITO to a thickness of about 150 nm, a hole injection layer using tris naphthyl phenyl amino triphenylamine (TNATA) to a thickness of about 60 nm, a hole transport layer using the compound according to the example or the comparative example to a thickness of about 30 nm, an emission layer using dinaphthyl anthracene (ADN) doped with 3% tetra-t-butylperylene (TBP) to a thickness of about 25 nm, an electron transport layer using tris(8-hydroxyquinolinato)aluminum (Alq₃) to a thickness of about 25 nm, an electron injection layer using LiF to a thickness of about 1 nm, and a second electrode using A₁ to a thickness of about 100 nm. Each layer was formed by a vacuum deposition method.

Then, the emission efficiency of the light emitting element thus manufactured was evaluated. The emission efficiency was measured as a relative emission efficiency ratio of each example and comparative example when considering the emission efficiency of a light emitting element of Comparative Example 3 as 100%.

TABLE 1

| | | Emission Efficiency |
|---|---|---|
| Device | | (Relative Ratio To |
| Manufacturing | Hole Transport Layer | Comparative |
| Example | Material | Example 3) |
| Example 1 | Example Compound 1 | 110% |
| Example 2 | Example Compound 12 | 108% |
| Example 3 | Example Compound 38 | 106% |
| Comparative | Comparative Compound | 70% |
| Example 1 | c1 | |
| Comparative | Comparative Compound | 98% |
| Example 2 | c2 | |
| Comparative | Comparative Compound | 100% |
| Example 3 | c3 | |
| Comparative | Comparative Compound | 92% |
| Example 4 | c4 | |
| Comparative | Comparative Compound | 65% |
| Example 5 | c5 | |
| Comparative | Comparative Compound | 68% |
| Example 6 | c6 | |

Referring to the results of Table 1, emission efficiency was improved for Examples 1 to 3 when compared to that of Comparative Examples 1 to 6. From the results in Table 1, it may be found that light emitting elements including the compounds according to example embodiments may attain high emission efficiency.

In Examples 1 to 3, a monoamine compound including a phenyl group as an adjacent group to a position where a phenanthryl group and an arylene linker are connected is included. Without being bound by theory, it is believed that a volume near a phenanthryl group in which a LUMO orbital which is related to electron transportation is distributed, is increased, while maintaining amine properties, and the transportation of electrons from an emission layer to a hole transport layer becomes difficult, and thus, the concentration of excitons in the emission layer is increased to increase the emission efficiency.

In Comparative Example 3, a compound in which a phenanthryl group and an amine group are connected via an arylene linker is included, but the phenanthryl group in the compound included in Comparative Example 3 is not substituted with a phenyl group. Accordingly, without being bound by theory, it is believed that blocking effect of electrons transported from an emission layer to a hole transport layer is not attained, and emission efficiency is lower than that of the examples.

In Comparative Example 4, a compound in which a phenyl group is substituted at a phenanthryl group is included, but the phenyl group is not substituted at a position adjacent to a position where the phenanthryl group and an arylene linker are connected, that is, not at the carbon of position 10 of the phenanthryl group, but at the carbon of position 3. Accordingly, without being bound by theory, it is believed that the volume increase at an active position, where a LUMO orbital which is related to electron transportation is distributed, is not attained, and emission efficiency is reduced when compared to that of the examples.

In Comparative Examples 5 and 6, the devices included a diamine compound having a low HOMO energy level, such that hole injection from a hole injection layer to a hole transport layer may be deteriorated. Accordingly, emission efficiency was lower when compared to that of the examples.

### Device Manufacturing Examples 2

Light emitting elements according to Examples 4 to 12 were manufactured using Compounds 1, 11, 12, 16, 38, 73, 103, 104, and 122 as second hole transport materials.

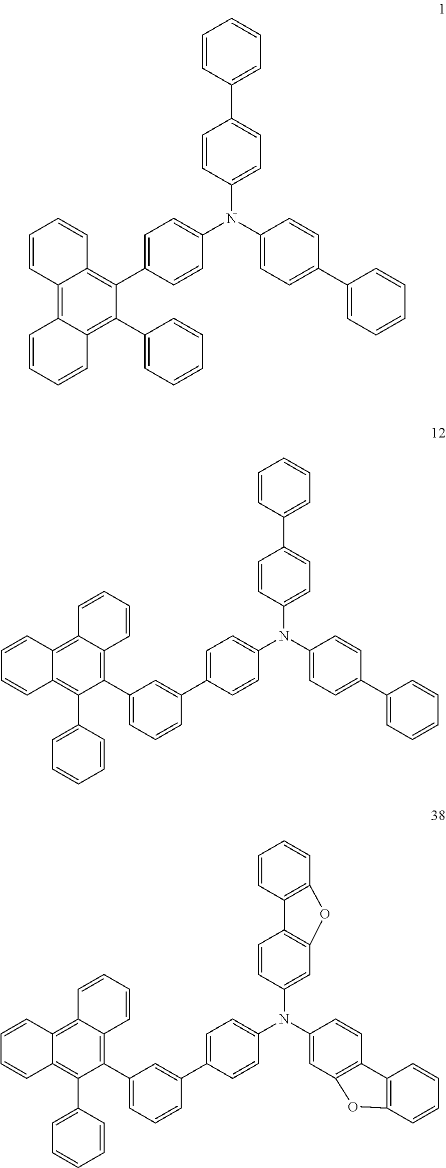 [SMILES: c1ccc(-c2ccc(N(c3ccc(-c4ccccc4)cc3)c3ccc(-c4c(-c5ccccc5)c5ccccc5c5ccccc45)cc3)cc2)cc1 ; c1ccc(-c2ccc(N(c3ccc(-c4ccccc4)cc3)c3ccc(-c4cccc(-c5c(-c6ccccc6)c6ccccc6c6ccccc56)c4)cc3)cc2)cc1 ; c1ccc(-c2c(-c3cccc(-c4ccc(N(c5ccc6c(c5)oc5ccccc56)c5ccc6c(c5)oc5ccccc56)cc4)c3)c3ccccc3c3ccccc23)cc1]

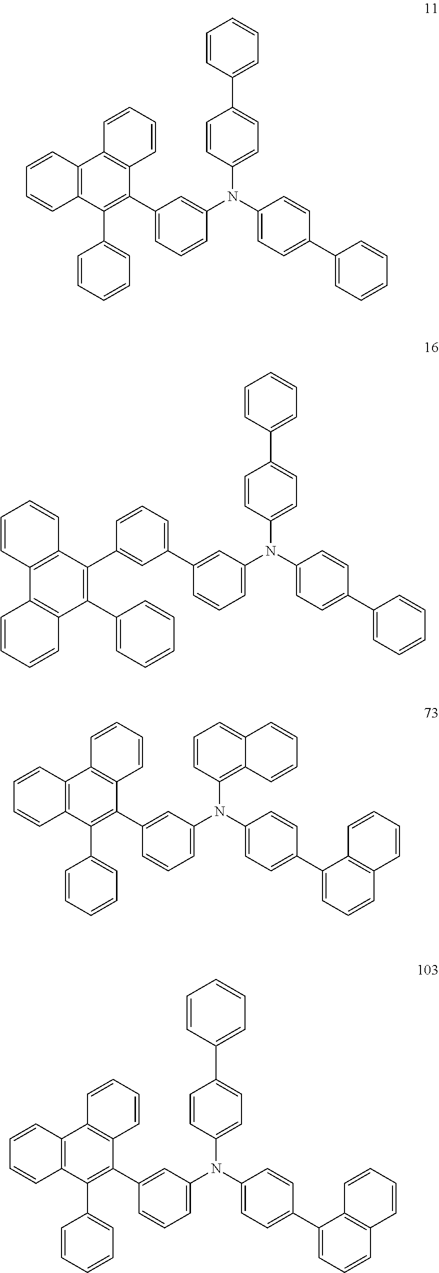 [SMILES: c1ccc(-c2ccc(N(c3ccc(-c4ccccc4)cc3)c3cccc(-c4c(-c5ccccc5)c5ccccc5c5ccccc45)c3)cc2)cc1 ; c1ccc(-c2ccc(N(c3ccc(-c4ccccc4)cc3)c3cccc(-c4cccc(-c5c(-c6ccccc6)c6ccccc6c6ccccc56)c4)c3)cc2)cc1 ; c1ccc(-c2c(-c3cccc(N(c4ccc(-c5cccc6ccccc56)cc4)c4cccc5ccccc45)c3)c3ccccc3c3ccccc23)cc1 ; c1ccc(-c2ccc(N(c3ccc(-c4cccc5ccccc45)cc3)c3cccc(-c4c(-c5ccccc5)c5ccccc5c5ccccc45)c3)cc2)cc1]

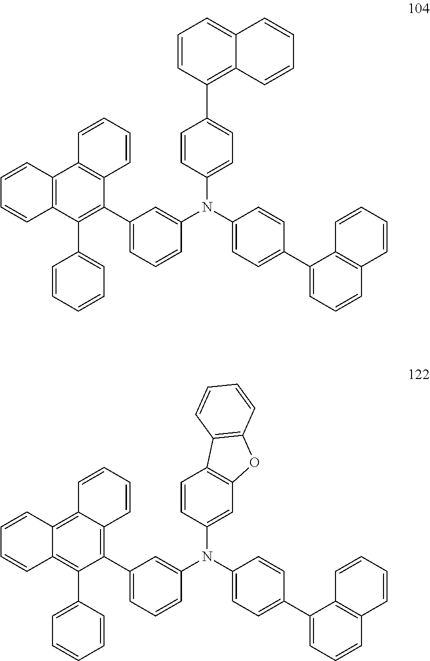 [SMILES: c1ccc(-c2c(-c3cccc(N(c4ccc(-c5cccc6ccccc56)cc4)c4ccc(-c5cccc6ccccc56)cc4)c3)c3ccccc3c3ccccc23)cc1 ; c1ccc(-c2c(-c3cccc(N(c4ccc(-c5cccc6ccccc56)cc4)c4ccc5c(c4)oc4ccccc45)c3)c3ccccc3c3ccccc23)cc1]

Light emitting elements according to Comparative Examples 7 to 15 were manufactured using Comparative Compounds c1 to c9 as hole transport materials.

### Comparative Compounds

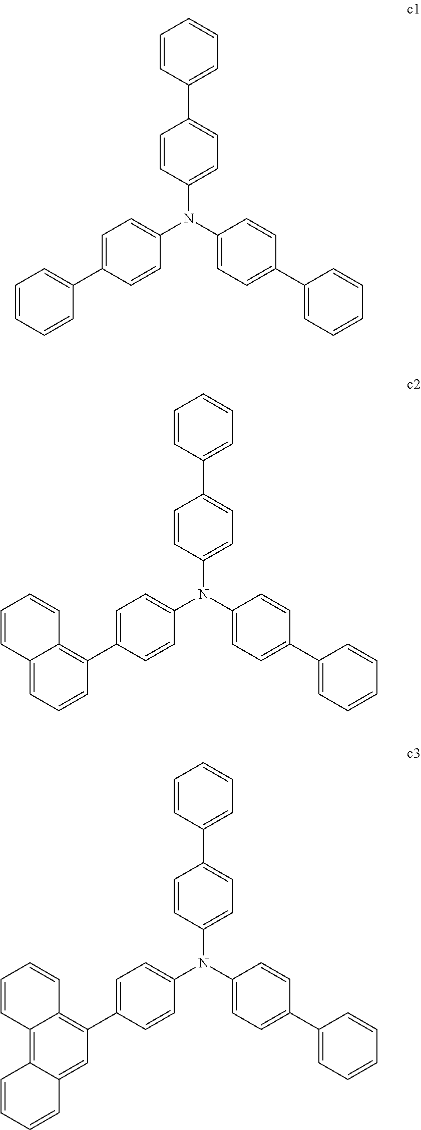 [SMILES: c1ccc(-c2ccc(N(c3ccc(-c4ccccc4)cc3)c3ccc(-c4ccccc4)cc3)cc2)cc1 ; c1ccc(-c2ccc(N(c3ccc(-c4ccccc4)cc3)c3ccc(-c4cccc5ccccc45)cc3)cc2)cc1 ; c1ccc(-c2ccc(N(c3ccc(-c4ccccc4)cc3)c3ccc(-c4cc5ccccc5c5ccccc45)cc3)cc2)cc1]

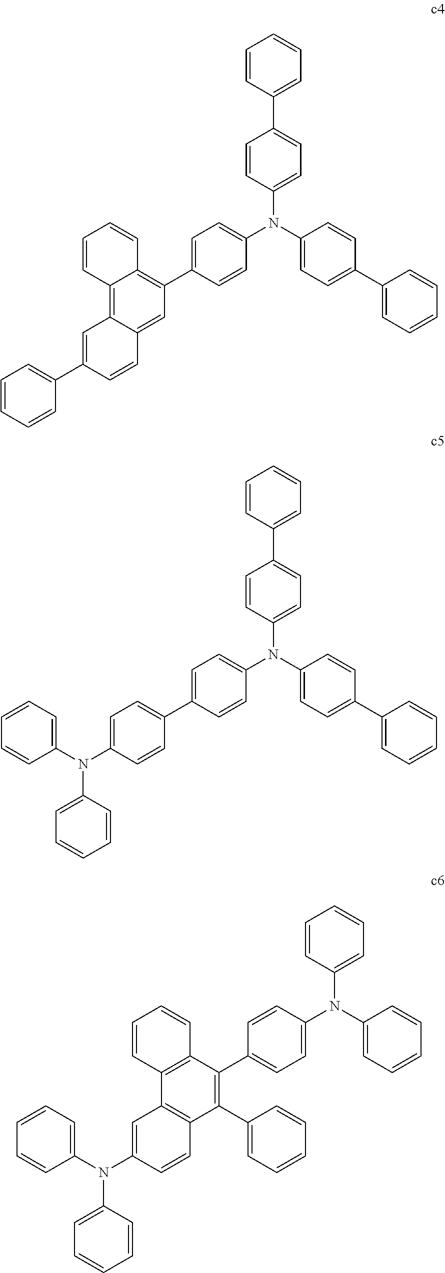 [SMILES: c1ccc(-c2ccc(N(c3ccc(-c4ccccc4)cc3)c3ccc(-c4cc5ccc(-c6ccccc6)cc5c5ccccc45)cc3)cc2)cc1 ; c1ccc(-c2ccc(N(c3ccc(-c4ccccc4)cc3)c3ccc(-c4ccc(N(c5ccccc5)c5ccccc5)cc4)cc3)cc2)cc1 ; c1ccc(-c2c(-c3ccc(N(c4ccccc4)c4ccccc4)cc3)c3ccccc3c3cc(N(c4ccccc4)c4ccccc4)ccc23)cc1]

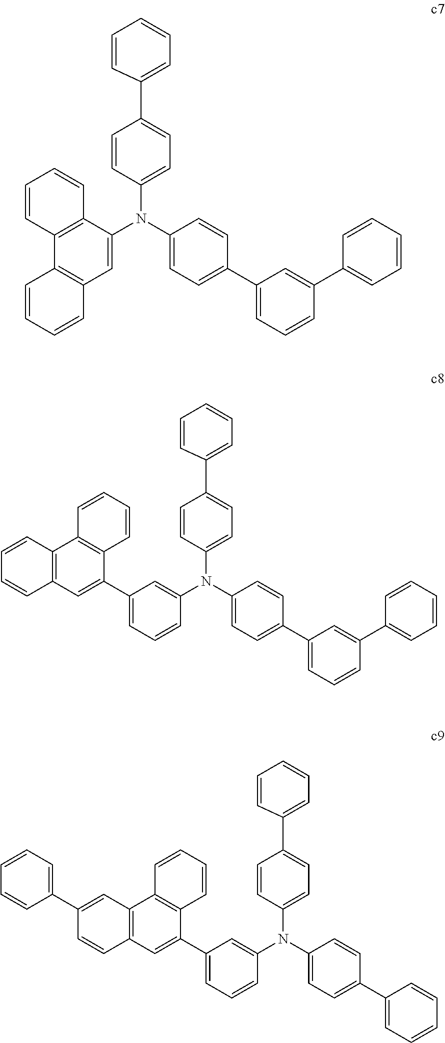 [SMILES: c1ccc(-c2ccc(N(c3ccc(-c4cccc(-c5ccccc5)c4)cc3)c3cc4ccccc4c4ccccc34)cc2)cc1 ; c1ccc(-c2ccc(N(c3ccc(-c4cccc(-c5ccccc5)c4)cc3)c3cccc(-c4cc5ccccc5c5ccccc45)c3)cc2)cc1 ; c1ccc(-c2ccc(N(c3ccc(-c4ccccc4)cc3)c3cccc(-c4cc5ccc(-c6ccccc6)cc5c5ccccc45)c3)cc2)cc1]

Light emitting elements according to Examples 4 to 11 and Comparative Examples 7 to 15 were manufactured by forming a first electrode using ITO to a thickness of about 150 nm, a hole injection layer using dipyrazino[2,3-f:2′,3′-h]quinoxaline-2,3,6,7,10,11-hexacarbonitrile (HAT-CN) to a thickness of about 10 nm, a first hole transport layer using N-([1,1′-biphenyl]-4-yl)-9,9-dimethyl-N-(4-(9-phenyl-9H-carbazol-3-yl)phenyl)-9H-fluoren-2-amine to a thickness of about 70 nm, a second hole transport layer using the example compounds or comparative compounds to a thickness of about 10 nm, an emission layer using (9-(4-(naphthalene-1-yl)phenyl)-10-perdeuterophenyl)anthracene doped with 3% N¹,N⁶-di(naphthalene-1-yl)-N¹,N⁶-diphenylpyrene-1,6-diamine to a thickness of about 25 nm, an electron transport layer using tris(8-hydroxyquinolinato)aluminum (Alq₃) to a thickness of about 25 nm, an electron injection layer using LiF to a thickness of about 1 nm, and a second electrode using A₁ to a thickness of about 100 nm. Each layer and the second electrode were formed by a vacuum deposition method.

Then, the emission efficiency of the light emitting element thus manufactured was evaluated. The emission efficiency was measured as a relative emission efficiency ratio of each example and comparative example when considering the emission efficiency of a light emitting element of Comparative Example 9 as 100%.

TABLE 2

| | | Emission Efficiency |
|---|---|---|
| Device | | (Relative Ratio To |
| Manufacturing | Second Hole Transport | Comparative |
| Example | Layer | Example 9) |
| Example 4 | Example Compound 1 | 110% |
| Example 5 | Example Compound 11 | 110% |
| Example 6 | Example Compound 12 | 110% |
| Example 7 | Example Compound 16 | 113% |
| Example 8 | Example Compound 38 | 108% |
| Example 9 | Example Compound 73 | 119% |
| Example 10 | Example Compound 103 | 113% |
| Example 11 | Example Compound 104 | 121% |
| Example 12 | Example Compound 122 | 113% |
| Comparative | Comparative Compound | 80% |
| Example 7 | c1 | |
| Comparative | Comparative Compound | 95% |
| Example 8 | c2 | |
| Comparative | Comparative Compound | 100% |
| Example 9 | c3 | |
| Comparative | Comparative Compound | 94% |
| Example 10 | c4 | |
| Comparative | Comparative Compound | 60% |
| Example 11 | c5 | |
| Comparative | Comparative Compound | 63% |
| Example 12 | c6 | |
| Comparative | Comparative Compound | 95% |
| Example 13 | c7 | |
| Comparative | Comparative Compound | 101% |
| Example 14 | c8 | |
| Comparative | Comparative Compound | 99% |
| Example 15 | c9 | |

Referring to the results of Table 2, it may be found that emission efficiency was improved for Examples 4 to 12 when compared to that of Comparative Examples 7 to 15. From the results in Table 2, it may be found that light emitting elements including the compounds according to example embodiments may attain high emission efficiency.

In Examples 4 to 12, a monoamine compound including a phenyl group as an adjacent group to a position where a phenanthryl group and an arylene linker are connected is included. Without being bound by theory, it is believed that a volume near a phenanthryl group in which a LUMO orbital which is related to electron transportation is distributed, is increased, while maintaining amine properties, and the transportation of electrons from an emission layer to a hole transport layer becomes difficult, and thus, the concentration of excitons in the emission layer is increased to increase the emission efficiency.

In Comparative Examples 9, 13, and 14, a compound in which a phenanthryl group and an amine group are connected via an arylene linker is included, but the phenanthryl group in the compounds included in Comparative Examples 9, 13, and 14 is not substituted with a phenyl group. Accordingly, without being bound by theory, it is believed that blocking effect of electrons transported from an emission layer to a hole transport layer is not attained, and emission efficiency is lower than that of the examples.

In Comparative Examples 10 and 15, a compound in which a phenyl group is substituted at a phenanthryl group is included, but the phenyl group is not substituted at an adjacent position to a position where the phenanthryl group and an arylene linker are connected, that is, not at the carbon of position 10 of the phenanthryl group, but at the carbon of position 3. Accordingly, without being bound by theory, it is believed that the volume increase at an active position, where a LUMO orbital which is related to electron transportation is distributed, is not attained, and emission efficiency is reduced when compared to that of the examples.

In Comparative Examples 11 and 12, the devices included a diamine compound having a low HOMO energy level, such that hole injection from a hole injection layer to a hole transport layer may be deteriorated. Accordingly, emission efficiency was lower when compared to that of the examples.

By way of summation and review, a light emitting element may be, for example, an organic device having a first electrode, a hole transport layer disposed on the first electrode, an emission layer disposed on the hole transport layer, an electron transport layer disposed on the emission layer, and a second electrode disposed on the electron transport layer. Holes are injected from the first electrode, and the injected holes move via the hole transport layer and injected into the emission layer. Meanwhile, electrons are injected from the second electrode, and the injected electrons move via the electron transport layer and injected into the emission layer. By recombining the injected holes and electrons into the emission layer, excitons are generated in the emission layer. A light emitting element emits light using light emitted during the transition of the excitons back to a ground state. The configuration of a light emitting element is not limited thereto, and various modifications may be possible.

As described above, a monoamine compound according to an example embodiment may be used as a material for a light emitting element. The light emitting element including the monoamine compound according to an example embodiment may attain high emission efficiency. The present disclosure provides a monoamine compound used in a light emitting element having high emission efficiency. The present disclosure also provides a light emitting element having high emission efficiency

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope of the present invention as set forth in the following claims.

What is claimed is:

1. An organic electroluminescence device, comprising:
a first electrode;
a hole transport region provided on the first electrode;
an emission layer provided on the hole transport region;
an electron transport region provided on the emission layer; and
a second electrode provided on the electron transport region,
wherein at least one of the hole transport region, the emission region, and the electron transport region includes a monoamine compound represented by the following Formula 1: 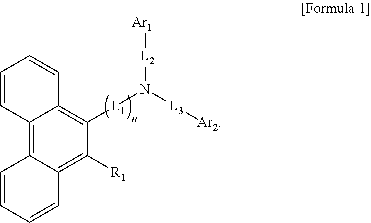
where L₁ is a substituted or unsubstituted arylene group having 6 to 30 carbon atoms for forming a ring, or a substituted or unsubstituted heteroarylene group having 2 to 30 carbon atoms for forming a ring,
n is 1 or 2,
L₂ and L₃ are each independently a direct linkage, a substituted or unsubstituted arylene group having 6 to 30 carbon atoms for forming a ring, or a substituted or unsubstituted heteroarylene group having 2 to 30 carbon atoms for forming a ring,
R₁ is a substituted or unsubstituted alkyl group having 1 to 10 carbon atoms, a substituted or unsubstituted aryl group having 6 to 30 carbon atoms for forming a ring, a substituted or unsubstituted heteroaryl group having 2 to 30 carbon atoms for forming a ring, or a substituted or unsubstituted aryl silyl group,
when R₁ is substituted, the substituent is a deuterium atom, a cyano group, a substituted or unsubstituted alkyl group having 1 to 10 carbon atoms, or a substituted or unsubstituted aryl group having 6 to 30 carbon atoms for forming a ring,
Ar₁ and Ar₂ are each independently a substituted or unsubstituted aryl group having 6 to 30 carbon atoms for forming a ring, or a substituted or unsubstituted dibenzofuranyl group, a substituted or unsubstituted dibenzothiophenyl group, a substituted or unsubstituted benzonaphthofuranyl group, or a substituted or unsubstituted benzonaphthothiophenyl group, and
wherein when at least one of L₂, L₃, Ar₁ and Ar₂ is substituted with a heterocycle, the heteroaryl group, the heteroaryl group is a substituted or unsubstituted dibenzofuranyl group, a substituted or unsubstituted dibenzothiophenyl group, a substituted or unsubstituted benzonaphthofuranyl group, or a substituted or unsubstituted benzonaphthothiophenyl group.

2. The organic electroluminescence device as claimed in claim 1, wherein the hole transport region includes the monoamine compound represented by Formula 1.

3. The organic electroluminescence device as claimed in claim 2, wherein the hole transport region includes:
a hole injection layer disposed on the first electrode; and
a hole transport layer disposed on the hole injection layer, and
wherein the hole transport layer includes the monoamine compound represented by Formula 1.

4. The organic electroluminescence device as claimed in claim 2, wherein the hole transport region includes:
a hole injection layer disposed on the first electrode;
a first hole transport layer disposed on the hole injection layer; and
a second hole transport layer disposed on the first hole transport layer, the second hole transport layer being adjacent to the emission layer,
wherein the second hole transport layer includes the monoamine compound represented by Formula 1.

5. The organic electroluminescence device as claimed in claim 1, wherein the monoamine compound represented by Formula 1 is represented by the following Formula 2-1 or 2-2: 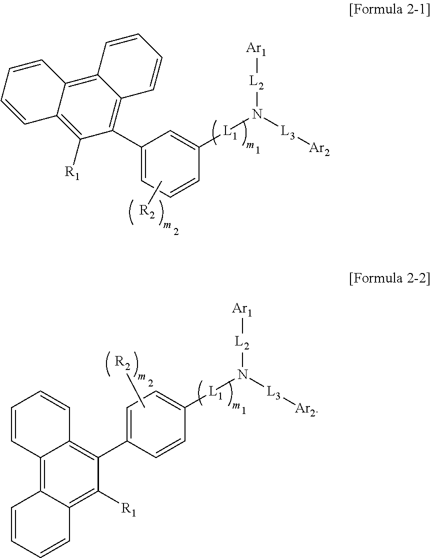
where m₁ is 0 or 1,
m₂ is an integer of 0 to 2,
R₂ is a hydrogen atom, a deuterium atom, a substituted or unsubstituted alkyl group having 1 to 10 carbon atoms, a substituted or unsubstituted aryl group having 6 to 30 carbon atoms for forming a ring, a substituted or unsubstituted heteroaryl group having 2 to 30 carbon atoms for forming a ring, or a substituted or unsubstituted aryl silyl group, or combines with an adjacent group to form a ring, and
Ar₁, Ar₂, L₁, L₂, L₃, and R₁ are the same as defined for Formula 1.

6. The organic electroluminescence device as claimed in claim 5, wherein the monoamine compound represented by Formula 2-1 or 2-2 is represented by one of the following Formulae 2-1-1 to 2-2-3: 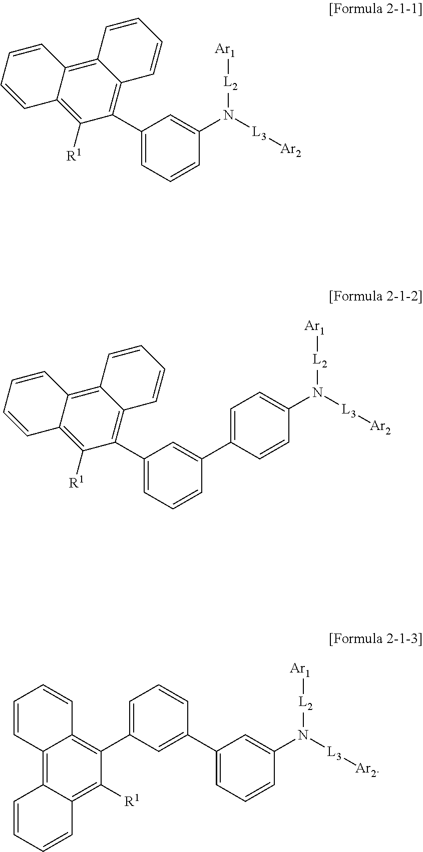
Ar₁ and Ar₂, L₂ and L₃, and R₁ are the same as defined for Formula 1.

7. The organic electroluminescence device of claim 1, wherein Ar₁ and Ar₂ are each independently represented by the following Formula 4: 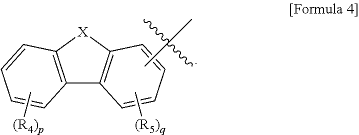
in Formula 4,
X is O or S,
R₄ and R₅ are each independently a hydrogen atom, a deuterium atom, a substituted or unsubstituted alkyl group having 1 to 10 carbon atoms, a substituted or unsubstituted aryl group having 6 to 30 carbon atoms for forming a ring, a substituted or unsubstituted heteroaryl group having 2 to 30 carbon atoms for forming a ring, or a substituted or unsubstituted aryl silyl group,
p is an integer of 0 to 4, and
q is an integer of 0 to 3.

8. The organic electroluminescence device of claim 1, the hole transport region further includes a monoamine compound represented by the following Formula H-1: 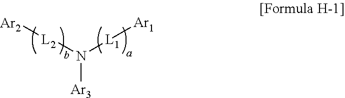
in Formula H-1,
L₁ and L₂ are each independently a direct linkage, a substituted or unsubstituted arylene group having 6 to 30 carbon atoms for forming a ring, or a substituted or unsubstituted heteroarylene group having 2 to 30 carbon atoms for forming a ring,
a and b are each independently an integer of 0 to 10,
Ar₁ and Ar₂ are each independently a substituted or unsubstituted aryl group having 6 to 30 carbon atoms for forming a ring, or a substituted or unsubstituted heteroaryl group having 2 to 30 carbon atoms for forming a ring, and
Ar₃ may be a substituted or unsubstituted aryl group having 6 to 30 carbon atoms for forming a ring, or a substituted or unsubstituted heteroaryl group having 2 to 30 carbon atoms for forming a ring.

9. The organic electroluminescence device of claim 1, the emission region further includes a compound represented by the following Formula E-1: 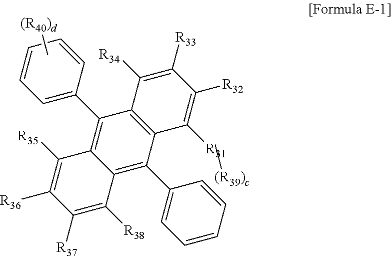
in Formula E-1,
R₃₁ to R₄₀ are each independently a hydrogen atom, a deuterium atom, a halogen atom, a substituted or unsubstituted silyl group, a substituted or unsubstituted thiol group, a substituted or unsubstituted oxy group, a substituted or unsubstituted alkyl group having 1 to 10 carbon atoms, a substituted or unsubstituted alkenyl group having 2 to 10 carbon atoms, a substituted or unsubstituted aryl group having 6 to 30 carbon atoms for forming a ring, or a substituted or unsubstituted heteroaryl group having 2 to 30 carbon atoms for forming a ring, or combined with an adjacent group to form a ring, and
c and d are each independently an integer of 0 to 5.

10. The organic electroluminescence device of claim 1, the emission region further includes a compound represented by the following Formula E-2a or Formula E-2b: 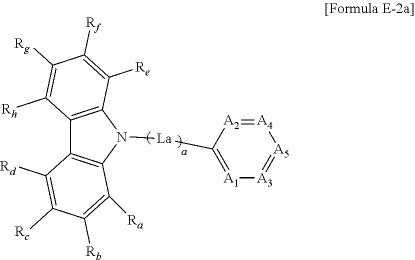
in Formula E-2a, a is an integer of 0 to 10,
Lₐ is a direct linkage, a substituted or unsubstituted arylene group having 6 to 30 carbon atoms for forming a ring, or a substituted or unsubstituted heteroarylene group having 2 to 30 carbon atoms for forming a ring,
A₁ to A₅ are each independently N or CRi,
Rₐ to Rᵢ are each independently a hydrogen atom, a deuterium atom, a substituted or unsubstituted amine group, a substituted or unsubstituted thiol group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 carbon atoms for forming a ring, or a substituted or unsubstituted heteroaryl group having 2 to 30 carbon atoms for forming a ring, or may be combined with an adjacent group to form a ring, and
two or three selected from A₁ to A₅ are N, and the remainder are CRi: 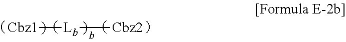
in Formula E-2b, Cbz1 and Cbz2 are each independently an unsubstituted carbazole group, or a carbazole group substituted with an aryl group having 6 to 30 carbon atoms for forming a ring, L_{b} is a direct linkage, a substituted or unsubstituted arylene group having 6 to 30 carbon atoms for forming a ring, or a substituted or unsubstituted heteroarylene group having 2 to 30 carbon atoms for forming a ring, and
b is an integer of 0 to 10.

11. The organic electroluminescence device of claim 1, the emission region further includes a compound represented by the following Formula M-a or Formula M-b: 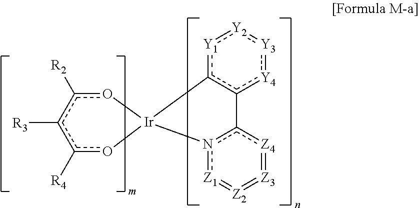
in Formula M-a,
Y₁ to Y₄, and Z₁ to Z₄ are each independently CR₁ or N,
R₁ to R₄ are each independently a hydrogen atom, a deuterium atom, a substituted or unsubstituted amine group, a substituted or unsubstituted thiol group, a substituted or unsubstituted oxy group, a substituted or unsubstituted alkyl group having 1 to 20 carbon atoms, a substituted or unsubstituted alkenyl group having 2 to 20 carbon atoms, a substituted or unsubstituted aryl group having 6 to 30 carbon atoms for forming a ring, or a substituted or unsubstituted heteroaryl group having 2 to 30 carbon atoms for forming a ring, or may be combined with an adjacent group to form a ring,
m is 0 or 1,
n is 2 or 3, and
if m is 0, n is 3, and if m is 1, n is 2: 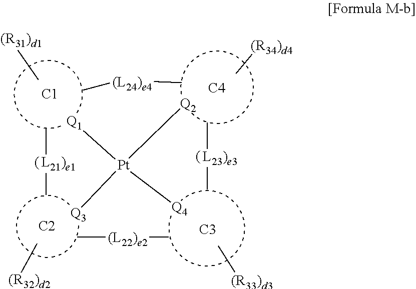
in Formula M-b, Q₁ to Q₄ are each independently C or N,
C1 to C4 are each independently a substituted or unsubstituted hydrocarbon ring having 5 to 30 carbon atoms for forming a ring, or a substituted or unsubstituted heterocycle having 2 to 30 carbon atoms for forming a ring,
L₂₁ to L₂₄ are each independently a direct linkage, 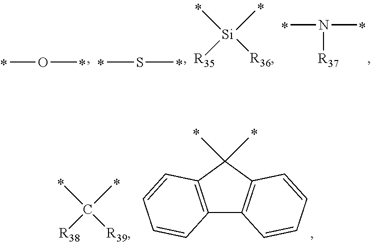
a substituted or unsubstituted divalent alkyl group having 1 to 20 carbon atoms, a substituted or unsubstituted arylene group having 6 to 30 carbon atoms for forming a ring, or a substituted or unsubstituted heteroarylene group having 2 to 30 carbon atoms for forming a ring,
e1 to e4 are each independently 0 or 1,
R₃₁ to R₃₉ are each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 carbon atoms for forming a ring, or a substituted or unsubstituted heteroaryl group having 2 to 30 carbon atoms for forming a ring, or are bonded to an adjacent group to form a ring, and
d1 to d4 are each independently an integer of 0 to 4.

12. The organic electroluminescence device of claim 1, the emission region further includes a compound represented by any one among the following Formula F-a to Formula F-c: 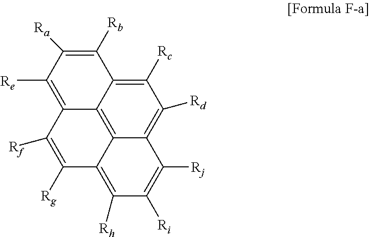
in Formula F-a,
two selected from among Rₐ to Rⱼ are independently be substituted with *—NAr₁Ar₂ the others, which are not substituted with *—NAr₁Ar₂ among Rₐ to Rⱼ are each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 carbon atoms for forming a ring, or a substituted or unsubstituted heteroaryl group having 2 to 30 carbon atoms for forming a ring, and
in *—NAr₁Ar₂, Ar₁ and Ar₂ are each independently a substituted or unsubstituted aryl group having 6 to 30 carbon atoms for forming a ring, or a substituted or unsubstituted heteroaryl group having 2 to 30 carbon atoms for forming a ring: 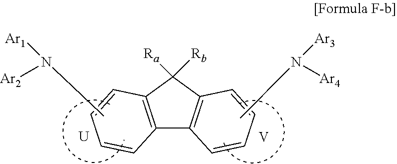
in Formula F-b above, Rₐ and R_{b} are each independently a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 carbon atoms for forming a ring, or a substituted or unsubstituted heteroaryl group having 2 to 30 carbon atoms for forming a ring, or may be bonded to an adjacent group to form a ring,
Ar₁ to Ar₄ are each independently a substituted or unsubstituted aryl group having 6 to 30 carbon atoms for forming a ring, or a substituted or unsubstituted heteroaryl group having 2 to 30 carbon atoms for forming a ring,
U and V are each independently a substituted or unsubstituted hydrocarbon ring having 5 to 30 carbon atoms for forming a ring, or a substituted or unsubstituted heterocycle having 2 to 30 carbon atoms for forming a ring, and
the number of rings represented by U and V are each independently 0 or 1: 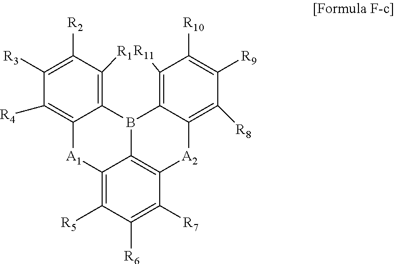
in Formula F-c, A₁ and A₂ are each independently O, S, Se, or NRₘ, or are bonded to an adjacent group to form a fused ring,
Rₘ is a hydrogen atom, a deuterium atom, a substituted or unsubstituted alkyl group having 1 to 20 carbon atoms, a substituted or unsubstituted aryl group having 6 to 30 carbon atoms for forming a ring, or a substituted or unsubstituted heteroaryl group having 2 to 30 carbon atoms for forming a ring, and
R₁ to R₁₁ are each independently a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 carbon atoms for forming a ring, or a substituted or unsubstituted heteroaryl group having 2 to 30 carbon atoms for forming a ring, or are bonded to an adjacent group to form a ring.

13. The organic electroluminescence device of claim 1, the emission region further includes a compound represented by the following Formula HT-1: 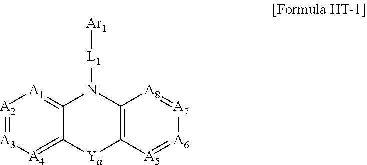
in Formula HT-1,
A₁ to A₈ are each independently N or CR₅₁,
L₁ is a direct linkage, a substituted or unsubstituted arylene group having 6 to 30 carbon atoms for forming a ring, or a substituted or unsubstituted heteroarylene group having 2 to 30 carbon atoms for forming a ring,
Yₐ is a direct linkage, CR₅₂R₅₃, or SiR₅₄R₅₅,
Ar₁ is a substituted or unsubstituted aryl group having 6 to 30 carbon atoms for forming a ring, or a substituted or unsubstituted heteroaryl group having 2 to 30 carbon atoms for forming a ring, and
R₅₁ to R₅₅ are each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 30 carbon atoms for forming a ring, or a substituted or unsubstituted heteroaryl group having 2 to 30 carbon atoms for forming a ring, or combined with an adjacent group to form a ring.

14. The organic electroluminescence device of claim 1, the emission region further includes a compound represented by the following Formula ET-1: 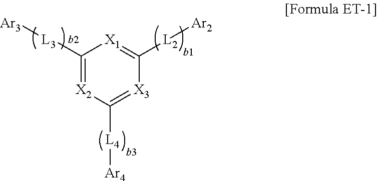
in Formula ET-1,
at least one among X₁ to X₃ is N, and the remainder is CR₅₆,
R₅₆ is a hydrogen atom, a deuterium atom, a substituted or unsubstituted alkyl group having 1 to 20 carbon atoms, a substituted or unsubstituted aryl group having 6 to 60 carbon atoms for forming a ring, or a substituted or unsubstituted heteroaryl group having 2 to 60 carbon atoms for forming a ring,
L₂ to L₄ are each independently a direct linkage, a substituted or unsubstituted arylene group having 6 to 30 carbon atoms for forming a ring, or a substituted or unsubstituted heteroarylene group having 2 to 30 carbon atoms for forming a ring, b1 to b3 are each independently an integer of 0 to 10, and
Ar₂ to Ar₄ are each independently a hydrogen atom, a deuterium atom, a substituted or unsubstituted alkyl group having 1 to 20 carbon atoms, a substituted or unsubstituted aryl group having 6 to 30 carbon atoms for forming a ring, or a substituted or unsubstituted heteroaryl group having 2 to 30 carbon atoms for forming a ring.

15. The organic electroluminescence device of claim 1, the emission region further includes a compound represented by the following Formula D-1: 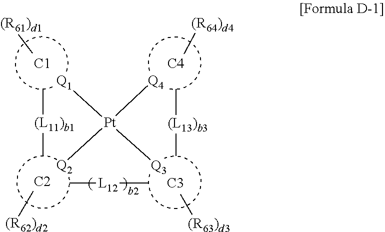
in Formula D-1
Q₁ to Q₄ are each independently C or N,
C1 to 04 are each independently a substituted or unsubstituted hydrocarbon ring of 5 to 30 carbon atoms for forming a ring, or a substituted or unsubstituted heterocycle of 2 to 30 carbon atoms for forming a ring,
L₁₁ to L₁₃ are each independently a direct linkage, 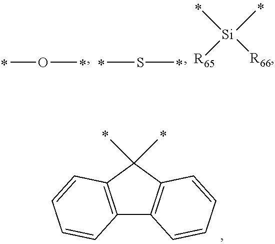
a substituted or unsubstituted divalent alkyl group having 1 to 20 carbon atoms, a substituted or unsubstituted arylene group having 6 to 30 carbon atoms for forming a ring, or a substituted or unsubstituted heteroarylene group having 2 to 30 carbon atoms for forming a ring,
b1 to b3 are each independently 0 or 1,
R₆₁ to R₆₆ are each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 60 carbon atoms for forming a ring, or a substituted or unsubstituted heteroaryl group having 2 to 60 carbon atoms for forming a ring, or combined with an adjacent group to form a ring, and
d1 to d4 are each independently an integer of 0 to 4.

16. The organic electroluminescence device of claim 1, the emission region further includes a compound represented by the following Formula HT-1, a compound represented by the following Formula ET-1, a compound represented by the following Formula D-1, and a compound represented by any one among the following Formula F-a to Formula F-c: 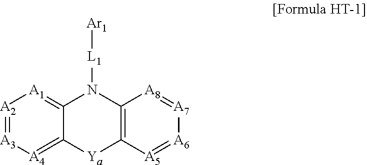
in Formula HT-1,
A₁ to A₈ are each independently N or CR₅₁,
L₁ is a direct linkage, a substituted or unsubstituted arylene group having 6 to 30 carbon atoms for forming a ring, or a substituted or unsubstituted heteroarylene group having 2 to 30 carbon atoms for forming a ring,
Yₐ is a direct linkage, CR₅₂R₅₃, or SiR₅₄R₅₅,
Ar₁ is a substituted or unsubstituted aryl group having 6 to 30 carbon atoms for forming a ring, or a substituted or unsubstituted heteroaryl group having 2 to 30 carbon atoms for forming a ring, and
R₅₁ to R₅₅ are each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 30 carbon atoms for forming a ring, or a substituted or unsubstituted heteroaryl group having 2 to 30 carbon atoms for forming a ring, or combined with an adjacent group to form a ring, 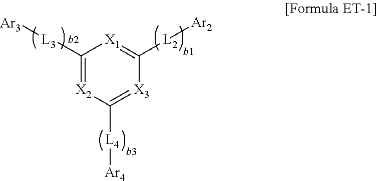
in Formula ET-1,
at least one among X₁ to X₃ is N, and the remainder is CR₅₆,
R₅₆ is a hydrogen atom, a deuterium atom, a substituted or unsubstituted alkyl group having 1 to 20 carbon atoms, a substituted or unsubstituted aryl group having 6 to 60 carbon atoms for forming a ring, or a substituted or unsubstituted heteroaryl group having 2 to 60 carbon atoms for forming a ring,
L₂ to L₄ are each independently a direct linkage, a substituted or unsubstituted arylene group having 6 to 30 carbon atoms for forming a ring, or a substituted or unsubstituted heteroarylene group having 2 to 30 carbon atoms for forming a ring,
b1 to b3 are each independently an integer of 0 to 10, and
Ar₂ to Ar₄ are each independently a hydrogen atom, a deuterium atom, a substituted or unsubstituted alkyl group having 1 to 20 carbon atoms, a substituted or unsubstituted aryl group having 6 to 30 carbon atoms for forming a ring, or a substituted or unsubstituted heteroaryl group having 2 to 30 carbon atoms for forming a ring, 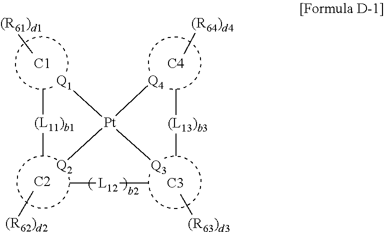
in Formula D-1,
Q₁ to Q₄ are each independently C or N,
C1 to C4 are each independently a substituted or unsubstituted hydrocarbon ring of 5 to 30 carbon atoms for forming a ring, or a substituted or unsubstituted heterocycle of 2 to 30 carbon atoms for forming a ring,
L₁₁ to L₁₃ are each independently a direct linkage, 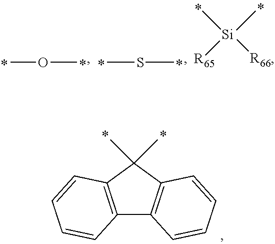
a substituted or unsubstituted divalent alkyl group having 1 to 20 carbon atoms, a substituted or unsubstituted arylene group having 6 to 30 carbon atoms for forming a ring, or a substituted or unsubstituted heteroarylene group having 2 to 30 carbon atoms for forming a ring,
b1 to b3 are each independently 0 or 1,
R₆₁ to R₆₆ are each independently a hydrogen atom, a deuterium atom, a halogen atom, a cyano group, a substituted or unsubstituted silyl group, a substituted or unsubstituted thio group, a substituted or unsubstituted oxy group, a substituted or unsubstituted amine group, a substituted or unsubstituted boron group, a substituted or unsubstituted alkyl group having 1 to 20 carbon atoms, a substituted or unsubstituted alkenyl group having 2 to 20 carbon atoms, a substituted or unsubstituted aryl group having 6 to 60 carbon atoms for forming a ring, or a substituted or unsubstituted heteroaryl group having 2 to 60 carbon atoms for forming a ring, or combined with an adjacent group to form a ring, and
d1 to d4 are each independently an integer of 0 to 4, 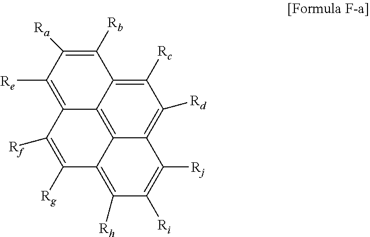
in Formula F-a,
two selected from among Rₐ to Rⱼ are independently be substituted with *—NAr₁Ar₂ the others, which are not substituted with *—NAr₁Ar₂ among Rₐ to Rⱼ are each independently a hydrogen atom, a deuterium atom, a halogen atom, a cyano group, a substituted or unsubstituted amine group, a substituted or unsubstituted alkyl group having 1 to 20 carbon atoms, a substituted or unsubstituted aryl group having 6 to 30 carbon atoms for forming a ring, or a substituted or unsubstituted heteroaryl group having 2 to 30 carbon atoms for forming a ring, and
in *—NAr₁Ar₂, Ar₁ and Ar₂ are each independently a substituted or unsubstituted aryl group having 6 to 30 carbon atoms for forming a ring, or a substituted or unsubstituted heteroaryl group having 2 to 30 carbon atoms for forming a ring: 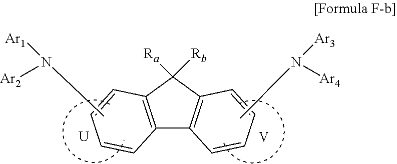
in Formula F-b above, Rₐ and R_{b} are each independently a hydrogen atom, a deuterium atom, a substituted or unsubstituted alkyl group having 1 to 20 carbon atoms, a substituted or unsubstituted alkenyl group having 2 to 20 carbon atoms, a substituted or unsubstituted aryl group having 6 to 30 carbon atoms for forming a ring, or a substituted or unsubstituted heteroaryl group having 2 to 30 carbon atoms for forming a ring, or may be bonded to an adjacent group to form a ring,
Ar₁ to Ar₄ are each independently a substituted or unsubstituted aryl group having 6 to 30 carbon atoms for forming a ring, or a substituted or unsubstituted heteroaryl group having 2 to 30 carbon atoms for forming a ring,
U and V are each independently a substituted or unsubstituted hydrocarbon ring having 5 to 30 carbon atoms for forming a ring, or a substituted or unsubstituted heterocycle having 2 to 30 carbon atoms for forming a ring, and
the number of rings represented by U and V are each independently 0 or 1: 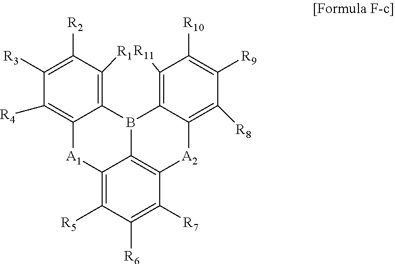
in Formula F-c, A₁ and A₂ are each independently O, S, Se, or NRₘ, or are bonded to an adjacent group to form a fused ring,
Rₘ is a hydrogen atom, a deuterium atom, a substituted or unsubstituted alkyl group having 1 to 20 carbon atoms, a substituted or unsubstituted aryl group having 6 to 30 carbon atoms for forming a ring, or a substituted or unsubstituted heteroaryl group having 2 to 30 carbon atoms for forming a ring, and
R₁ to R₁₁ are each independently a hydrogen atom, a deuterium atom, a halogen atom, a cyano group, a substituted or unsubstituted amine group, a substituted or unsubstituted boryl group, a substituted or unsubstituted oxy group, a substituted or unsubstituted thio group, a substituted or unsubstituted alkyl group having 1 to 20 carbon atoms, a substituted or unsubstituted aryl group having 6 to 30 carbon atoms for forming a ring, or a substituted or unsubstituted heteroaryl group having 2 to 30 carbon atoms for forming a ring, or are bonded to an adjacent group to form a ring.
